(19) United States
(12) Patent Application Publication
(10) Pub. No.: US 2011/0150897 A1
(43) Pub. Date: Jun. 23, 2011

(54) INFLUENZA TARGETS

(76) Inventors: Thomas F. Meyer, Falkensee (DE); Alexander Karlas, Nurnberg (DE); Nikolaus Machuy, Bad Soden-Salmunster (DE); Jan David Manntz, Berlin (DE); Elke Muller, Aachen (DE); Thomas Rudel, Berlin (DE)

(21) Appl. No.: 12/445,228

(22) PCT Filed: Oct. 11, 2007
(86) PCT No.: PCT/EP07/08852

§ 371 Date: Feb. 4, 2011

Related U.S. Application Data
(60) Provisional application No. 60/850,651, filed on Oct. 11, 2006.

(51) Int. Cl. A61K 39/395; A61K 31/7088; A61K 38/02; A61K 31/711; A61K 31/7105; C12Q 1/70; G01N 33/53; C12Q 1/68; A61P 31/16; B82Y 5/00

(52) U.S. Cl. 424/158.1; 514/44 R; 514/3.7; 424/130.1; 514/44 A; 435/5; 435/7.1; 435/6.13; 977/773; 977/906

(57) ABSTRACT

The present invention relates to pharmaceutical compositions comprising modulators of kinases, kinase binding polypeptides or/and an inhibitor for influenza virus replication for the prevention or/and treatment of influenza.

The present invention relates to a pharmaceutical composition comprising an inhibitor of influenza virus replication. In particular, the present invention relates to a pharmaceutical composition comprising a modulator of a kinase or/and a kinase binding polypeptide for the prevention or/and treatment of influenza. Further, the present invention concerns a screening method for identification of a modulator of a kinase or/and a kinase binding polypeptide, which modulator is suitable for prevention or/and treatment of influenza. Yet another aspect is a screening method for identification of new targets for the prevention, alleviation or/and treatment of influenza.

In view of the threatening influenza pandemic, there is an acute need to develop and make available lastingly effective drugs. In Germany alone the annual occurrence of influenza causes between 5,000 and 20,000 deaths a year (source: Robert-Koch Institute). The recurring big influenza pandemics are especially feared. The first big pandemic, the so-called “Spanish Flu”, cost about 40 million lives in the years 1918-1919 including a high percentage of healthy, middle-aged people. A similar pandemic could be caused by the H5N1 influenza virus, which at the moment replicates mainly in birds, if acquired mutations enable the virus to be transmitted from person to person. The probability of a human pandemic has recently grown more acute with the spreading of bird flu (H5N1) worldwide and the infection of domestic animals. It is only a question of time until a highly pathogenic human influenza-recombinant emerges. The methods available at the moment for prophylaxis or therapy of an influenza infection, such as vaccination with viral surface proteins or the use of antiviral drugs (neuraminidase inhibitors or ion channel blockers), have various disadvantages. Already at this early stage resistance is appearing against one of our most effective preparations (Tamiflu), which may make it unsuitable to contain a pandemic. A central problem in the use of vaccines and drugs against influenza is the variability of the pathogen. Up to now the development of effective vaccines has required accurate prediction of the pathogen variant. Drugs directed against viral components can rapidly lose their effectiveness because of mutations of the pathogen.

An area of research which has received little attention up to now is the identification of critical target structures in the host cell. Viruses are dependent on certain cellular proteins to be able to replicate within the host. The knowledge of such cellular factors that are essential for viral replication but dispensable (at least temporarily) for humans could lead to the development of novel drugs. Rough estimates predict about 500 genes in the human genome which are essential for viral multiplication. Of these, 10% at least are probably dispensable temporarily or even permanently for the human organism. Inhibition of these genes and their products, which in contrast to the viral targets are constant in their structure, would enable the development of a new generation of antiviral drugs in the shortest time. Inhibition of such gene products could overcome the development of viral escape mutants that are not longer sensitive to antiviral drugs. Amongst other gene families kinases that are important regulatory proteins within the cell are often hijacked by viruses to manipulate the constitution of the host cell.

It is the object of the present invention to provide a screening method for genes involved in influenza virus replication. Another object is a screening method for identification of modulators of such genes.

In the context of the present invention, it was surprisingly found that inhibition of particular genes or the modulation of kinases or/and kinase binding polypeptides leads to reduction of influenza virus replication.

Table 1a, 1b and 4 describe targets for the prevention, alleviation or/and treatment of an influenza virus infection.

Examples of genes which upon downregulation increase the influenza virus replication are described in Table 1a. Thus, by increasing expression or/and activity of these genes or/and gene products, the influenza virus replication can be reduced.

Examples of genes which upon downregulation decrease the influenza virus replication are described in Table 1b and 4. Thus, by decreasing expression or/and activity of these genes or/and gene products, the influenza virus replication can be reduced.

Subject of the present invention is thus a screening method covering different aspects related to influenza virus infection, in particular influenza virus replication.

In a first aspect the screening method is a screening method for identification of a compound suitable for modulation of a kinase or/and a kinase binding polypeptide, comprising the steps
(a) providing a kinase or/and a kinase binding polypeptide,
(b) contacting a compound with the kinase or/and a kinase binding polypeptide of (a),
(c) determining the activity of the kinase or/and the kinase binding polypeptide,
(d) selecting a compound which modulates the activity of the kinase or/and the kinase binding polypeptide.

The screening method of the present invention may comprise a cellular screening assay or/and a molecular screening assay. A cellular screening assay includes the determination of the activity or/and expression of a kinase or/and of a kinase binding polypeptide in a cell or/and in a non human organism. A molecular screening assay includes determination of the activity of an isolated kinase or/and of an isolated kinase binding polypeptide.

The screening assay in the screening method of the present invention may be performed in vivo or/and in vitro.

In the screening method of the present invention the kinase or/and the kinase binding polypeptide may be provided in an isolated form. “Isolated” in the context of the present invention refers to any protein or polypeptide isolation procedure known by a person skilled in the art. “Isolated form” or “isolated kinase or/and isolated kinase binding polypeptide” includes essentially pure or crude preparations or formulations of the kinase or/and kinase binding polypeptide.

In the screening method of the present invention, the kinase or/and the kinase binding polypeptide may be provided by a cell or/and a non-human organism capable of expressing the kinase or/and the kinase binding polypeptide.

In yet another aspect the screening method is a screening method for identification of a kinase modulator suitable for prevention, alleviation or/and treatment of an influenza virus infection, comprising the steps
(I) providing a cell or/and a non-human organism capable of being infected with an influenza virus and capable of expressing a kinase or/and a kinase binding polypeptide,
(II) contacting the cell or/and the organism of (I) with an influenza virus and with a compound known to be capable of modulating the expression or/and activity of the kinase or/and kinase binding polypeptide,
(III) determining the amount of influenza virus produced by the cell or/and the organism, and
(IV) selecting a compound which reduces the amount of the influenza virus produced by the cell or/and the organism.

In a further aspect the screening method is a screening method for identification of compound suitable for the prevention, alleviation or/and treatment of an influenza virus infection, comprising the steps
(A) providing a cell or/and a non-human organism capable of being infected with an influenza virus and capable of expressing a gene, wherein the gene or/and gene product thereof is capable of modulating an influenza virus replication,
(B) contacting the cell or/and the organism of (A) with an influenza virus and with a compound known to be capable of modulating the expression or/and activity of the gene of (A) or/and the gene product thereof,
(C) determining the amount of influenza virus produced by the cell or/and the organism, and
(D) selecting a compound which reduces the amount of the influenza virus produced by the cell or/and the organism.

The gene of (A) is preferably selected from Table 1A, 1B and 4, in more preferred embodiments from one of the Tables 1A, 1B or 4. “Modulation” in (A) and (B) may be “activation” or “inhibition”, in particular “inhibition”.

In a further aspect, the screening method is a screening method for identification of a compound suitable for prevention, alleviation or/and treatment of an influenza virus infection, comprising the steps
(i) providing a cell or/and a non-human organism capable of expressing a kinase or/and a kinase binding polypeptide,
(ii) contacting a compound with the cell or/and the organism of (i),
(iii) determining the amount or/and the activity of kinase or/and a kinase binding polypeptide, and
(iv) selecting a compound which modulates the amount or/and the activity of the kinase or/and the kinase binding polypeptide.

In a further aspect, the screening method is a screening method for identification of a compound suitable for prevention, alleviation or/and treatment of an influenza virus infection, comprising the steps
i. providing a cell or/and a non-human organism capable of expressing a gene, wherein the gene or/and gene product thereof is capable of modulating an influenza virus replication,
ii. contacting a compound with the cell or/and the organism of i.,
iii. determining the amount or/and the activity of gene product of the gene of (i), and
iv. selecting a compound which modulates the amount or/and the activity of the gene product of i.

The gene of (i) is preferably selected from table 1A, 1B and 4, in more preferred embodiments from one of the Tables 1A, 1B or 4. “Modulation” in i. and ii. may be “activation” or “inhibition”, in particular “inhibition”.

In yet another aspect of the present invention, the screening method is a screening method for identification of genes suitable as targets for the prevention, alleviation or/and prevention of an influenza virus infection, comprising the steps
(1) providing a cell or/and a transgenic non-human animal capable of expressing, particularly over- or underexpressing a gene,
(2) contacting the cell or/and the organism of (1) with an influenza virus,
(3) modulating the expression or/and activity of the gene and determining the amount of influenza virus produced by the cell or/and the organism, and
(4) selecting a gene the expression or/and activity of which is positively or negatively correlated with reduction of the amount of the influenza virus produced by the cell or/and the organism.

The at least one kinase or/and kinase binding polypeptide employed in the screening method of the present invention is preferably encoded by a nucleic acid or/and gene selected from Table 1A and Table 1B. Nucleic acids comprising at least one sequence of Tables 1a, 1b, 2 or/and 4 and fragments thereof may be employed in the screening method of the present invention.

In the context of the present invention, a “target” includes a nucleotide sequence in a gene or/and a genome, a nucleic acid, or/and a polypeptide which is involved in regulation of influenza virus replication in a host cell. The target may be directly or indirectly involved in regulation of influenza virus replication. In particular, a target is suitable for reduction of influenza virus replication, either by activation of the target or by inhibition of the target.

Examples of targets are genes and partial sequence of genes, such as regulatory sequences. The term “target” also includes a gene product such as RNA, in particular mRNA, tRNA, rRNA, a polypeptide or/and a protein encoded by the target gene. Preferred gene products of a target gene are selected from mRNA, polypeptide(s) and protein(s) encoded by the target gene. The most preferred gene product is a polypeptide or protein encoded by the target gene. A target protein or a target polypeptide may be posttranslationally modified or not.

“Gene product” of a gene as used herein includes RNA (in particular mRNA, tRNA, rRNA), a polypeptide or/and a protein encoded by said gene.

In the context of the present invention, “activity” of the gene or/and gene product includes transcription, translation, posttranslational modification, modulation of the activity of the gene or/and gene product. The activity may be modulated by ligand binding, which ligand may be an activator or inhibitor.

A further subject of the present invention is a pharmaceutical composition comprising at least one inhibitor of influenza virus replication optionally together with pharmaceutically acceptable carriers, adjuvants, diluents or/and additives, for the prevention, alleviation or/and treatment of an influenza virus infection.

Yet another subject of the present invention is a pharmaceutical composition comprising at least one modulator of a kinase or/and at least one modulator of a kinase binding polypeptide optionally together with pharmaceutically acceptable carriers, adjuvants, diluents or/and additives, for the prevention, alleviation or/and treatment of an influenza virus infection.

The influenza virus infection may be an influenza A virus infection. The influenza A virus may be selected from influenza A viruses isolated so far from avian and mammalian organisms. In particular, the influenza A virus may be selected from H1N1, H1N2, H1N3, H1N4, H1N5, H1N6, H1N7, H1N9, H2N1, H2N2, H2N3, H2N4, H2N5, H2N7, H2N8, H2N9, H3N1, H3N2, H3N3, H3N4, H3N5, H3N6, H3N8, H4N1, H4N2, H4N3, H4N4, H4N5, H4N6, H4N8, H4N9, H5N1, H5N2, H5N3, H5N6, H5N7, H5N8, H5N9, H6N1, H6N2, H6N3, H6N4, H6N5, H6N6, H6N7, H6N8, H6N9, H7N1, H7N2, H7N3, H7N4, H7N5, H7N7, H7N8, H7N9, H9N1, H9N2, H9N3, H9N5, H9N6, H9N7, H9N8, H10N1, H10N3, H10N4, H10N6, H10N7, H10N8, H10N9, H11N2, H11N3, H11N6, H11N9, H12N1, H12N4, H12N5, H12N9, H13N2, H13N6, H13N8, H13N9, H14N5, H15N2, H15N8, H15N9 and H16N3. More particularly, the influenza A virus is strain Puerto Rico/8/34 or the Avian Influenza virus isolate H5N1. The influenza virus as described herein may be employed in the screening method of the present invention.

The inhibitor of influenza virus replication or/and the modulator employed in the pharmaceutical compositions of the present invention is preferably selected from the group consisting of nucleic acids, nucleic acid analogues such as ribozymes, peptides, polypeptides, and antibodies. The modulator as described herein may also be employed in the screening method of the present invention.

The inhibitor of influenza virus replication or/and the modulator may also be a compound having a molecular weight smaller than 1000 Dalton.

The pharmaceutical composition of the present invention may comprise at least one inhibitor of influenza virus replication, wherein the at least one inhibitor of influenza virus replication modulates the expression of a gene selected from Table 1A, Table 1B, and Table 4, or/and a gene product thereof. In particular, the at least one inhibitor of influenza virus replication inhibits the expression of a gene selected from Table 1B and Table 4 or/and a gene product thereof, or the at least one inhibitor of influenza virus replication activates the expression of a gene selected from Table 1A or/and a gene product thereof.

The pharmaceutical composition of the present invention may comprise a modulator of a kinase or/and a modulator of a kinase binding polypeptide, wherein the at least one kinase or/and kinase binding polypeptide is encoded by a nucleic acid or/and gene selected from Table 1A and Table 1B. Such a kinase or/and kinase binding polypeptide may thus be the target of the modulator.

The pharmaceutical composition of the present invention may comprise at least one modulator which is an activator. The pharmaceutical composition of the present invention may comprise at least one activator comprising
(a) a nucleotide sequence selected from Table 1A or/and a fragment thereof, or/and
(b) a fragment which is at least 70%, preferably at least 80%, more preferably at least 90% identical to the sequence of (a),
   or/and at least one activator capable of activating expression or/and gene product activity of a gene comprising sequence (a) or/and (b).

The pharmaceutical composition of the present invention may comprise at least one modulator which is an inhibitor. Suitable inhibitors are RNA molecules capable of RNA interference. The pharmaceutical composition of the present invention may comprise at least one inhibitor comprising
(a) a nucleotide sequence selected from Table 1B and 4 or/and a fragment thereof, or
(b) a sequence which is at least 70%, preferably at least 80%, more preferably at least 90% identical to the sequence of (a),
   or/and at least one inhibitor capable of inhibiting expression or/and gene product activity of a gene comprising sequence (a) or/and (b).

The activator or/and the inhibitor as described herein may also be employed in the screening methods of the present invention.

The pharmaceutical composition of the present invention preferably comprises a nucleic acid, wherein the nucleic acid comprises a nucleotide sequence selected from the sequences of Table 2 and Table 4 and fragments thereof. Preferably, the nucleic acid is RNA or DNA.

In a more preferred embodiment, the pharmaceutical composition of the present invention comprises
(i) an RNA molecule capable of RNA interference,
(ii) a precursor of the RNA molecule (i), or/and
(iii) a DNA molecule encoding the RNA molecule (i) or/and the precursor (ii).

RNA molecules capable of RNA interference are described in WO 02/44321 which is included herein by reference.

The RNA molecule of the present invention may be a double-stranded RNA molecule, preferably a double-stranded siRNA molecule with or without a single-stranded overhang alone at one end or at both ends. The RNA molecule may comprise at least one nucleotide analogue or/and deoxyribonucleotide.

The DNA molecule as employed in the present invention may be a vector.

The nucleic acid employed in the present invention may be an antisense nucleic acid or a DNA encoding the antisense nucleic acid.

The nucleic acid or/and nucleic acid fragment employed in the present invention may have a length of at least 15, preferably at least 17, more preferably at least 19, most preferably at least 21 nucleotides. The nucleic acid or/and the nucleic acid fragment may have a length of at the maximum 29, preferably at the maximum 27, more preferably at the maximum 25, especially more preferably at the maximum 23, most preferably at the maximum 21 nucleotides.

In another preferred embodiment, the pharmaceutical composition of the present invention comprises an antibody, wherein the antibody may be directed against a kinase or/and kinase binding polypeptide.

Preferably the antibody is directed against a kinase or/and kinase binding polypeptide comprising
(a) an amino acid sequence encoded by a nucleic acid or/and gene selected from Table 1A, and Table 1B, or/and
(b) an amino acid sequence which is at least 70%, preferably at least 80%, more preferably at least 90% identical to the sequence of (a).

In another preferred embodiment, the antibody is directed against a polypeptide comprising
(a) an amino acid sequence encoded by a nucleic acid or/and gene selected from Table 4, or/and
(b) an amino acid sequence which is at least 70%, preferably at least 80%, more preferably at least 90% identical to the sequence of (a).

The antibody of the present invention may be a monoclonal or polyclonal antibody, a chimeric antibody, a chimeric single chain antibody, a Fab fragment or a fragment produced by a Fab expression library.

Techniques of preparing antibodies of the present invention are known by a skilled person. Monoclonal antibodies may be prepared by the human B-cell hybridoma technique or by the EBV-hybridoma technique (Köhler et al., 1975, Nature 256:495-497, Kozbor et al., 1985, J. Immunol. Methods 81, 31-42, Cote et al., PNAS, 80:2026-2030, Cole et al., 1984, Mol. Cell Biol. 62:109-120). Chimeric antibodies (mouse/human) may be prepared by carrying out the methods of Morrison et al. (1984, PNAS, 81:6851-6855), Neuberger et al. (1984, 312:604-608) and Takeda et al. (1985, Nature 314:452-454). Single chain antibodies may be prepared by techniques known by a person skilled in the art.

Recombinant immunoglobulin libraries (Orlandi et al, 1989, PNAS 86:3833-3837, Winter et al., 1991, Nature 349:293-299) may be screened to obtain an antibody of the present invention. A random combinatory immunoglobulin library (Burton, 1991, PNAS, 88:11120-11123) may be used to generate an antibody with a related specifity having a different idiotypic composition.

Another strategy for antibody production is the in vivo stimulation of the lymphocyte population.

Furthermore, antibody fragments (containing F(ab′)₂ fragments) of the present invention can be prepared by protease digestion of an antibody, e.g. by pepsin. Reducing the disulfide bonding of such F(ab′)₂ fragments results in the Fab fragments. In another approach, the Fab fragment may be directly obtained from an Fab expression library (Huse et al., 1989, Science 254:1275-1281).

Polyclonal antibodies of the present invention may be prepared employing an amino acid sequence encoded by a nucleic acid or/and gene selected from Table 1A and Table 1B or immunogenic fragments thereof as antigen by standard immunization protocols of a host, e.g. a horse, a goat, a rabbit, a human, etc., which standard immunization protocols are known by a person skilled in the art.

The antibody may be an antibody specific for a gene product of a target gene, in particular an antibody specific for a polypeptide or protein encoded by a target gene.

The antibody as described herein may also be employed in the screening method of the present invention.

Fragments of polypeptides or/and peptides as employed in the present invention, in particular fragments of an amino acid sequence encoded by a nucleic acid or/and gene selected from Table 1A, Table 1B and Table 4 may have a length of at least 5 amino acid residues, preferably at least 10, more preferably at least 20 amino acid residues. The length of said fragments may be 200 amino acid residues at the maximum, preferably 100 amino acid residues at the maximum, more preferably 60 amino acid residues at the maximum, most preferably 40 amino acid residues at the maximum.

In the screening methods of the present invention, modulating the expression of a gene may be downregulation or upregulation, in particular of transcription or/and translation.

It can easily be determined by a skilled person if a gene is upregulated or downregulated. In the context of the present invention, upregulation of gene expression may be an upregulation by a factor of at least 2, preferably at least 4. Downregulation in the context of the present invention may be a reduction of gene expression by a factor of at least 2, preferably at least 4. Most preferred is essentially complete inhibition of gene expression, e.g. by RNA interference.

“Decrease (increase) of the amount” may be a downregulation (upregulation) of gene expression by a factor of at least 2, preferably at least 4. In the case of reduction, essentially complete inhibition of gene expression is most preferred, e.g. by RNA interference.

Modulation of the activity of a gene may be decreasing or increasing of the activity.

“Decrease (increase) of the activity” may be a decrease (increase) of activity of a gene or gene product by a factor of at least 2, preferably at least 4. In the case of activity reduction, essentially complete inhibition of activity is most preferred.

Modulation may be performed by a single nucleic acid species or by a combination of nucleic acids comprising 2, 3 4, 5, 6 or even more different nucleic acid species, which may be selected from Tables 1a, 1b or/and 2 and fragments thereof. Preferred combinations are described in Table 3 (also referred herein as “pools”). Table 3 includes combinations of at least two kinase or/and kinase binding polypeptide genes. It is also preferred that the combination modulates one gene, for instance selected from Table 1a and 1b. A combination of two nucleic acid species is preferred. More preferred is a combination of two nucleic acids of Table 2. Most preferred is a combination of two nucleic acids of Table 2, which modulate one gene.

Modulation may be a knock-down performed by RNA interference. The nucleic acid or the combination of nucleic acid species may be an siRNA, which may comprise a sequence selected from the sequences of Table 2 and Table 4 and fragments thereof. It is preferred that the combination knocks down one gene, for instance selected from Table 1b and Table 4. A combination of two siRNA species is preferred, which may be selected from those sequences of Table 2, which are derived from genes of Table 1b, and the sequences of Table 4, wherein the combination preferably knocks down one gene.

The gene employed in the various embodiments of the present invention may be selected from any of the Tables 1A, 1 B, 2, or 4, or any combination thereof. A gene which when expressed or up-regulated is capable of modulating influenza virus replication may be selected from Tables 1A and 1B, from Tables 1A, 1B and 4, from Table 1A, 1B, 2 and 4. A gene which when expressed or up-regulated is capable of inhibiting influenza virus replication may be selected from Table 1B, from Tables 1B and 4, or from Tables 1B, 2 and 4. A gene which when expressed or up-regulated is capable of activating influenza virus replication may be selected from Table 1A, or from Tables 1A and 2.

The cell employed in any of the screening methods of the present invention may be any cell capable of being infected with in influenza virus. Preferably the cell is a mammalian cell or an avian cell. More preferably the cell is a human cell. Even more preferred is an epithelial cell. Most preferred is a lung epithelial cell. The cell may be a cell line. A suitable lung epithelial cell line is A594. Another suitable cell is the human embryonic kidney cell line 293T.

The modulator of a target gene or/and a gene product thereof as described herein may be used for the manufacture of a pharmaceutical composition for prevention, alleviation or/and prevention of an influenza virus infection.

Another subject of the present invention is the use of a inhibitor of influenza virus replication as described herein, for the prevention, alleviation or/and prevention of an influenza virus infection. Preferred is the use of an inhibitor capable of inhibiting the expression of a gene selected from Table 1b and 4, for the manufacture of a medicament for the prevention, alleviation or/and prevention of an influenza virus infection. Also preferred is the use of an inhibitor capable of inhibiting a gene product of a gene selected from Table 1b and 4, for the manufacture of a medicament for the prevention, alleviation or/and prevention of an influenza virus infection.

Yet another subject of the present invention is the use of a kinase modulator or/and a kinase binding protein modulator for the manufacture of a medicament for the prevention, alleviation or/and prevention of an influenza virus infection.

Yet another aspect of the present invention is the use of a nucleic acid comprising a gene sequence or/and a nucleotide sequence of Table 1a, 1b, 2 or/and 4 and fragments thereof in a method for screening for compounds or/and targets suitable for the prevention, alleviation or/and treatment of an influenza virus infection.

The invention is further illustrated by the following figures, tables and examples.

### FIGURE AND TABLE LEGENDS

FIG. 1: The experimental setting of the siRNA kinase screen of the example.

FIG. 2: The effect of transfected (control)-siRNAs in regard to luminescence data. This diagram shows a typical screening result from one 96 well plate. During all experiments several controls were included in triplets, like uninfected, transfected with a siRNA against luciferase, mock treated and siRNAs against the viral nucleoprotein gene (NP) from influenza A viruses. The difference of the luminescence between cells treated with luciferase siRNAs and anti-NP siRNAs was set to 100% inhibition per definition.

FIG. 3: The inhibition of influenza virus replication shown for all siRNAs tested in the example.

FIG. 4: The values “% inhibition” from all analyzed siRNAs were used to calculate the z-scores. Highly efficient siRNAs are labelled in pink showing more than 50% inhibition compared to the luciferase siRNA transfected control cells.

FIG. 5: The experimental setup of the genome wide siRNA screen (see Example 4).

Table 1: Results of the siRNa kinase screen: a: activation (“negative” inhibition) of virus replication in %, normalized against the cell number, and the standard deviation calculated using four independent experiments. b: inhibition of virus replication in %, normalized against the cell number, and the standard deviation calculated using four independent experiments. Pool X, wherein X denotes the number of the pool, refers to combinations described in Table 3.

Table 2: Oligonucleotide sequences employed in the siRNA kinase screen of example 1. Knock-down of a particular gene was performed (a) by a combination of two oligonucleotide sequences (“target 1” and “target 2”) specific for said gene, or (b) by pooled oligonucleotides specific for different genes (“Pool X”, wherein X denotes the number of the pool described in Table 3).

Table 3: Oligonucleotide pools employed in the siRNA kinase screen of the example.

Table 4: Oligonucleotide sequences employed in the siRNA screen of example 4. Up to four oligonucleotide sequences (“target sequence 1”, “target sequence 2”, “target sequence 3”, and “target sequence 4”) specific for a gene were employed (each in a separate test).

### EXAMPLE 1

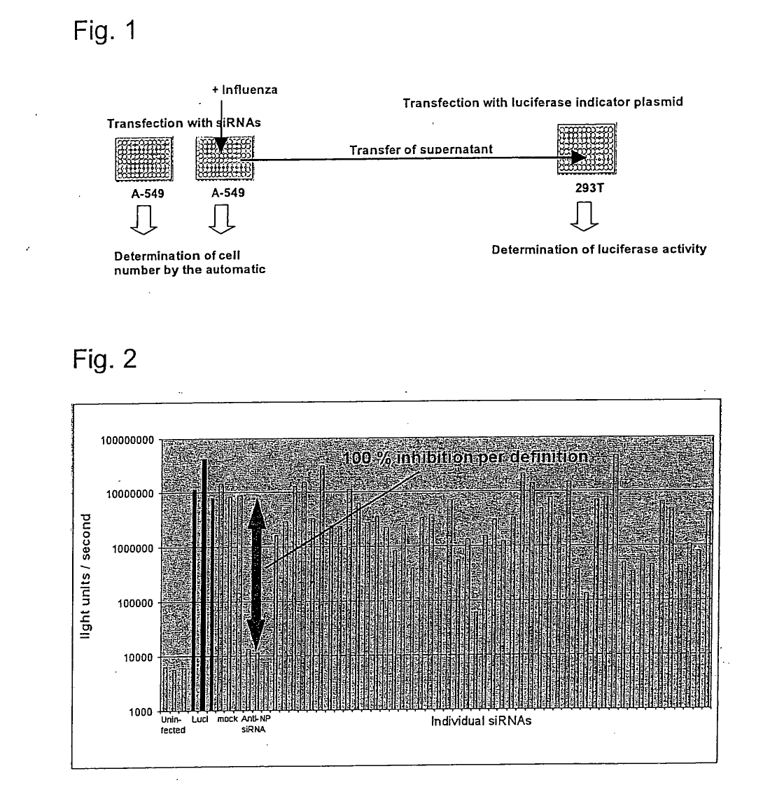

Since kinases are one of the most promising candidates that can influence virus progeny we used siRNAs against this group of genes to identify the individual role of each kinase or kinase binding polypeptide in respect of a modified replication of influenza viruses. All siRNAs were tested in four independent experiments. Since siRNAs against kinases can influence the replication of cells or are even cytotoxic, the effect of each individual siRNA transfection in regard to the cell number was analysed by using an automatic microscope. The amount of replication competent influenza viruses was quantified with an influenza reporter plasmid that was constructed using a RNA polymerase I promoter/terminator cassette to express RNA transcripts encoding the firefly luciferase flanked by the untranslated regions of the influenza A/WSN/33 nucleoprotein (NP) segment. Human embryonic kidney cells (293T) were transfected with this indicator plasmid one day before influenza infection. These cells were chosen, because they show a very strong amplification of the luciferase expression after influenza A virus infection. The cell based assay comprised the following steps (also FIG. 1 which describes the experimental setting of the siRNA kinase screen):
Day 1: Seeding of A549 cells (lung epithelial cells) in 96-well plates
Day 2: Transfection with siRNAs directed against kinases or kinase binding proteins
Day 3: Infection with influenza ANVSN/33+transfection of 293T cells with the influenza indicator plasmid
Day 4: Infection of 293T cells with the supernatant of A549 cells+determination of cell number by the automatic microscope
Day 5: Lysis of the indicator cells and performing the luciferase assay to quantify virus replication

For the identification of influenza relevant kinases the luminescence values were normalised against the cell number (measured after siRNA transfection and virus infection). Thereby unspecific effects due to the lower (or higher) cell numbers can be minimized.

Several controls were included to be able to demonstrate an accurate assay during the whole screening procedure (FIG. 2). The control siRNA against the viral nucleoprotein could nearly reduce the replication to levels of uninfected cells.

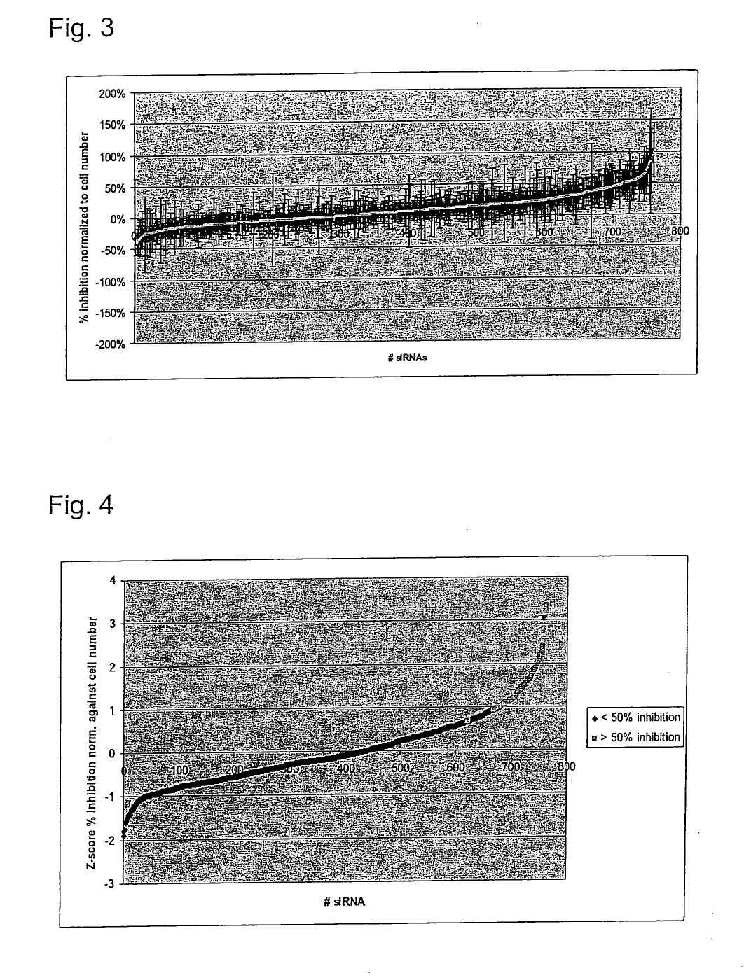

The illustration of the inhibition in percentage shows that some siRNAs can enhance the influenza virus replication, whereas others can inhibit the replication stronger (>113%) than the antiviral control siRNA against the influenza NP gene (FIG. 3). Thereby 47 siRNA decreased the replication more than 50%, 9 siRNAs showed more than 80% inhibition. The list of the results is provided in Table 1a and 1b, showing the activation (Table 1a, “negative” inhibition) and inhibition (Table 1b) of virus replication in %, normalized against the cell number, and the standard deviation calculated using four independent experiments.

Similar results were obtained using the calculation of z-scores. The z-score represents the distance between the raw score and the population mean in units of the standard deviation. The z-scores were calculated using the following equation:

$$z = \frac{X - μ}{σ} .$$

where X is a raw score to be standardized, σ is the standard deviation of the population, and μ is the mean of the population.

### EXAMPLE 2

In a future experiment the antiviral effect will be validated in more detail by using individual siRNAs instead of pooled siRNAs. Furthermore new siRNAs (at least two additional siRNAs per identified gene) will be tested using the experimental setting of Example 1. Those confirmed genes that seem to be important for the replication of influenza viruses will then be knocked down in mice using intranasally administered siRNAs. For the evaluation of this antiviral therapy it is of highest importance to determine the efficiency of transportation of compounds to lung epithelial tissue. The success of a therapy depends on the combination of high efficient kinase inhibitors and adequate transport system. A potentially compatible and cost efficient agent is chitosan which we are applying for the delivery of siRNAs in in vivo studies successfully. We will apply the compounds either intranasally or administer them directly into the lung.

Efficient siRNAs should lead to a decreased viral titre within the lung tissue and due to this animals should be protected against an otherwise lethal influenza infection.

For testing the biological effect of the kinase inhibitors, we will divide the experiments in four parts:
1. Analysis of the kinase inhibitor distribution in the respiratory apparatus after intranasal application of compound/chitosan nano particles. Optimisation of the compound/chitosan concentration for best effectiveness. Further tests will only be performed in case of success.
2. In LD50 tests the absolute pathogenicity of the virus isolates Influenza A/Puerto Rico/8/34 and the Avian Influenza isolate (for test 4) will be estimated.
3. Test of antiviral effect of selected siRNAs after intranasal application and infection with Influenza A/Puerto Rico/8/34 by analyzing virus titre in lung tissue or survival rate (in certain cases).
4. Test of antiviral effect of selected siRNAs after intranasal application and infection with highly pathogenic Avian Influenza virus isolate (such as H5N1) by analyzing the virus titre in lung tissue or survival rate (in certain cases).

The used virus isolate is dependant on current development and spreading of the Avian Influenza. We aim at inhibiting the replication of the current prevalent strain in vivo efficiently

Kinase inhibitors against the confirmed genes will also be tested in mice regarding to an impaired virus replication.

The Max-Planck-Institut für lnfektionsbiologie, Berlin, Germany, has genome-wide RNAi libraries that, in principle, enable the shutting-off of every single human gene in suitable cell cultures (A549 cells). So in the next level the screen will be expanded to a genome wide scale, because many additional cellular factors involved in the attachment, replication and budding of viruses are still unknown.

### EXAMPLE 3

Additional siRNAs (not only siRNAs against kinases or kinase binding proteins) will also be validated in regard to a decline of the replication of influenza A viruses. For the evaluation of these siRNAs the same experimental setting will be used as described in example 1, except that the cell number is quantified indirectly by using a commercial cell viability assay (instead of using an automated microscope) and that these siRNAs will be reverse transfected, i.e. cells will be added to the siRNA transfection mix already prepared in 384 well plates.

### EXAMPLE 4

Among the human genome hundreds of genes are presumably relevant for the replication of influenza viruses. Therefore the screening procedure of kinases and kinase binding factors (described in Example 1) was expanded to a genome wide scale analysing all known human genes by using about 59886 siRNAs.

The experimental setup was performed in a similar way as described in Example 1, except:
The screen was extended to genome wide level using 59886 siRNAs
Cells were seeded in 384 well plates.
Because of the huge number of transfected cells, not all cell numbers could be analysed by automated microscopy.
siRNAs were reversely transfected in freshly seeded A549 cells using the transfection reagent HiperFect (Qiagen, Hilden, Germany).
Knock-down of a particular gene was independently performed by up to four siRNAs (“target sequence 1”, “target sequence 2”, “target sequence 3”, and “target sequence 4” in Table 4) specific for a particular gene.
Additional controls were included: “AllStars Negative Control siRNA” (Qiagen, Hilden, Germany, Order No. 1027280) as negative control, siRNAs directed against PKMYT (GeneID: 9088, GenBank accession number: NM_target 182687, sequence: CTGGGAGGAACTTACCGTCTA) as positive control (cellular factor against influenza replication), siRNAs directed against PLK (GeneID: 5347, GenBank accessionnumber: BC014135, target sequence: CCGGATCAAGAAGAATGAATA) as transfection control (cytotoxic after transfection).
The infection rate of transfected A549 cells in selected wells is measured by automated microscopy to be able to dissect the inhibitory effects to early or late events during the infection process.
Results were analysed by the statistical R-package “ceIIHTS” software, developed by Michael Butros, Ligia Bras and Wolfgang Huber, using the B-score normalisation method (based on “Allstars Negative Control siRNA” transfected control wells).
Read-out is inhibition of virus replication.

The siRNAs and corresponding genes that showed a strong antiviral activity (z-scores<−2.0) are listed in Table 4.

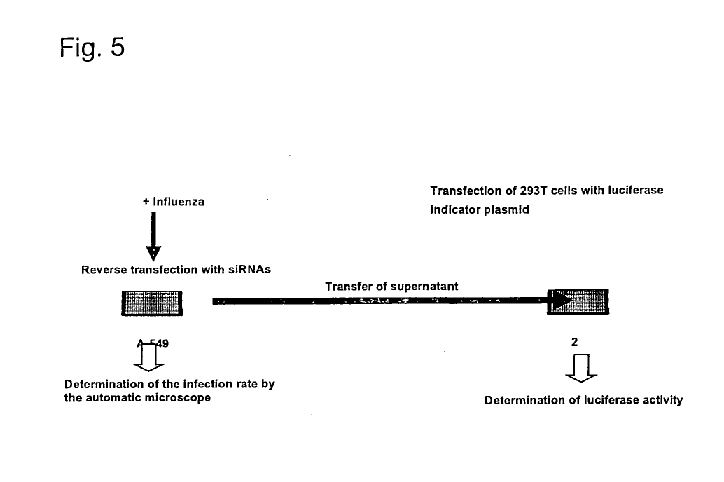

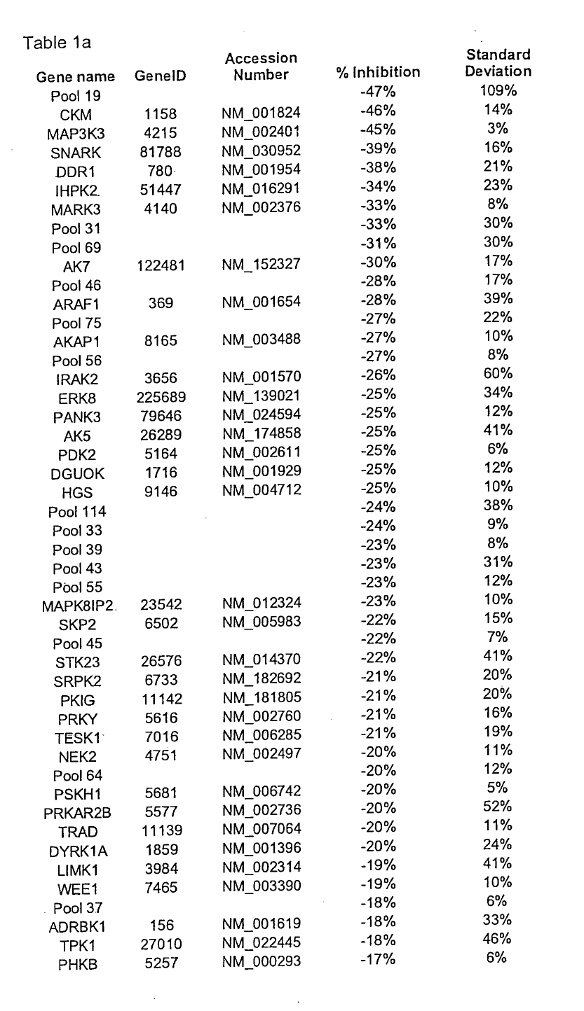

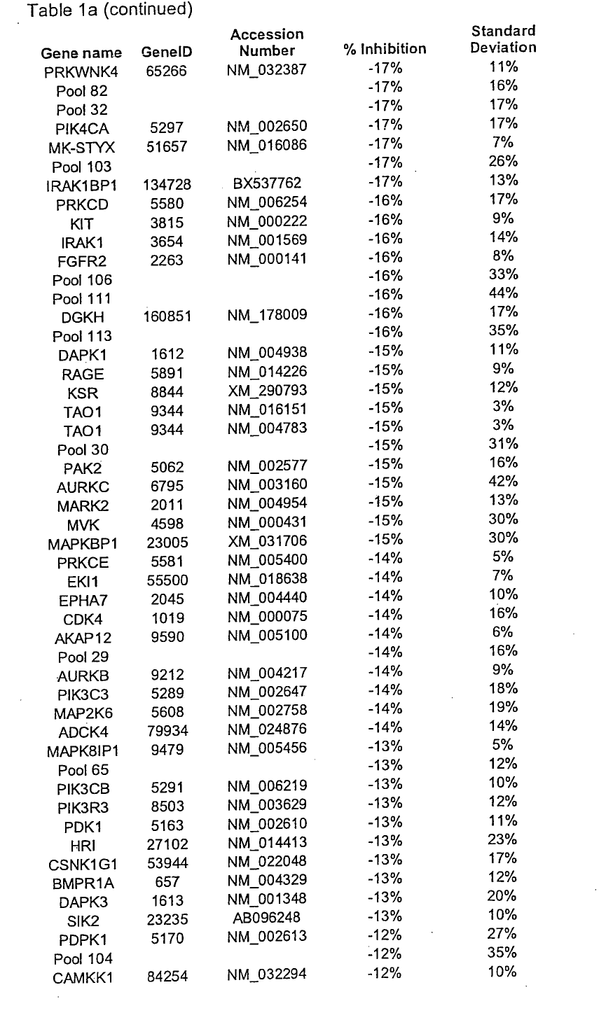

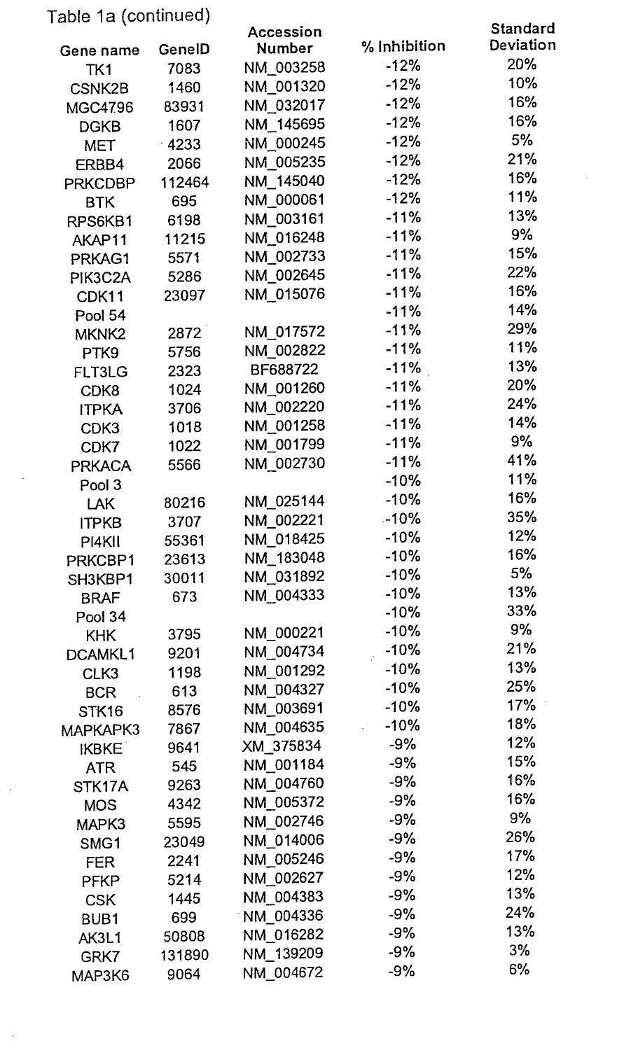

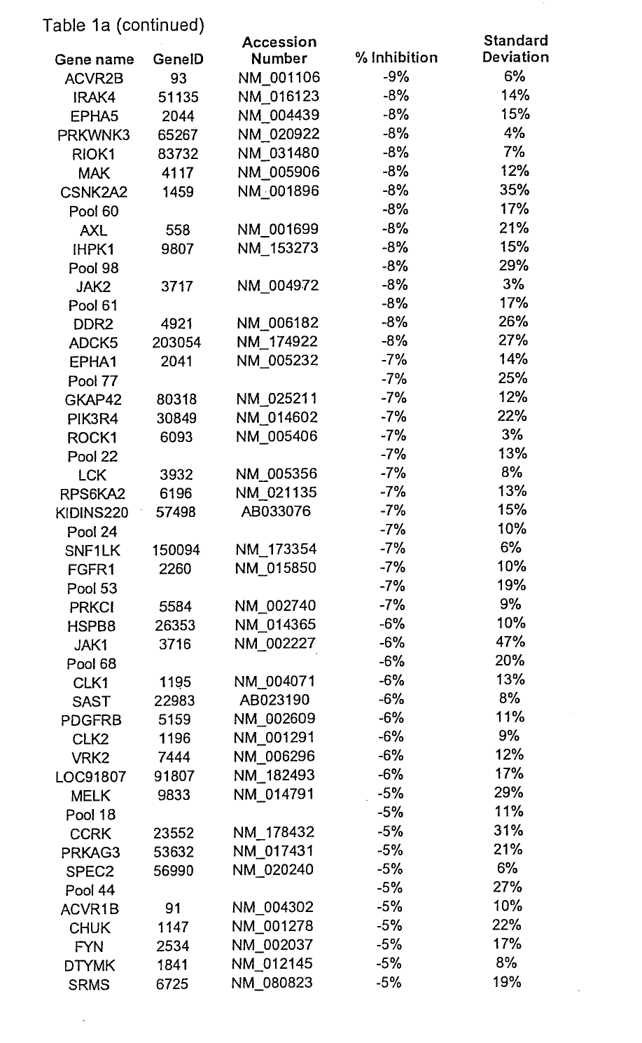

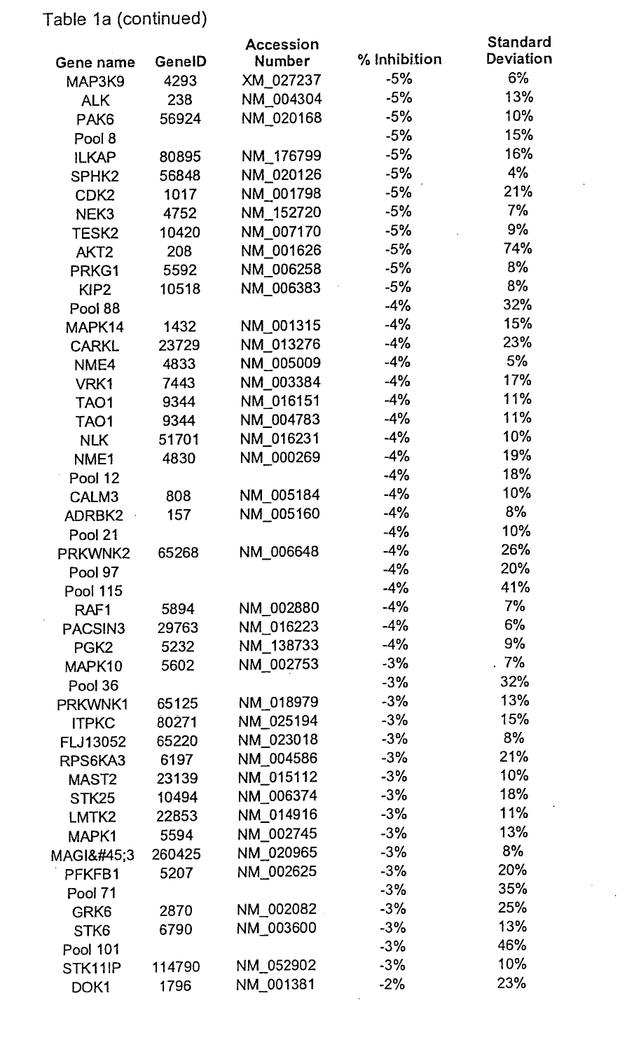

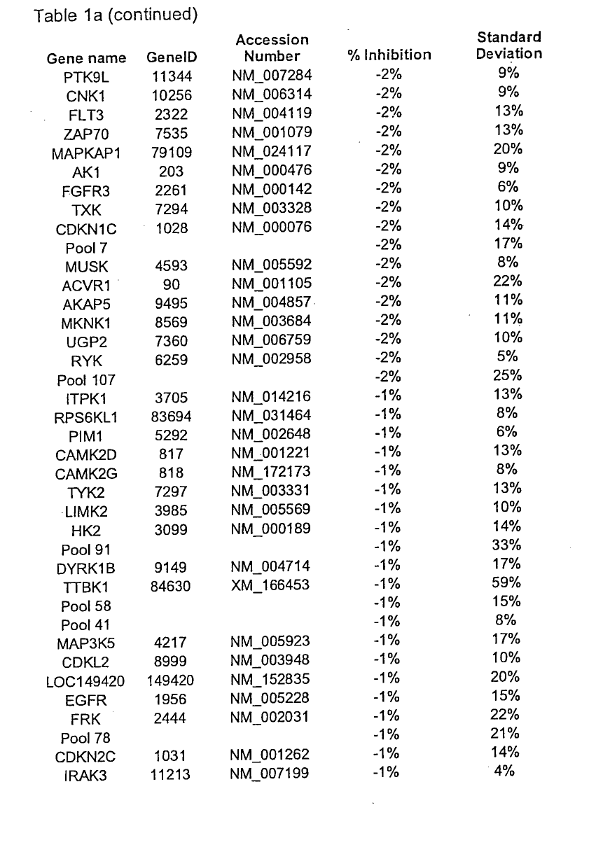

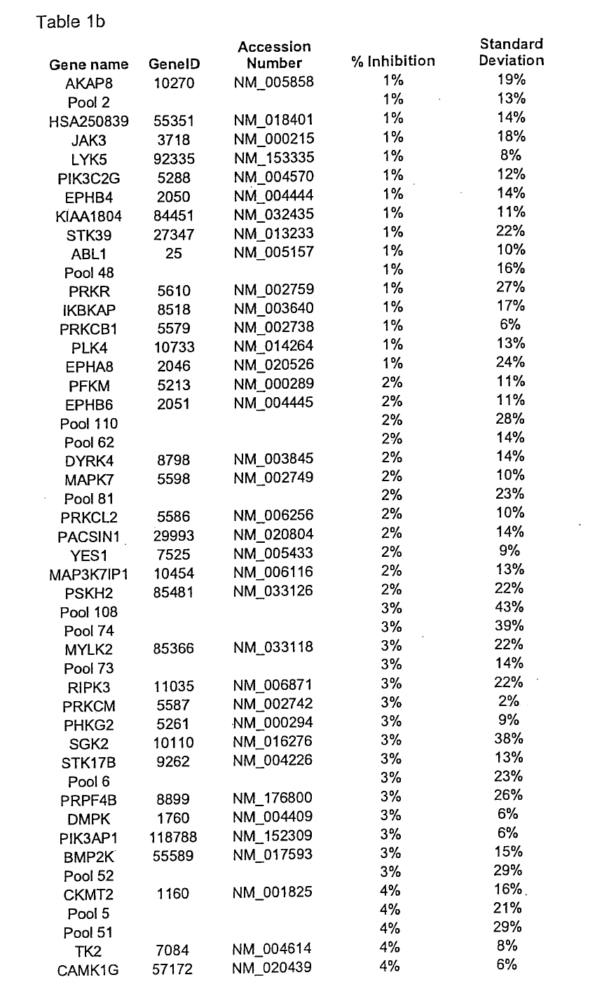

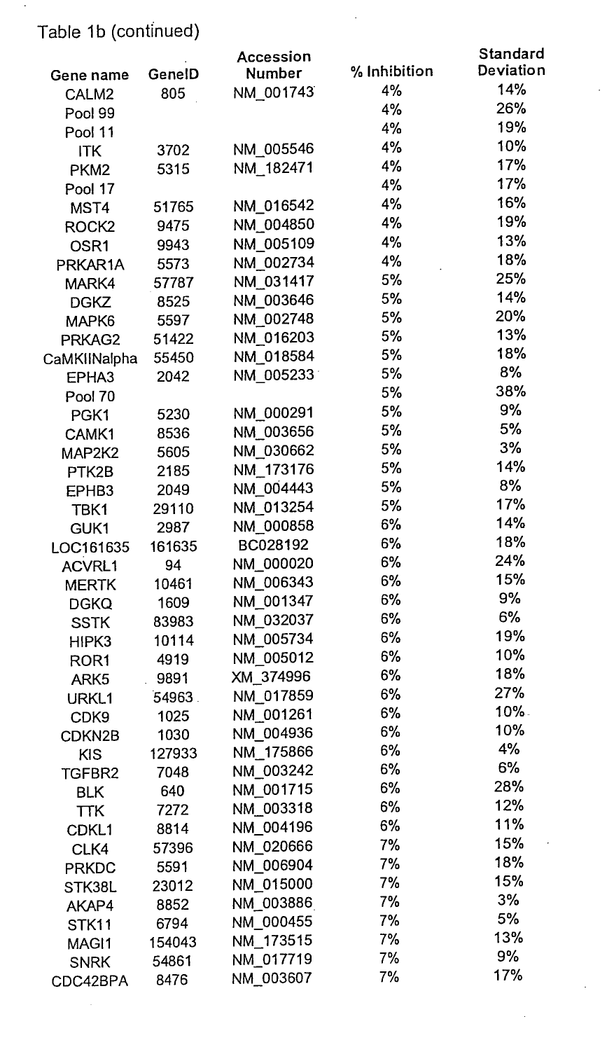

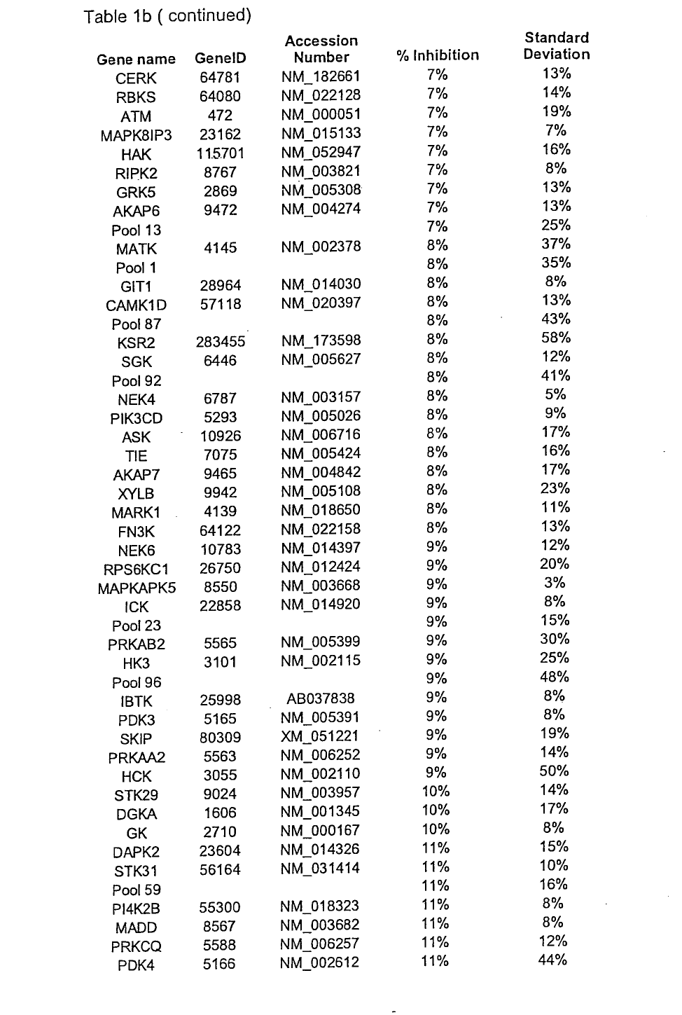

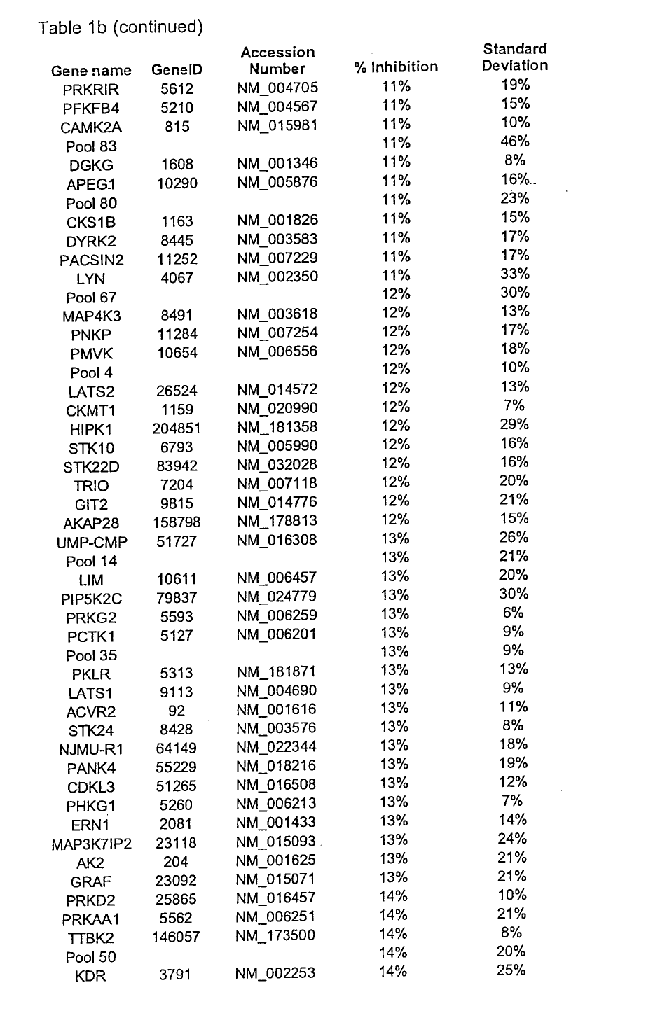

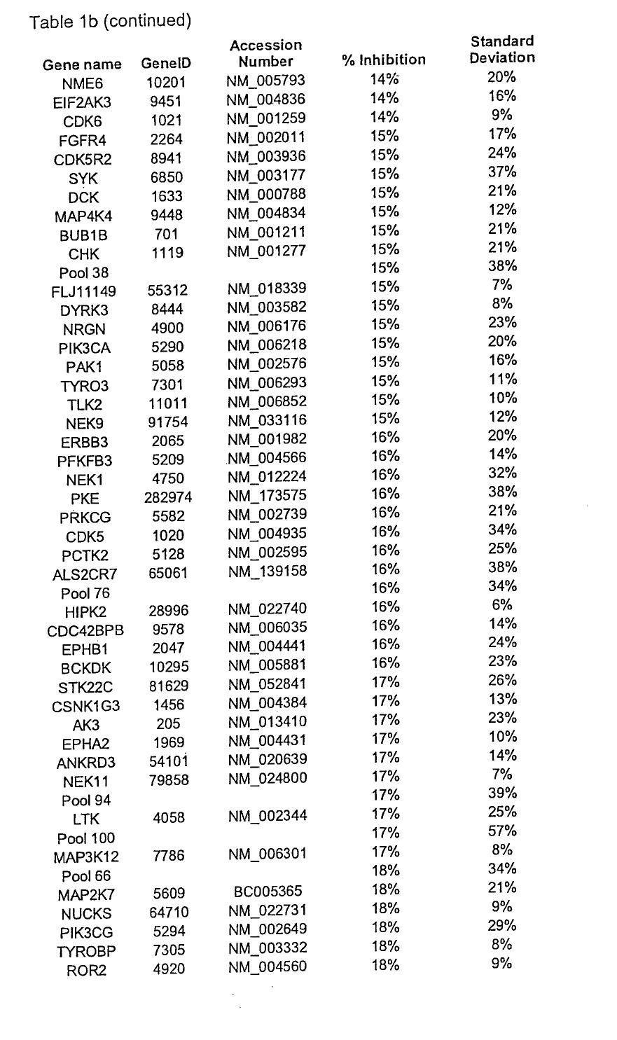

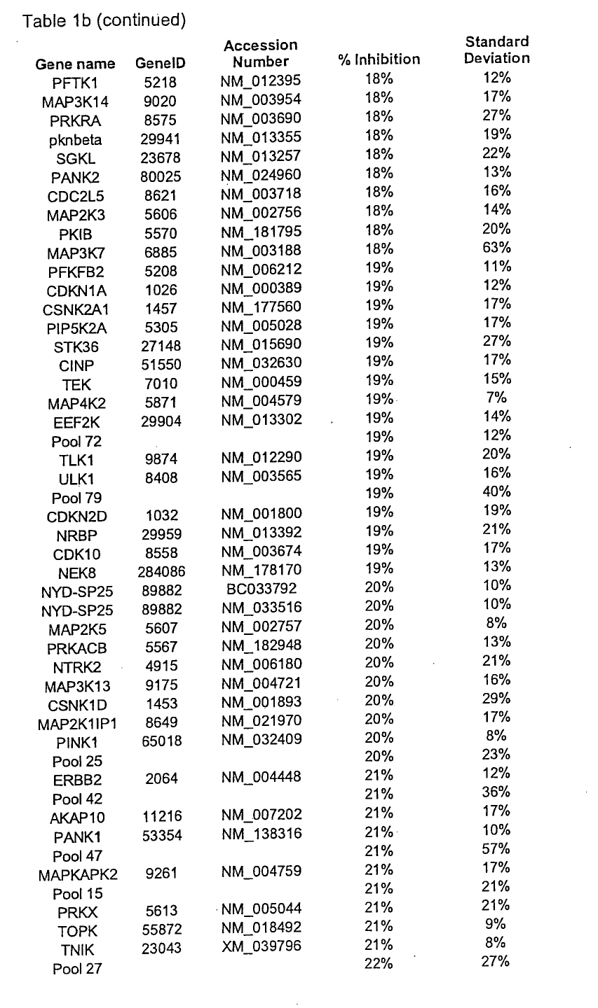

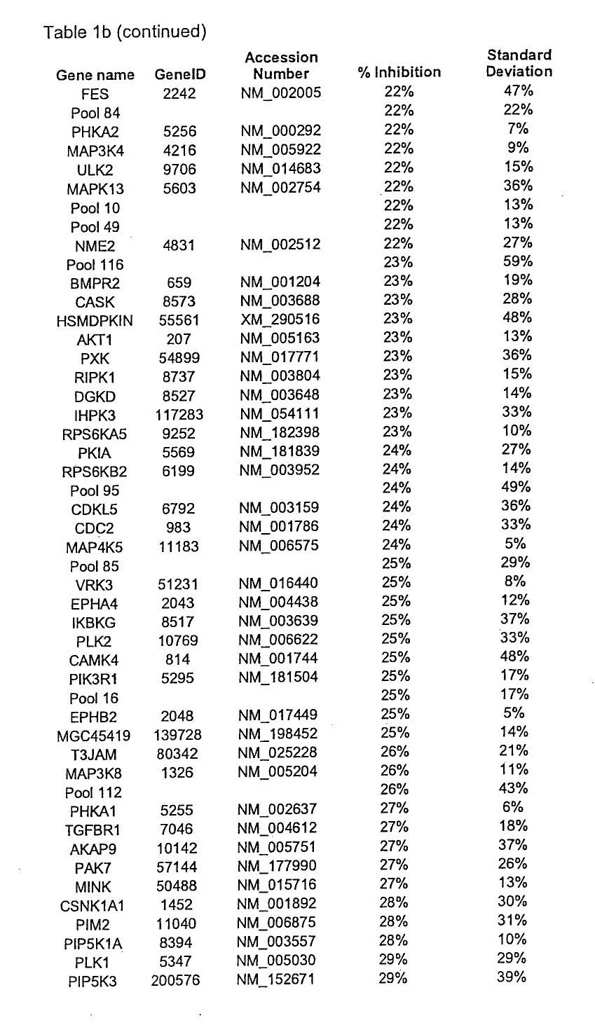

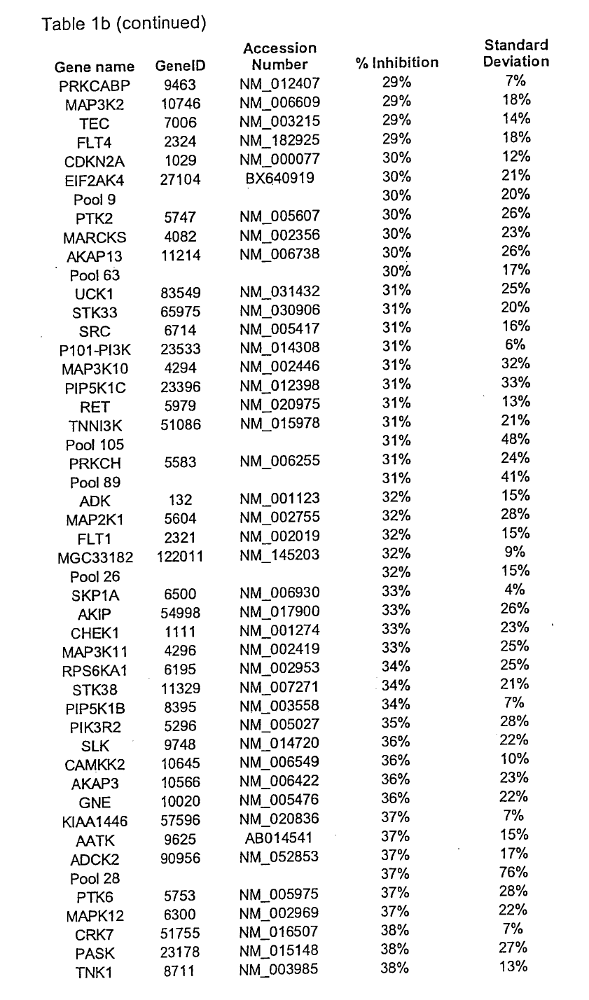

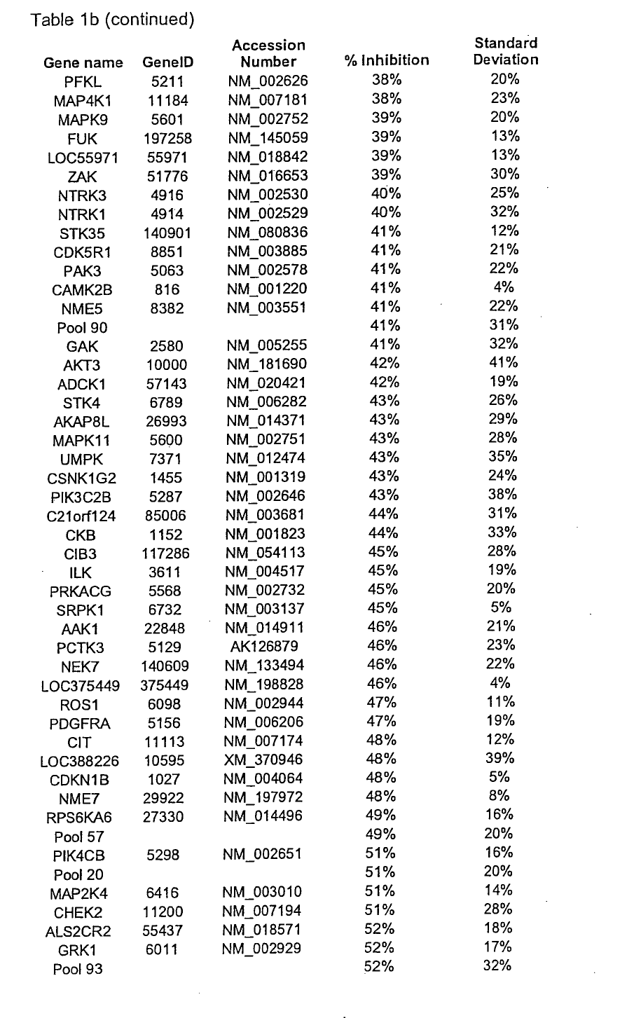

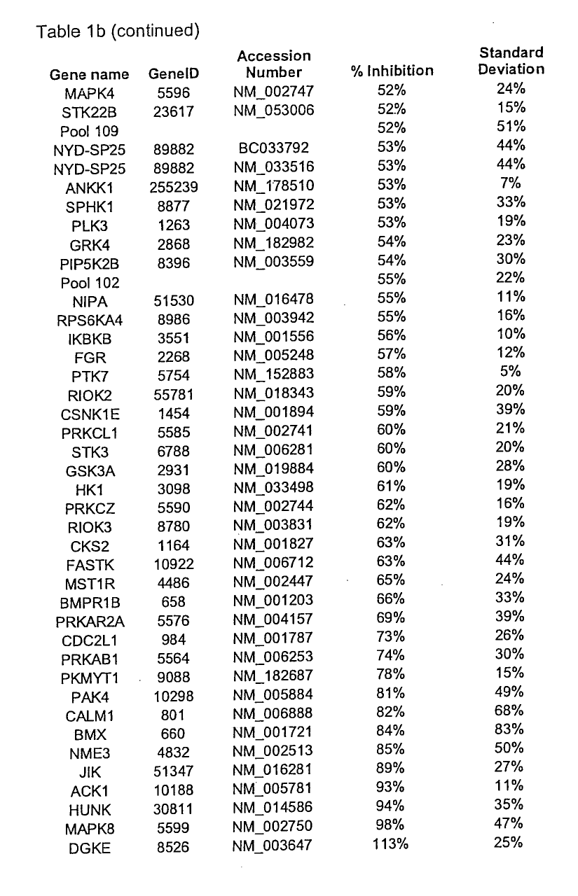

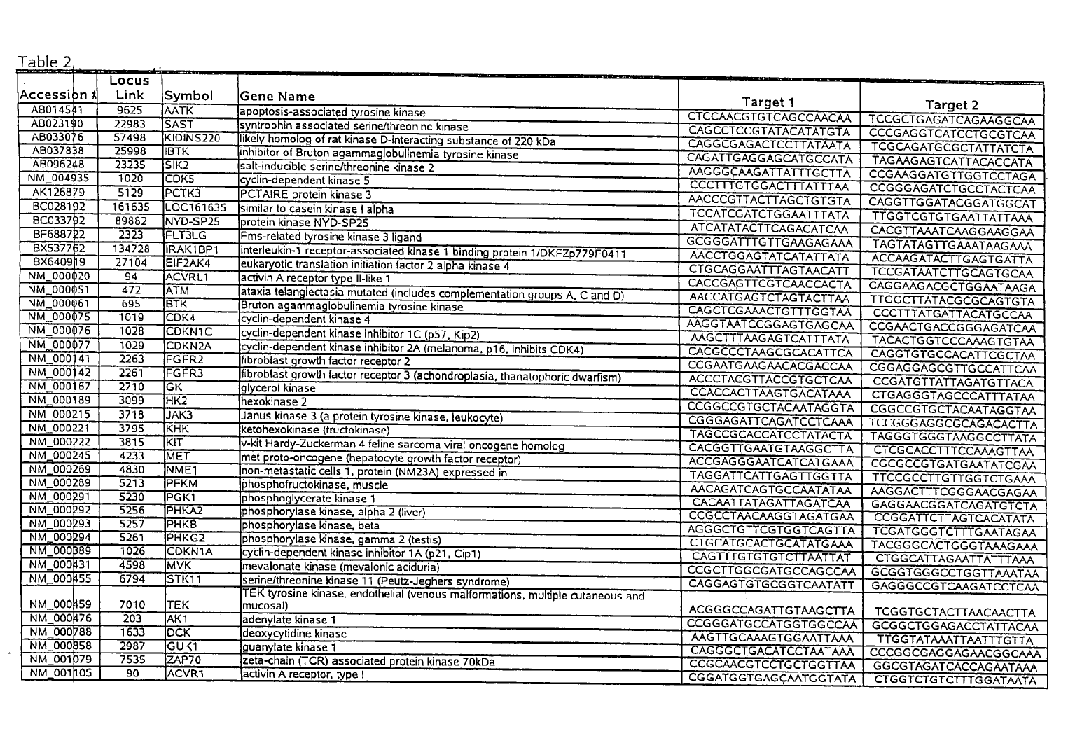

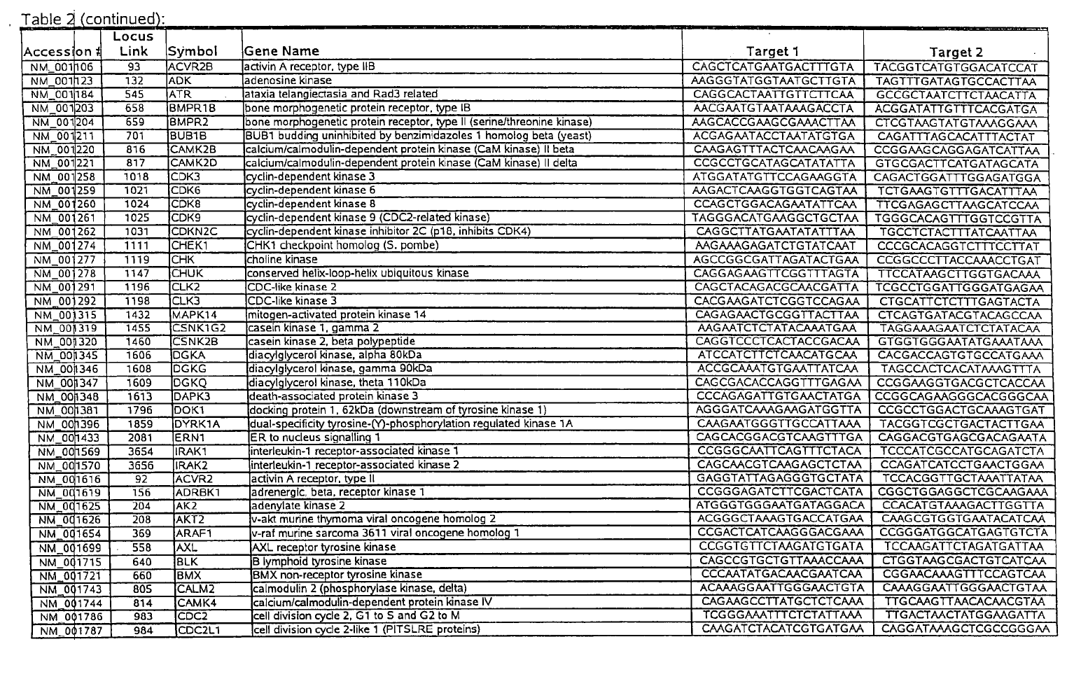

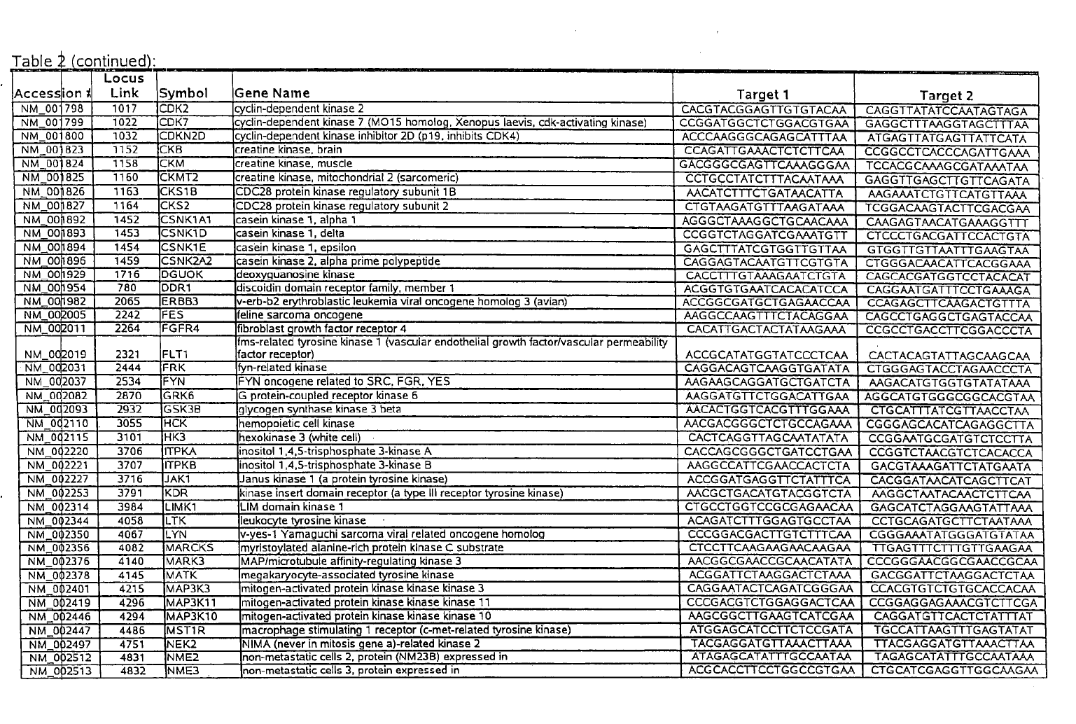

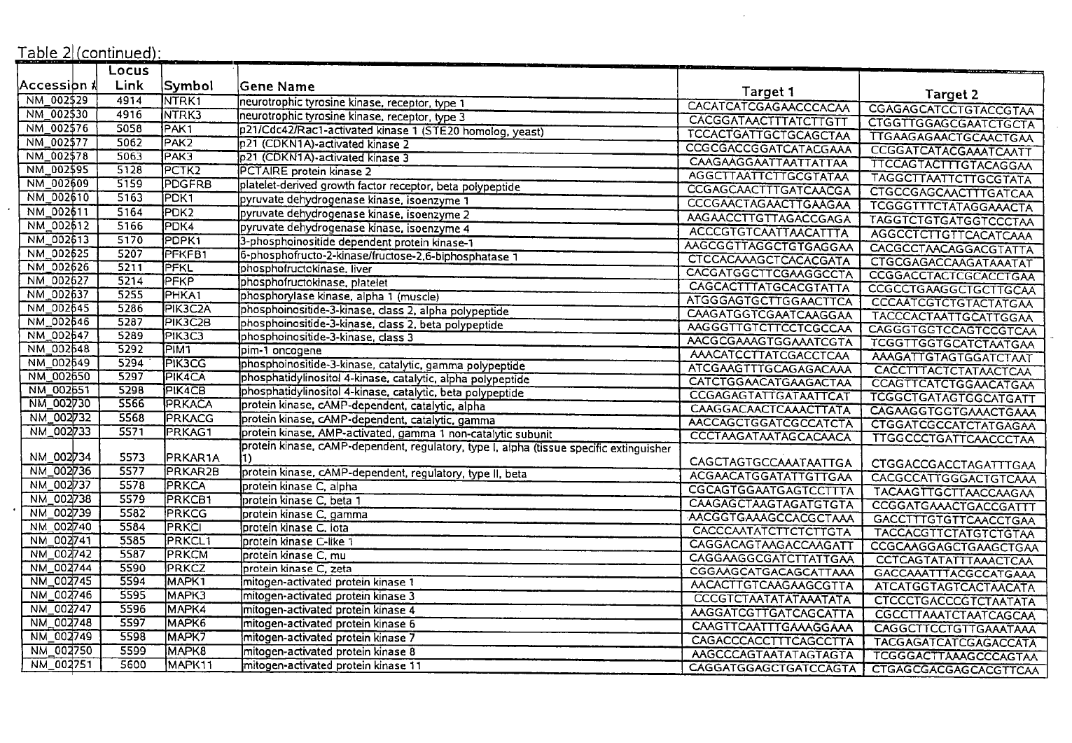

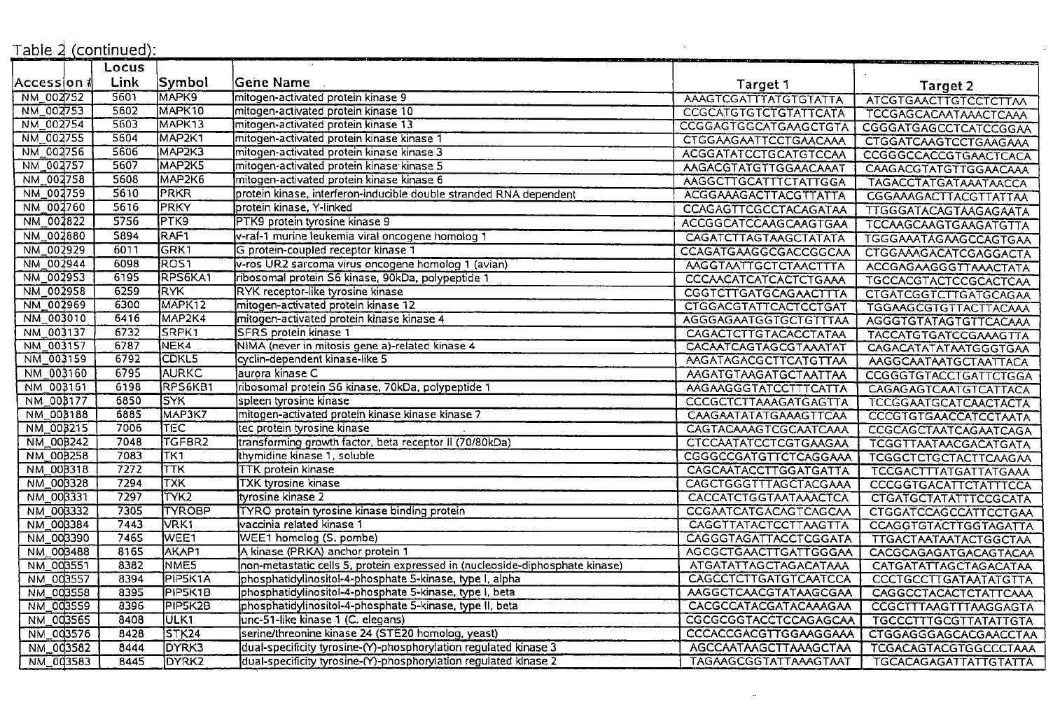

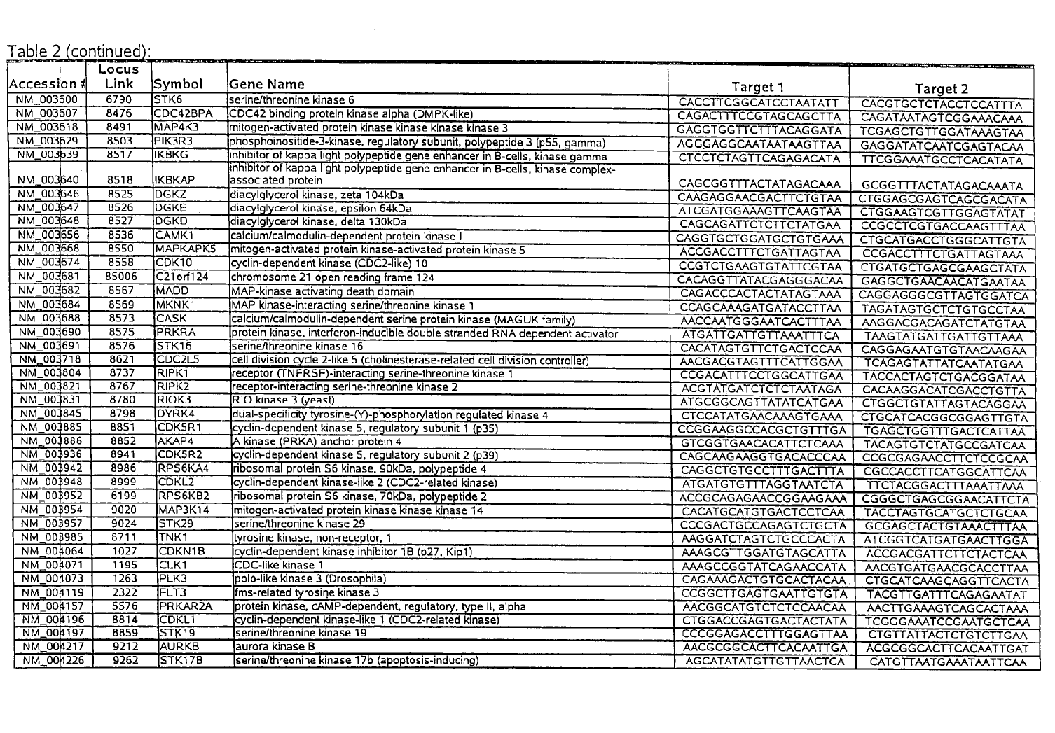

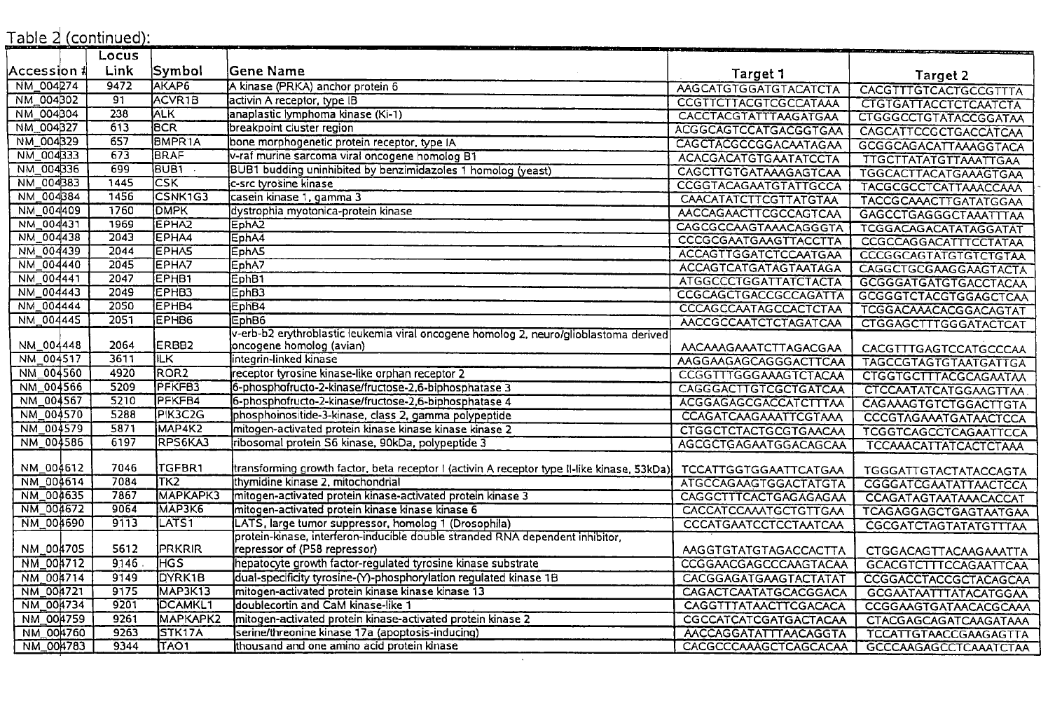

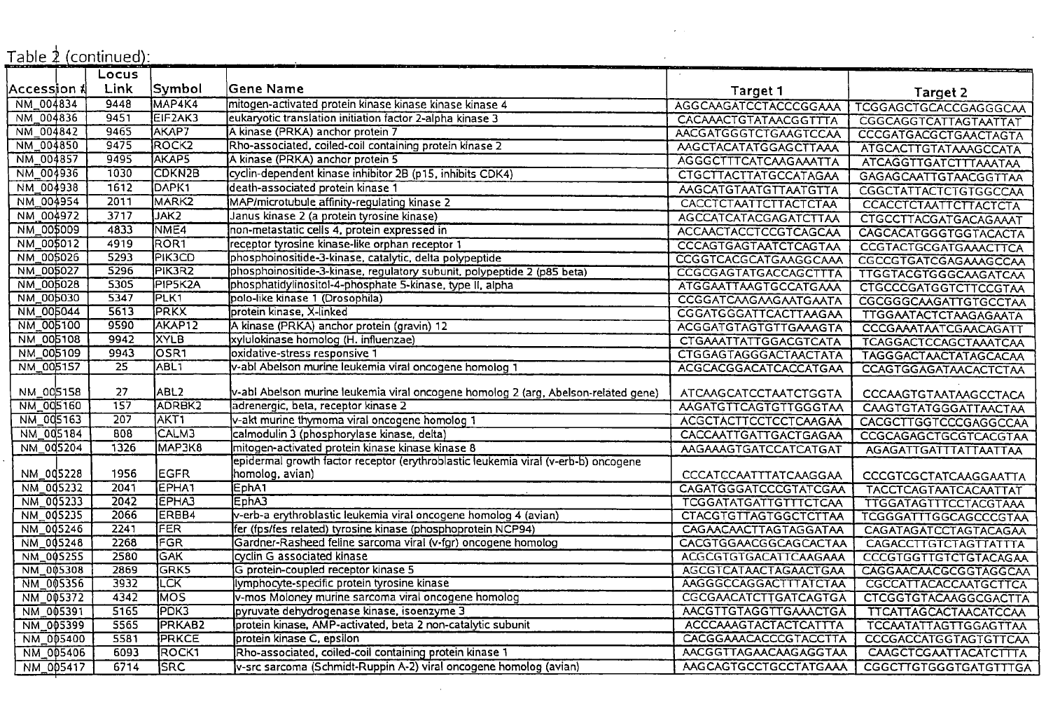

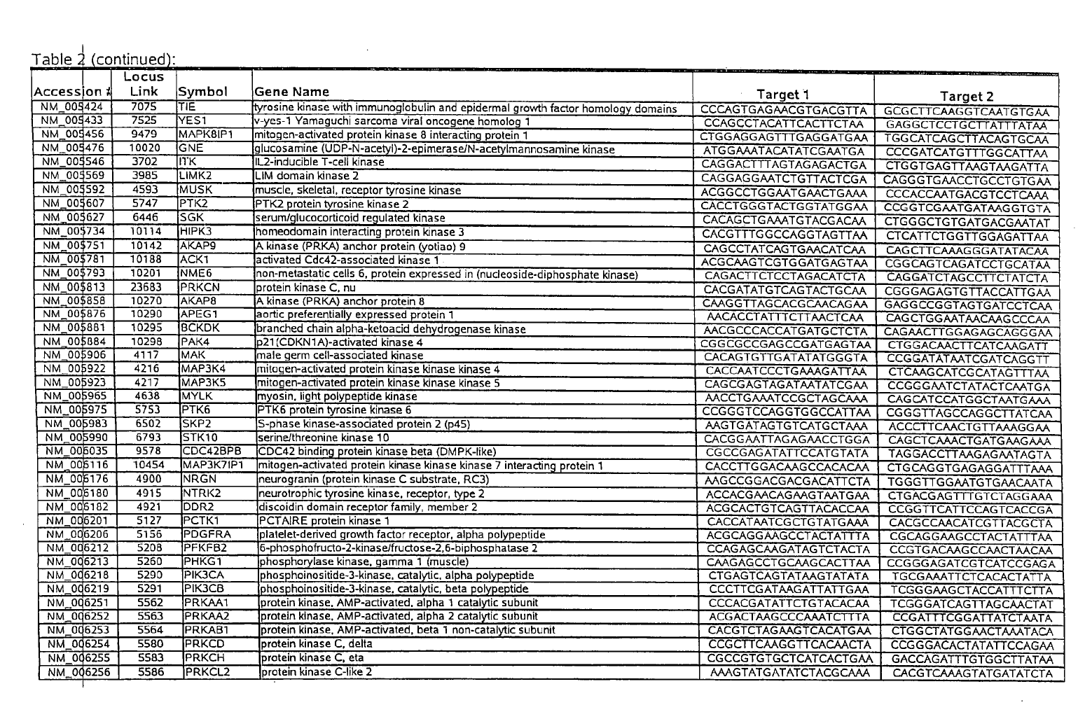

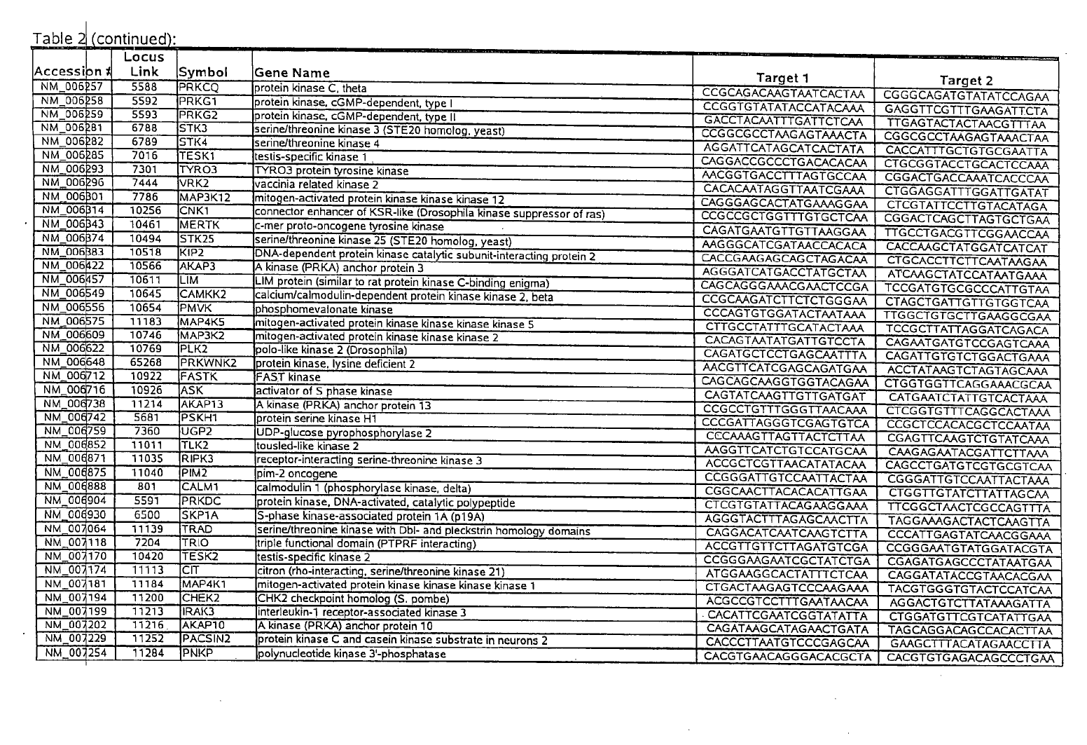

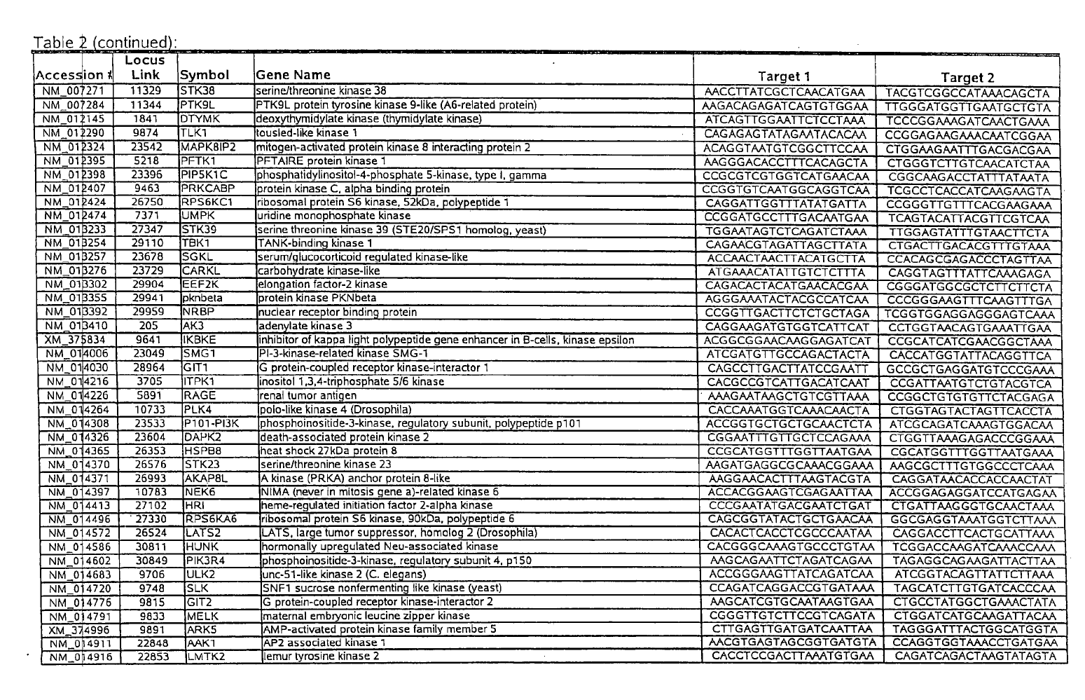

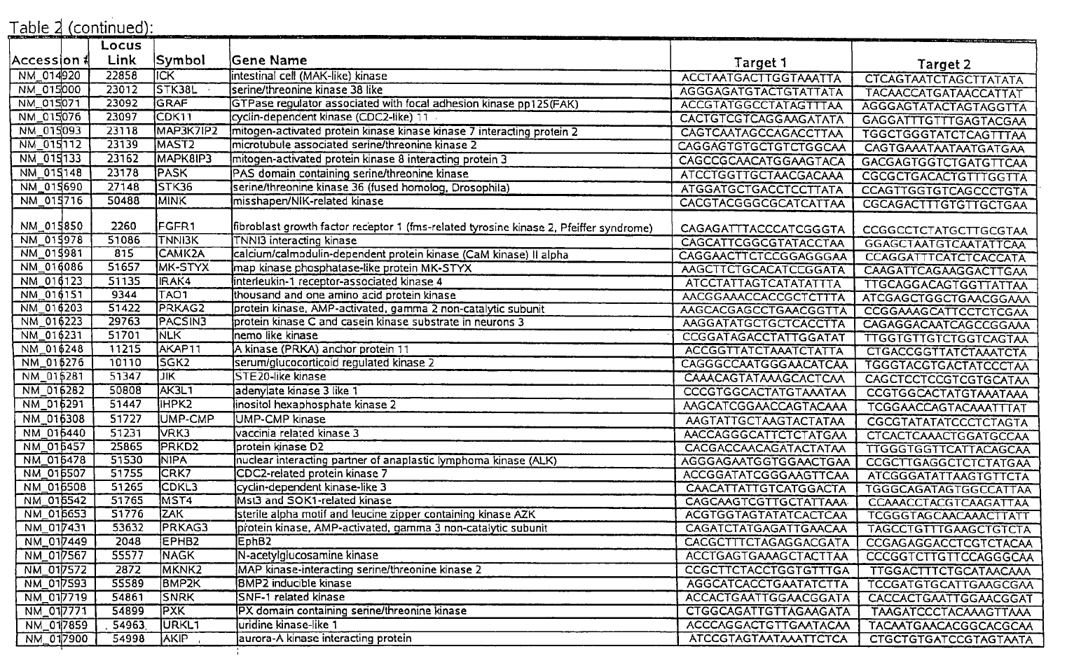

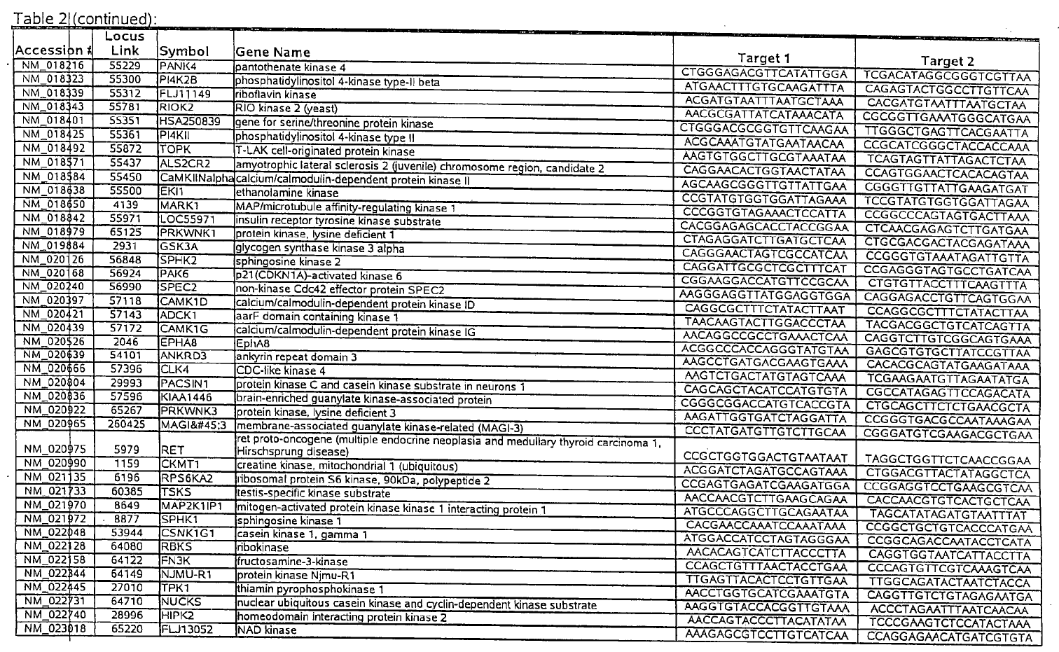

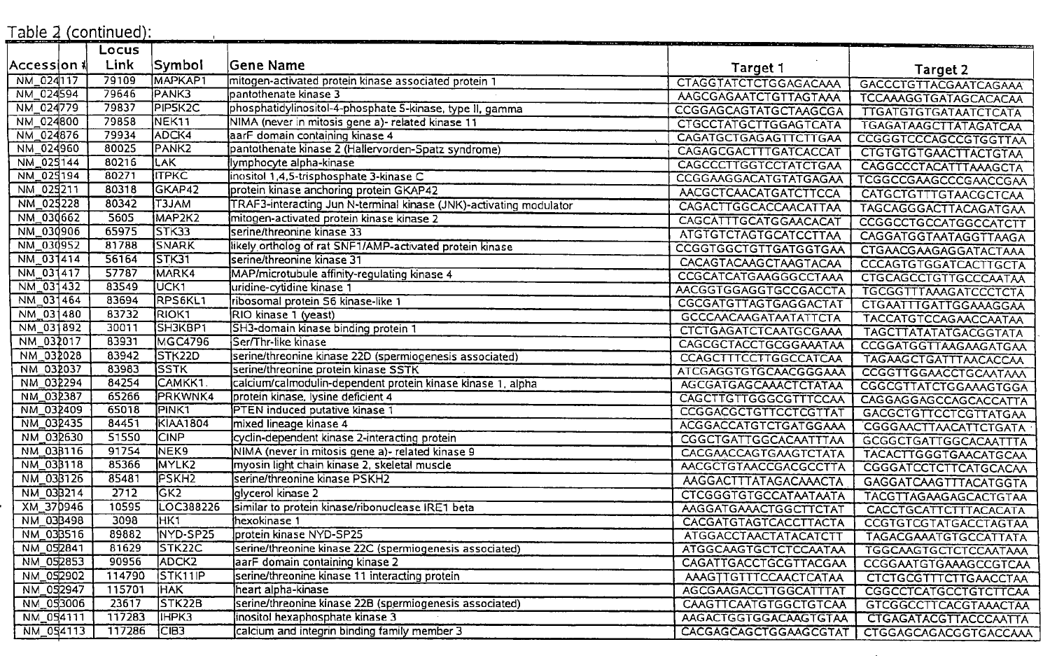

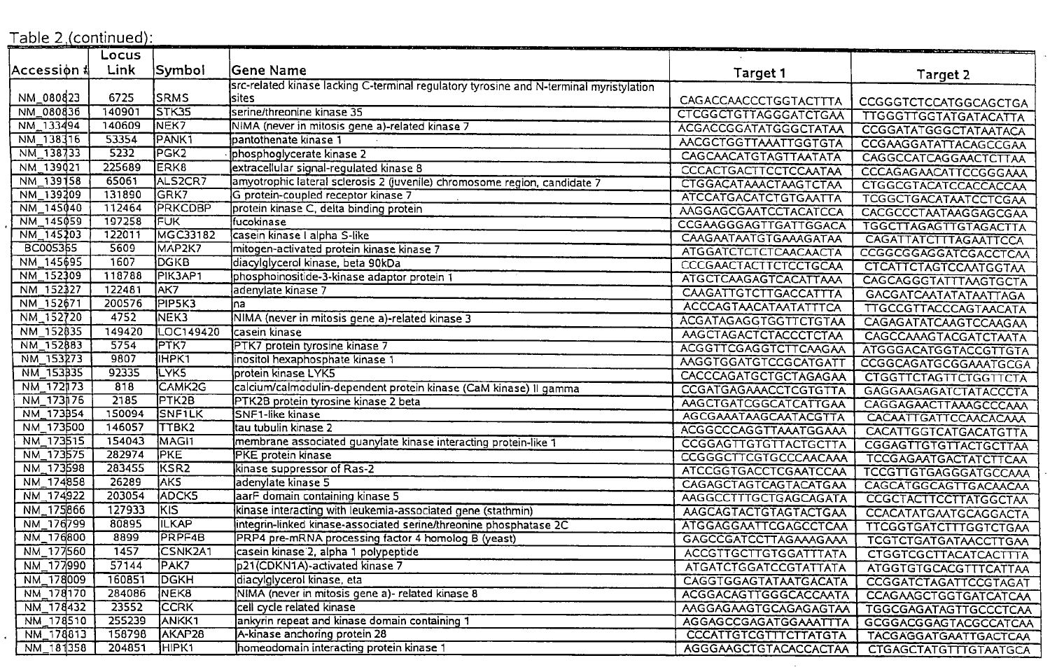

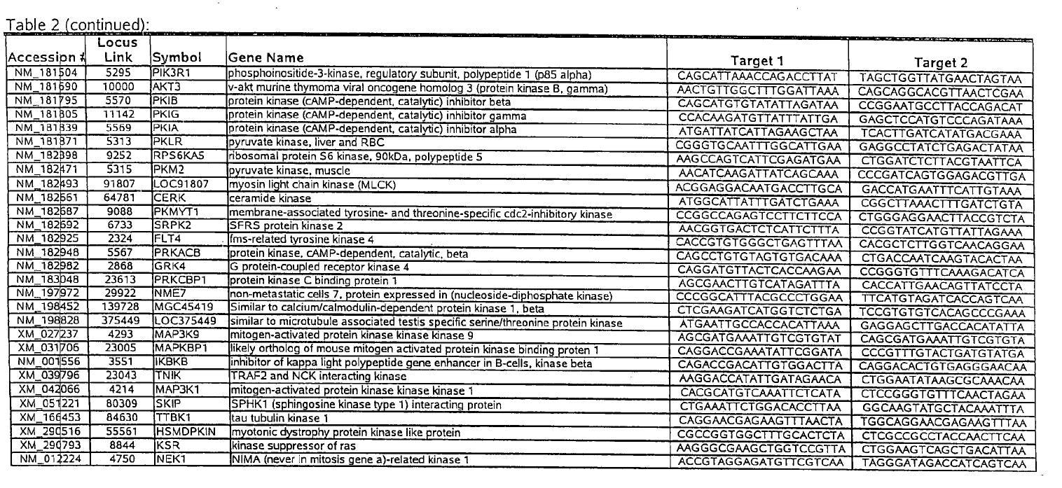

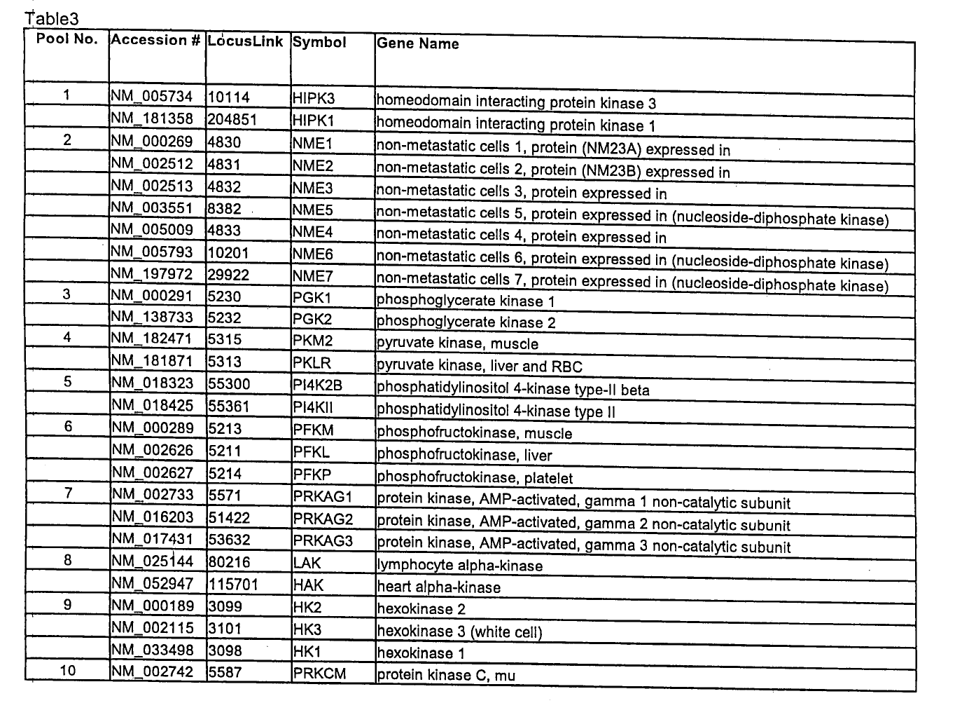

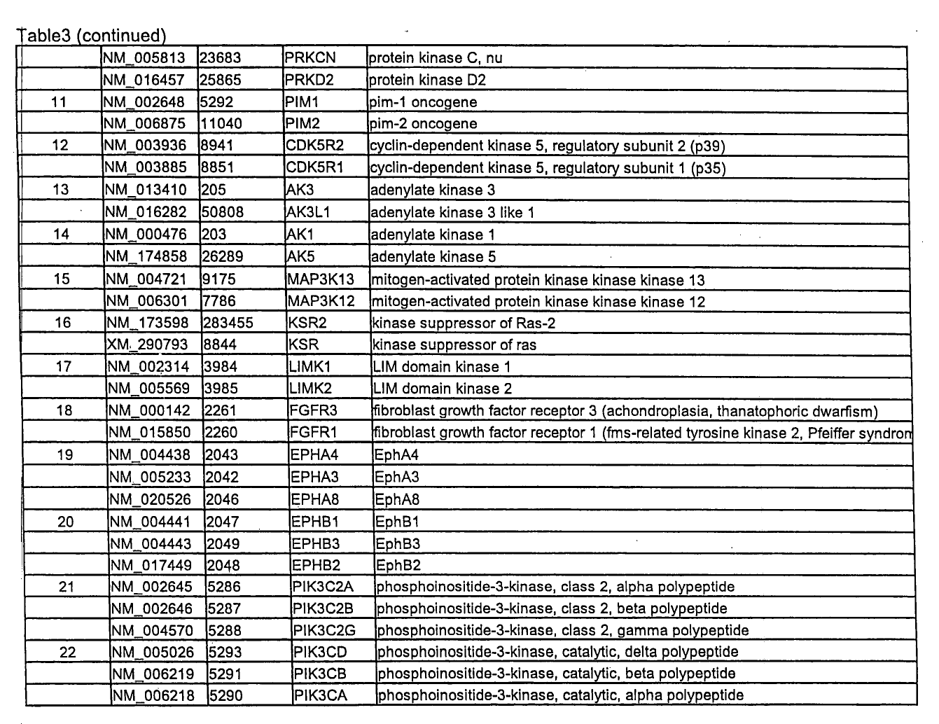

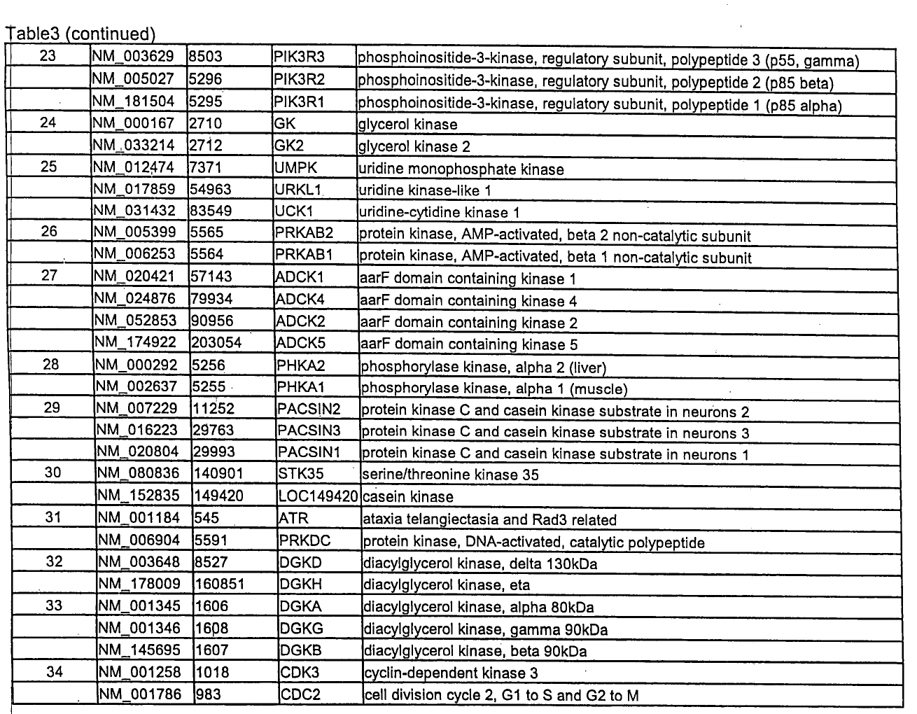

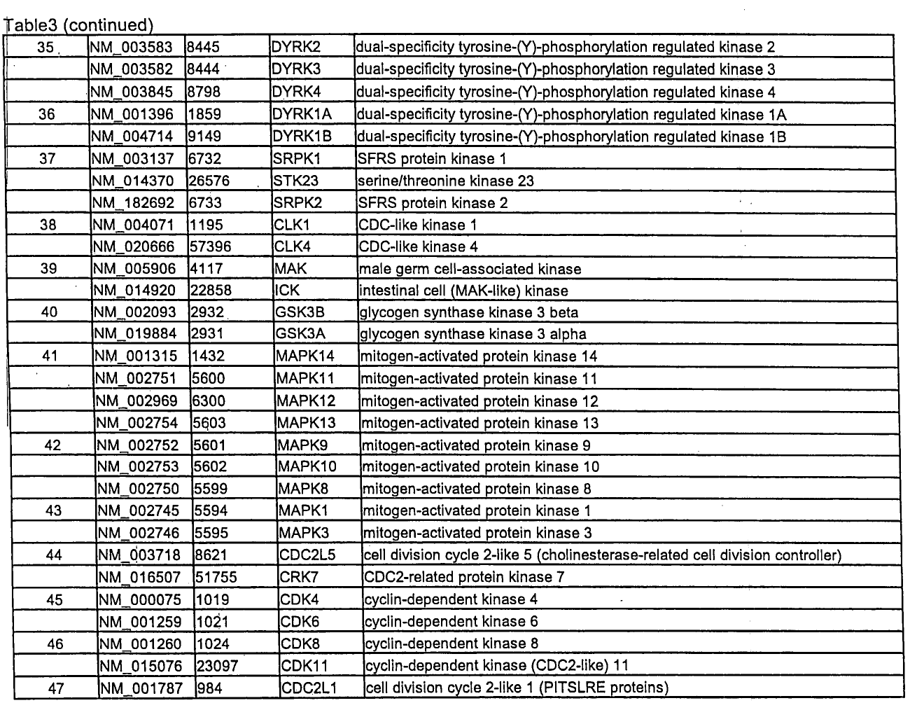

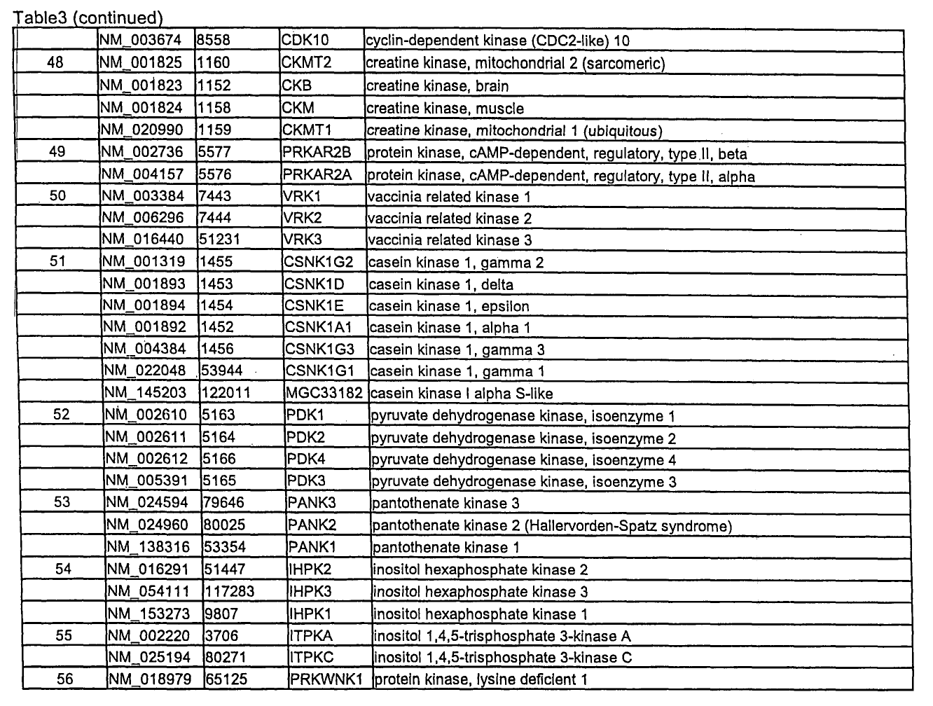

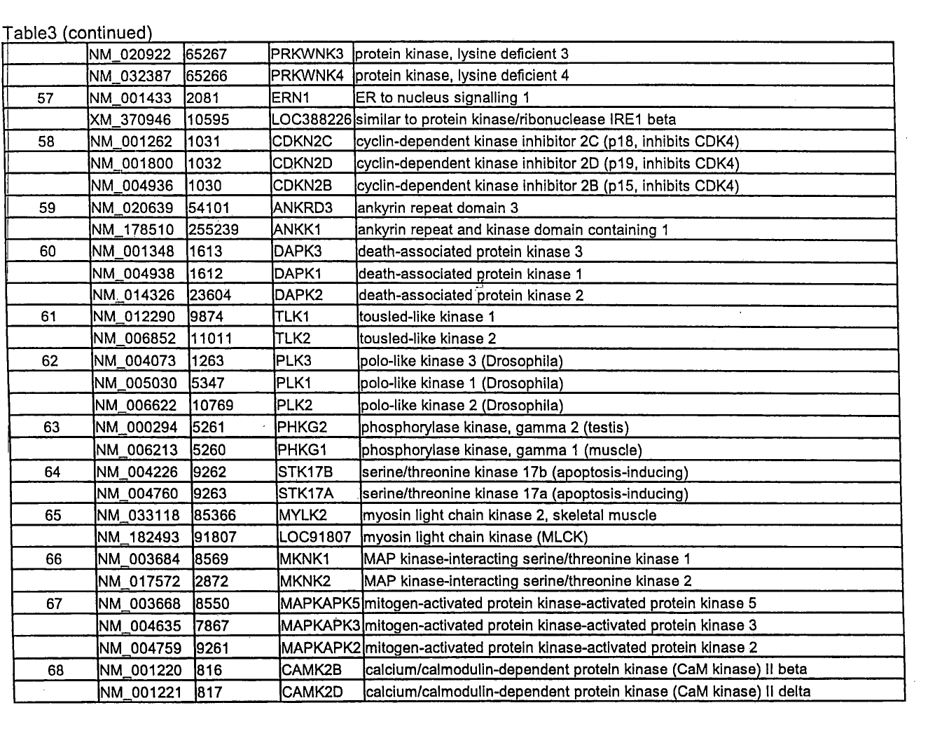

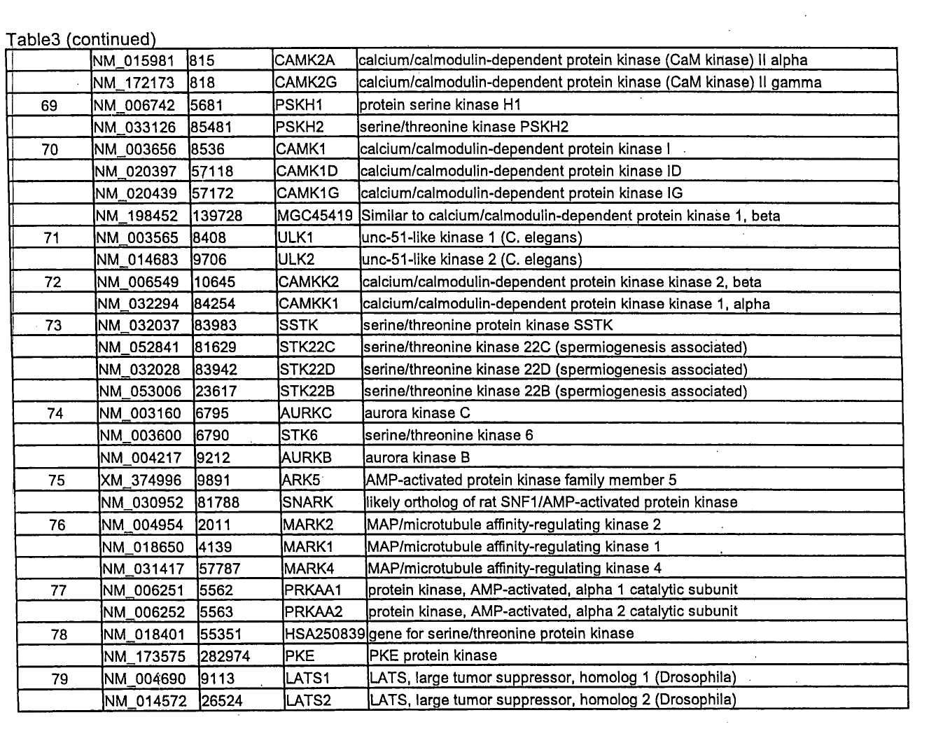

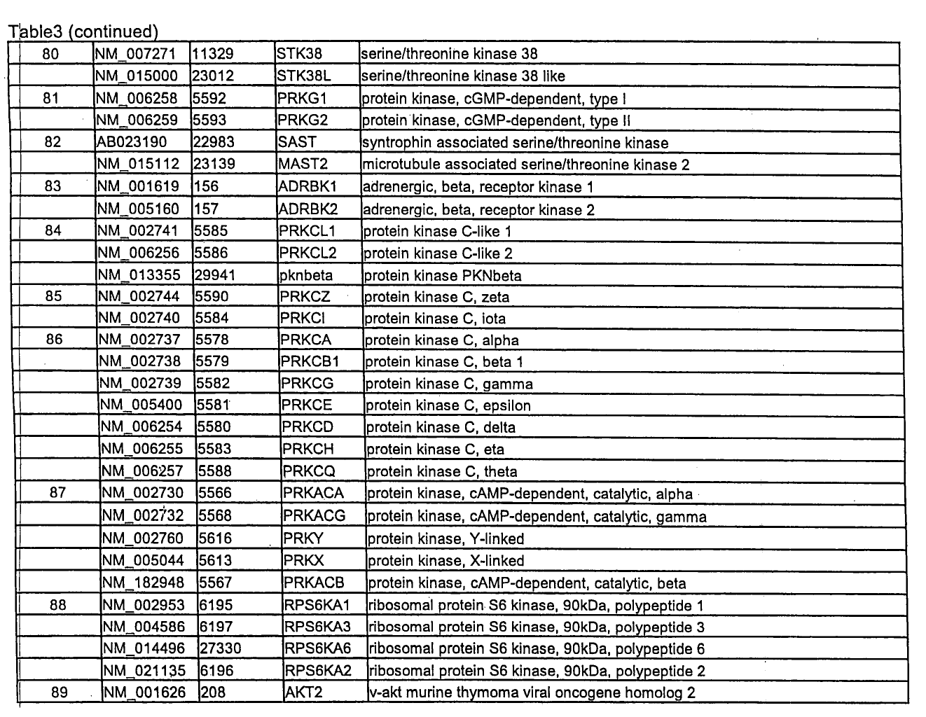

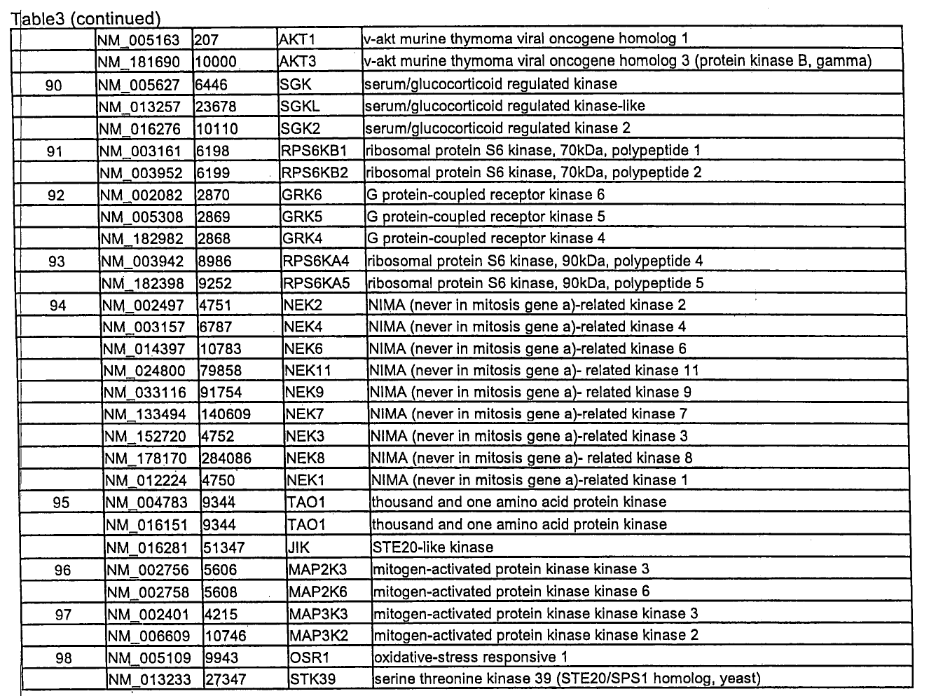

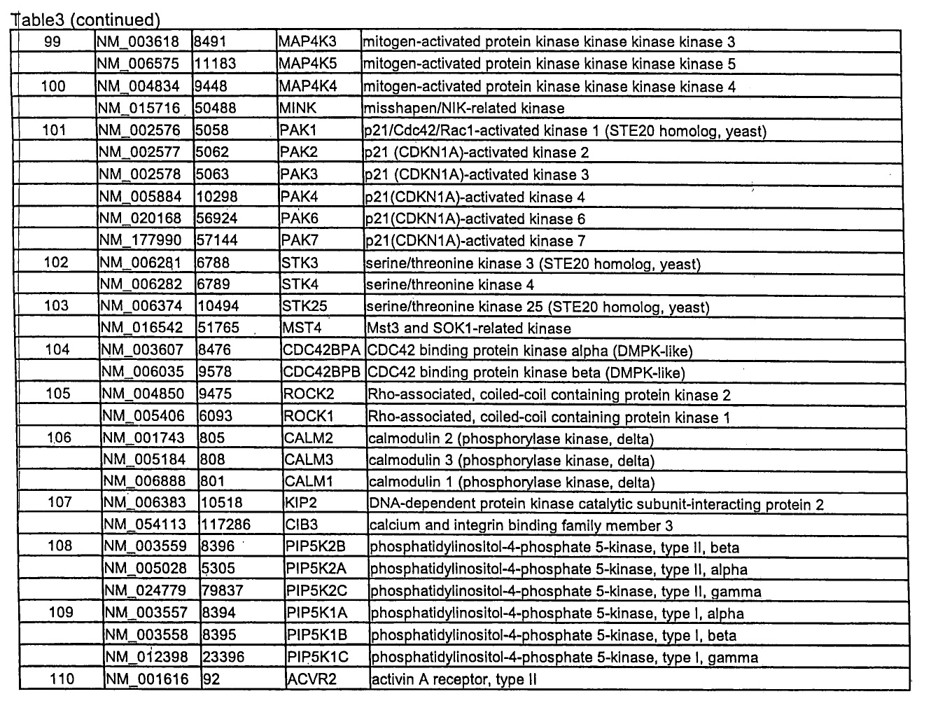

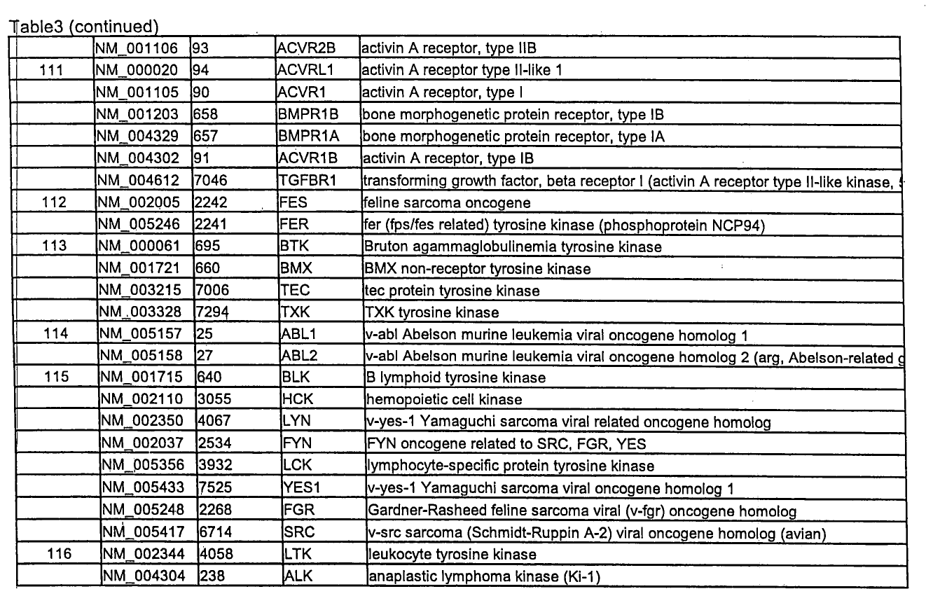

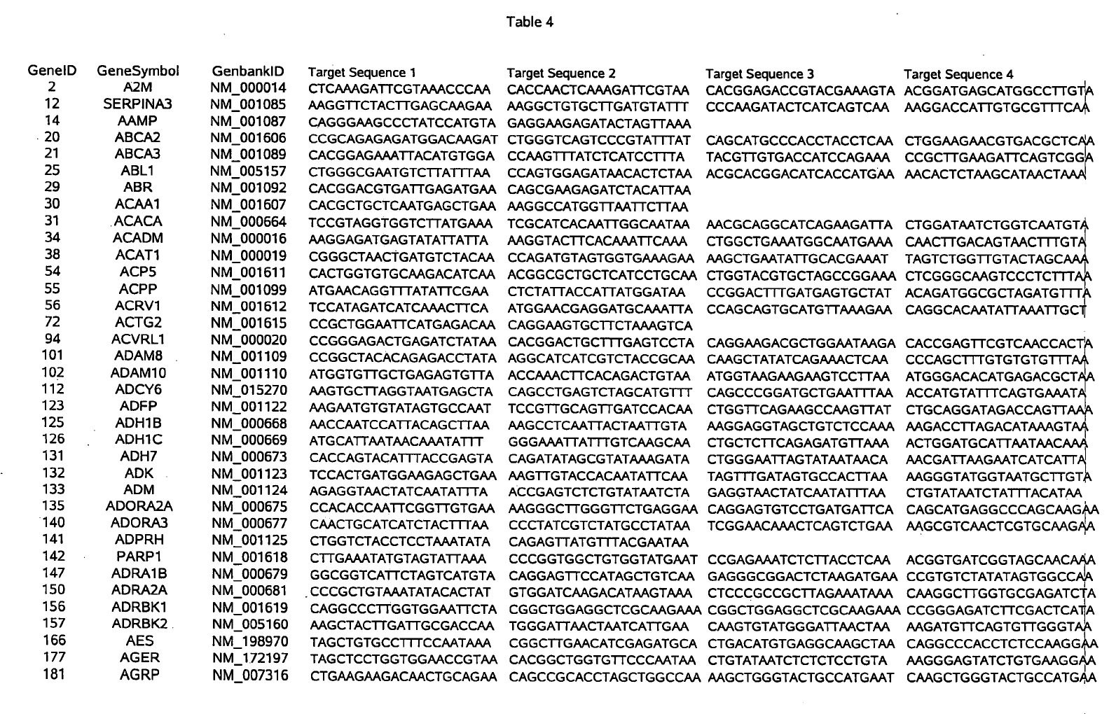

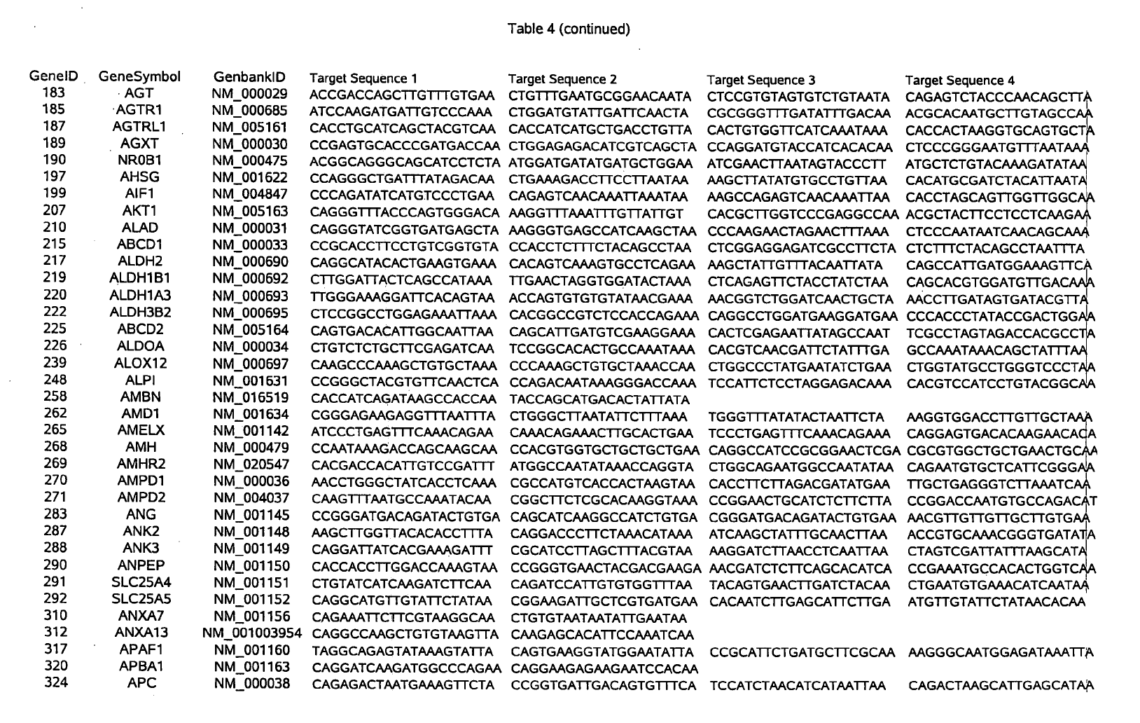

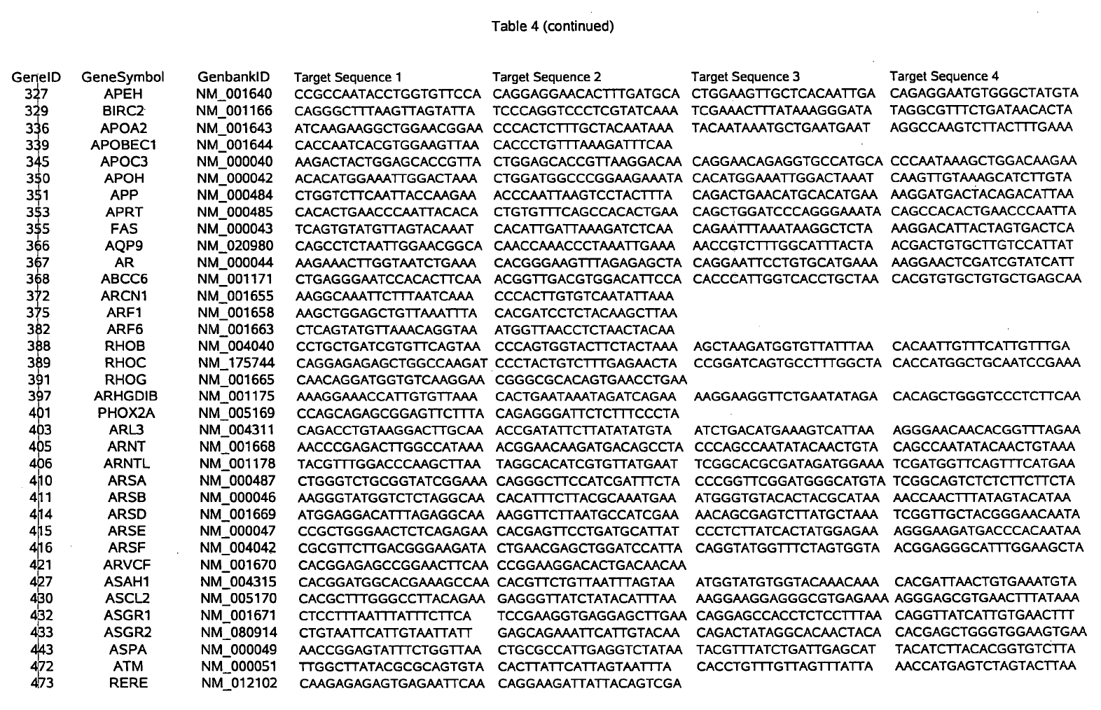

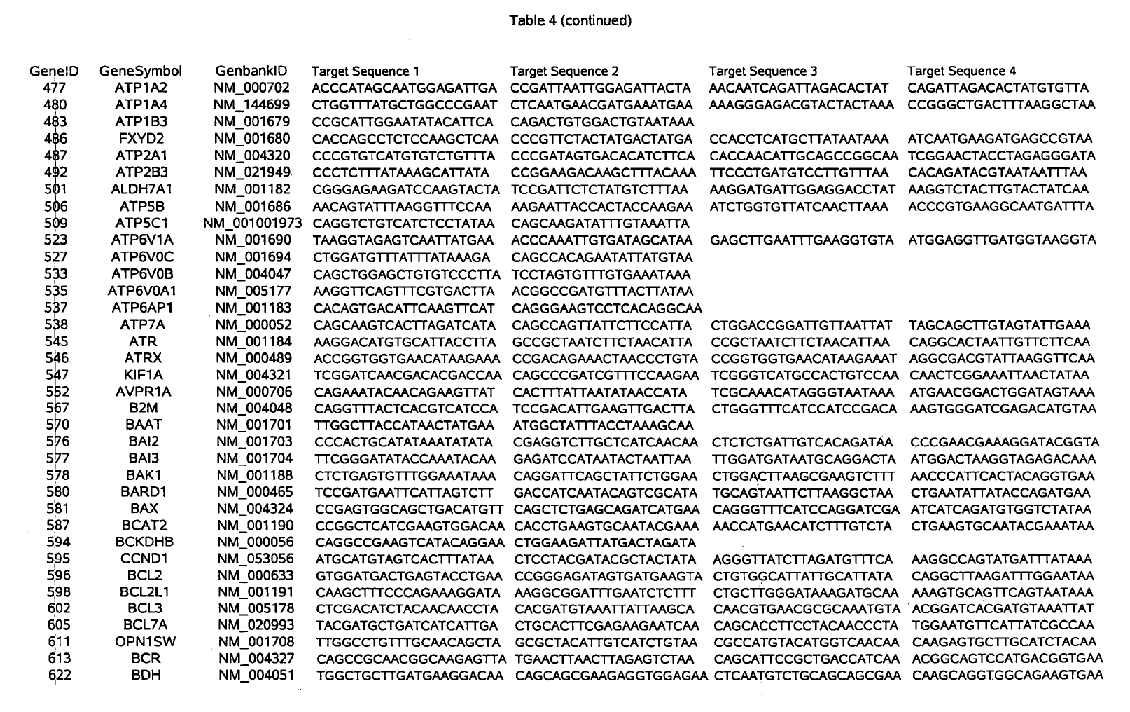

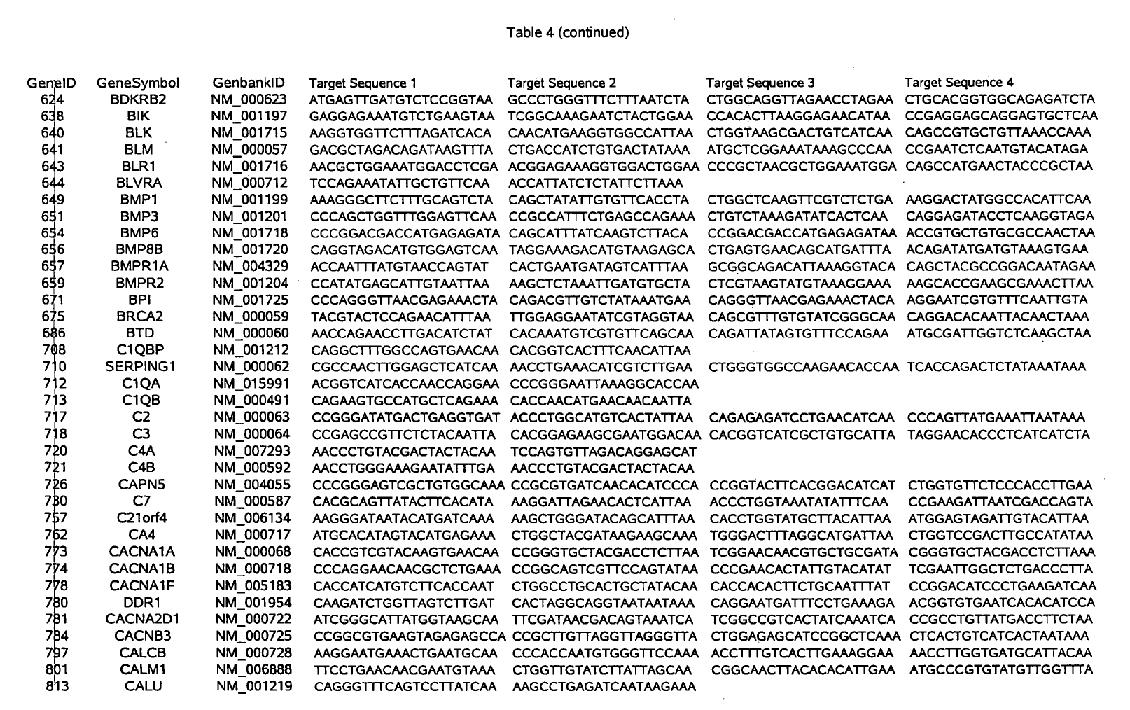

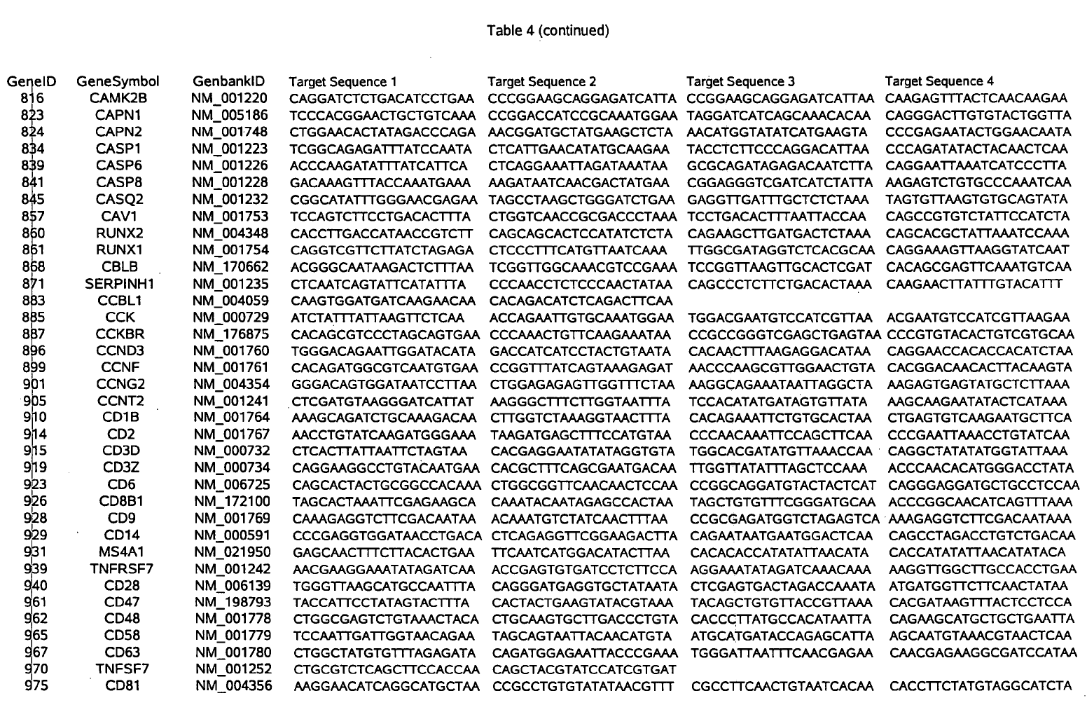

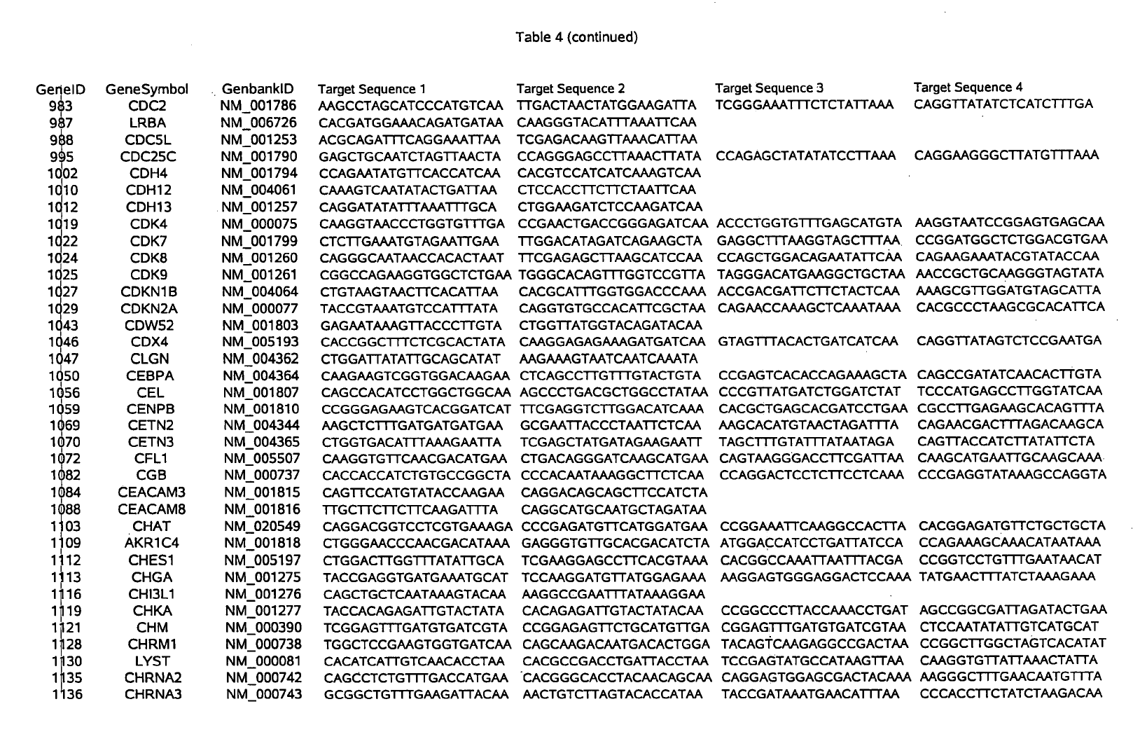

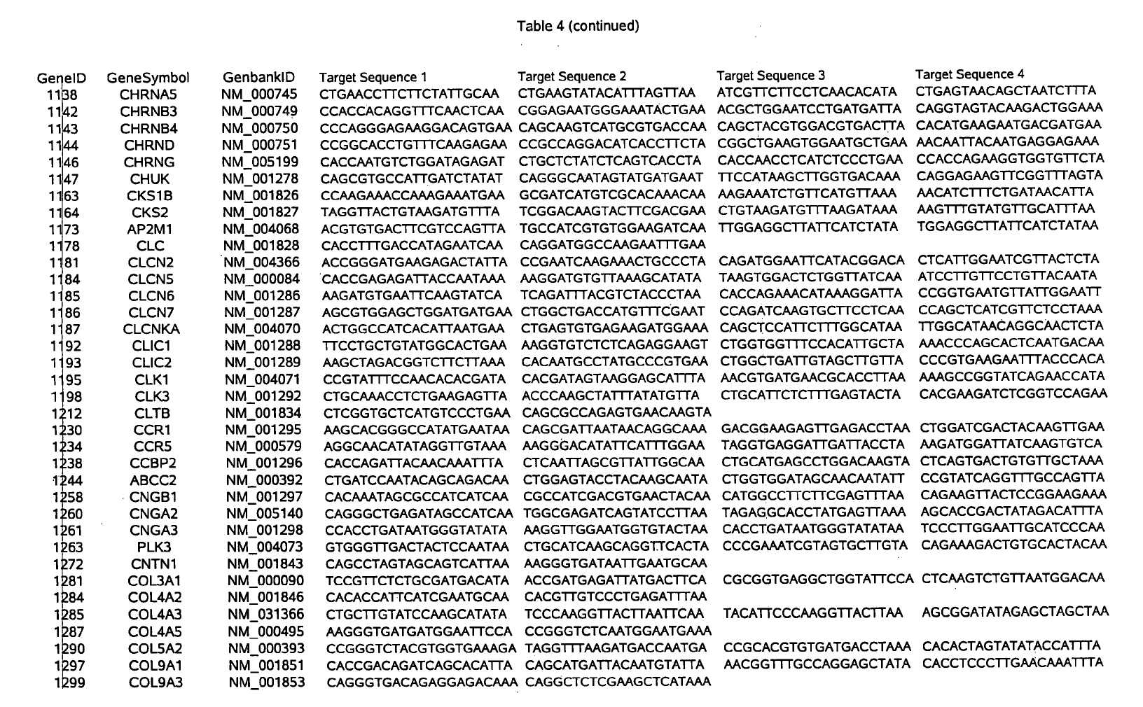

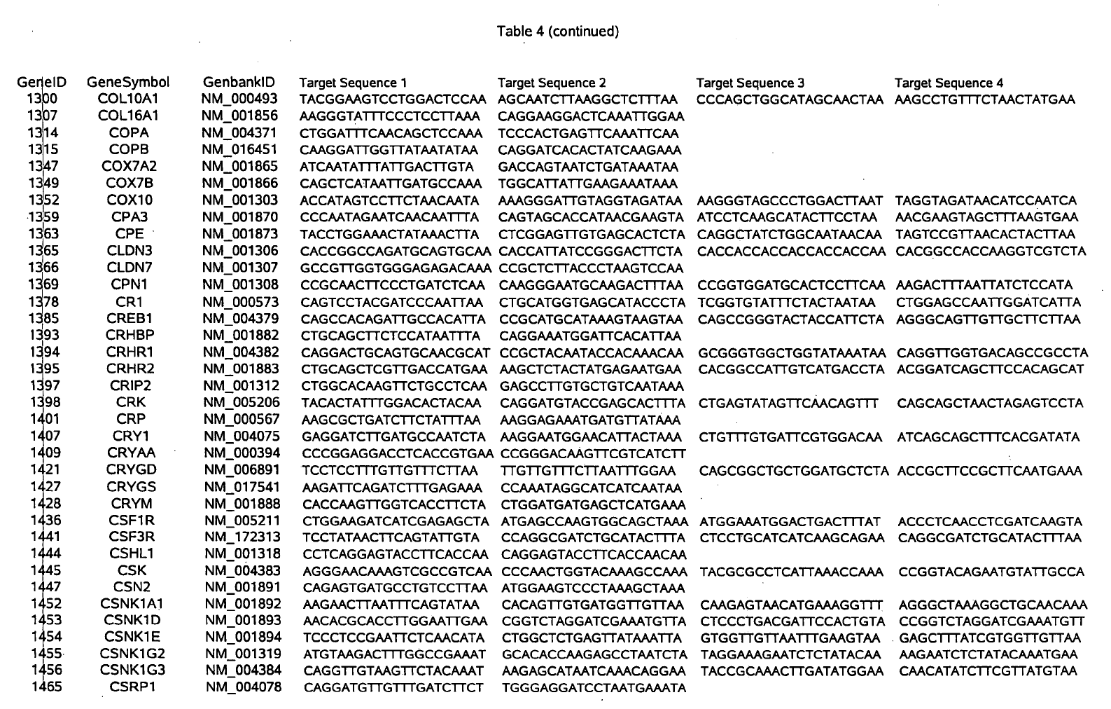

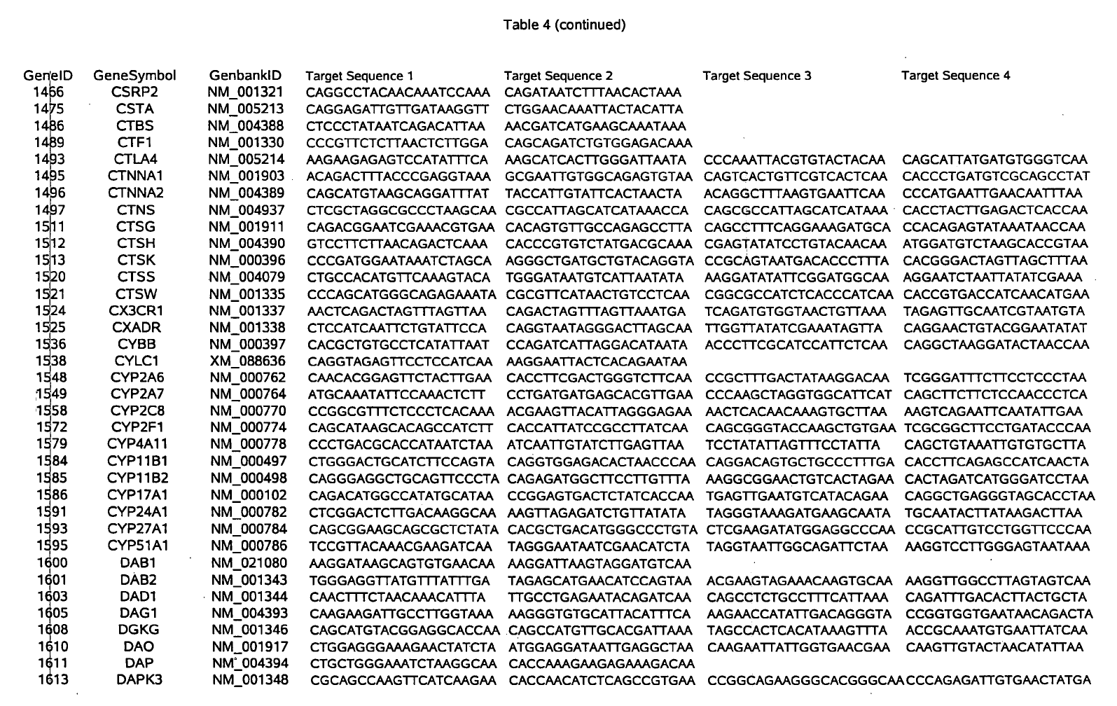

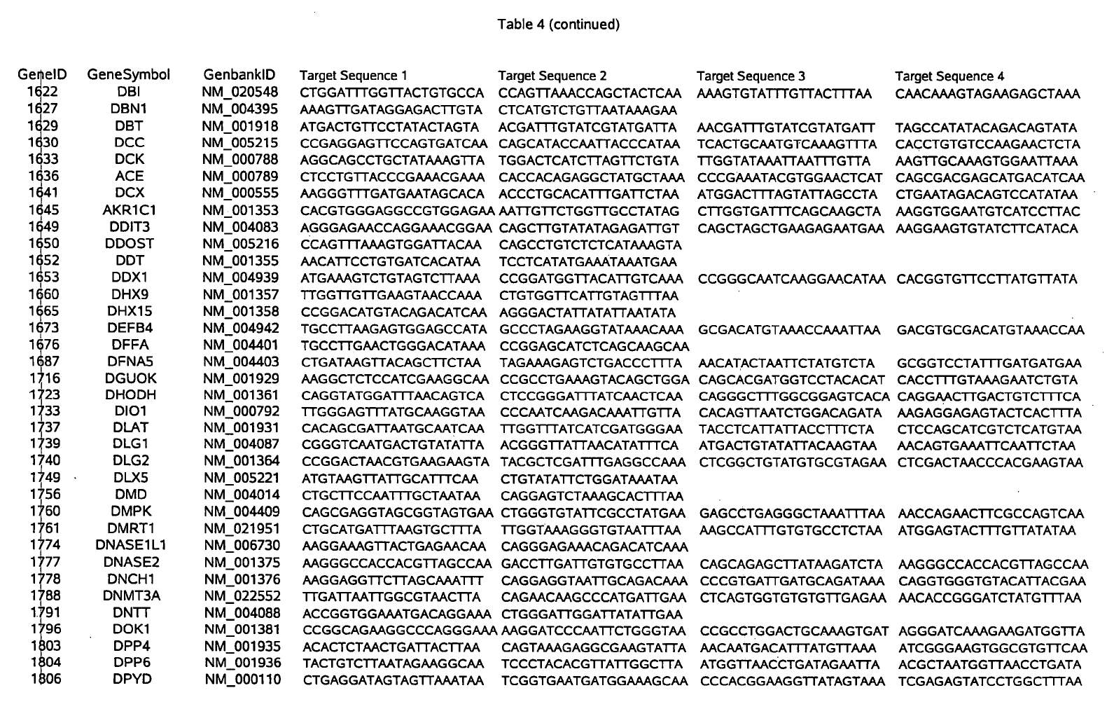

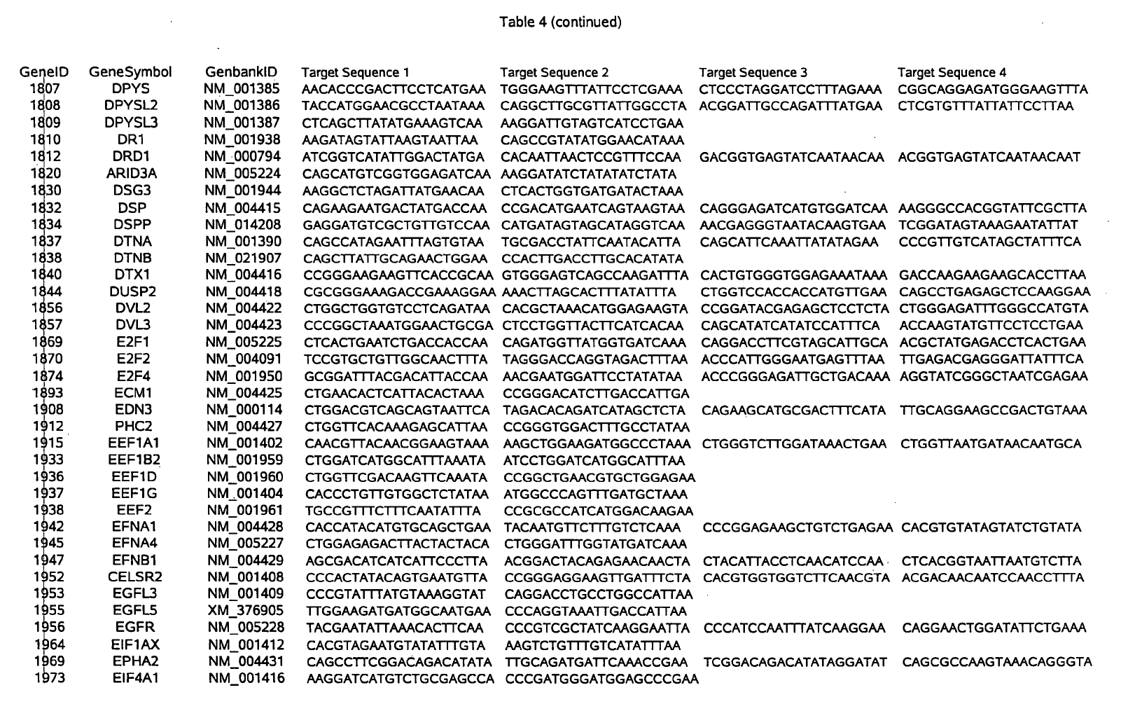

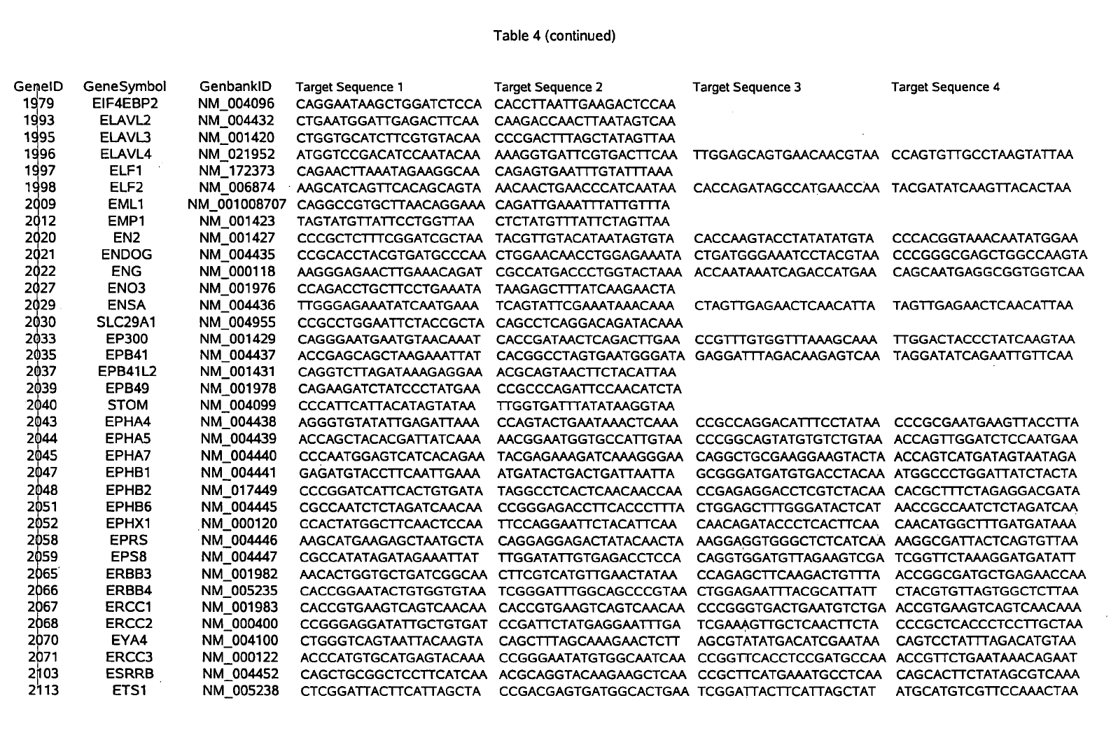

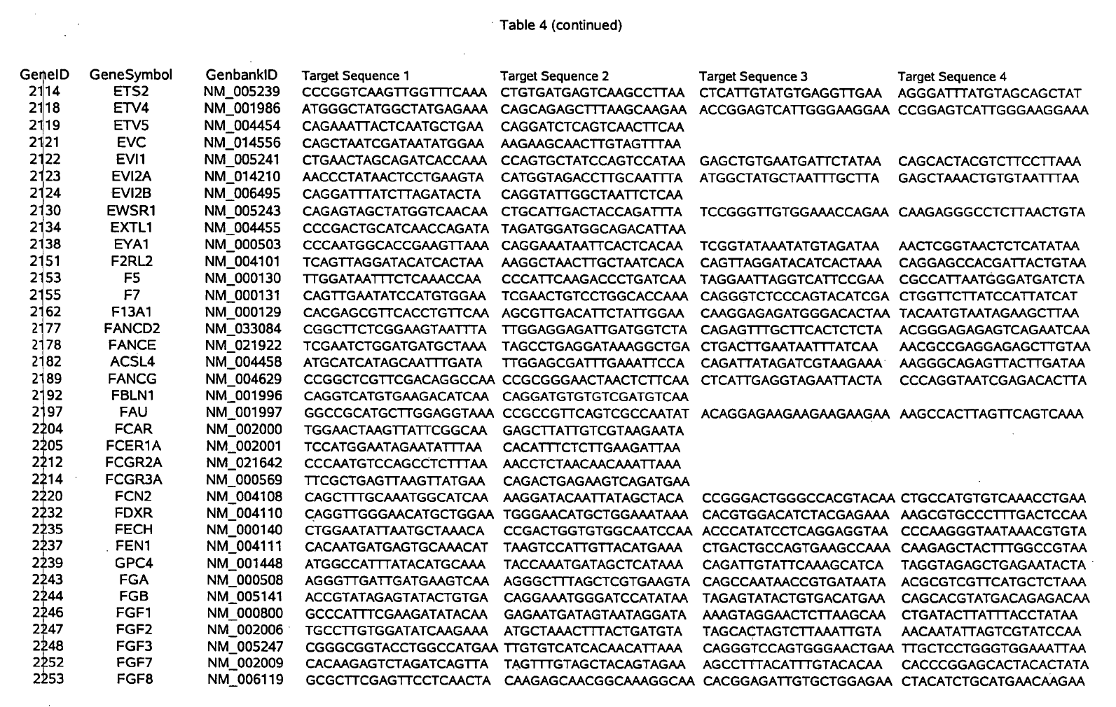

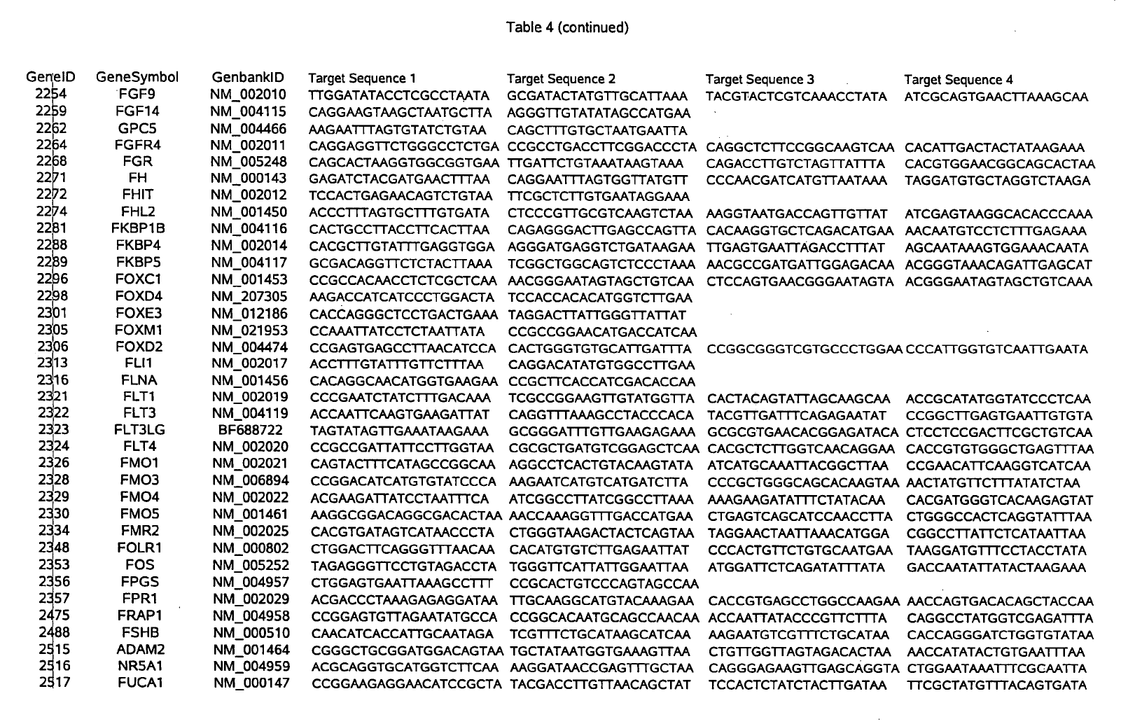

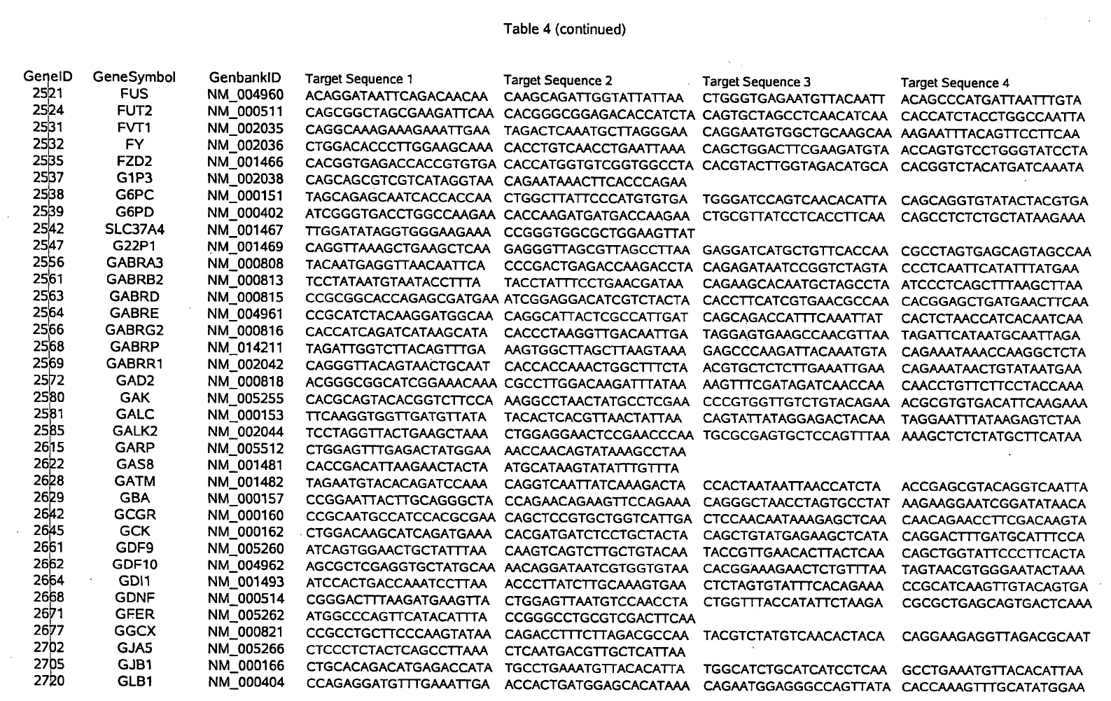

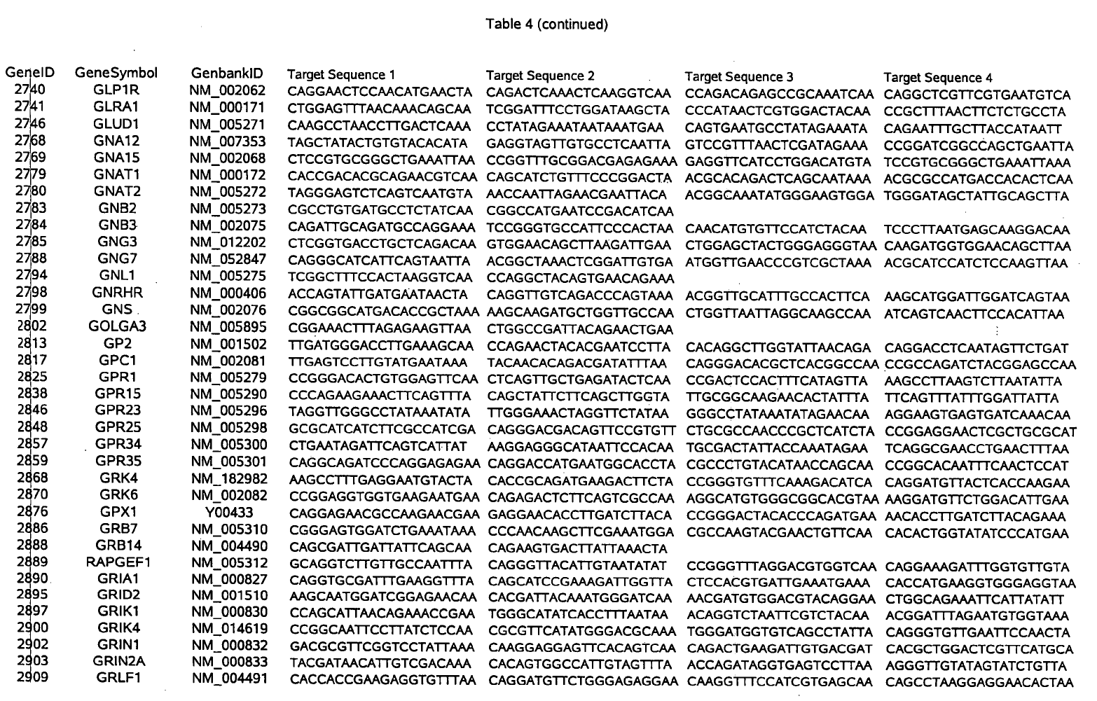

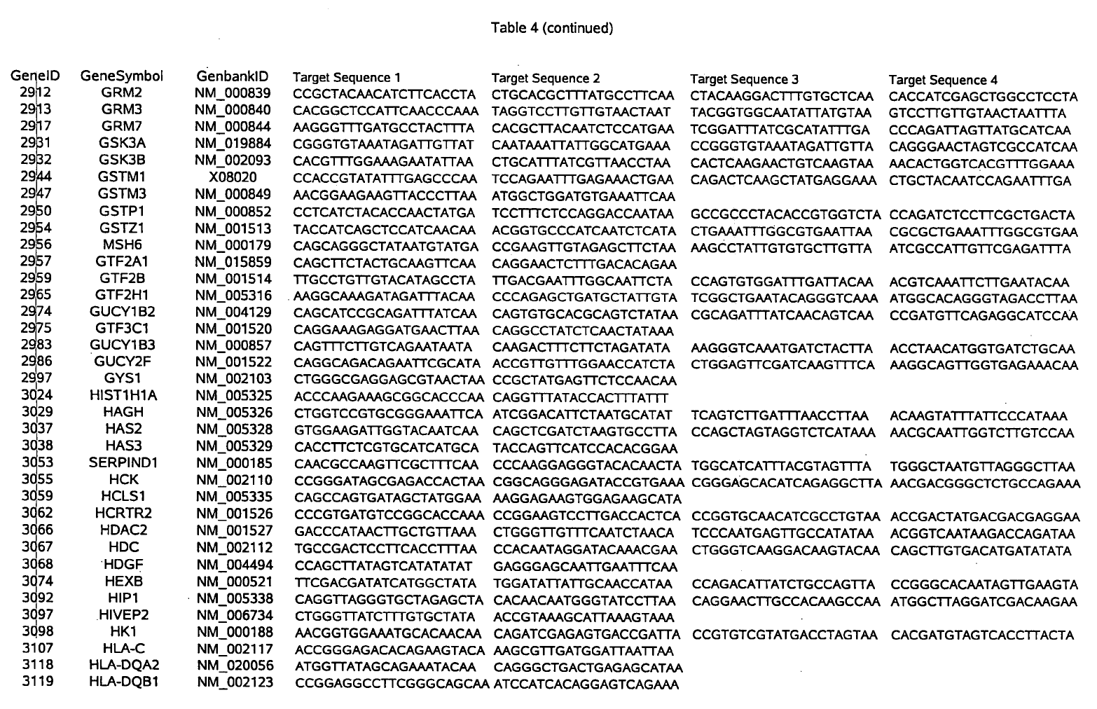

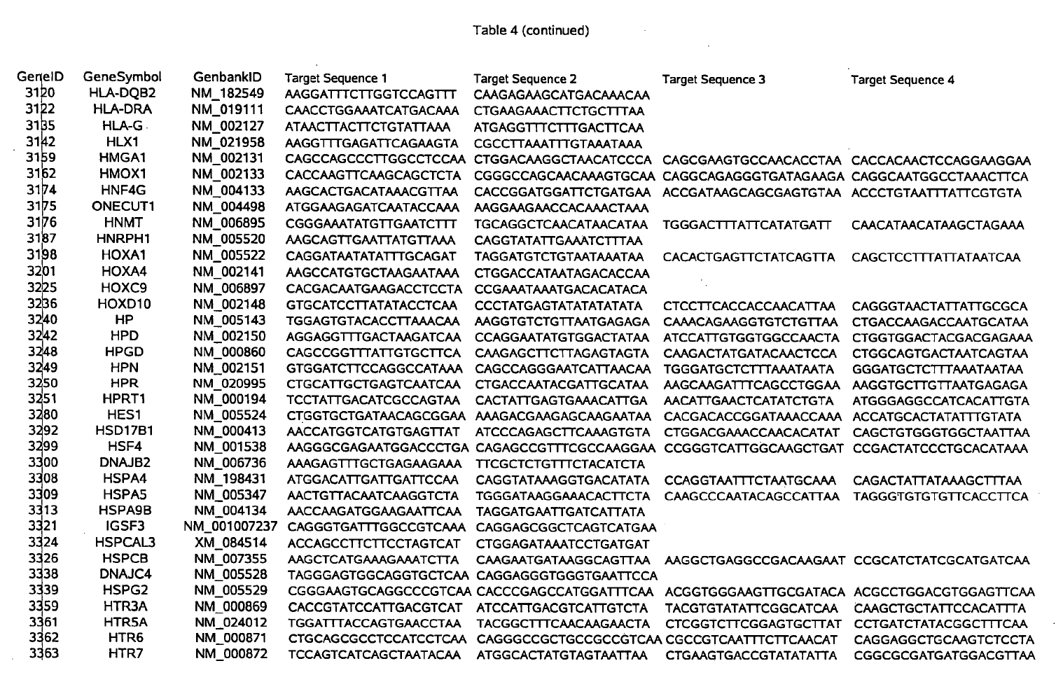

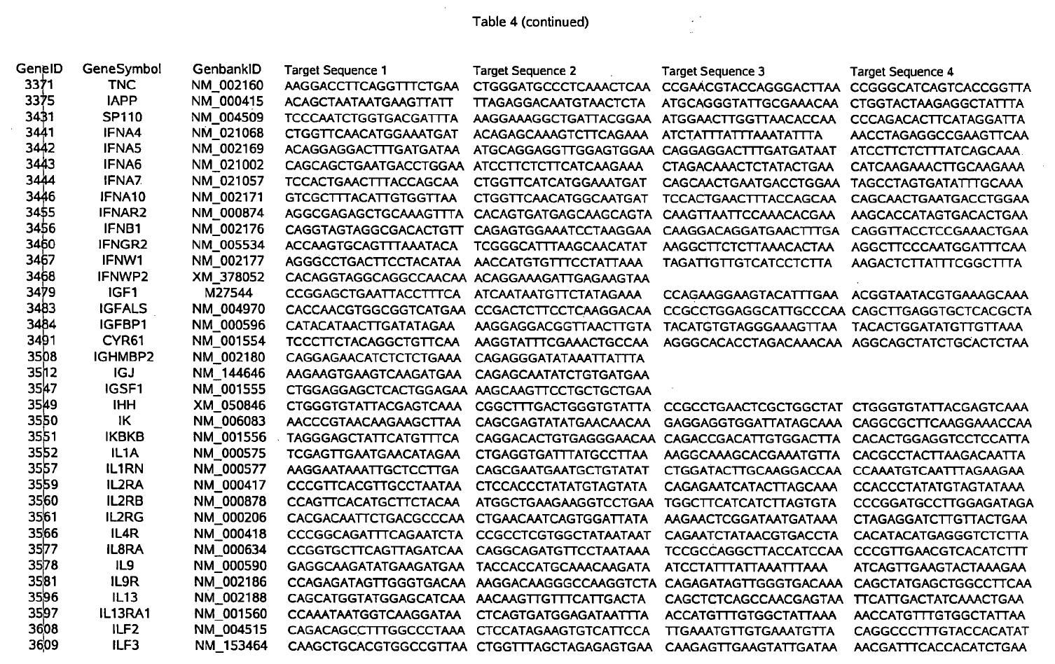

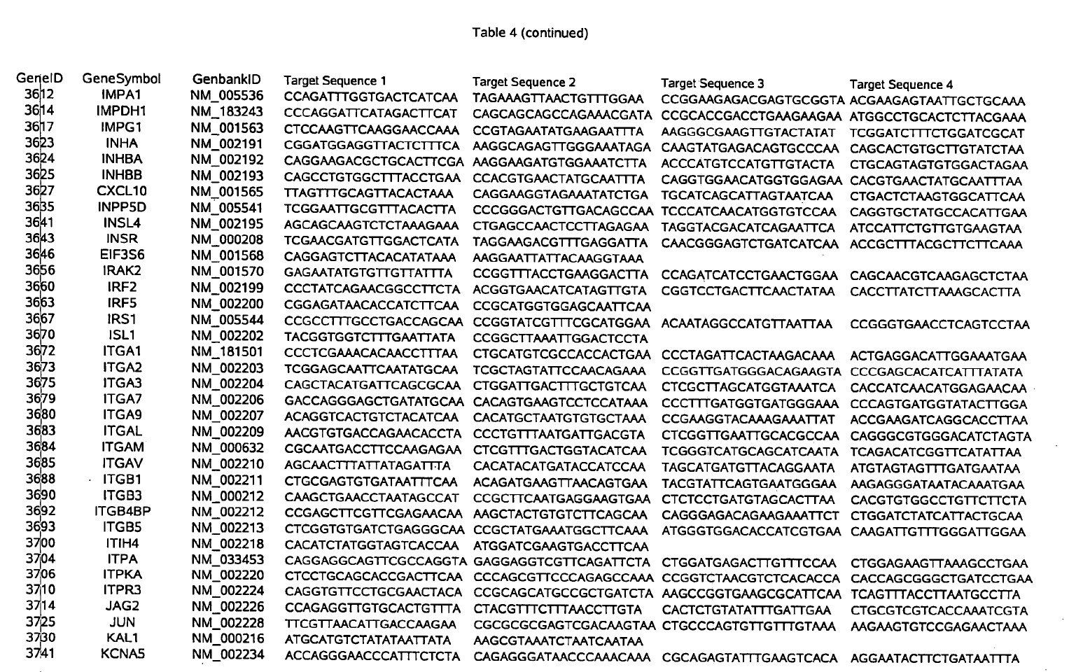

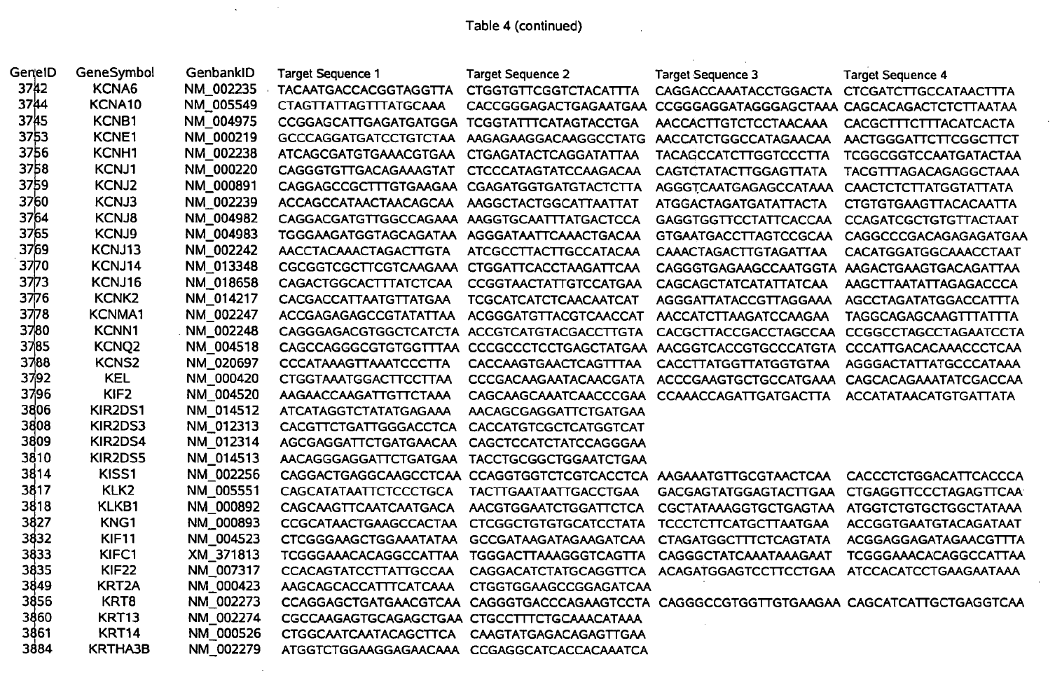

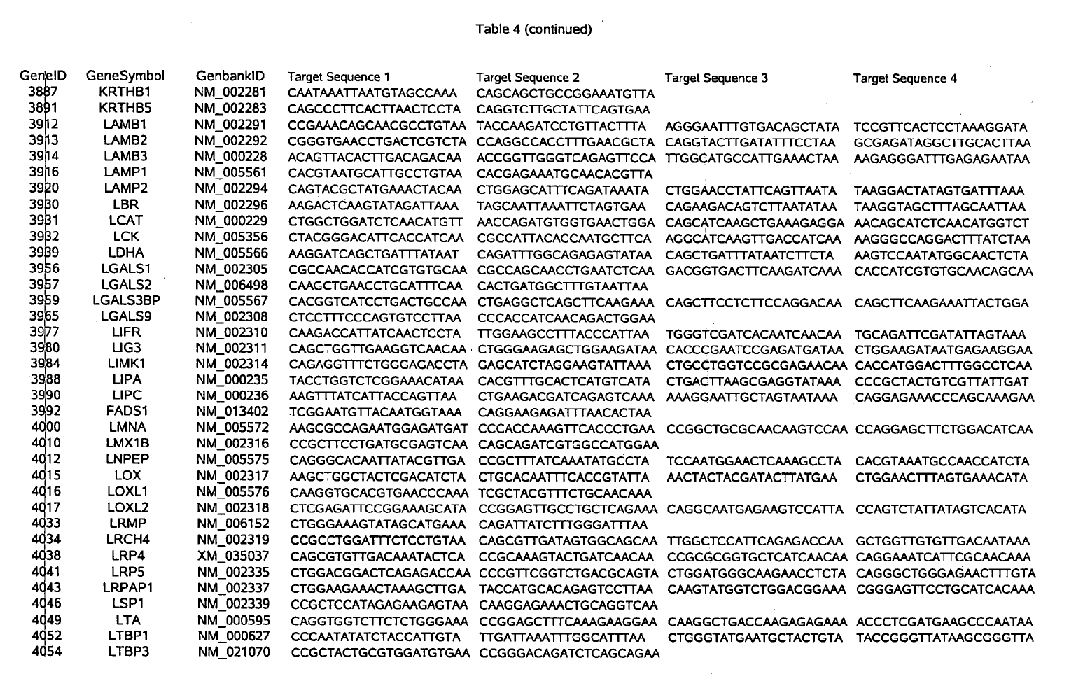

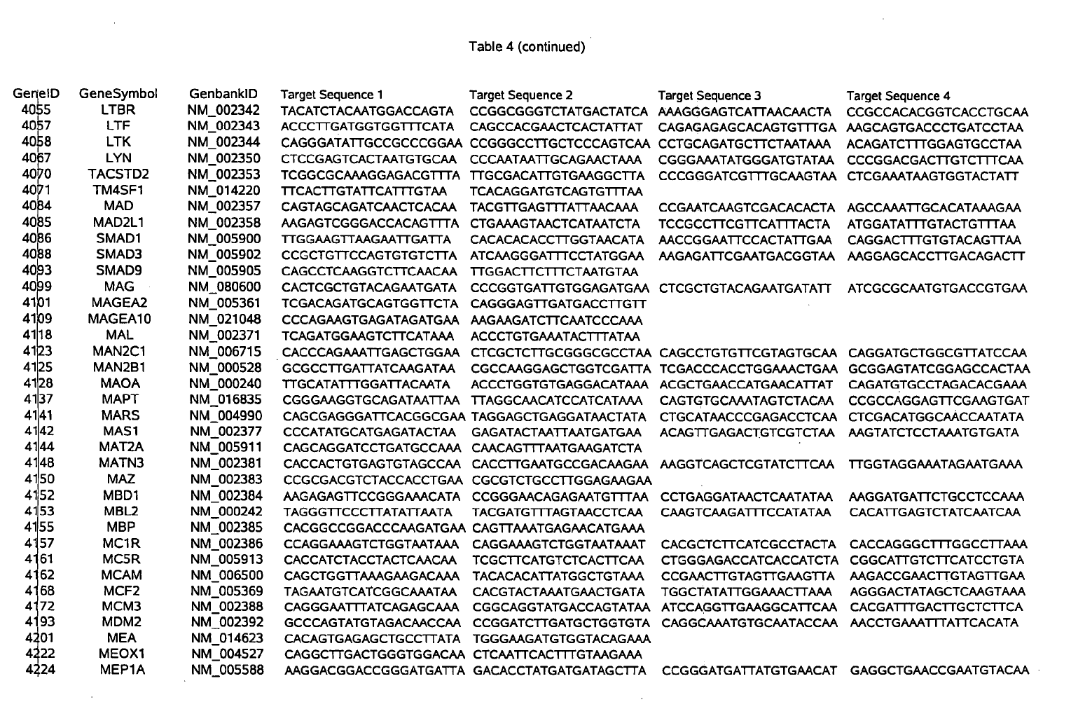

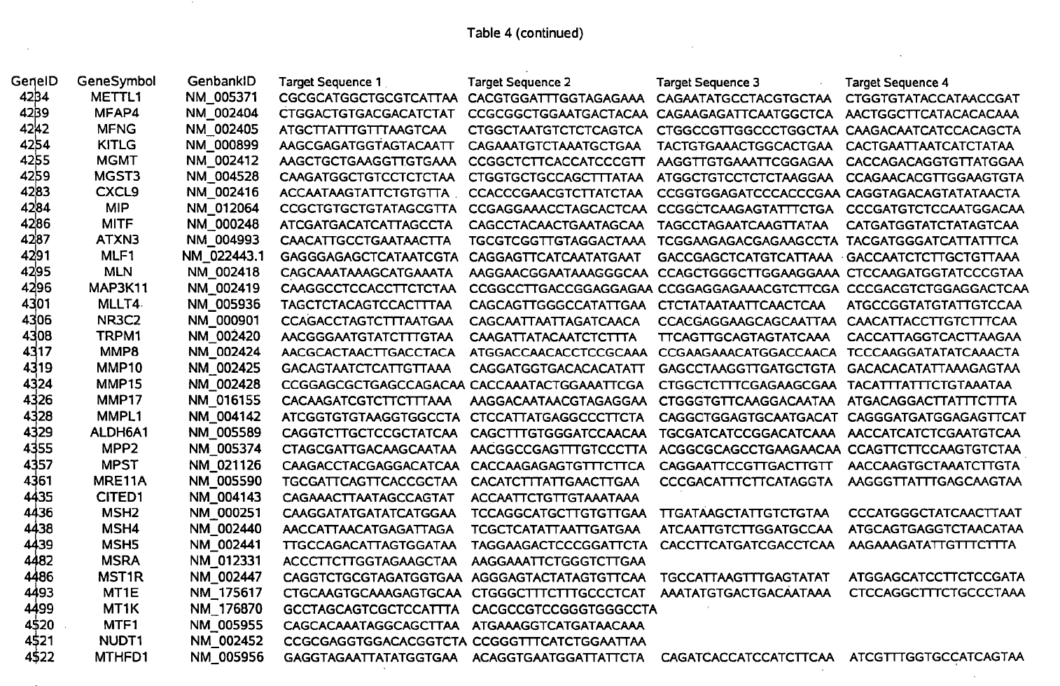

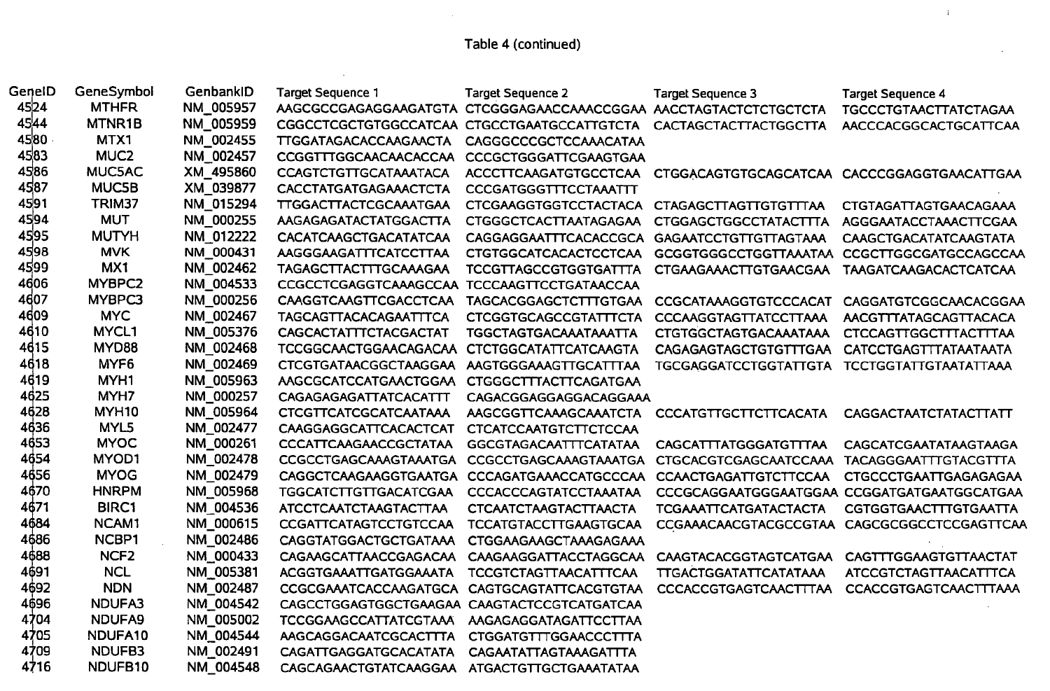

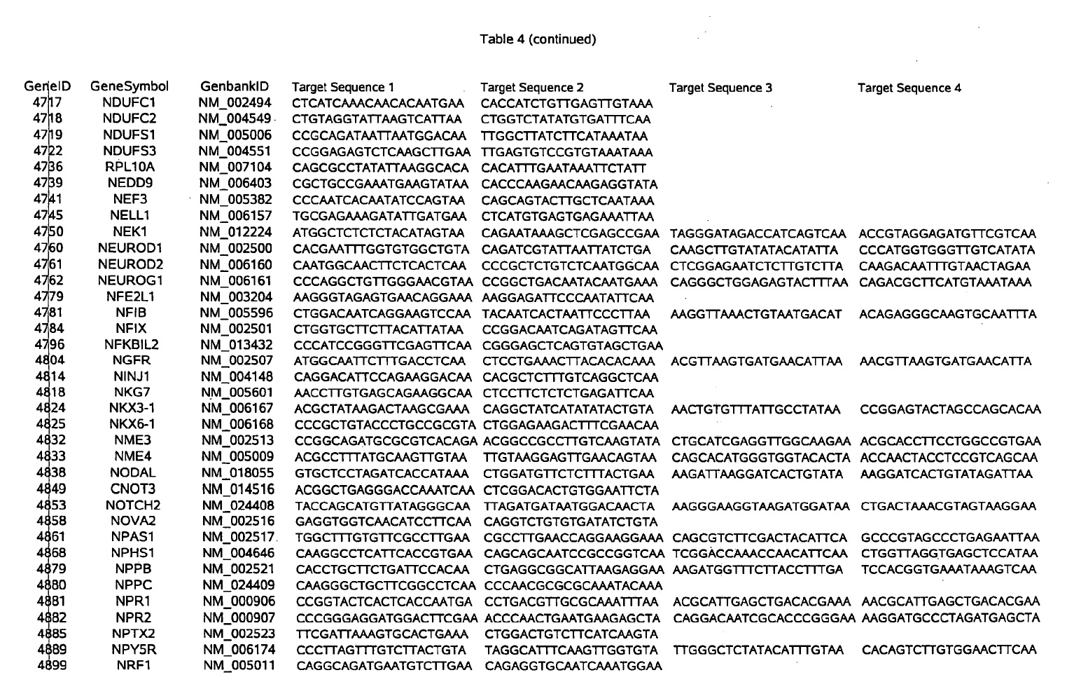

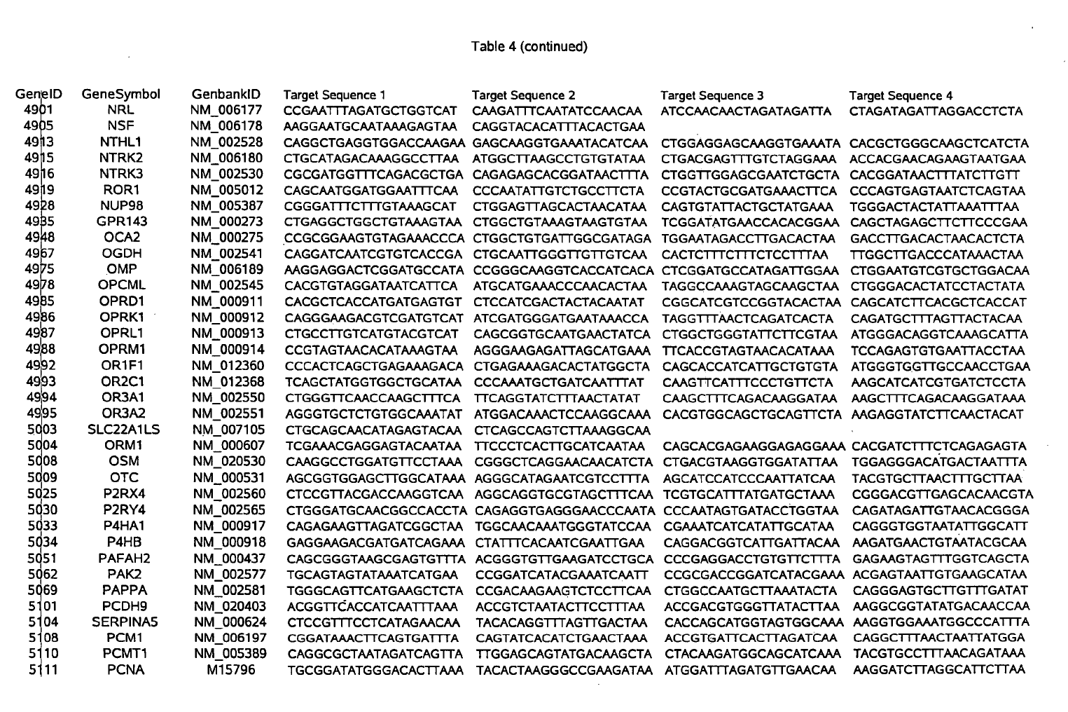

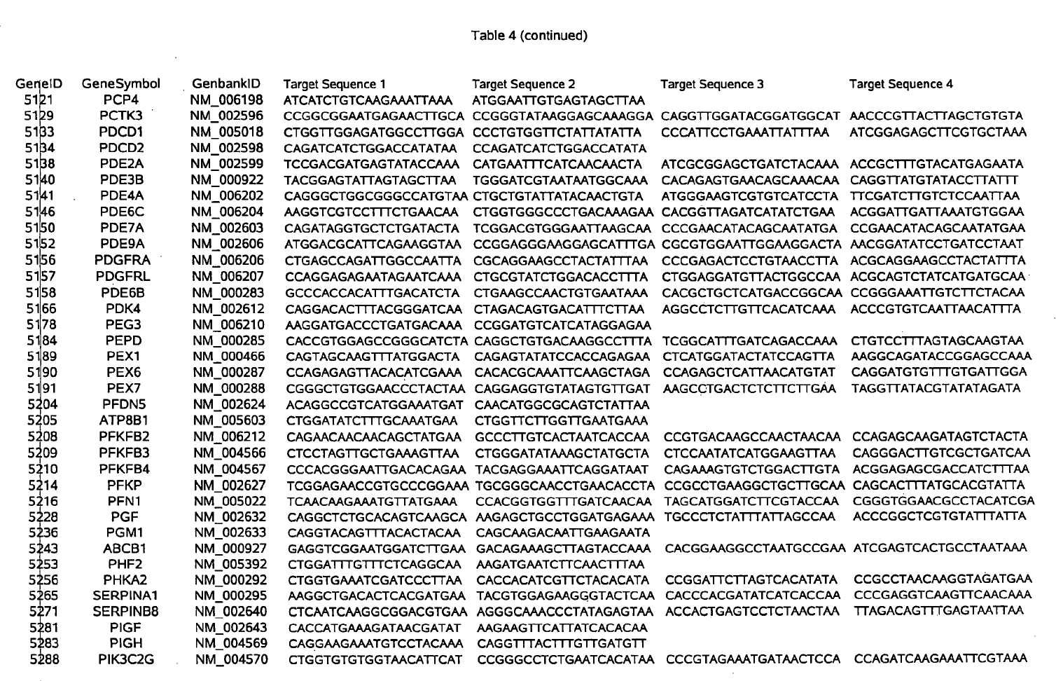

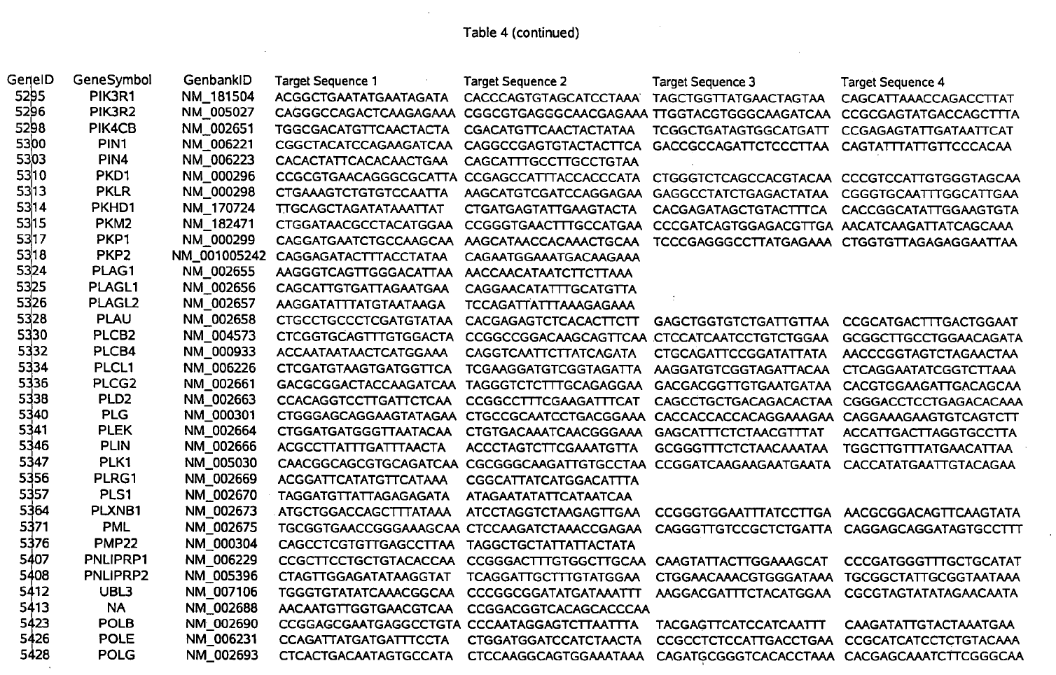

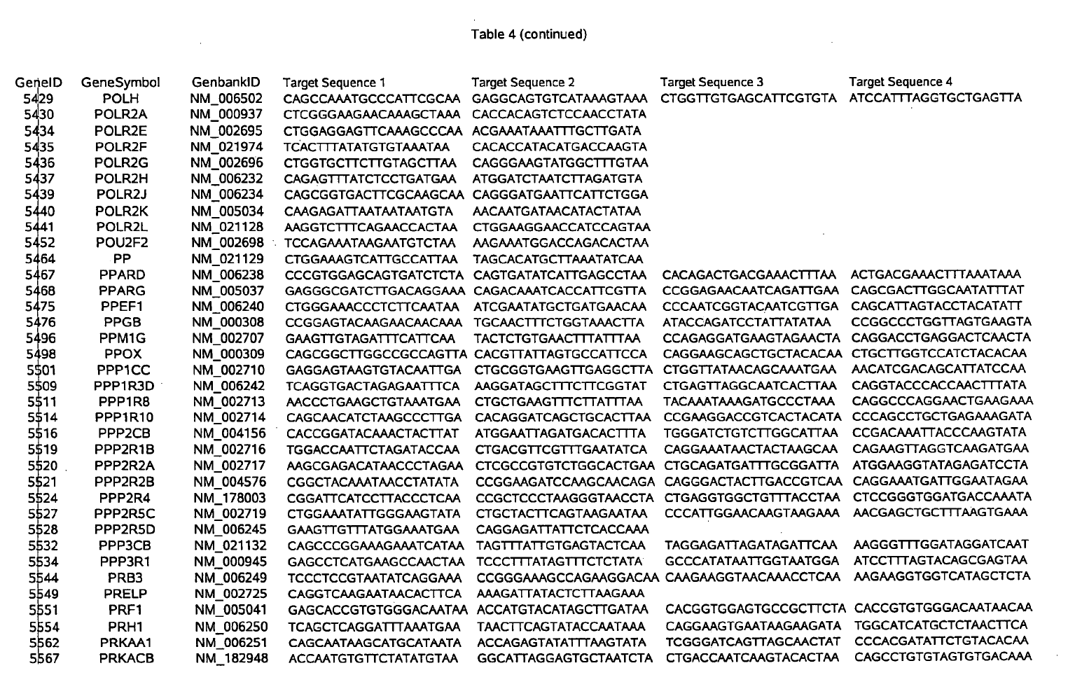

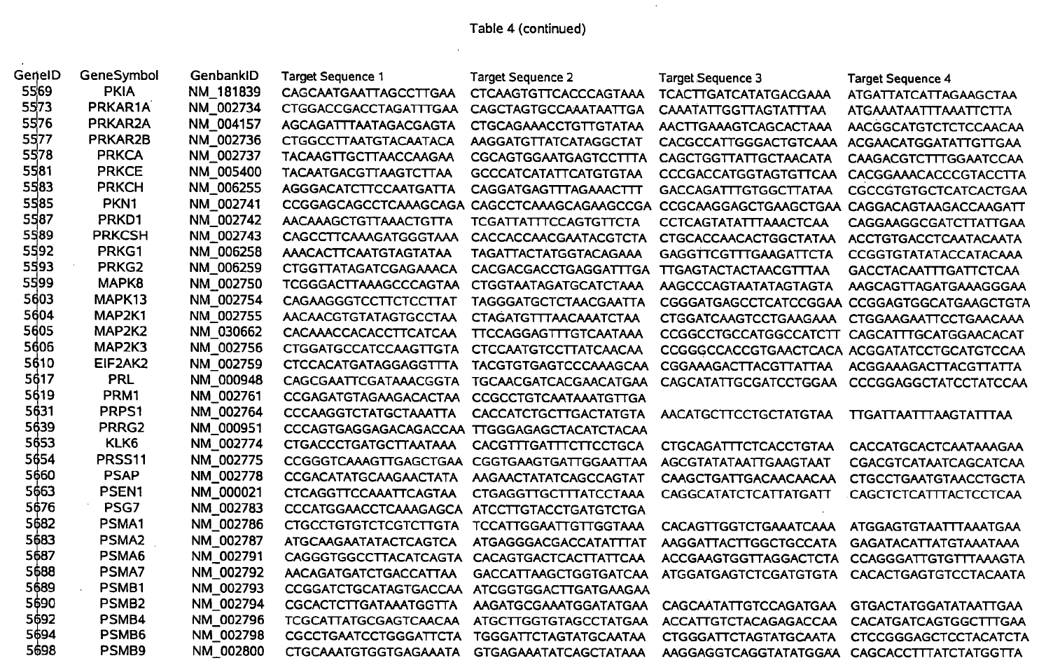

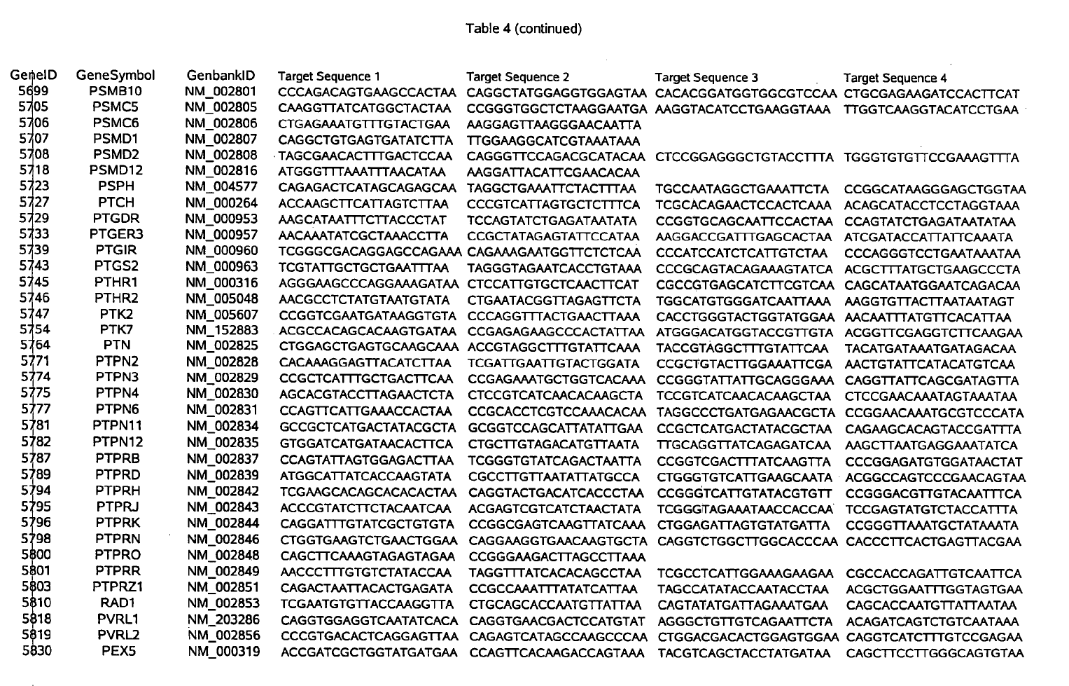

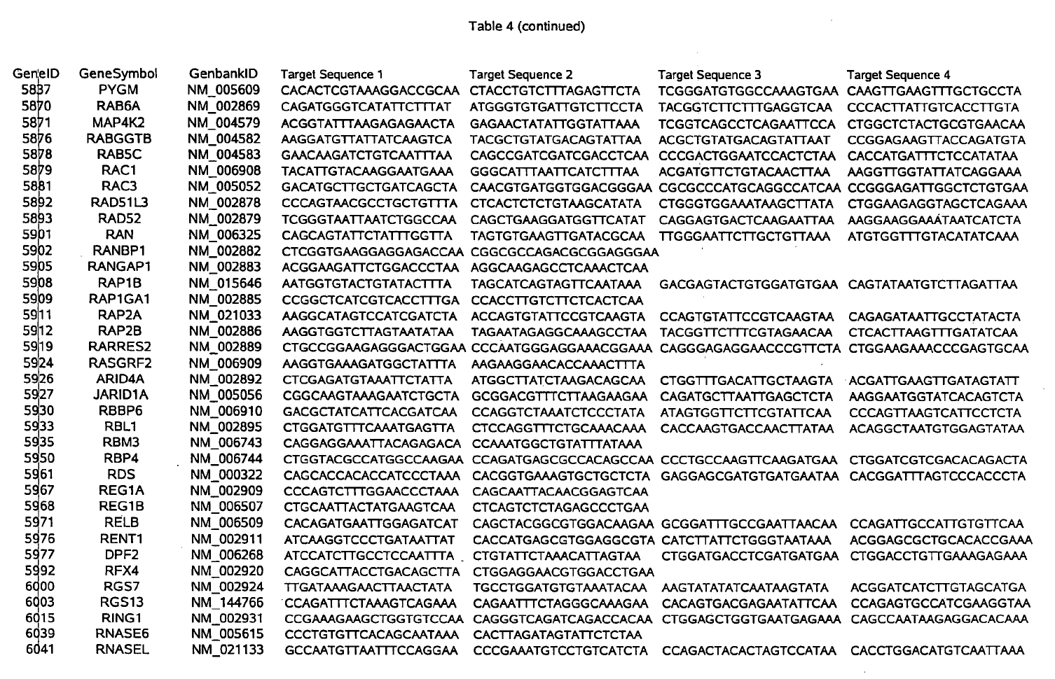

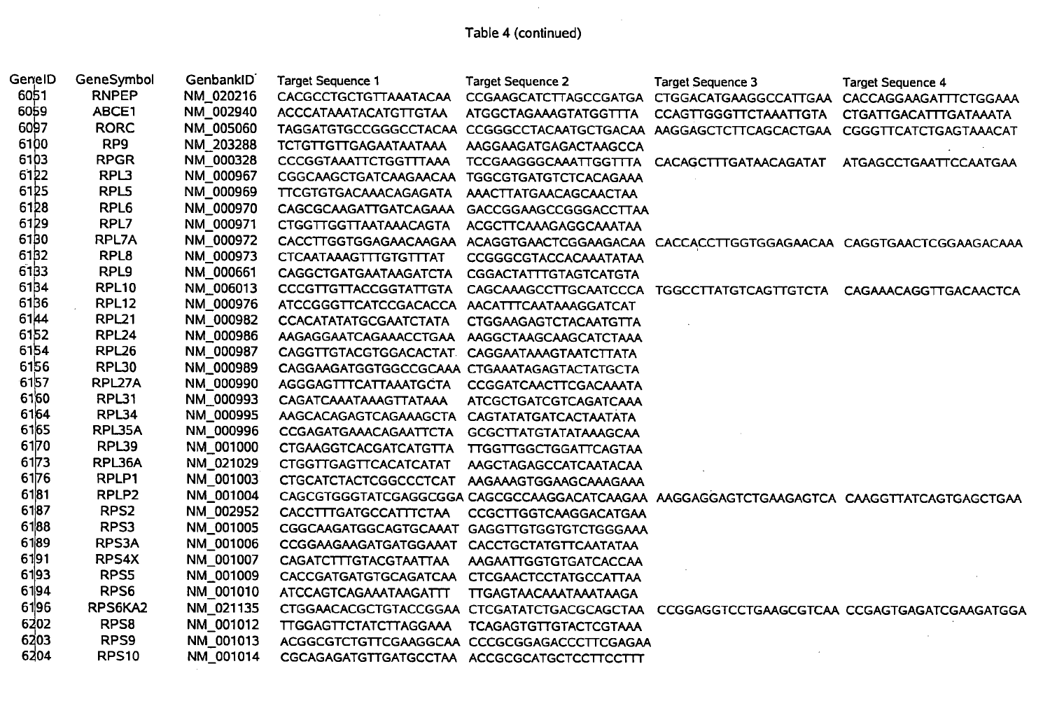

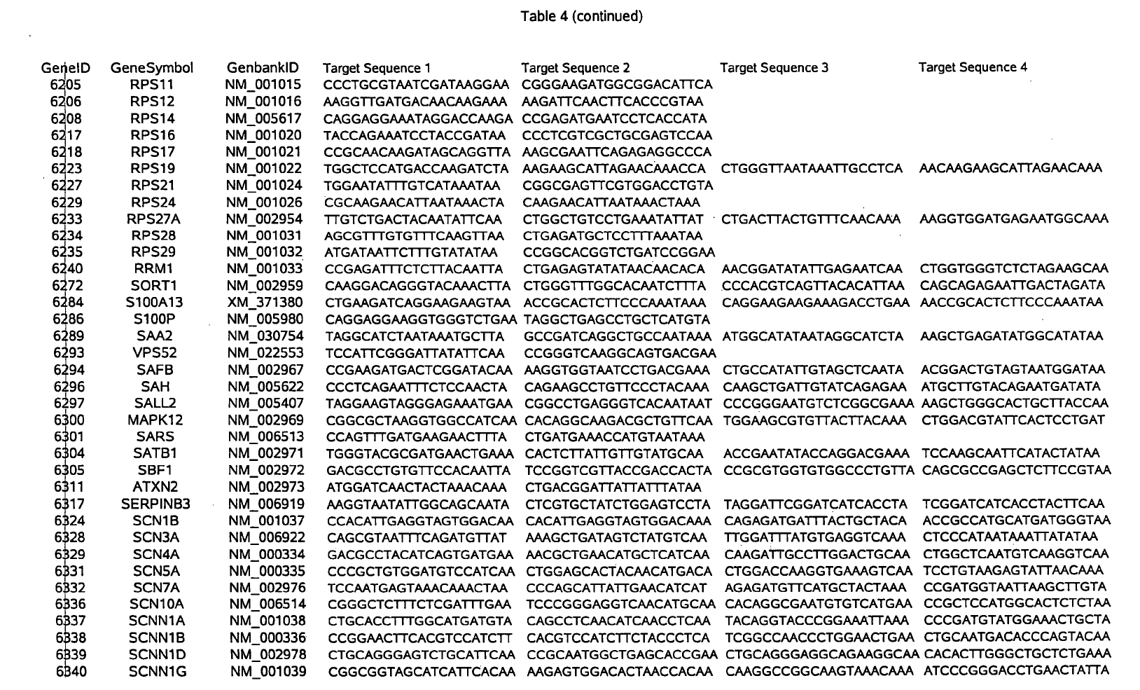

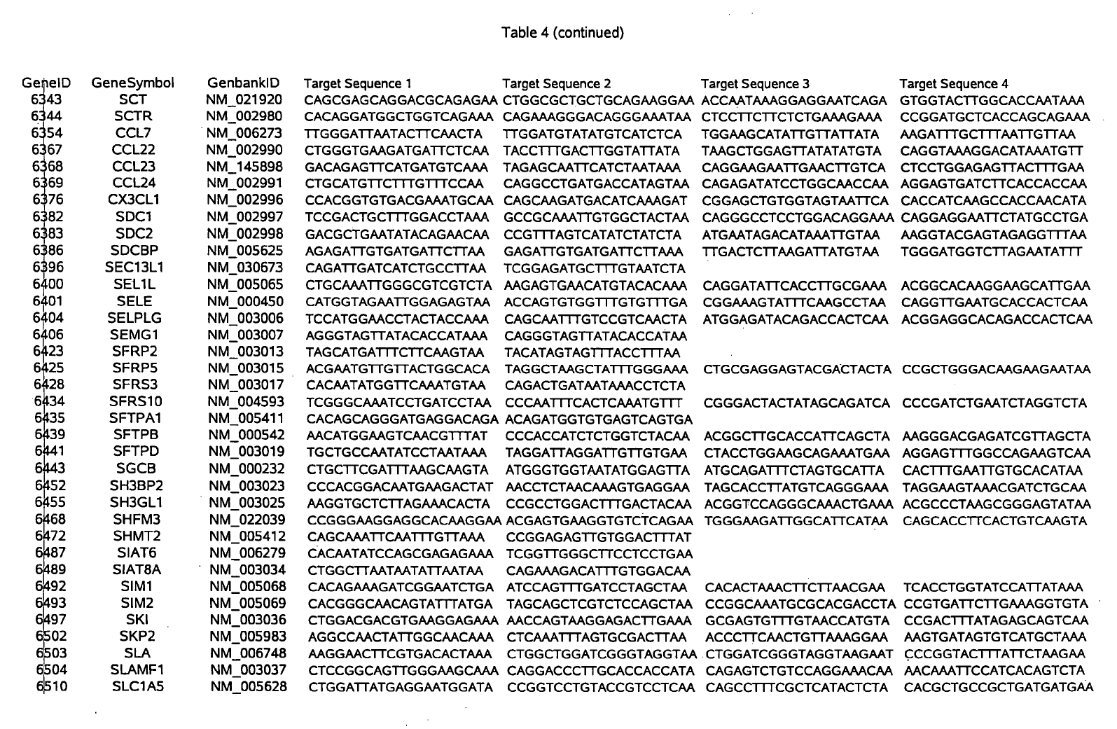

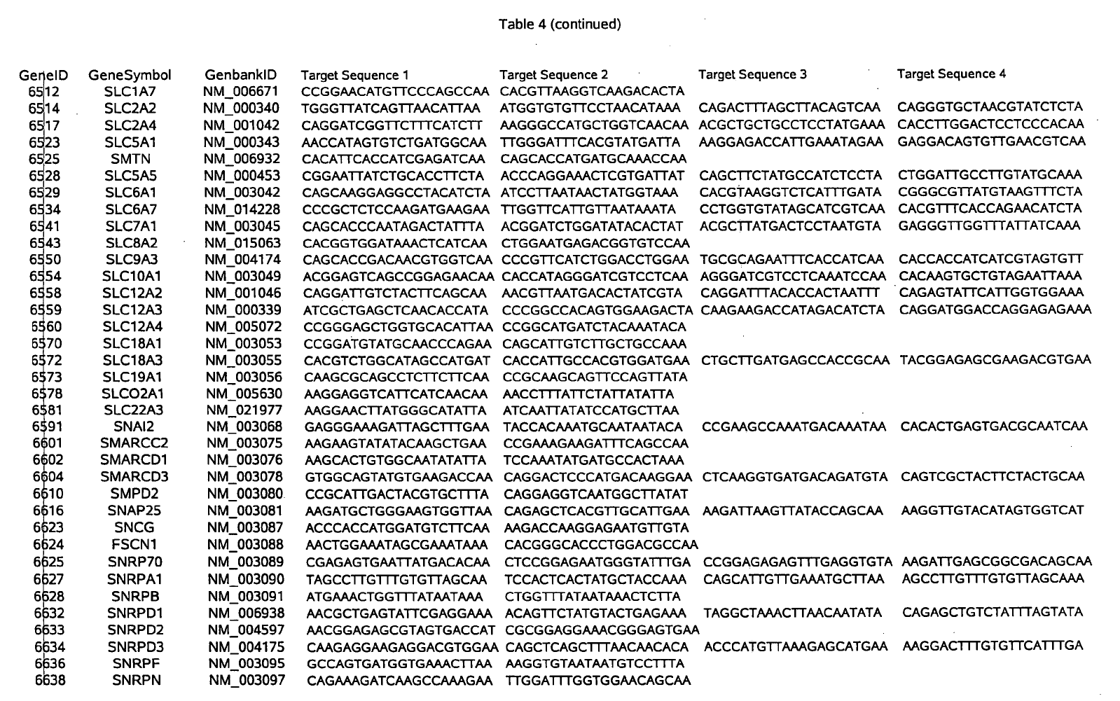

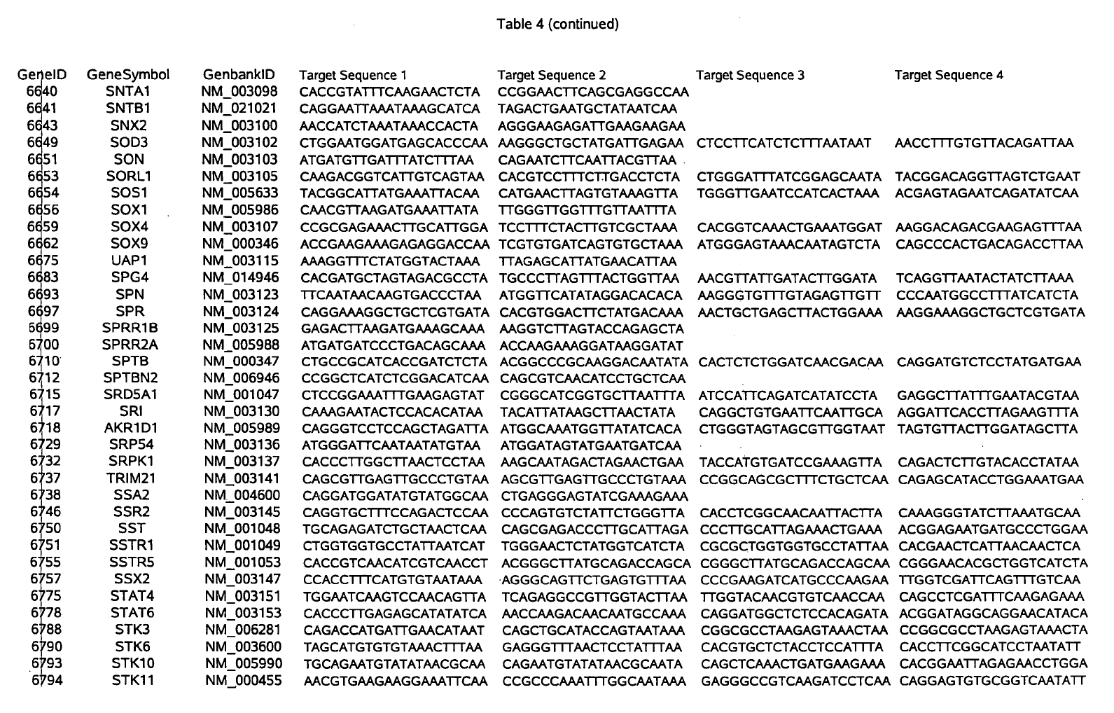

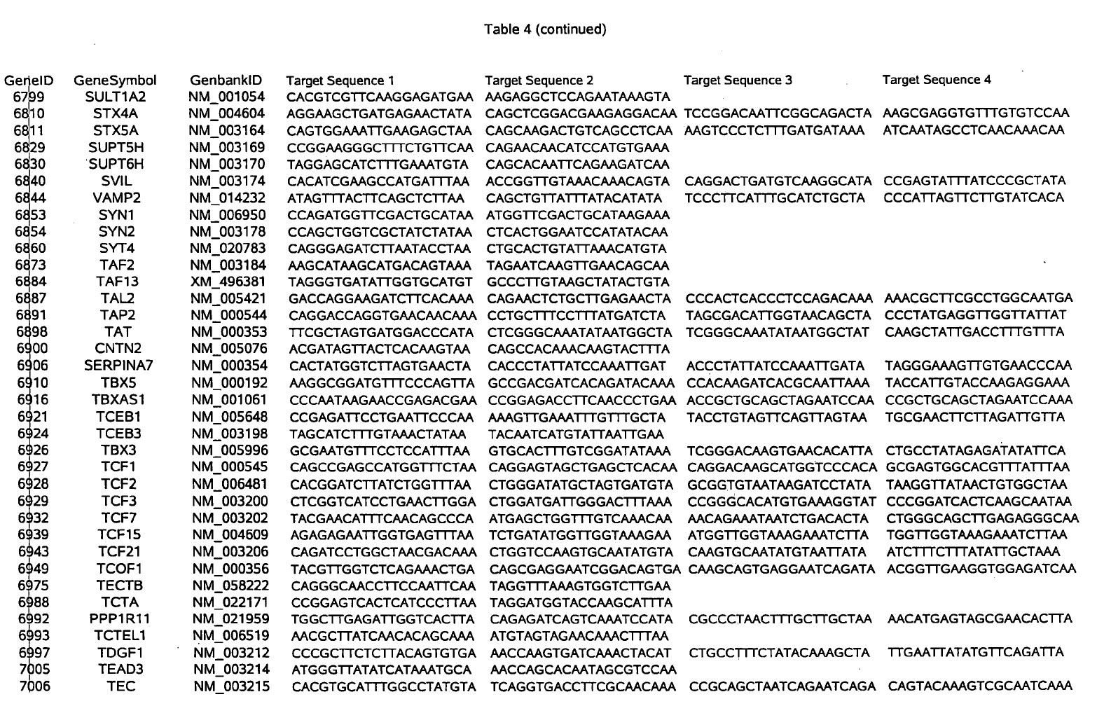

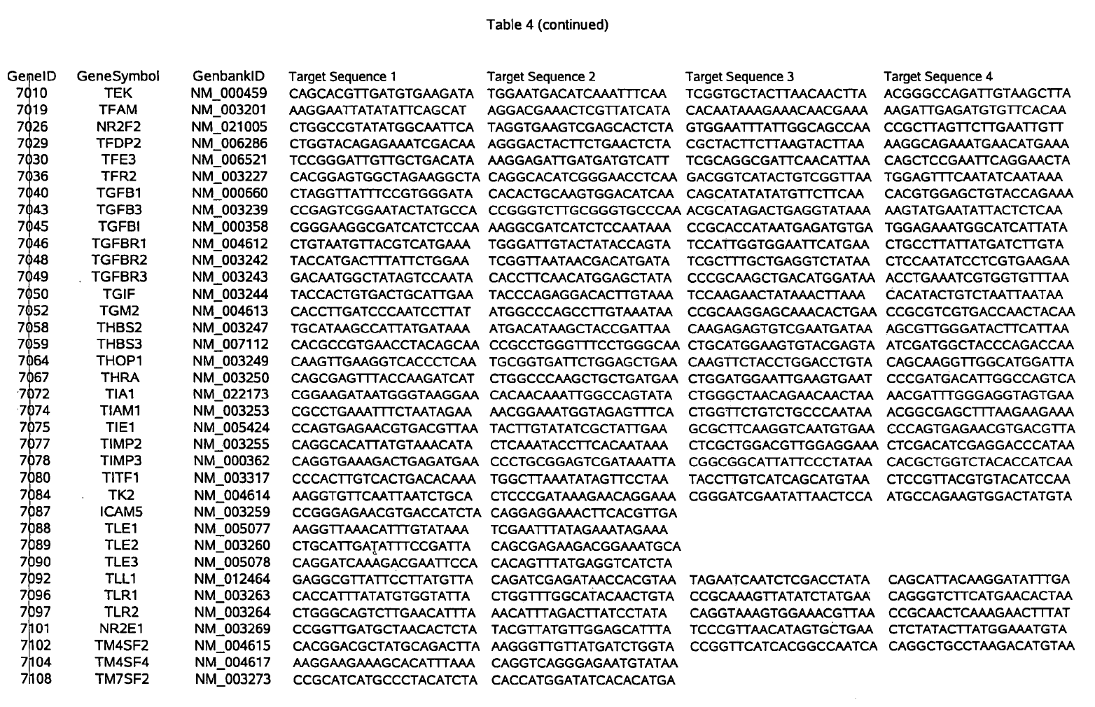

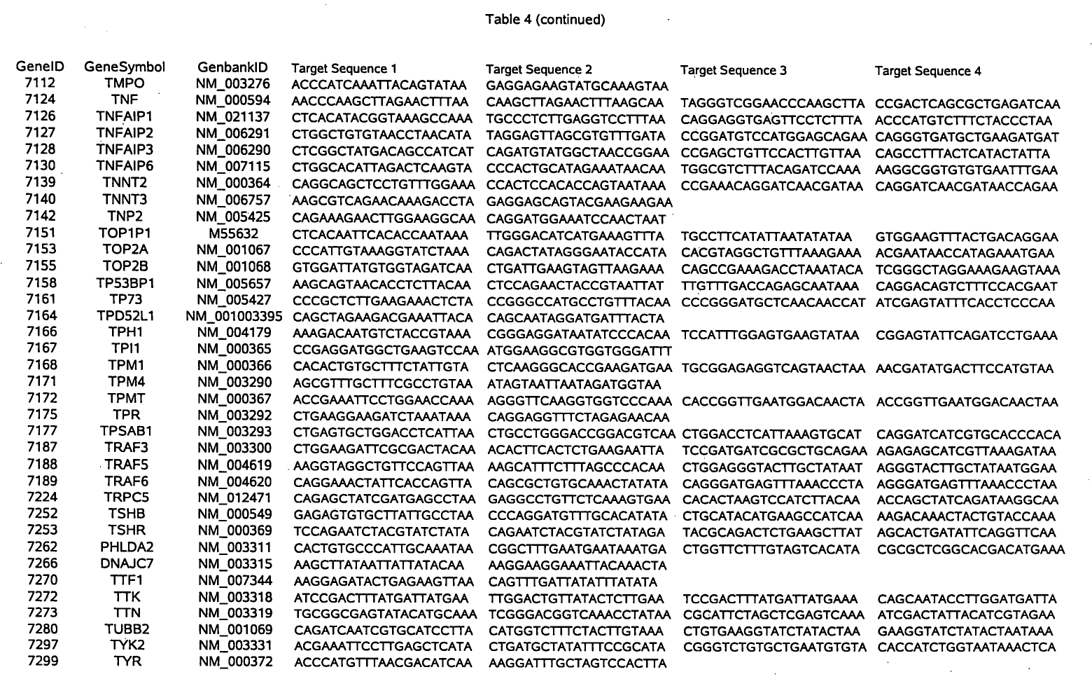

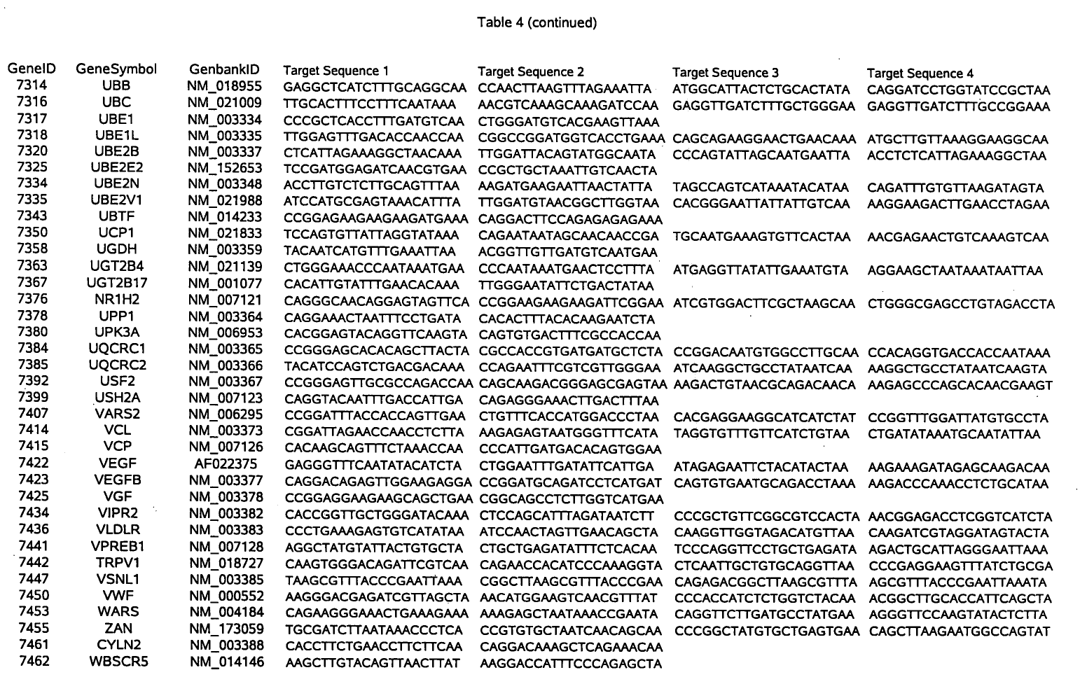

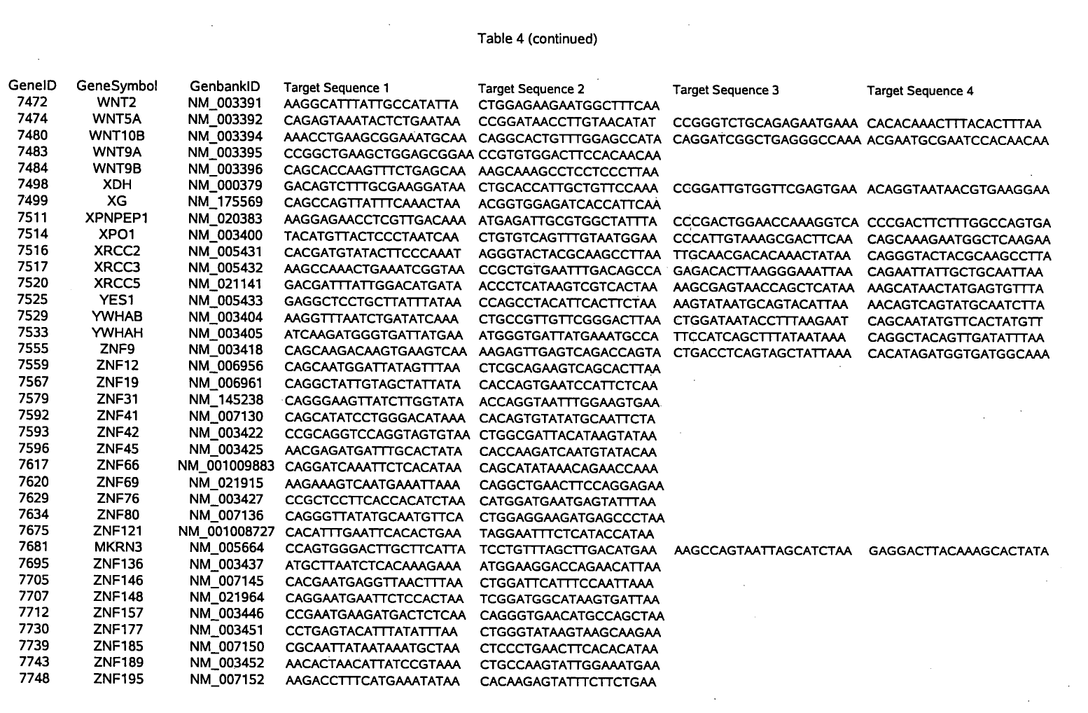

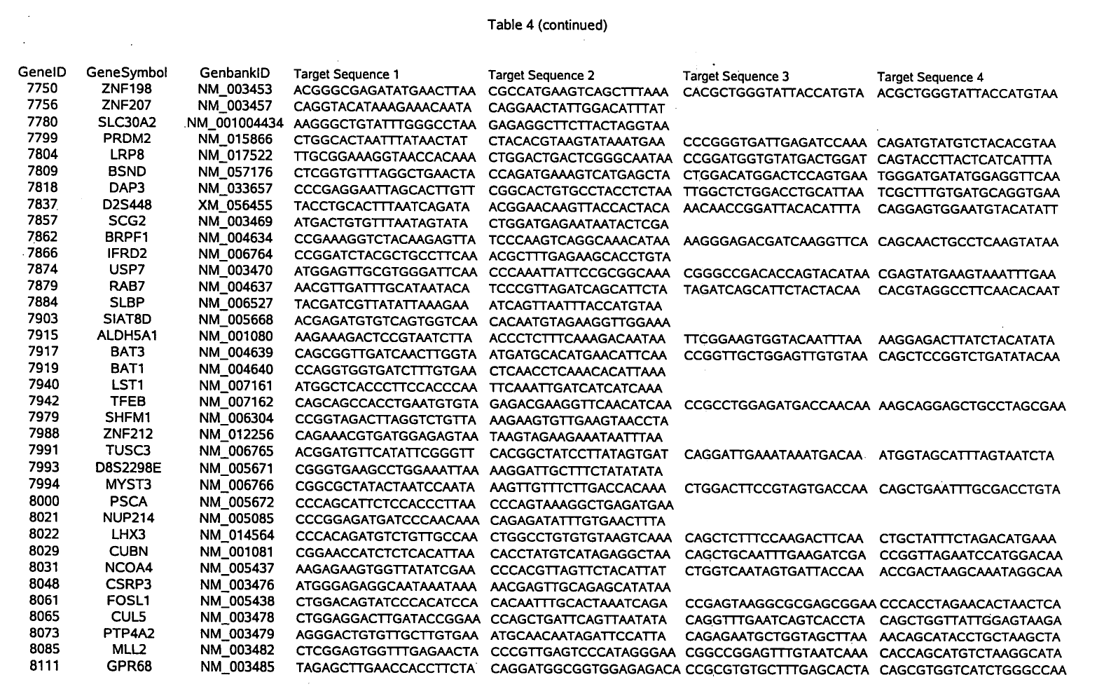

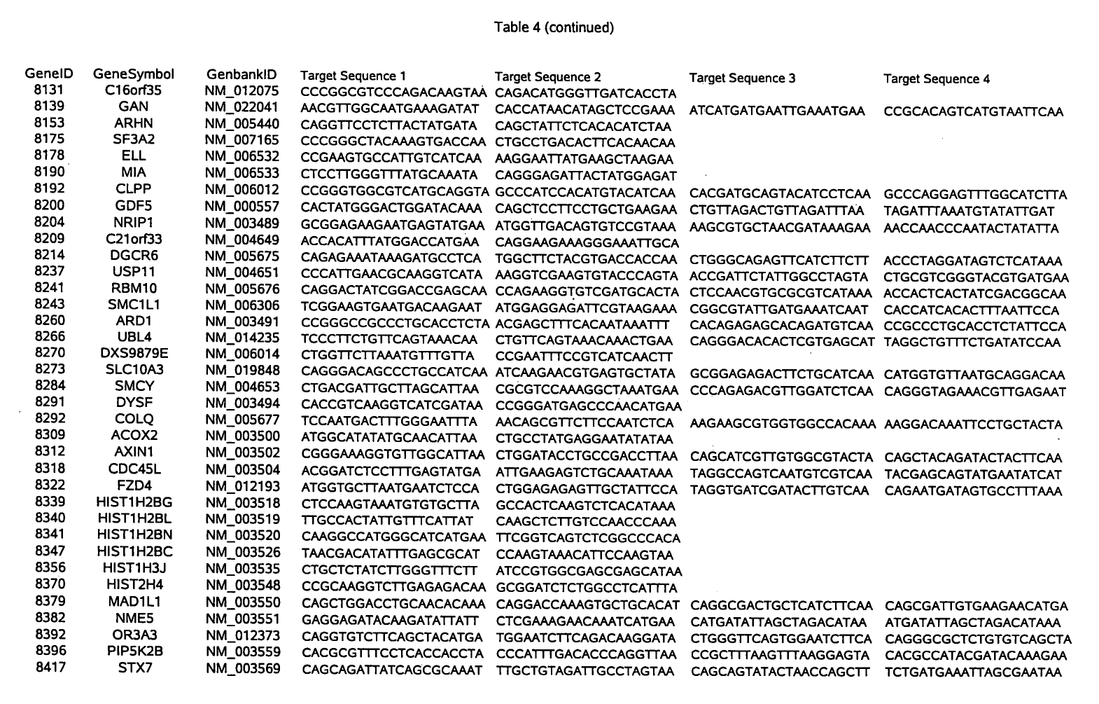

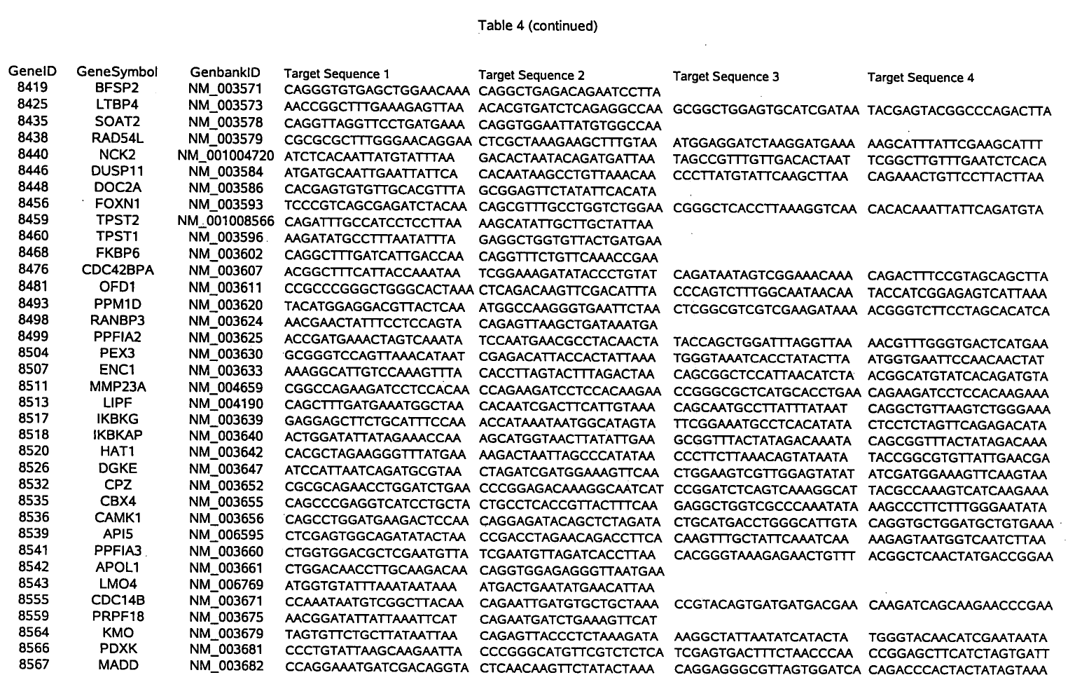

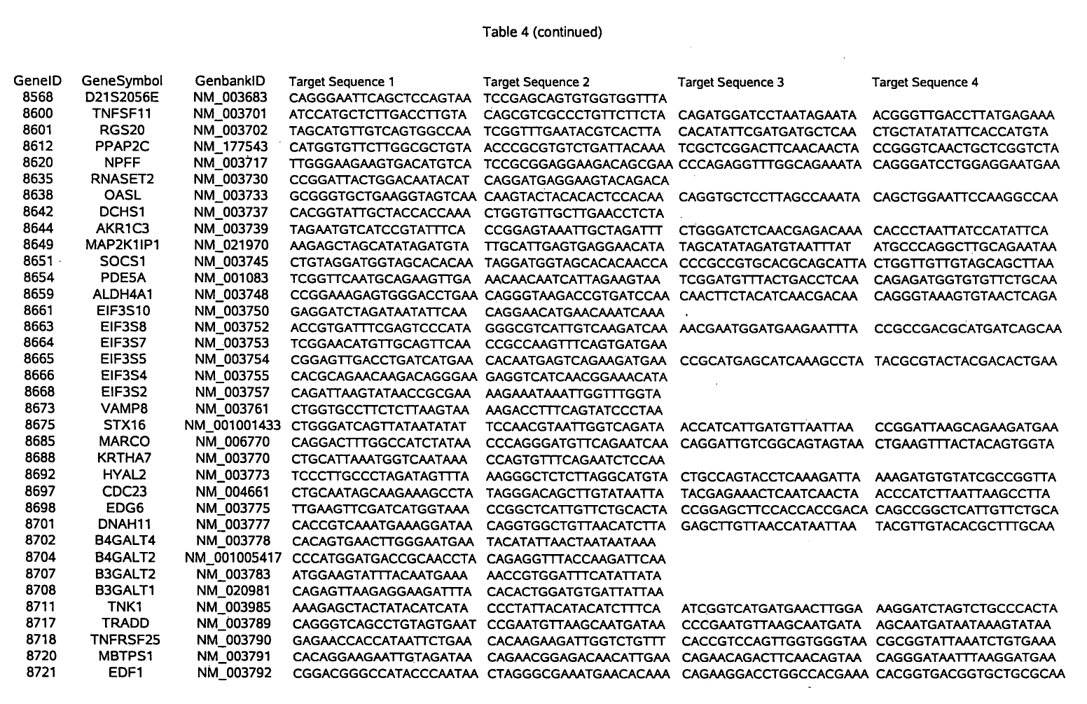

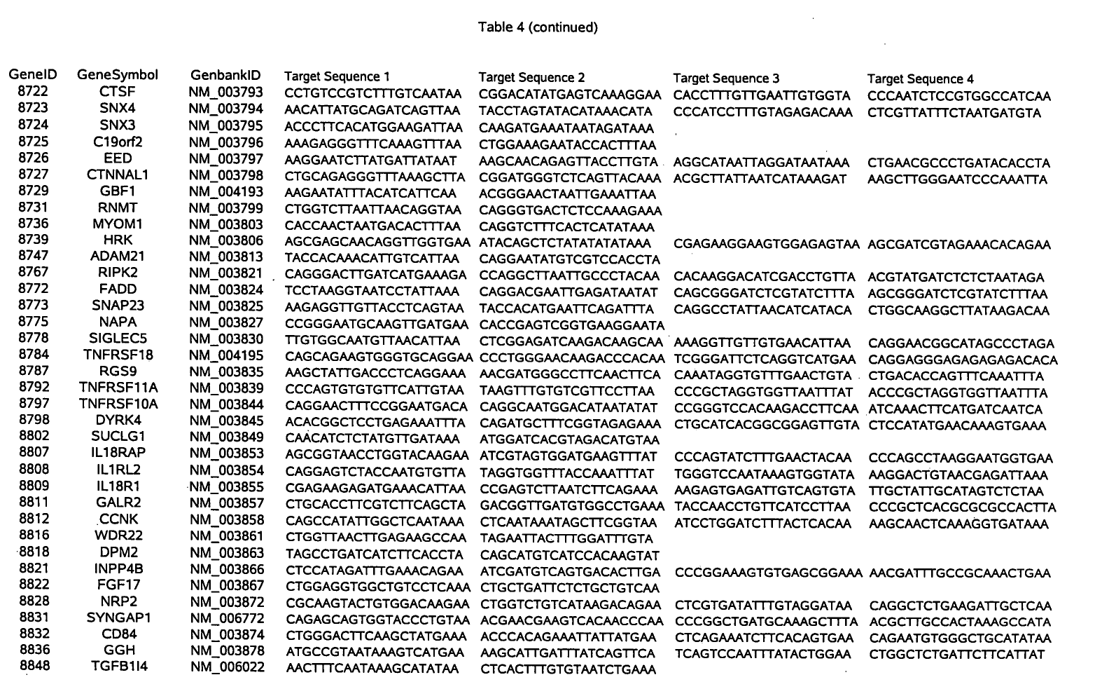

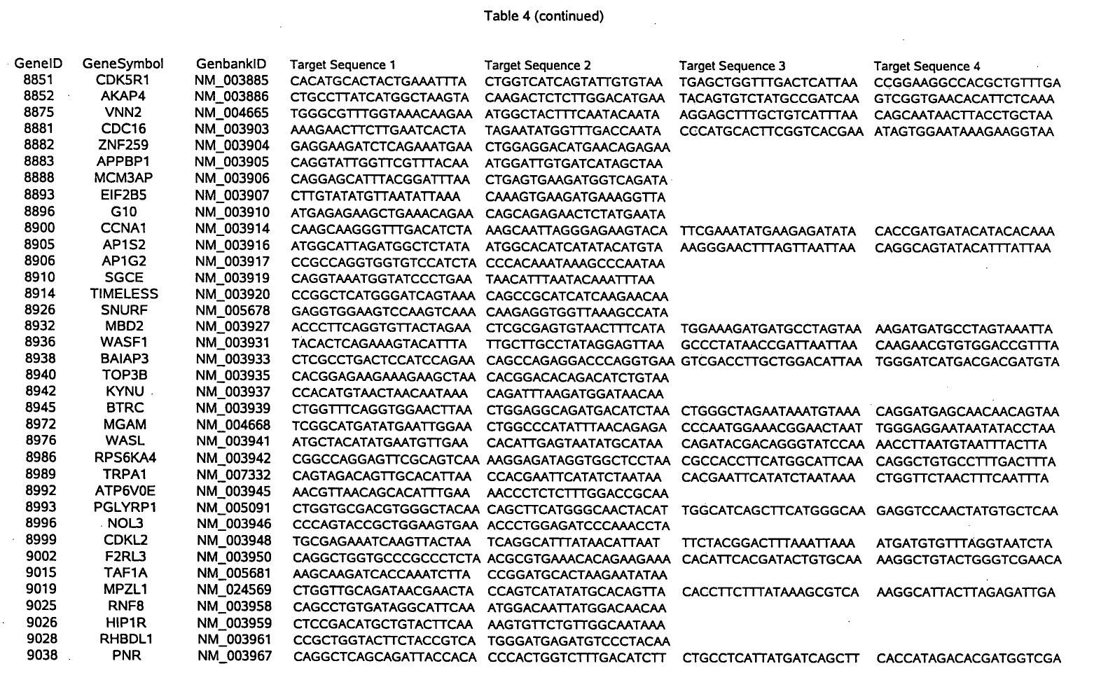

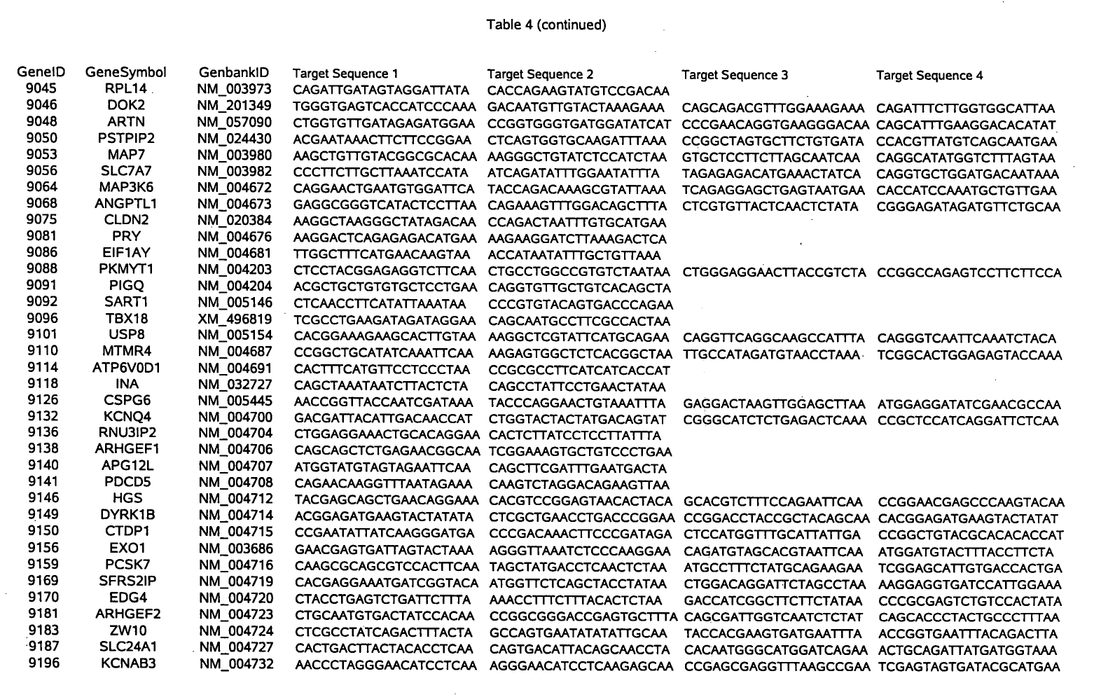

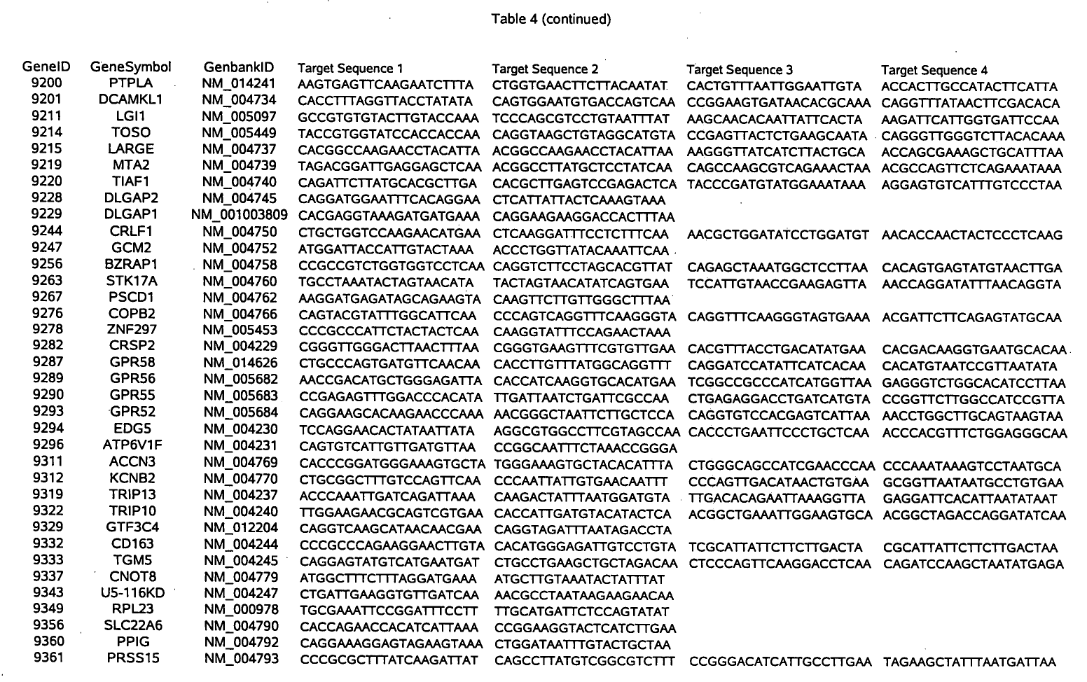

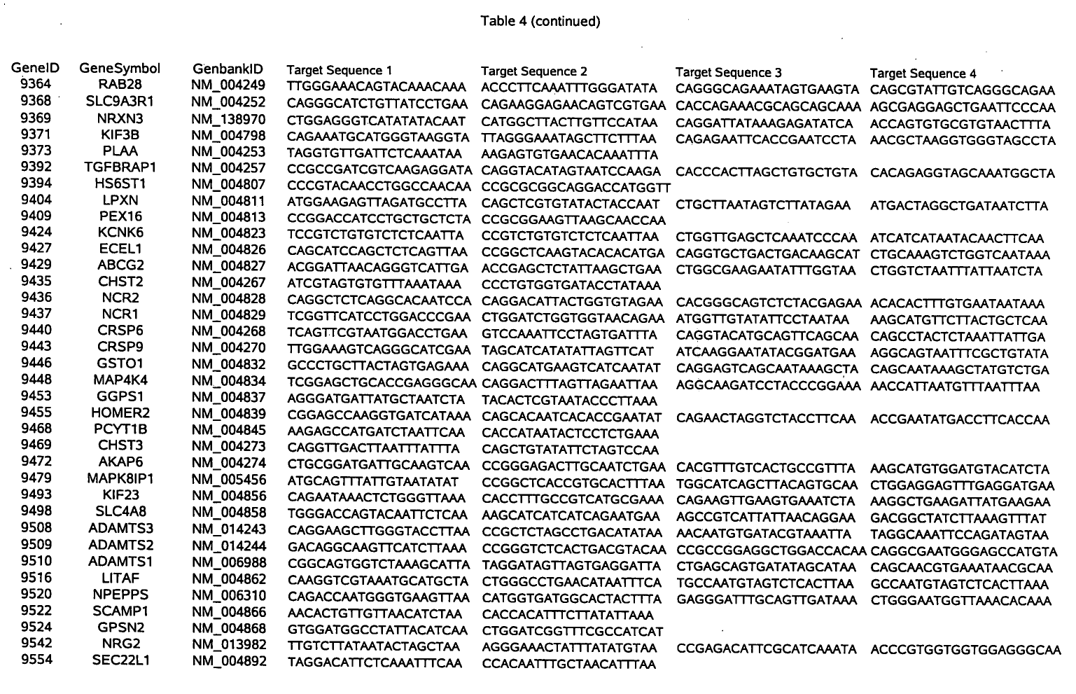

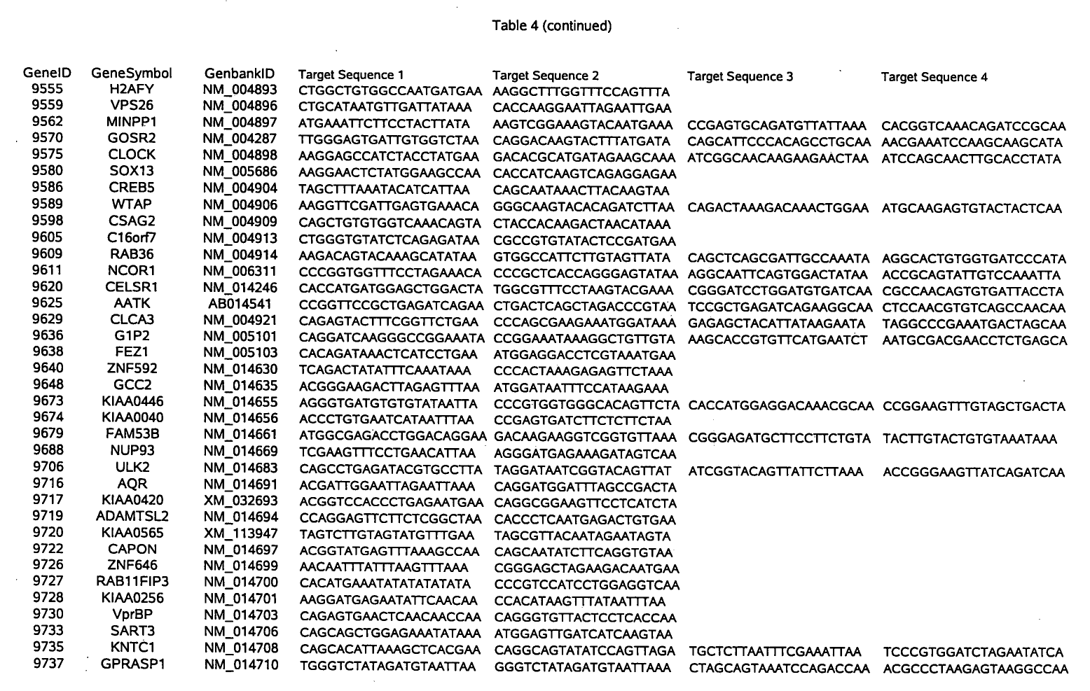

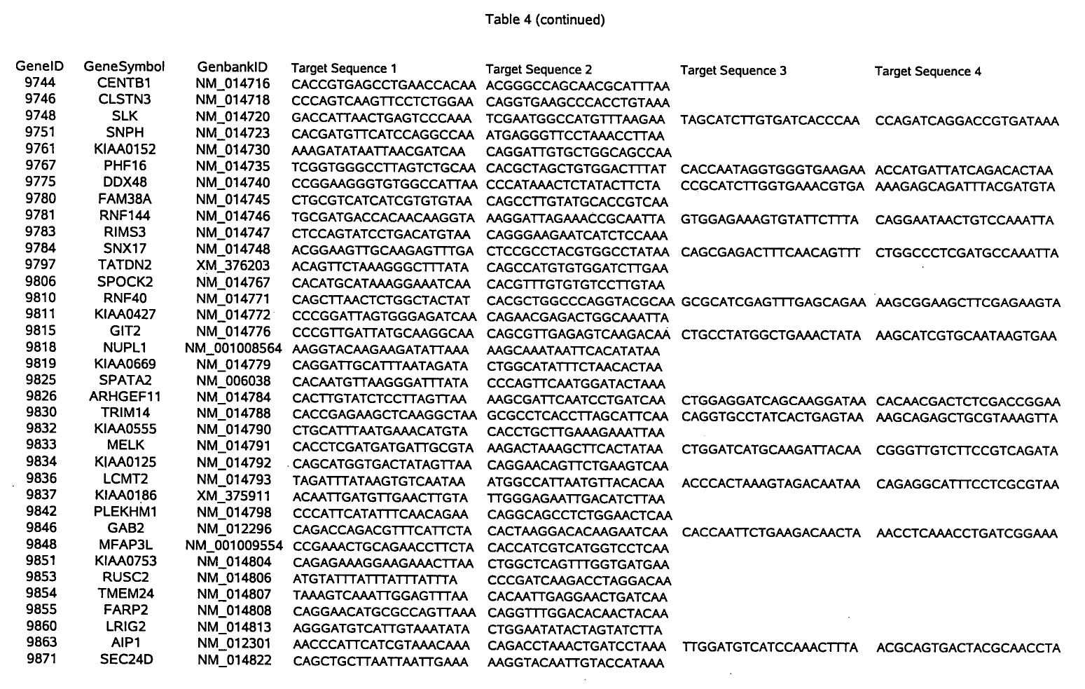

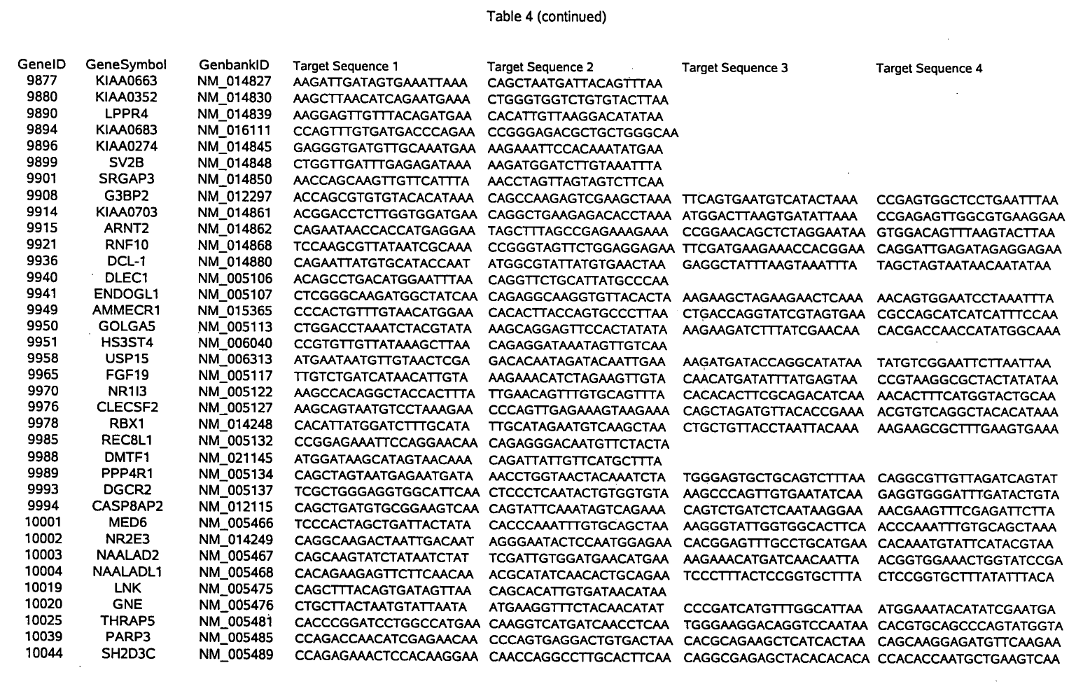

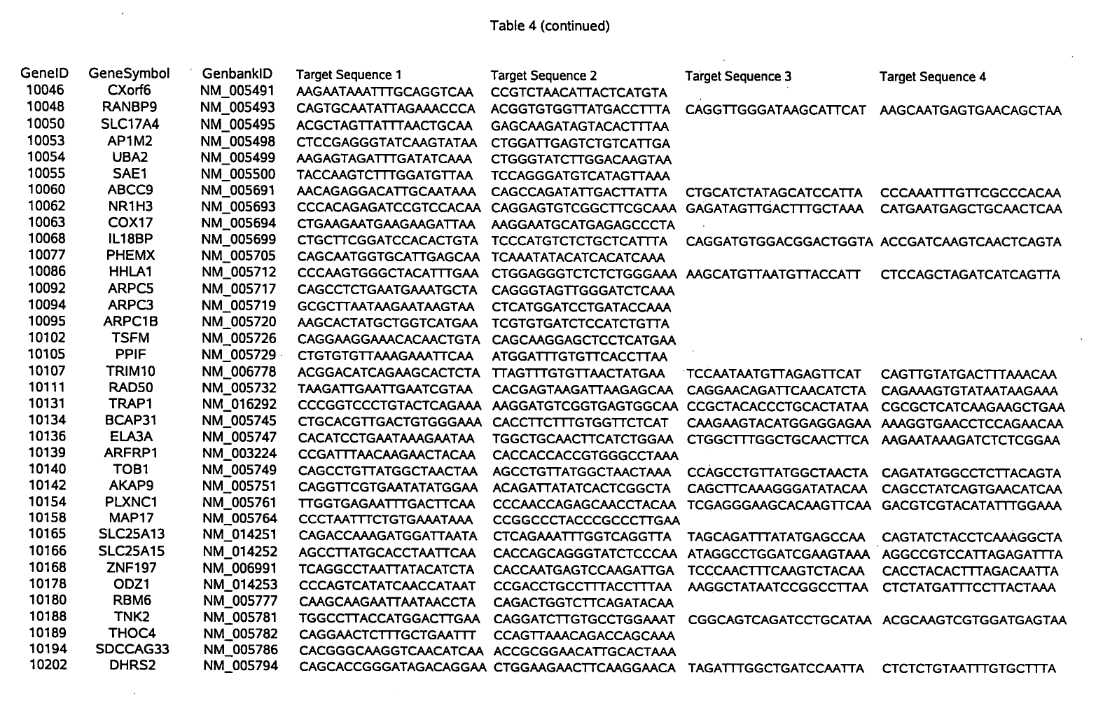

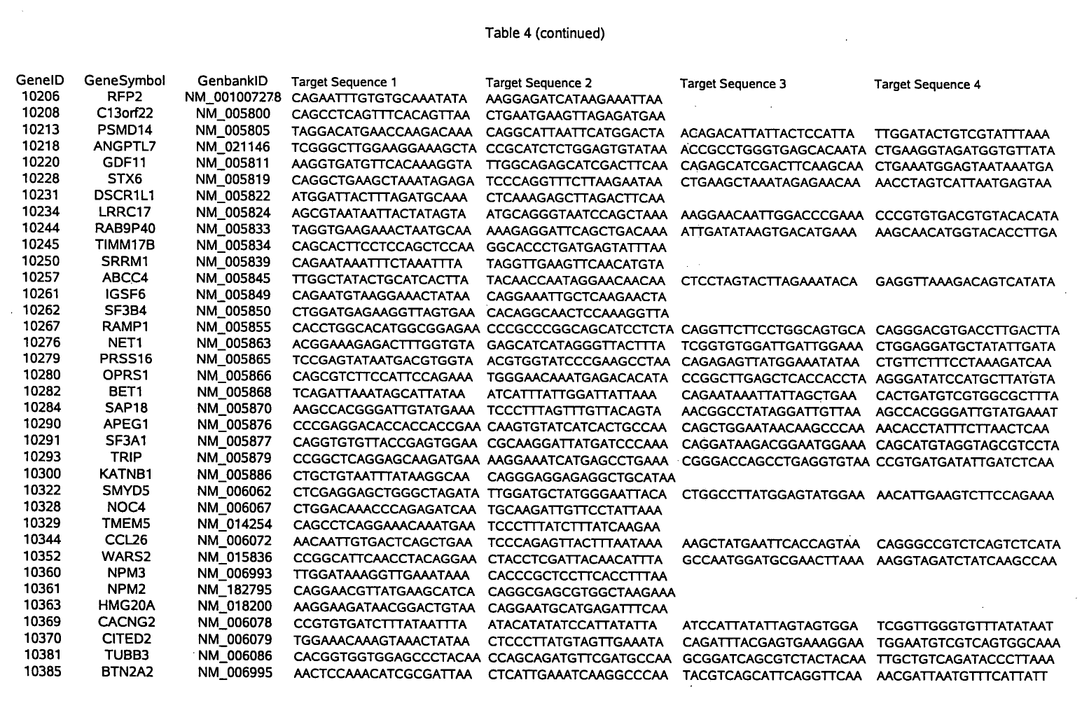

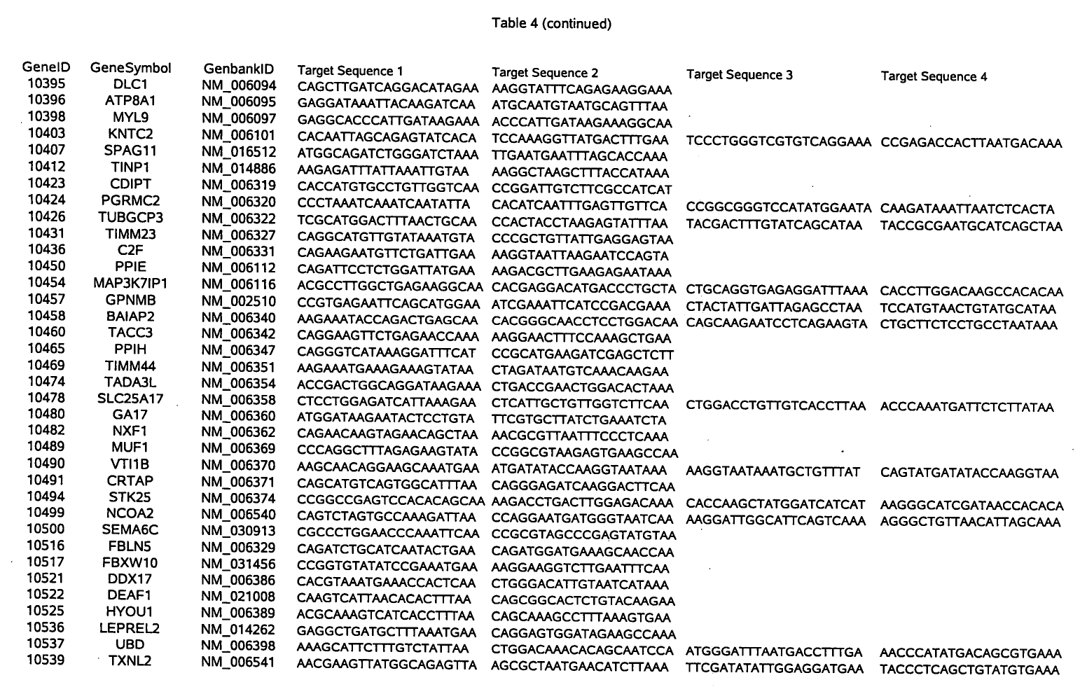

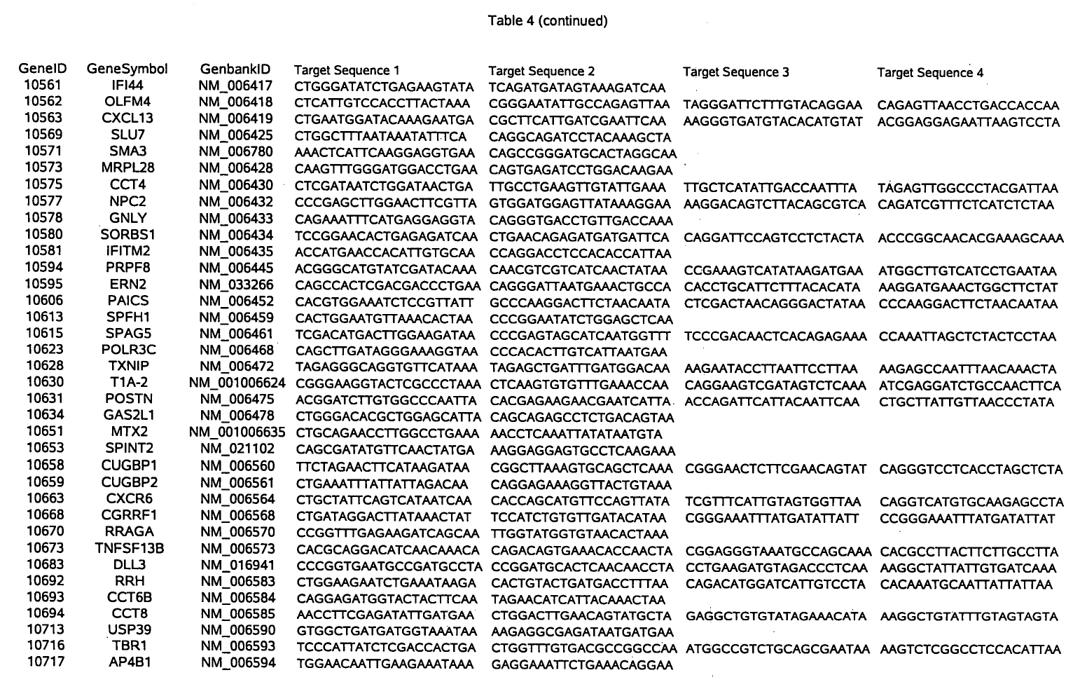

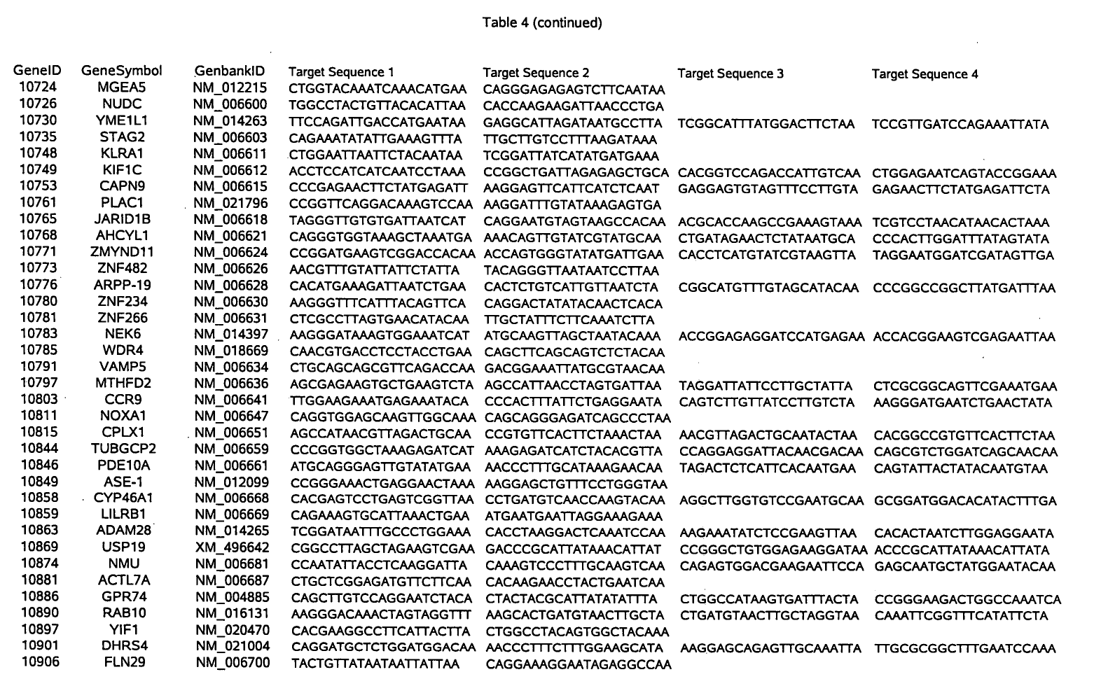

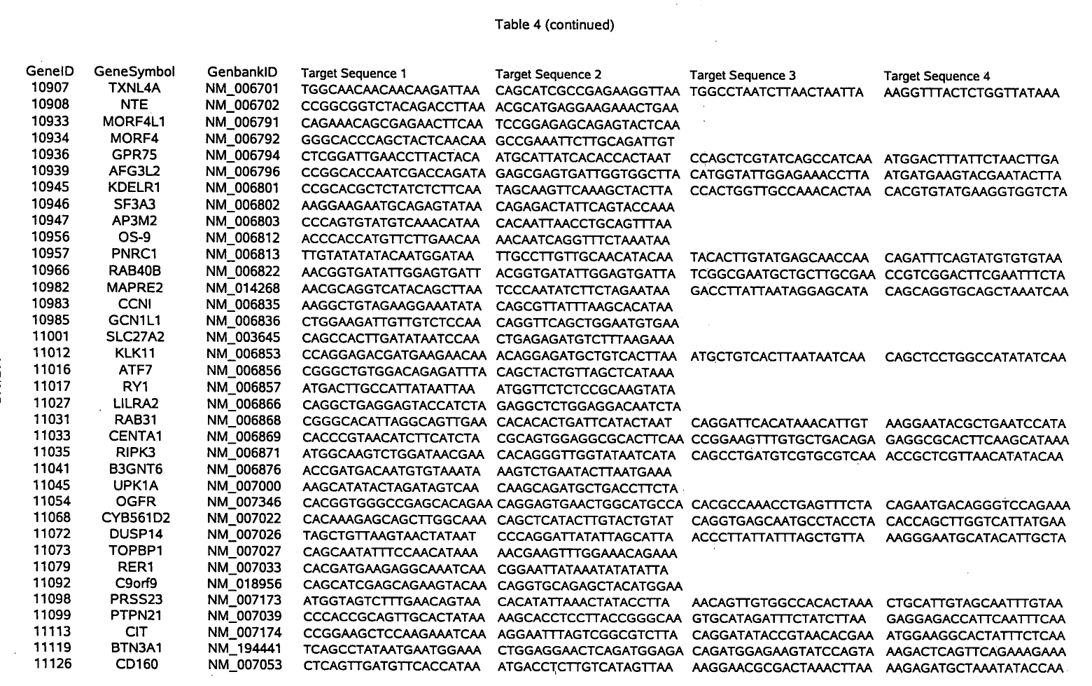

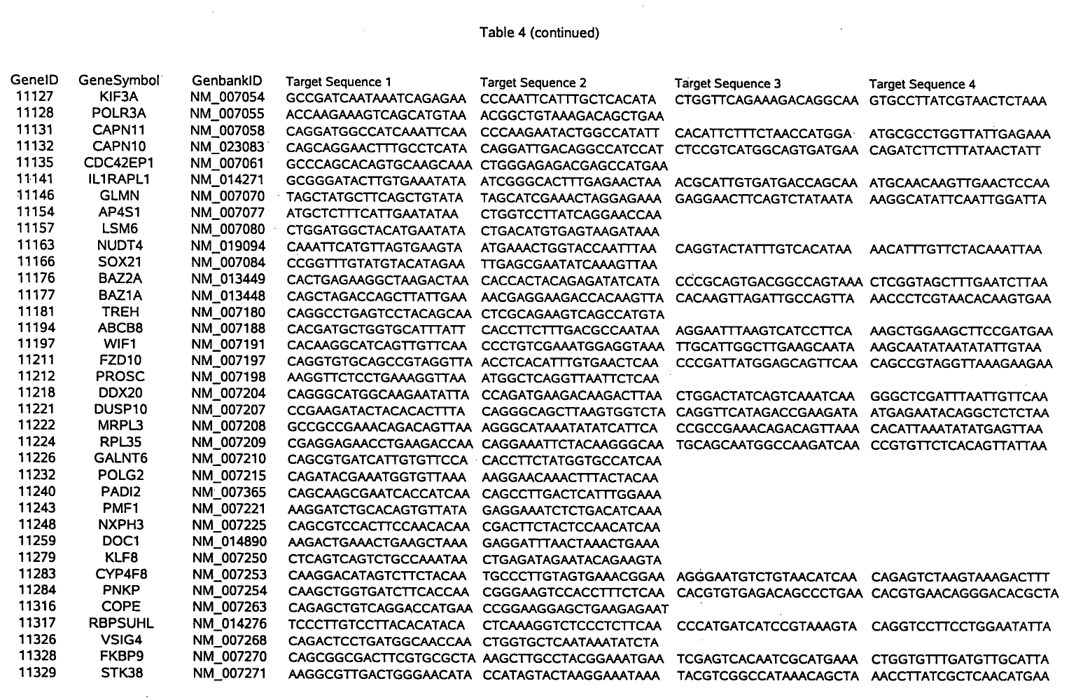

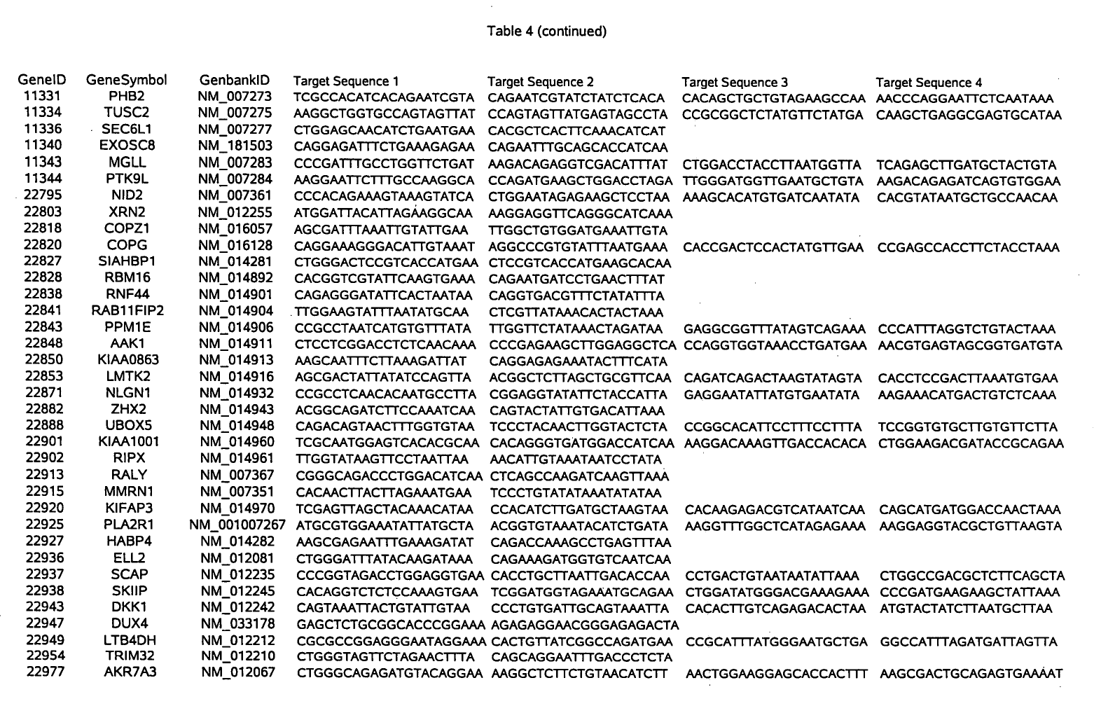

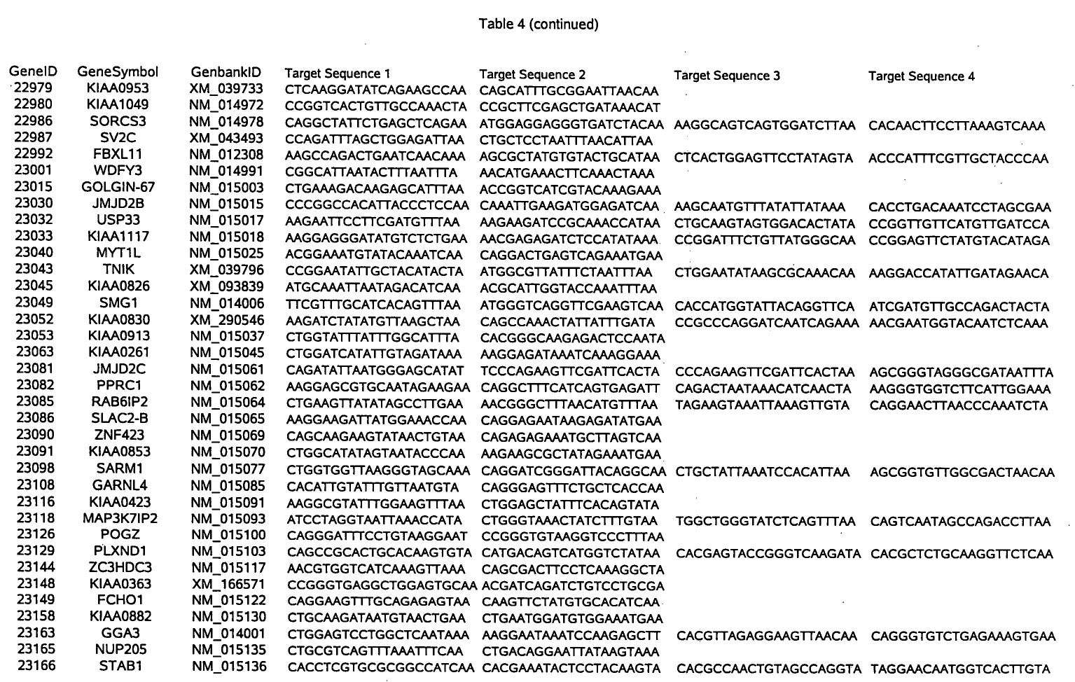

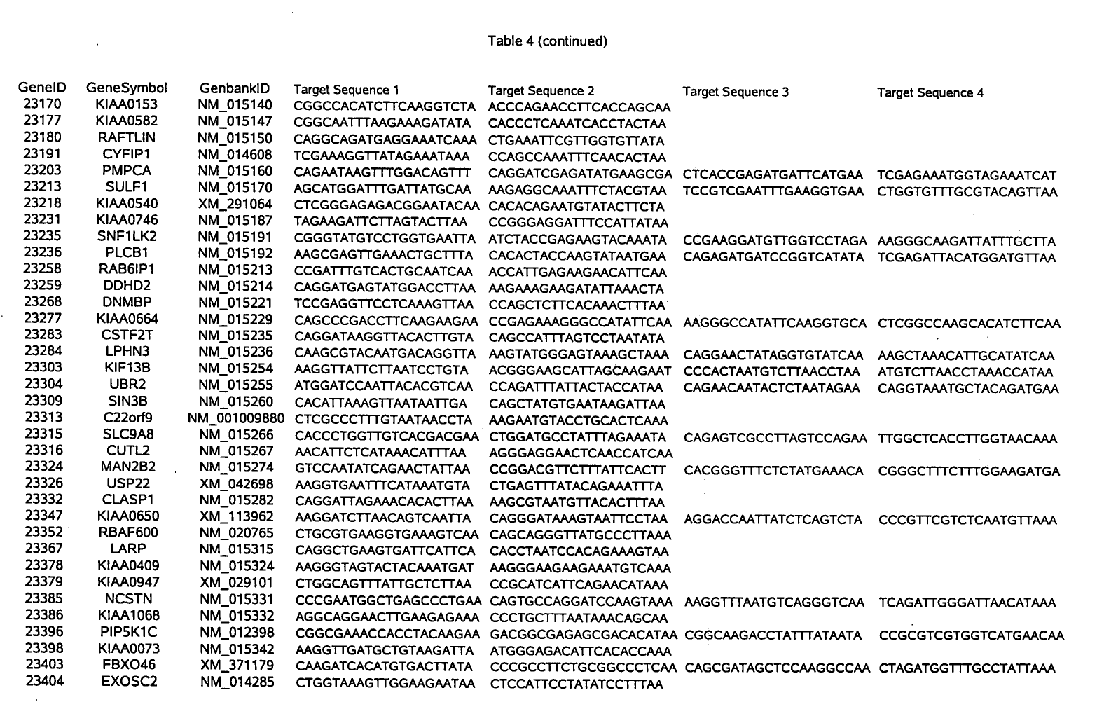

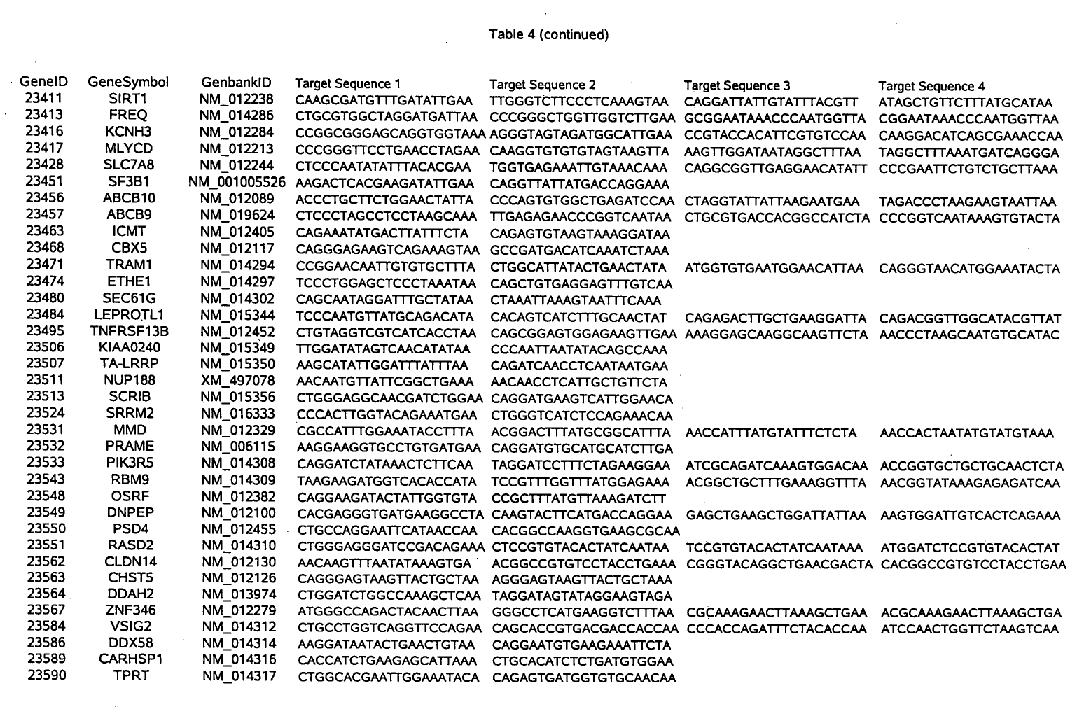

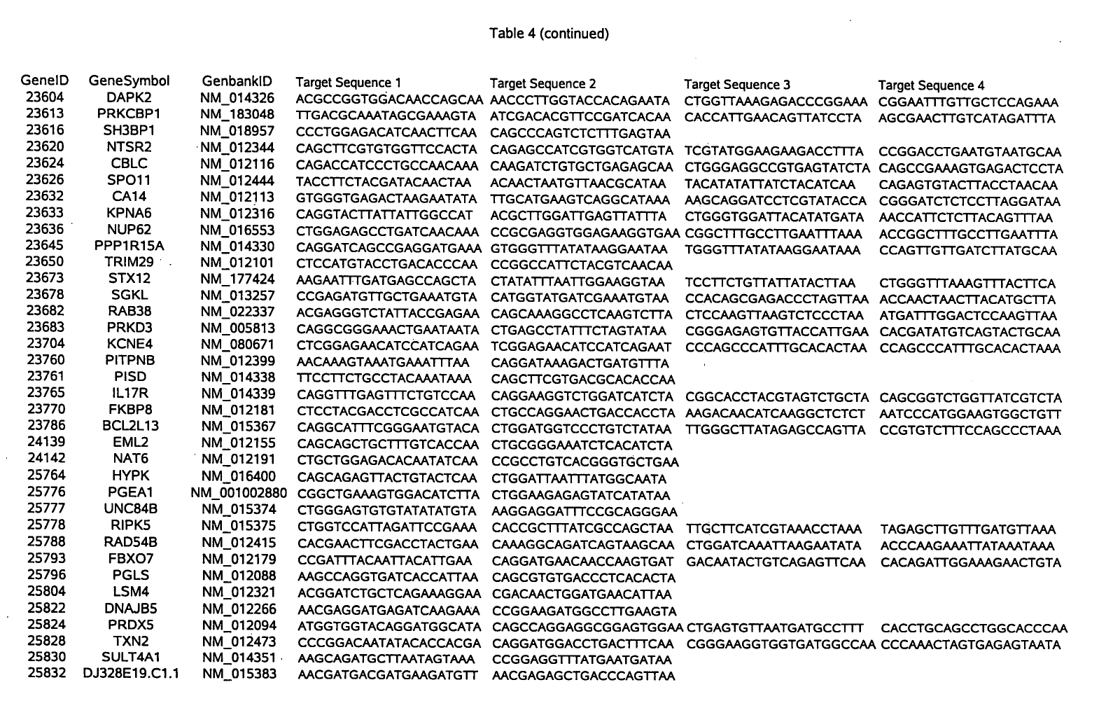

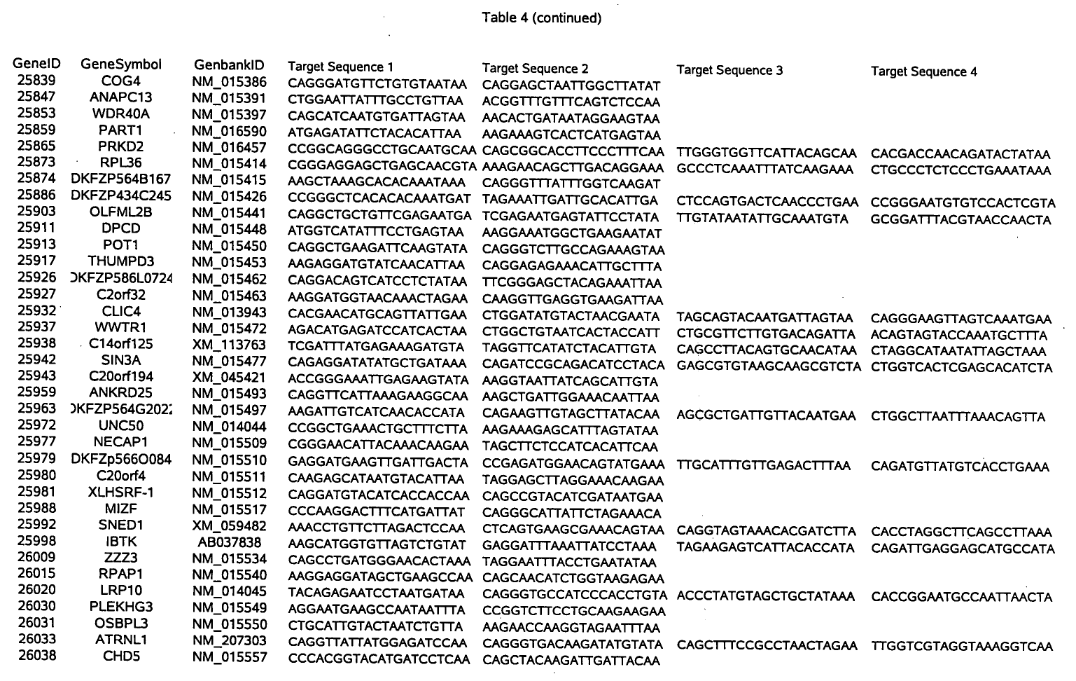

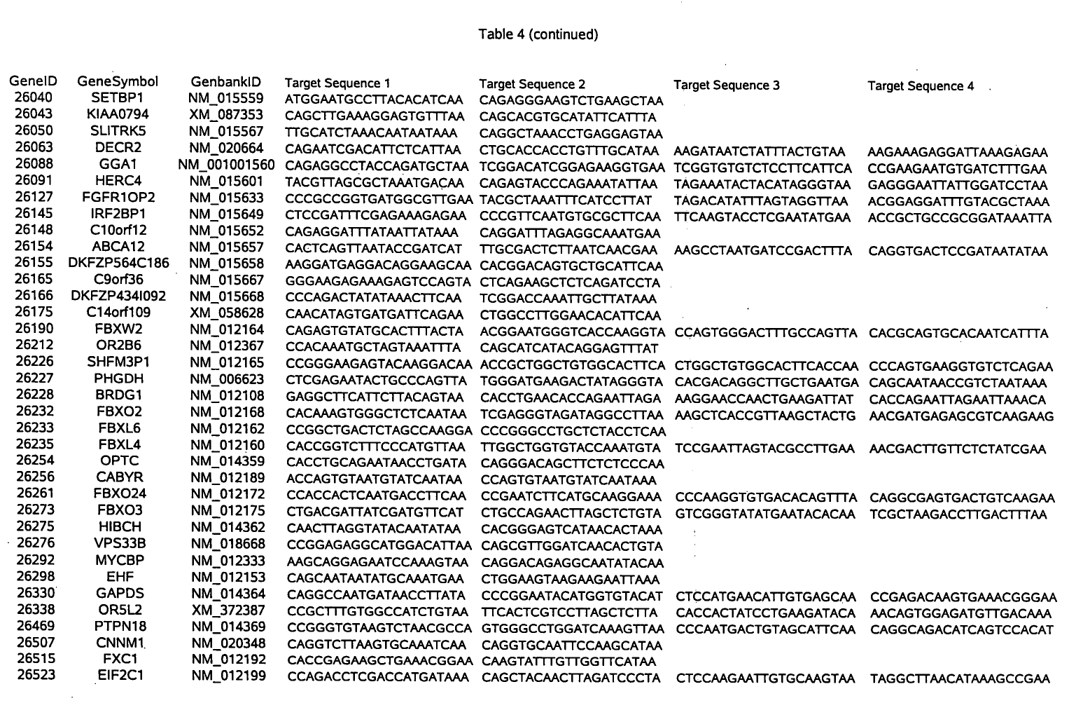

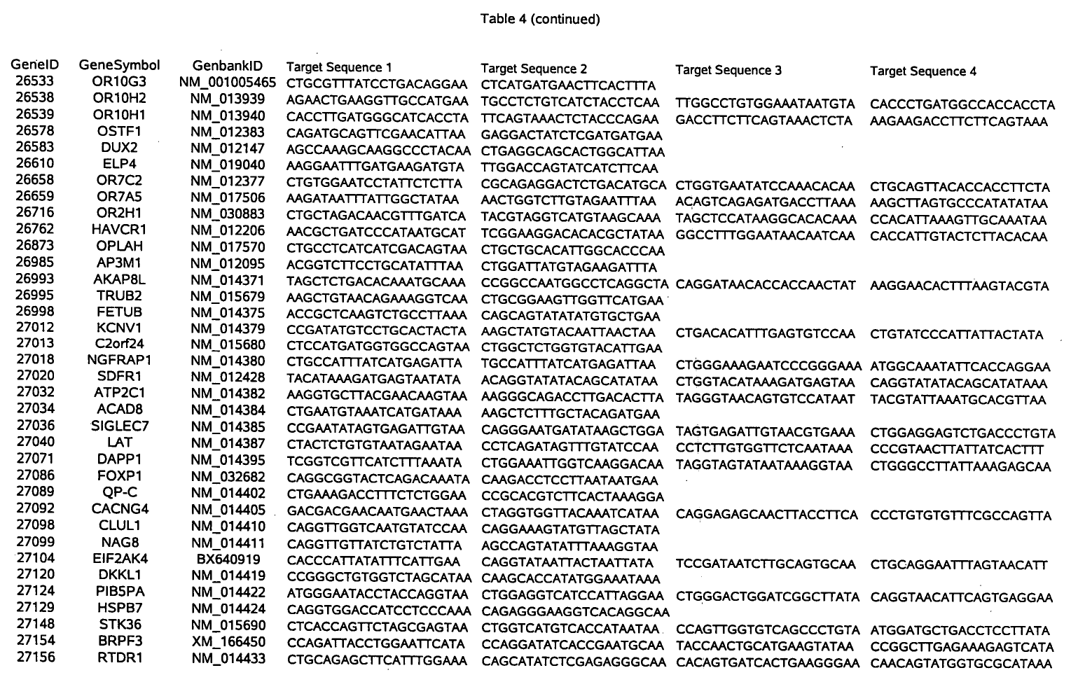

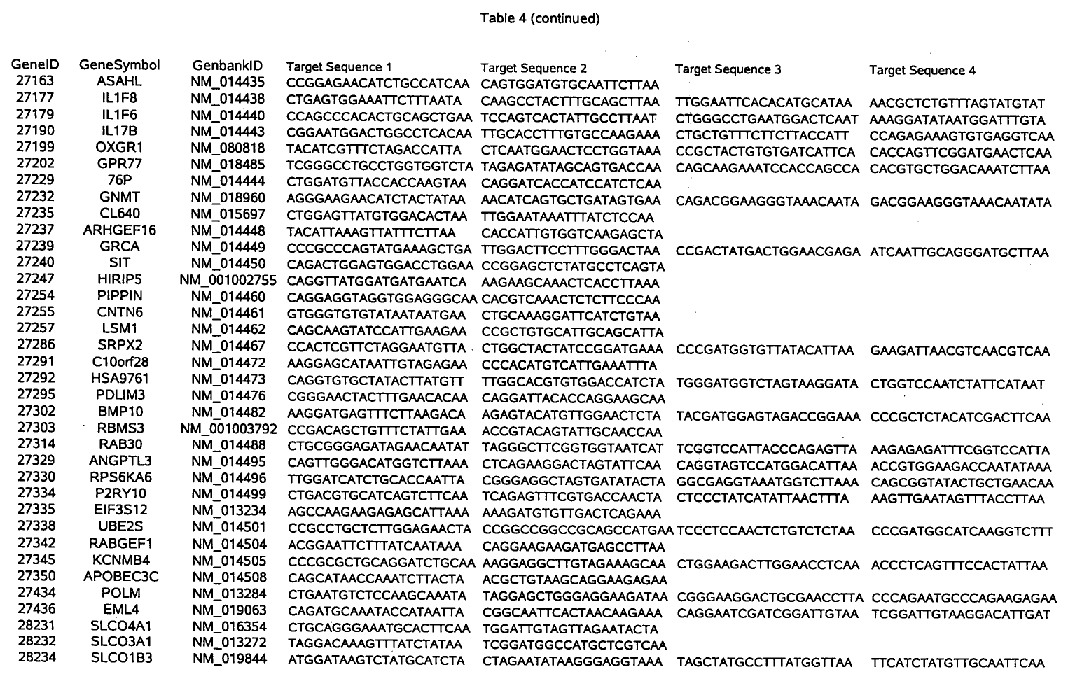

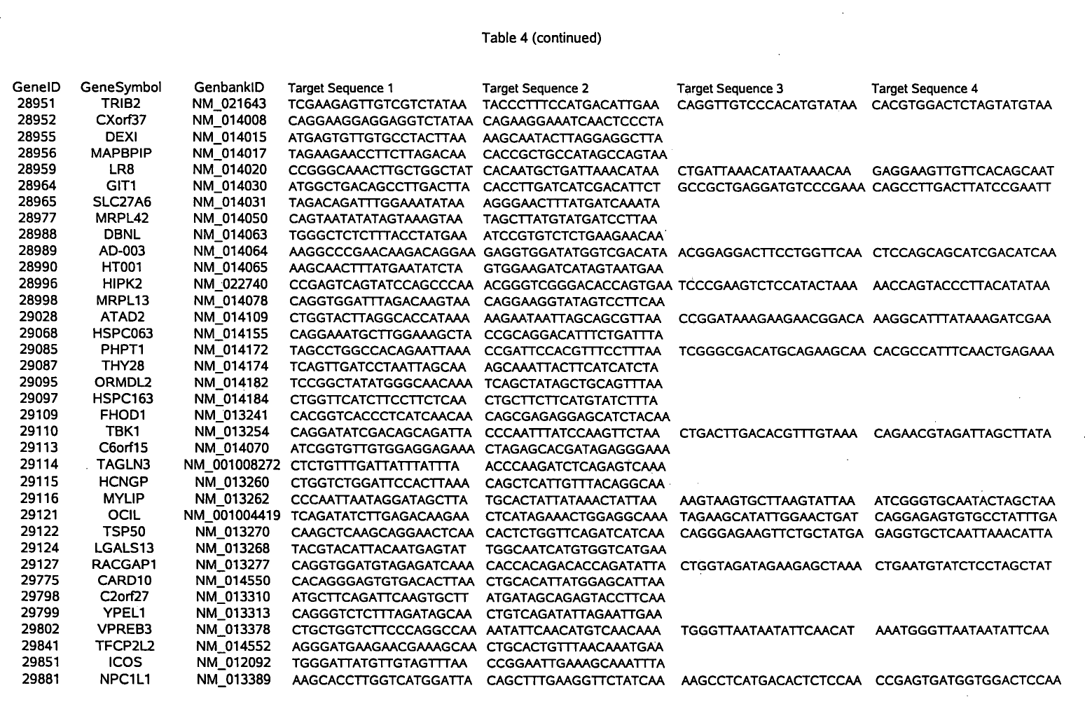

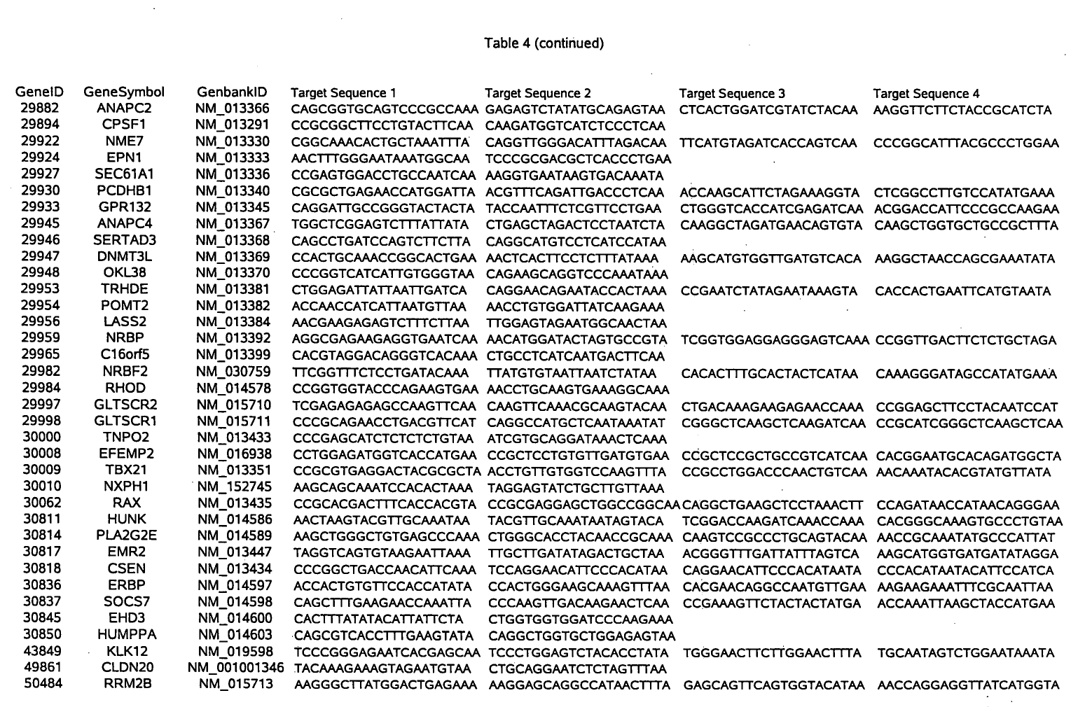

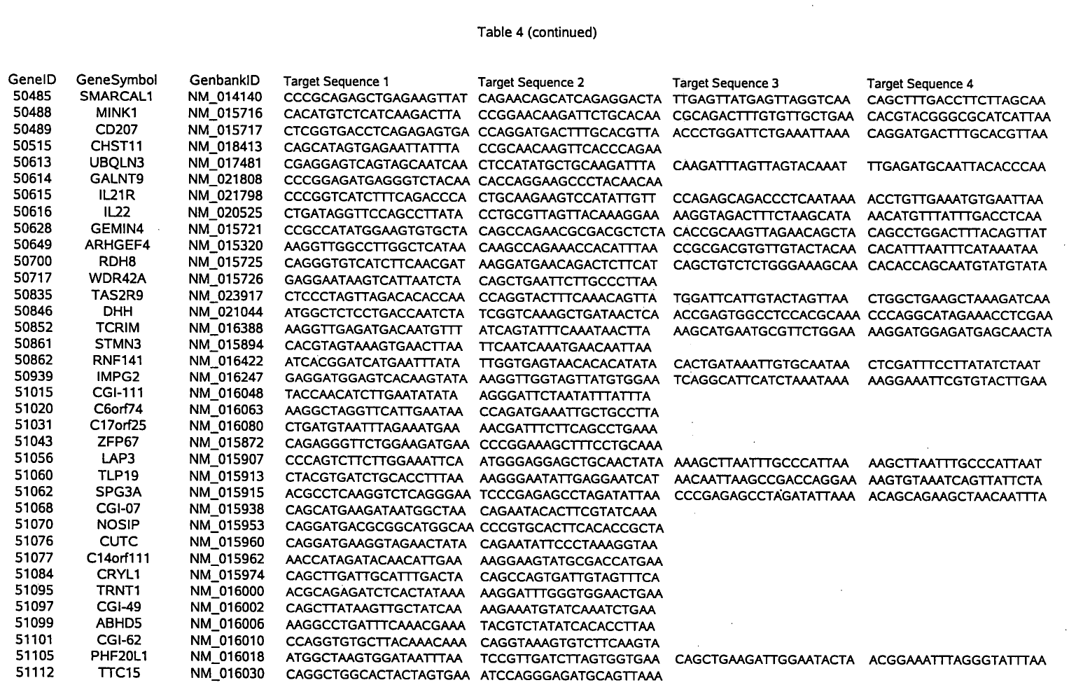

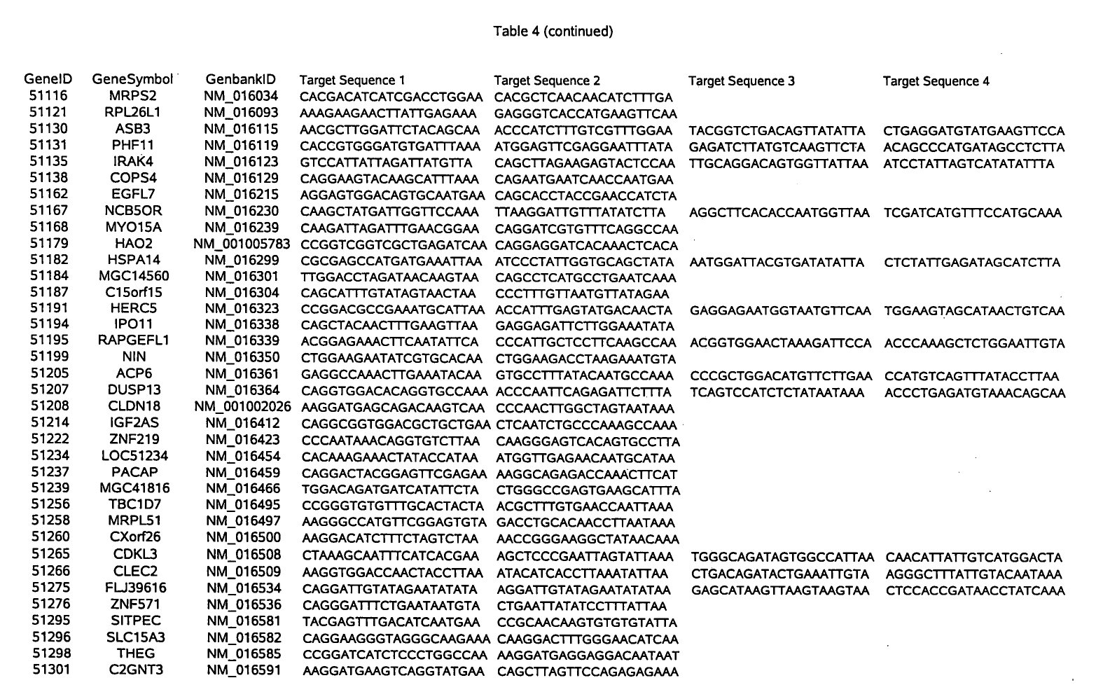

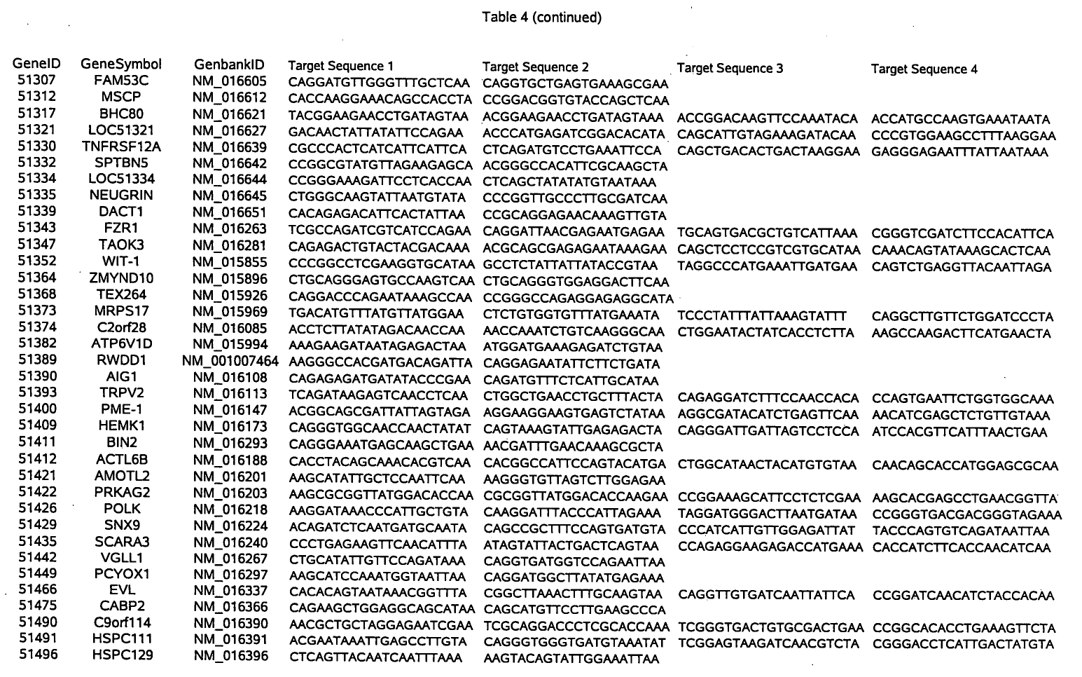

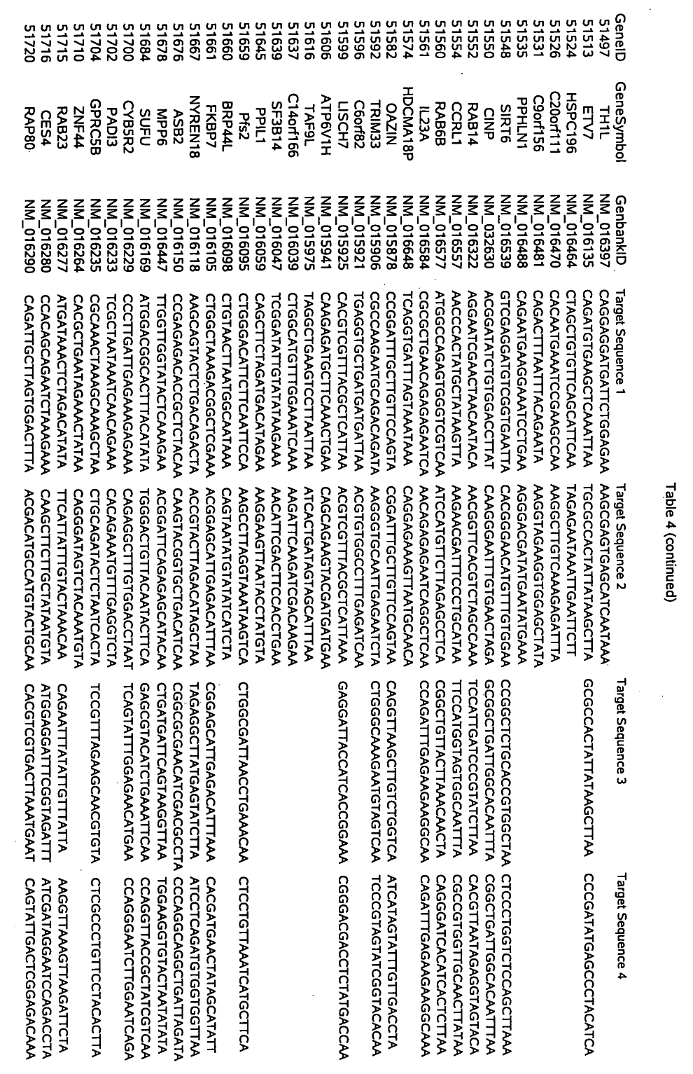

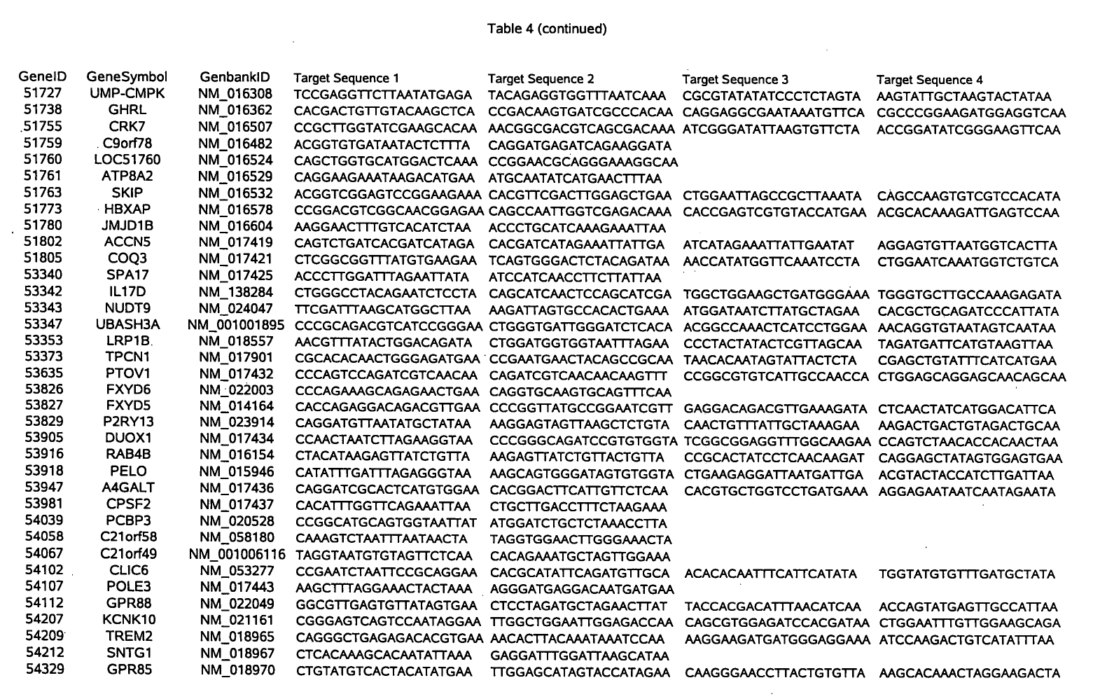

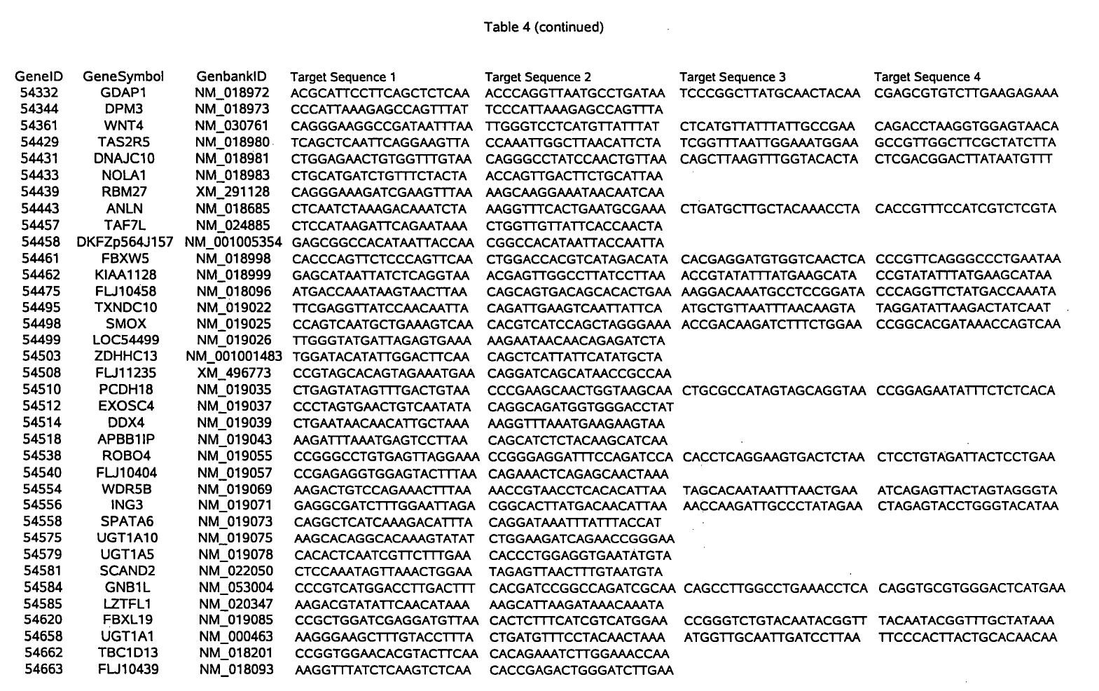

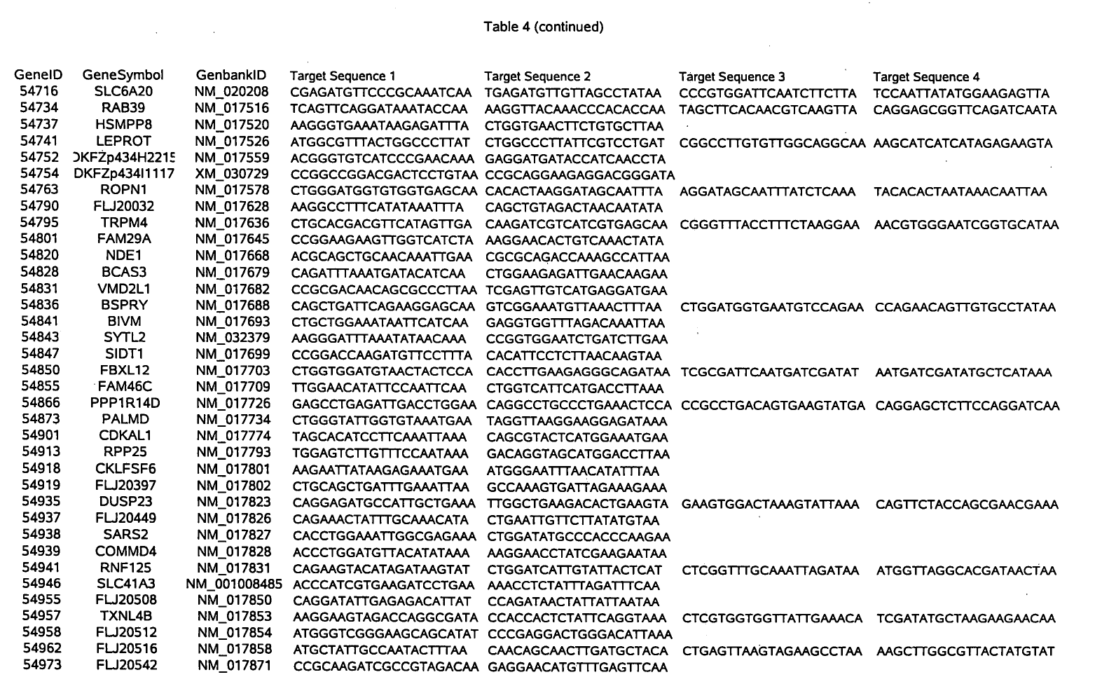

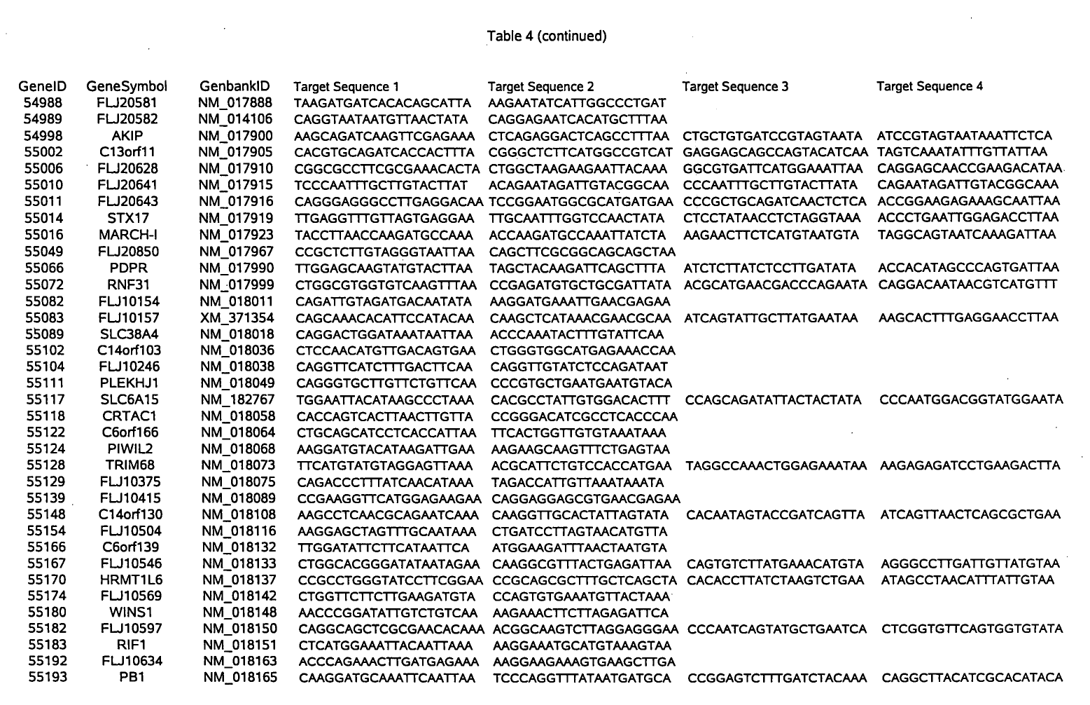

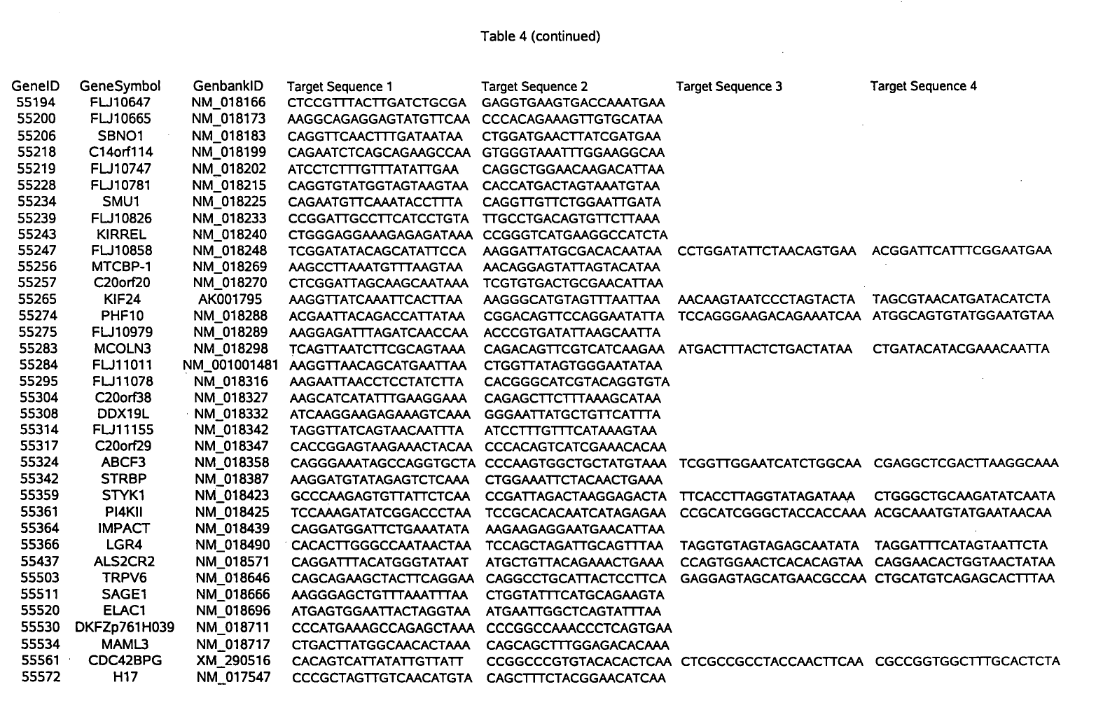

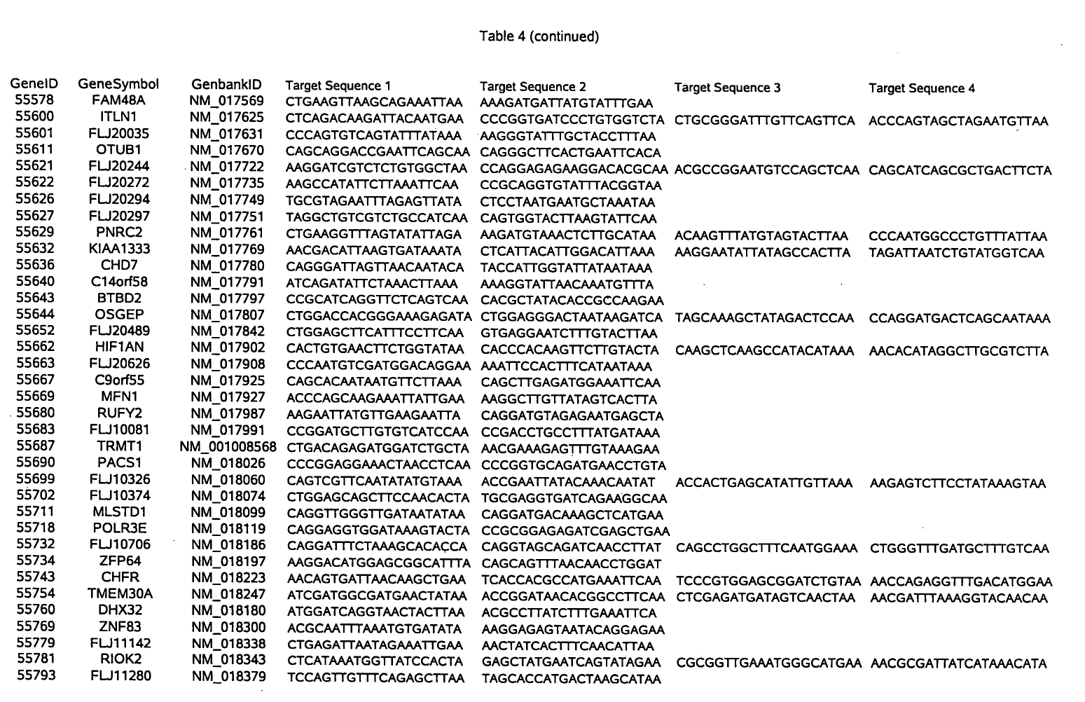

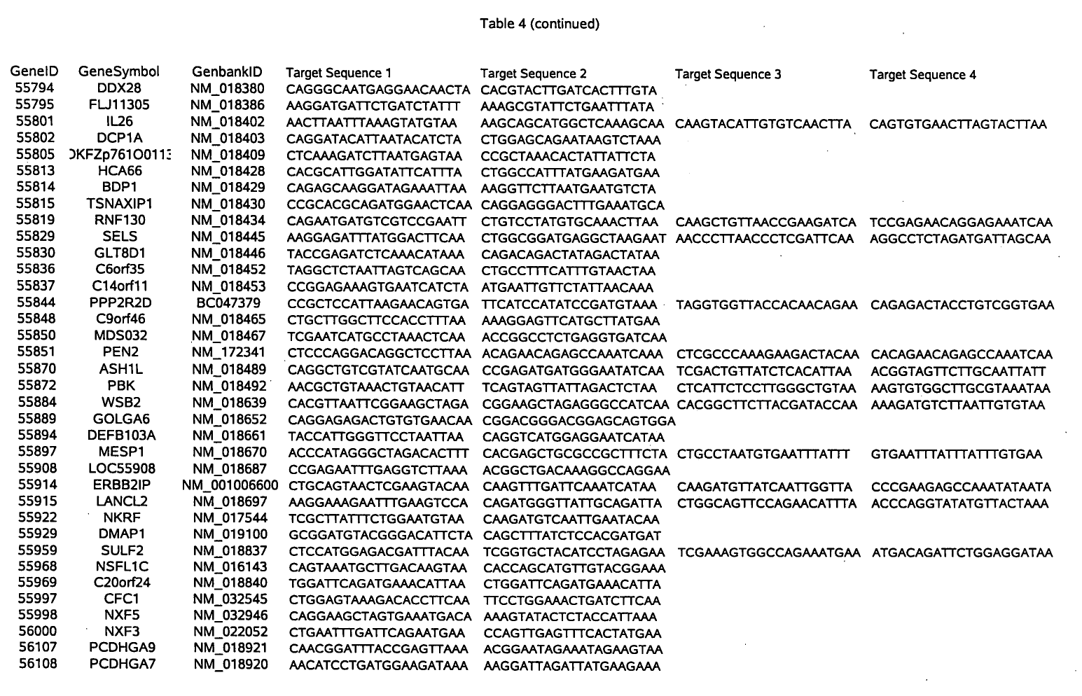

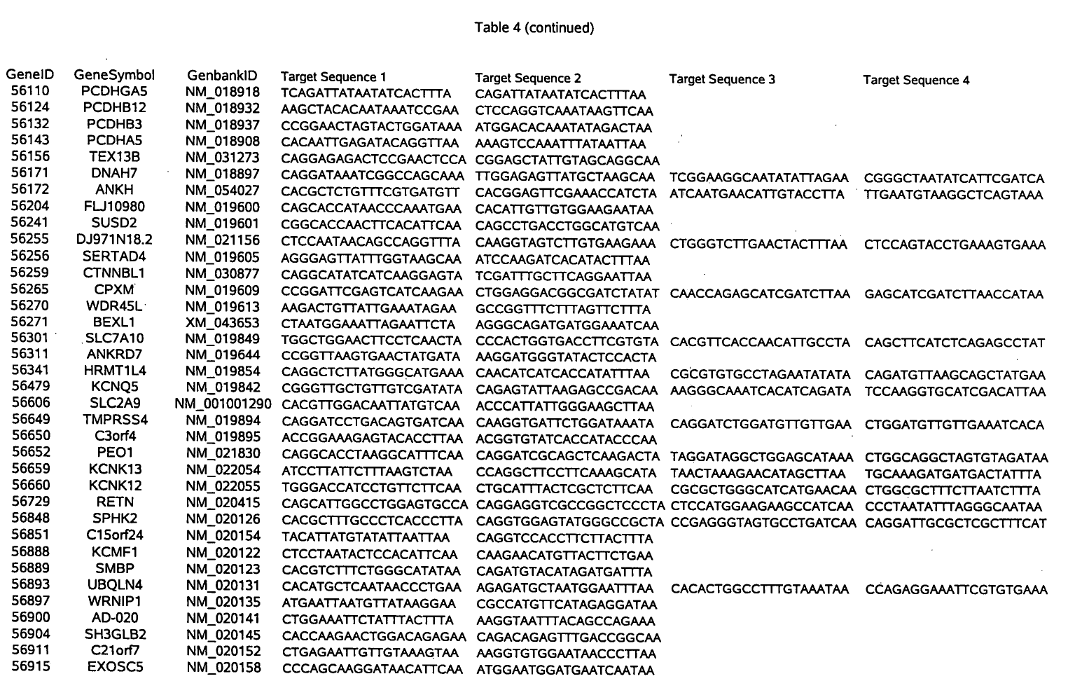

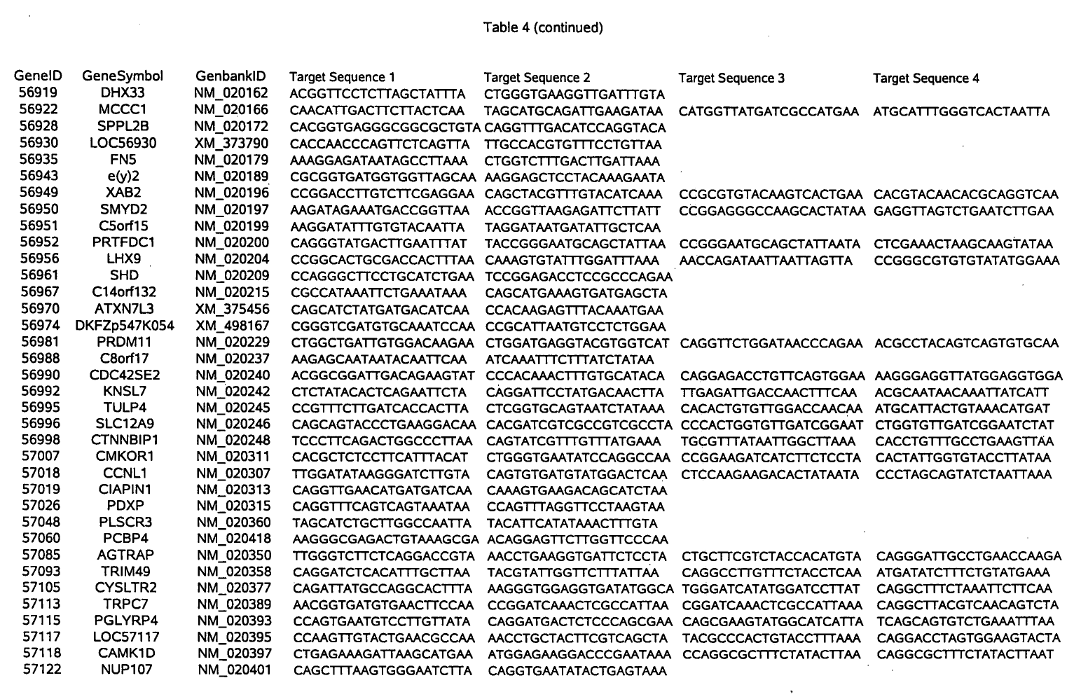

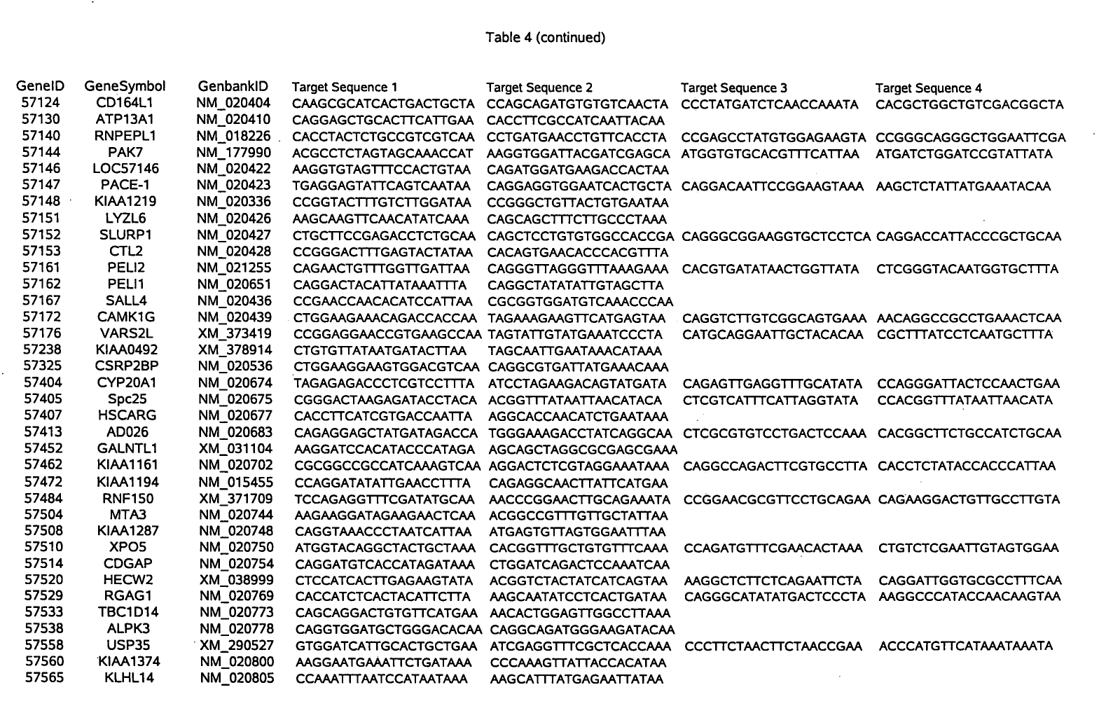

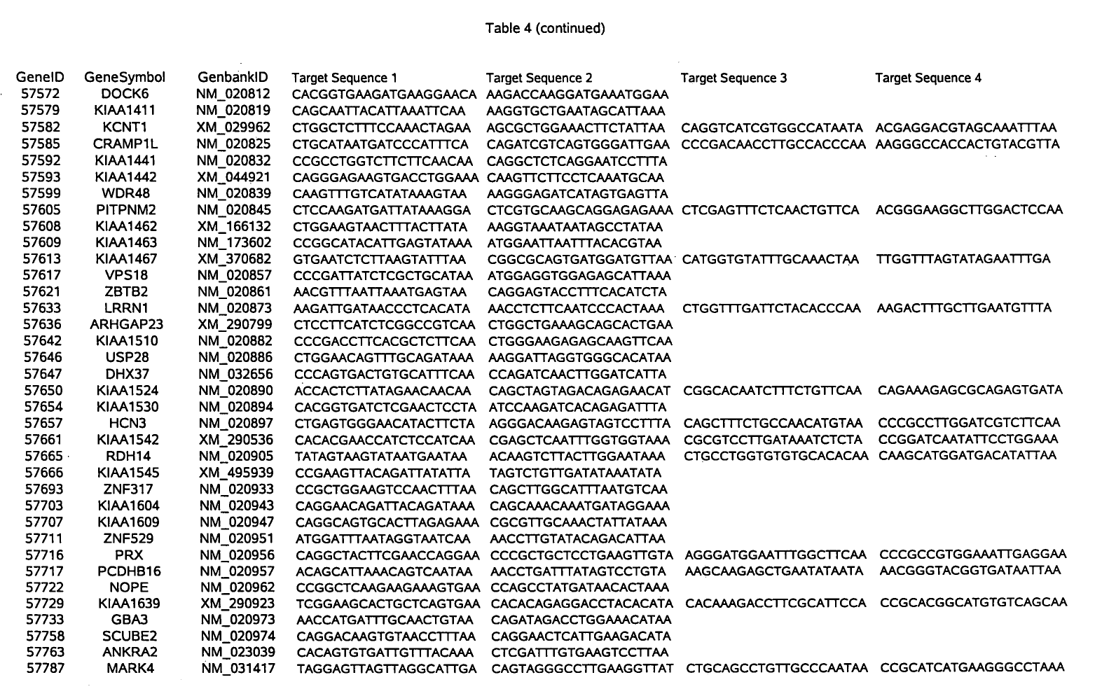

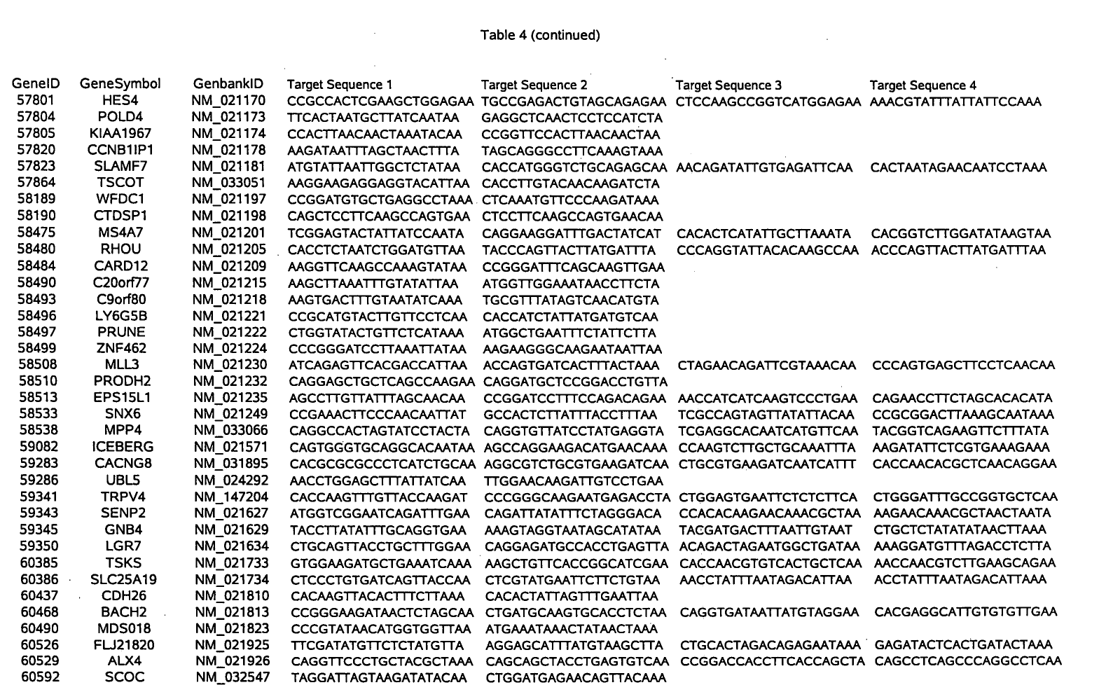

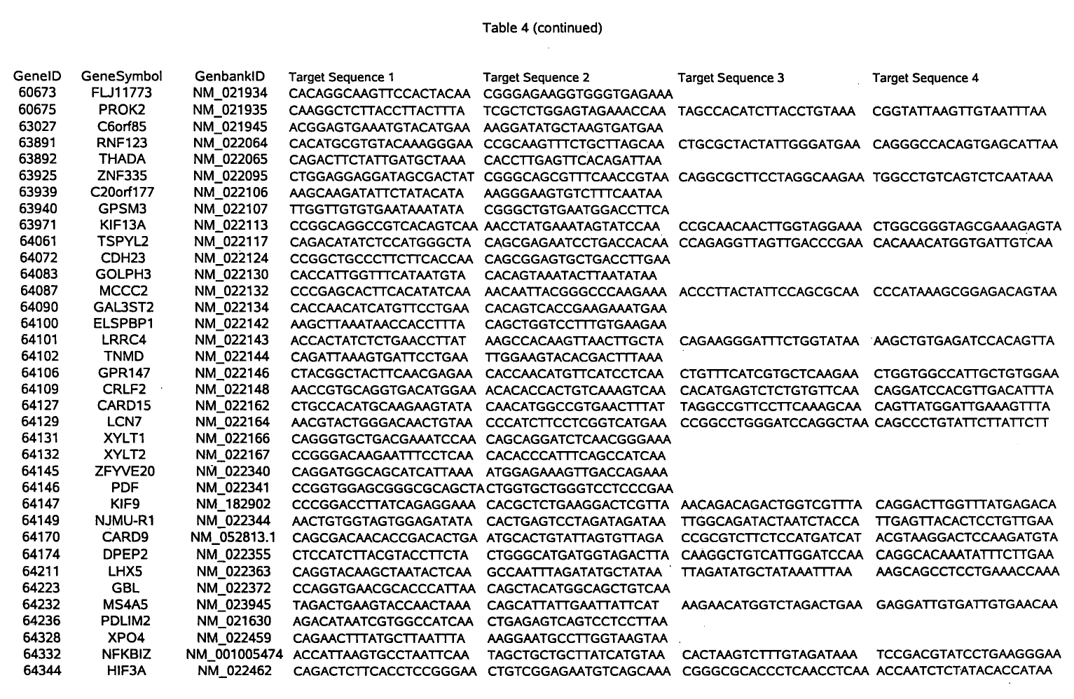

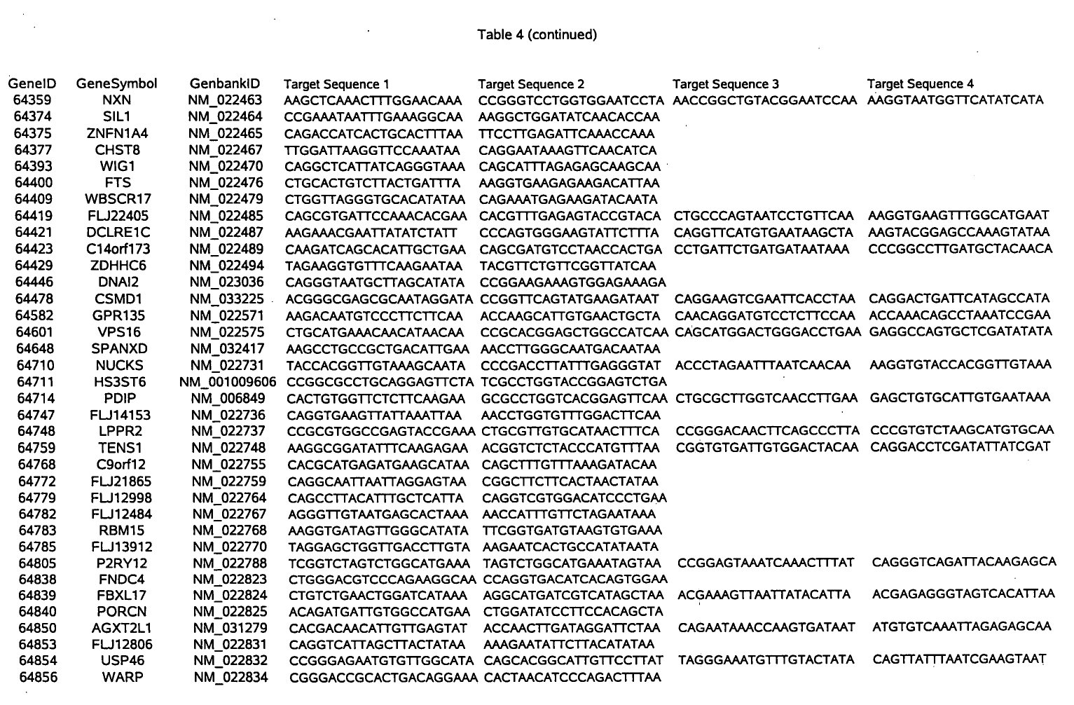

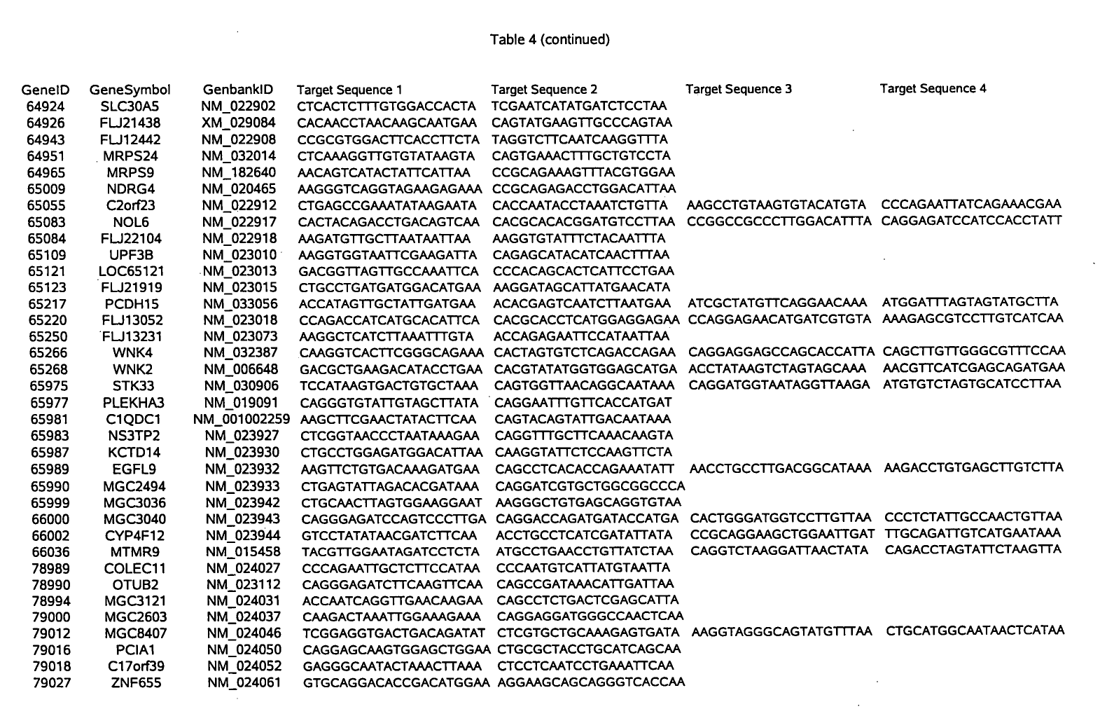

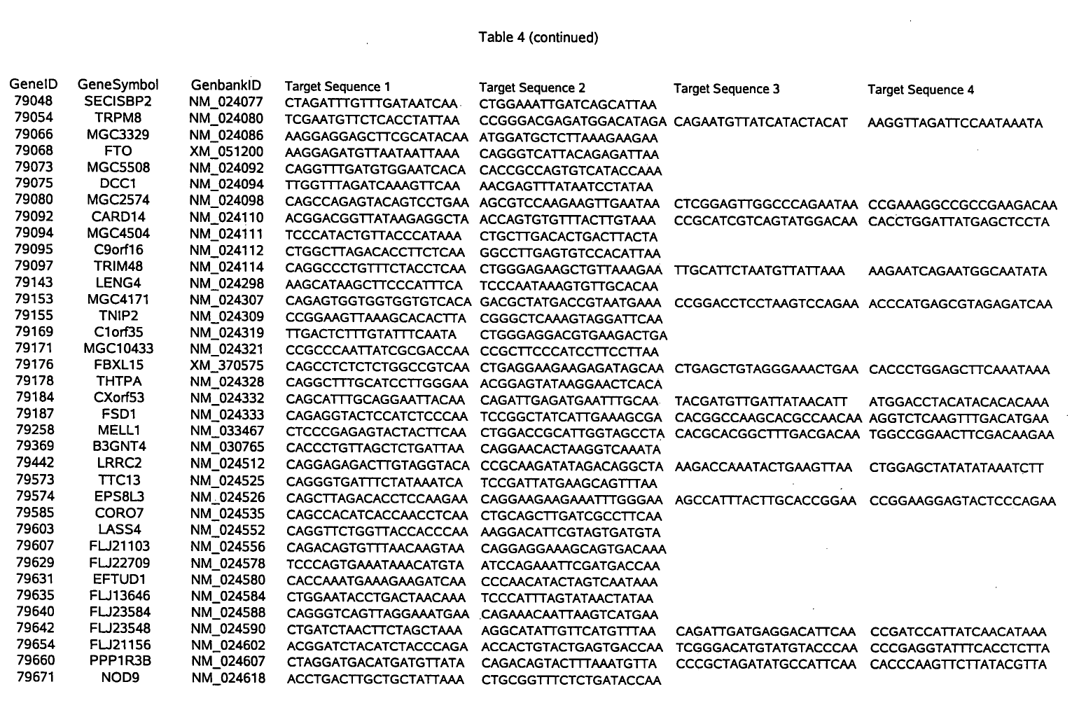

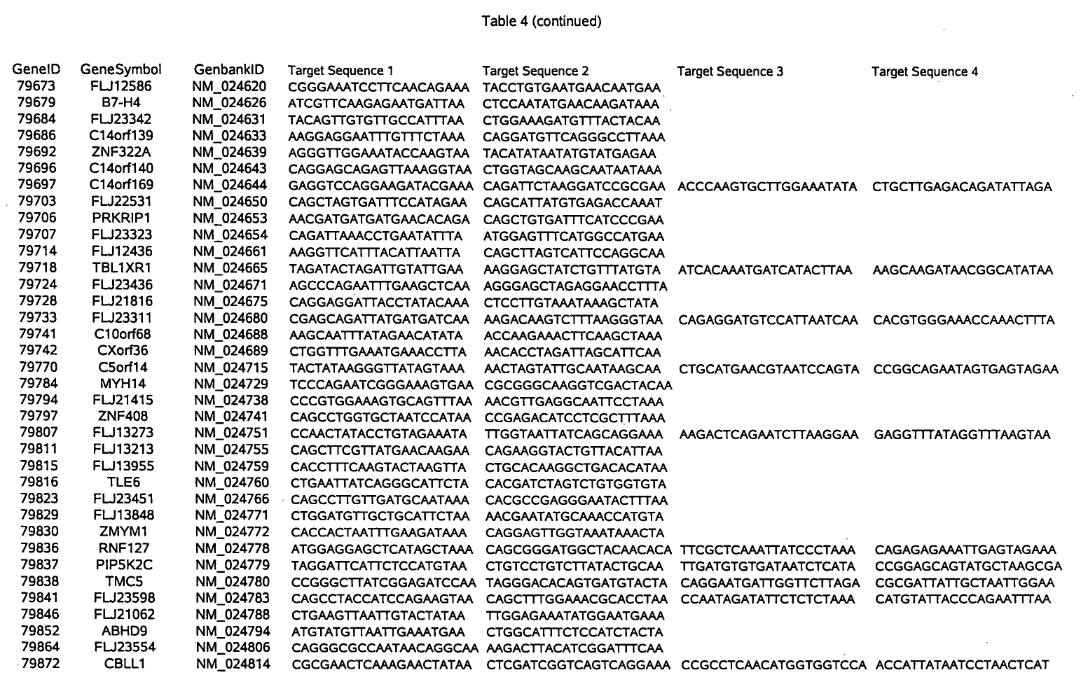

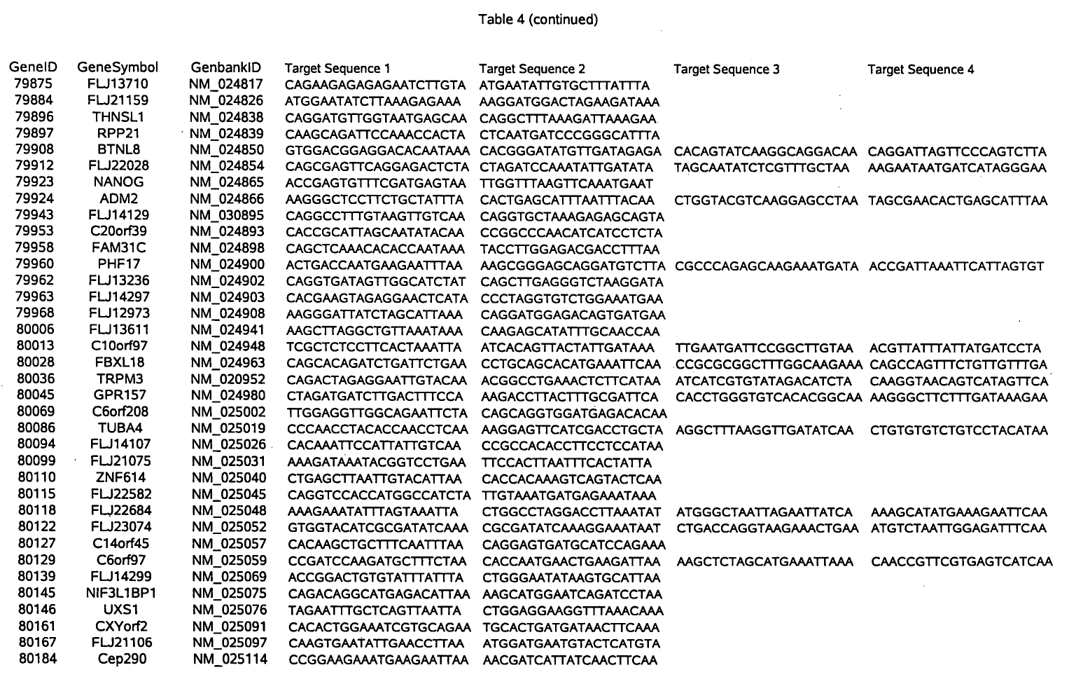

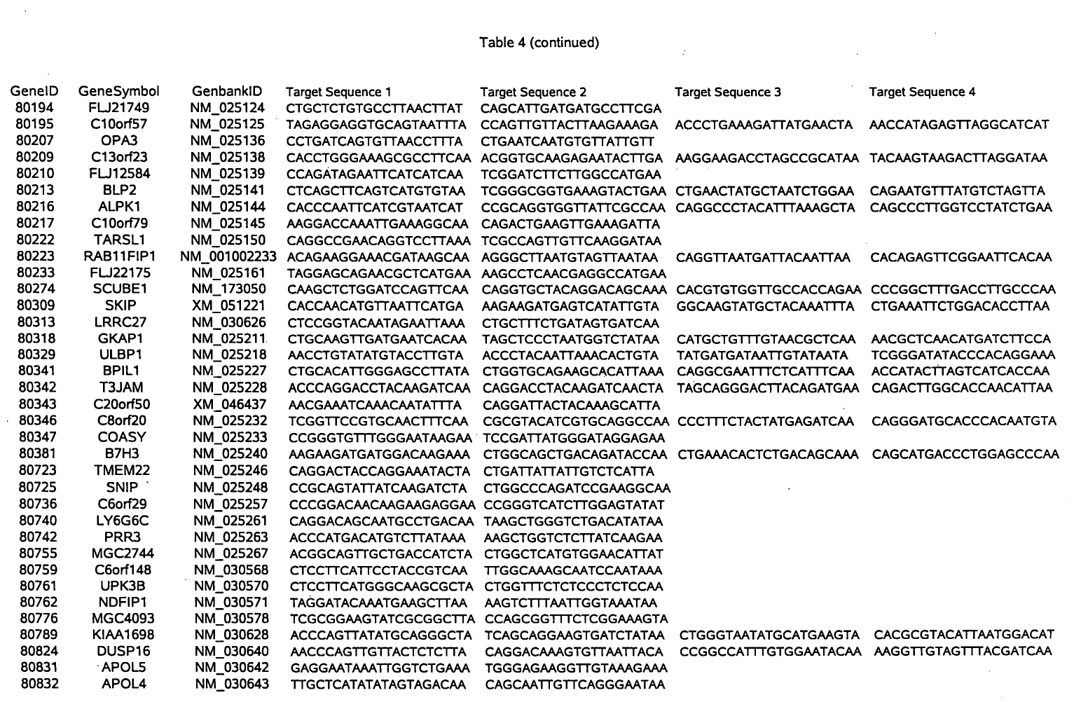

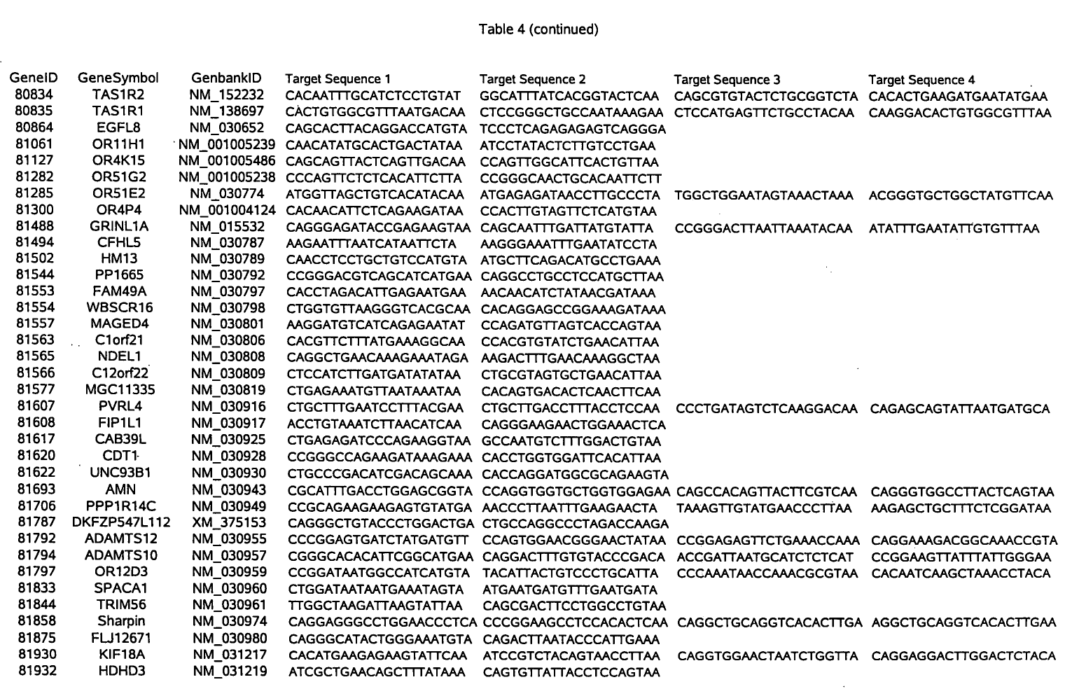

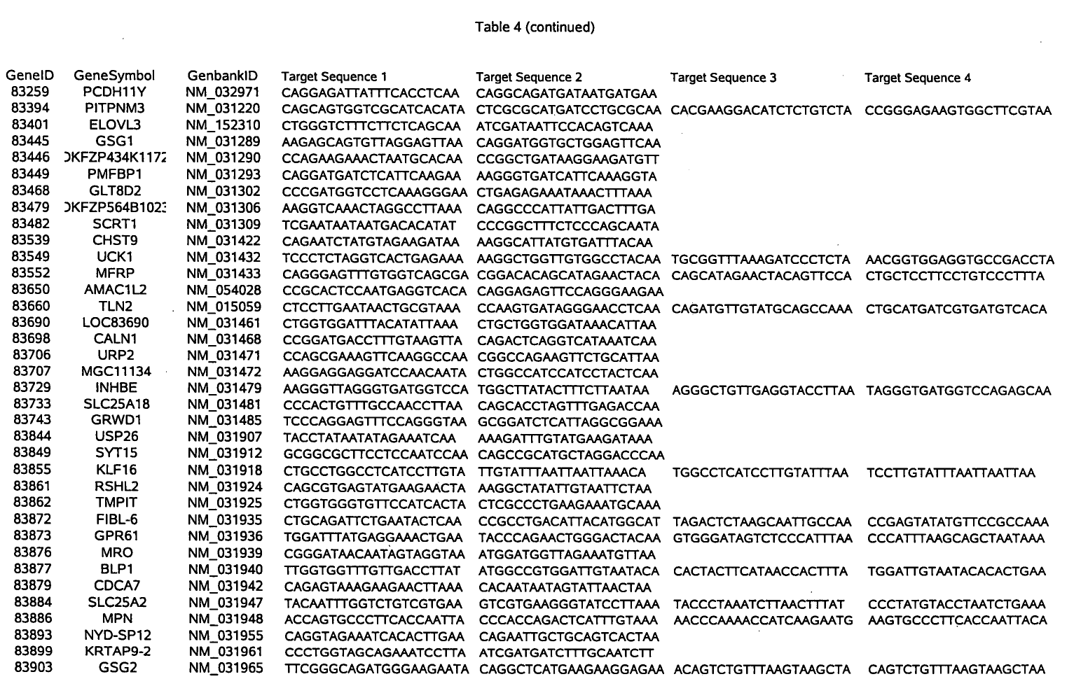

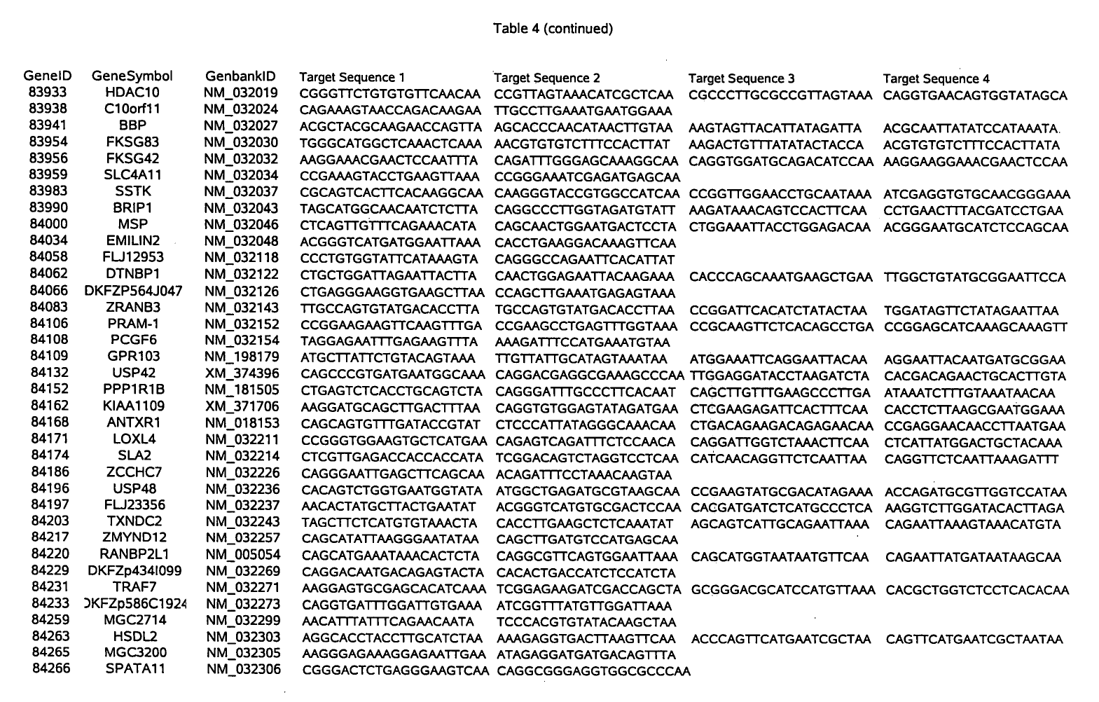

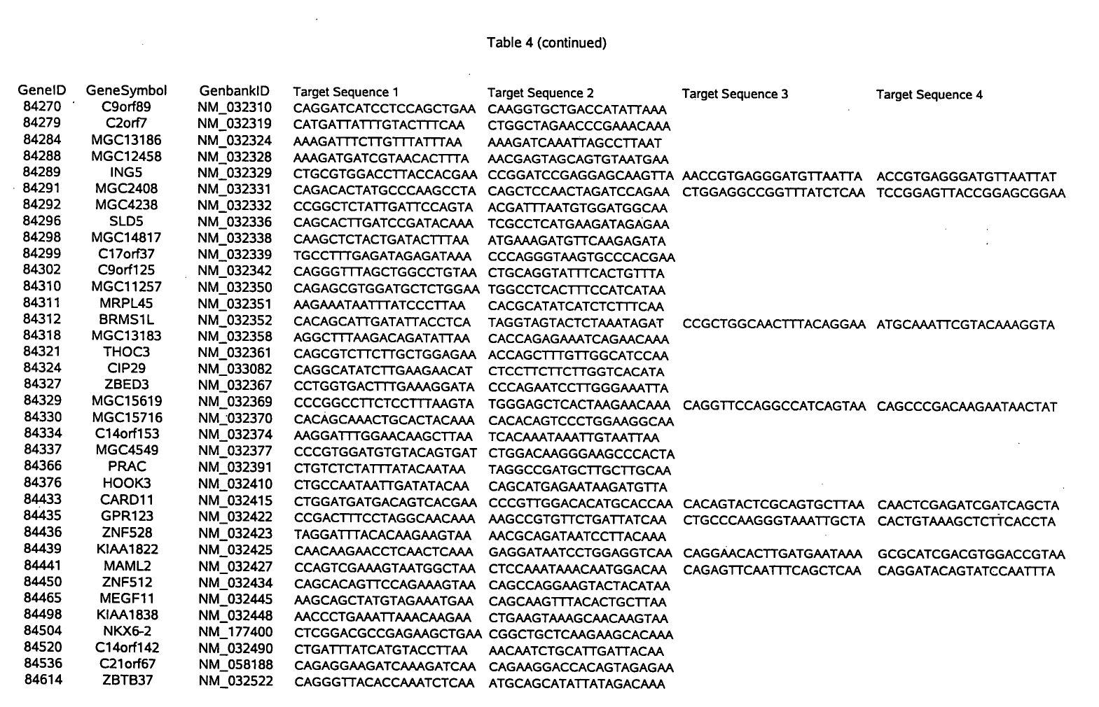

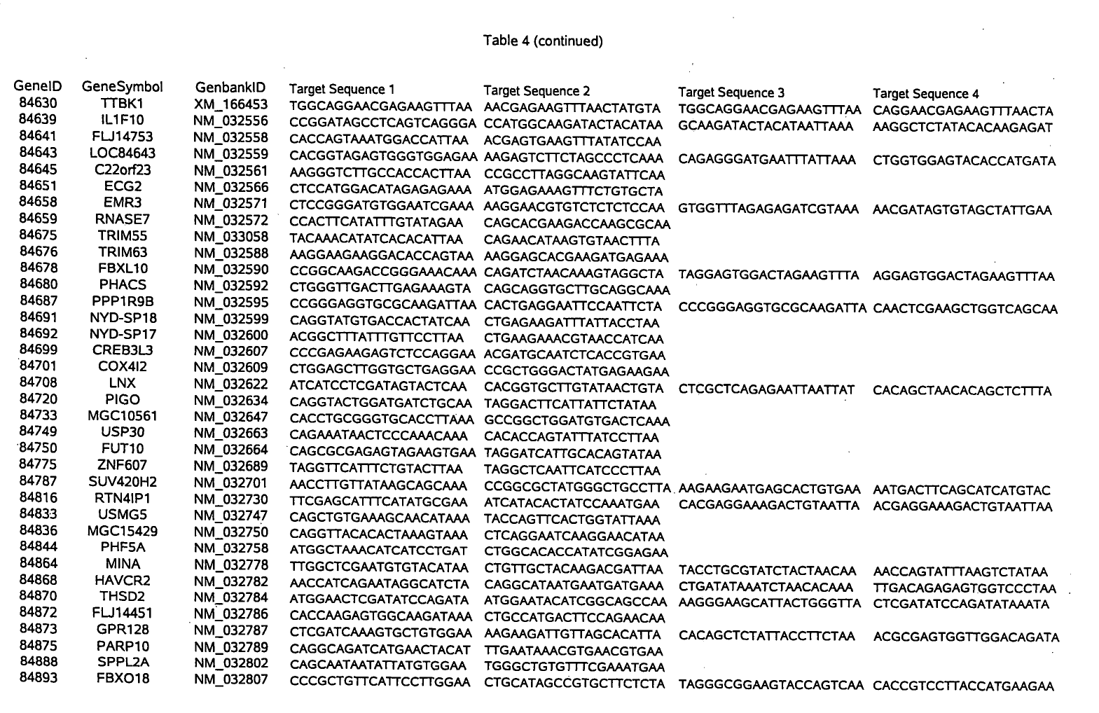

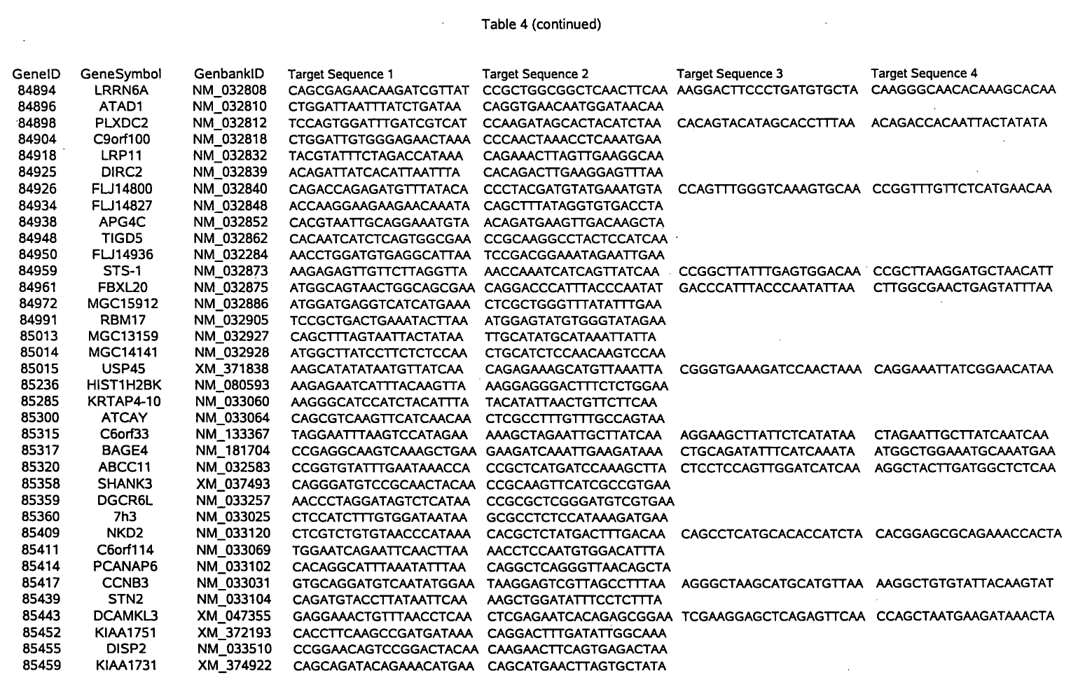

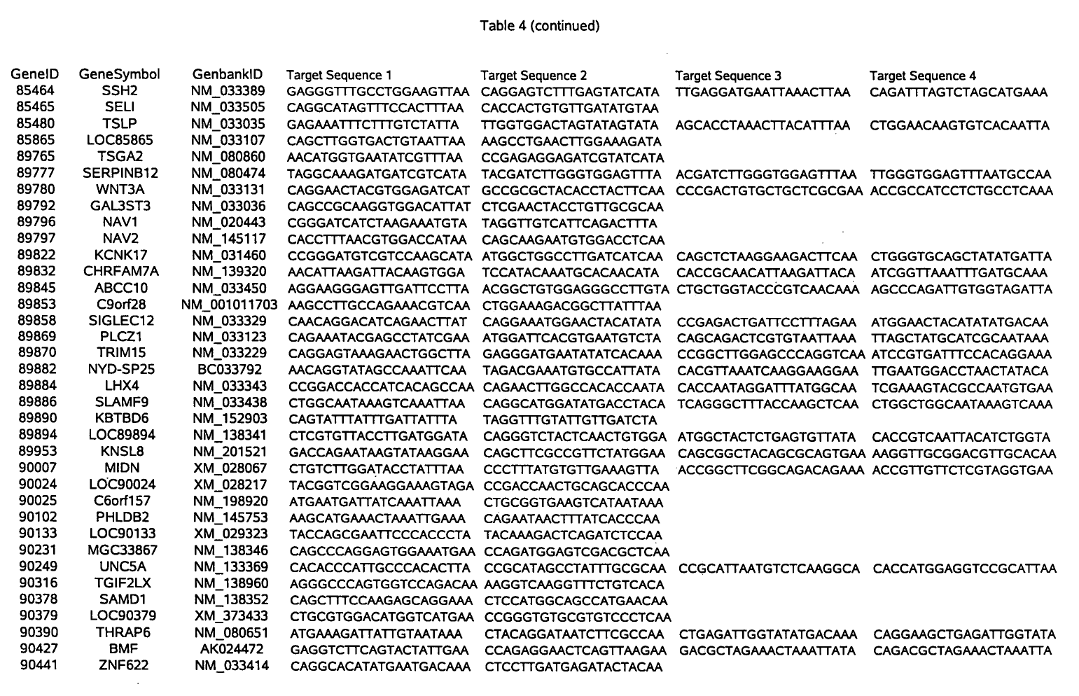

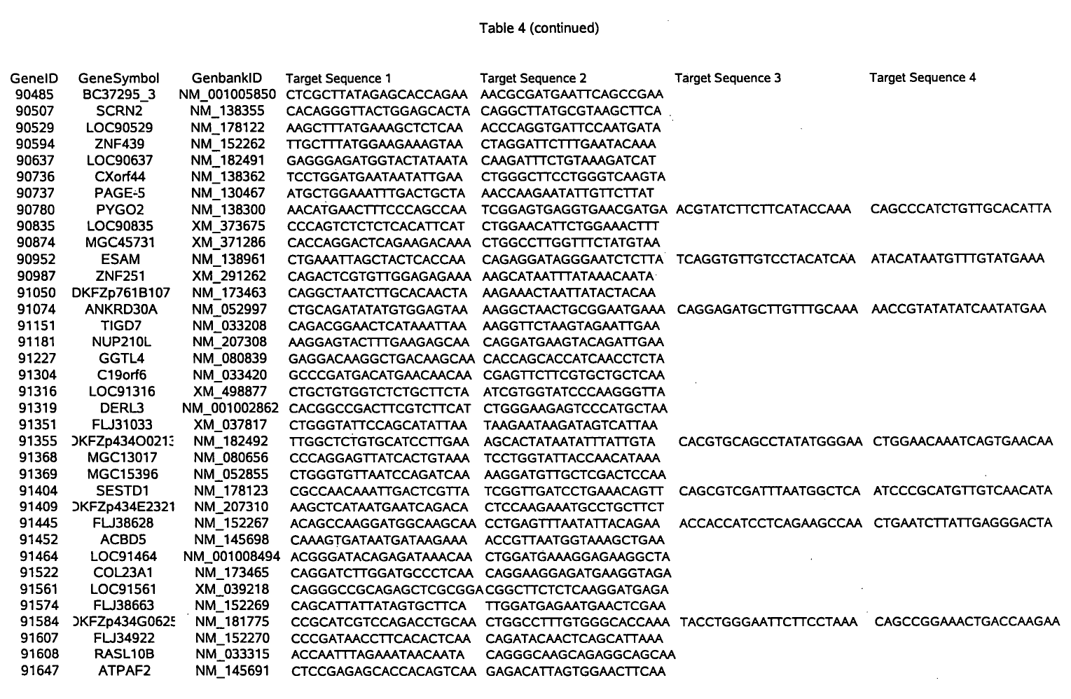

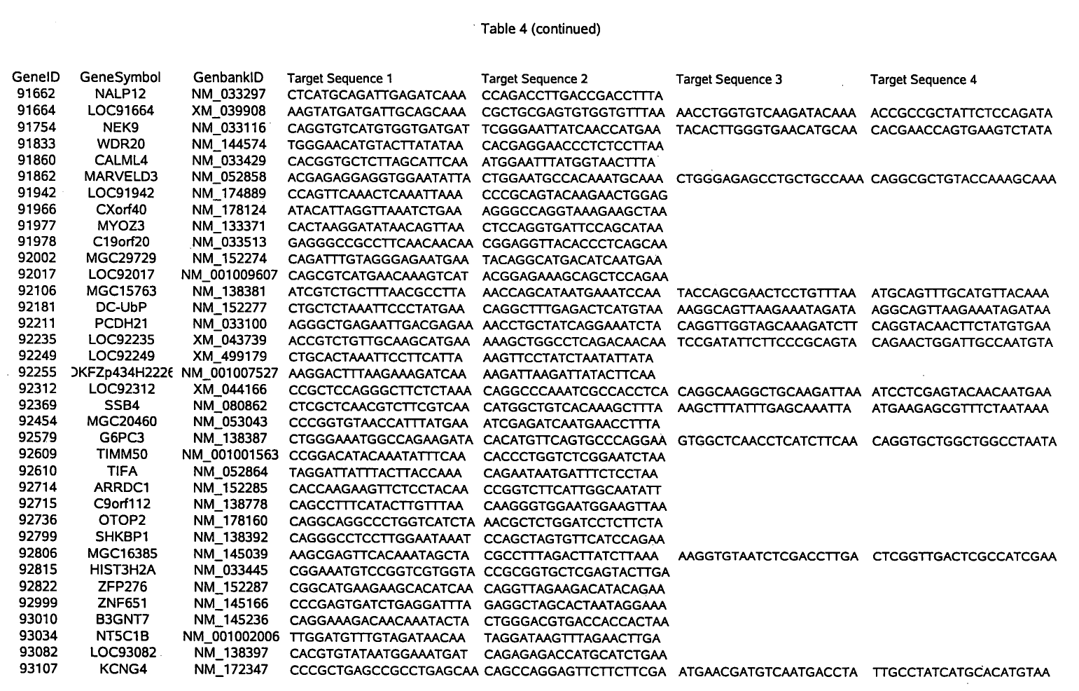

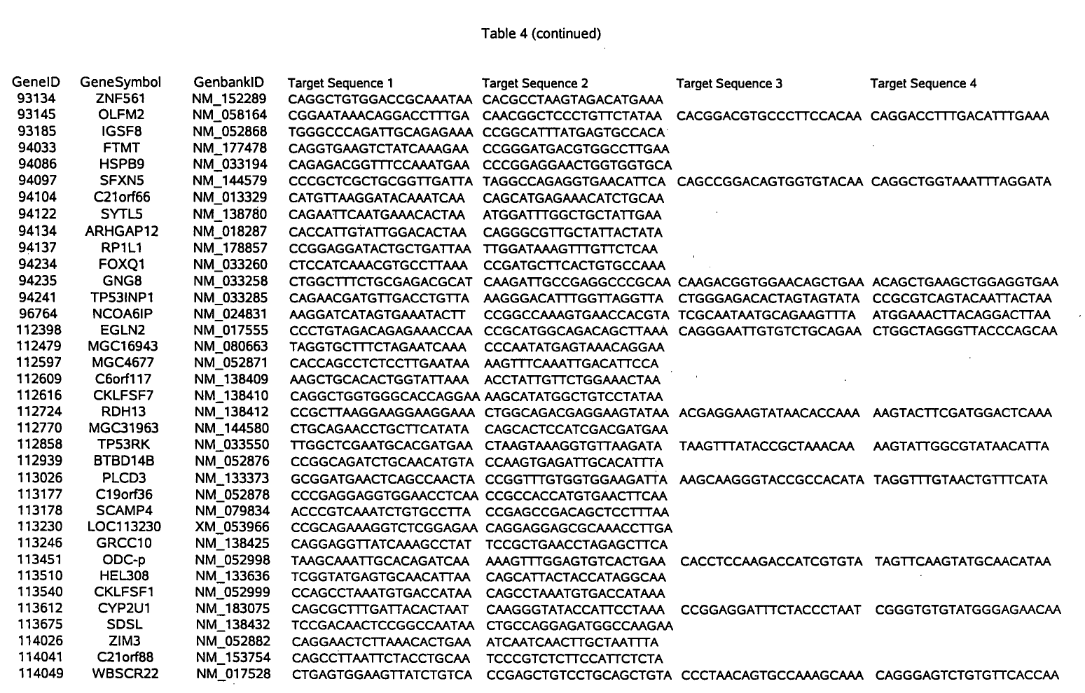

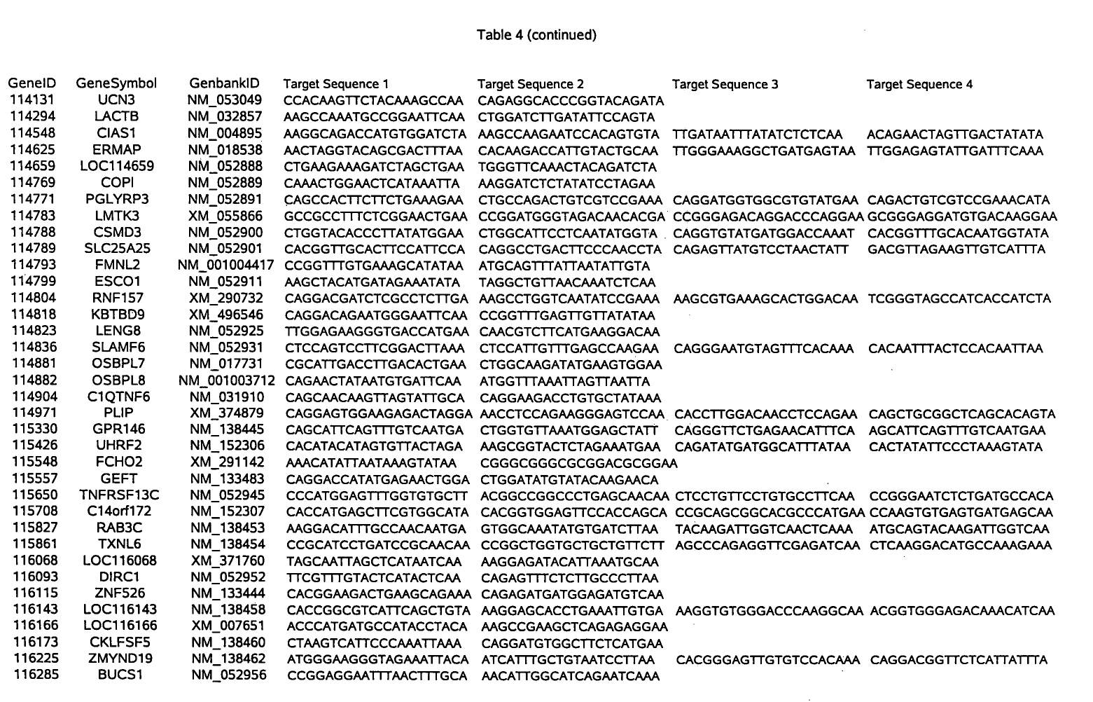

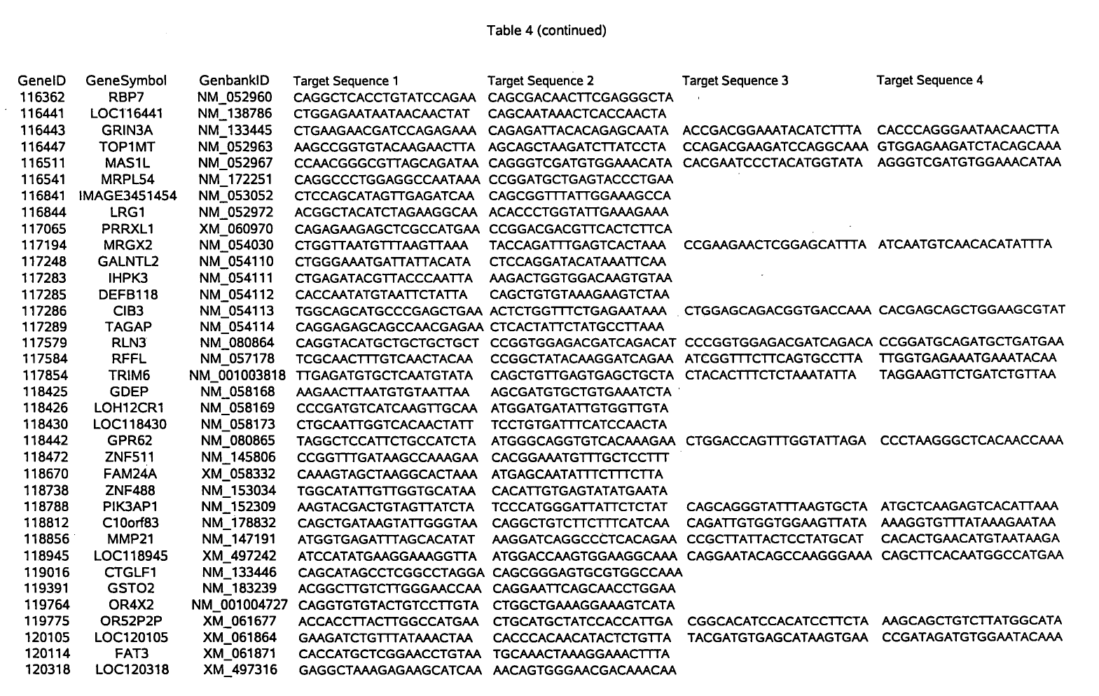

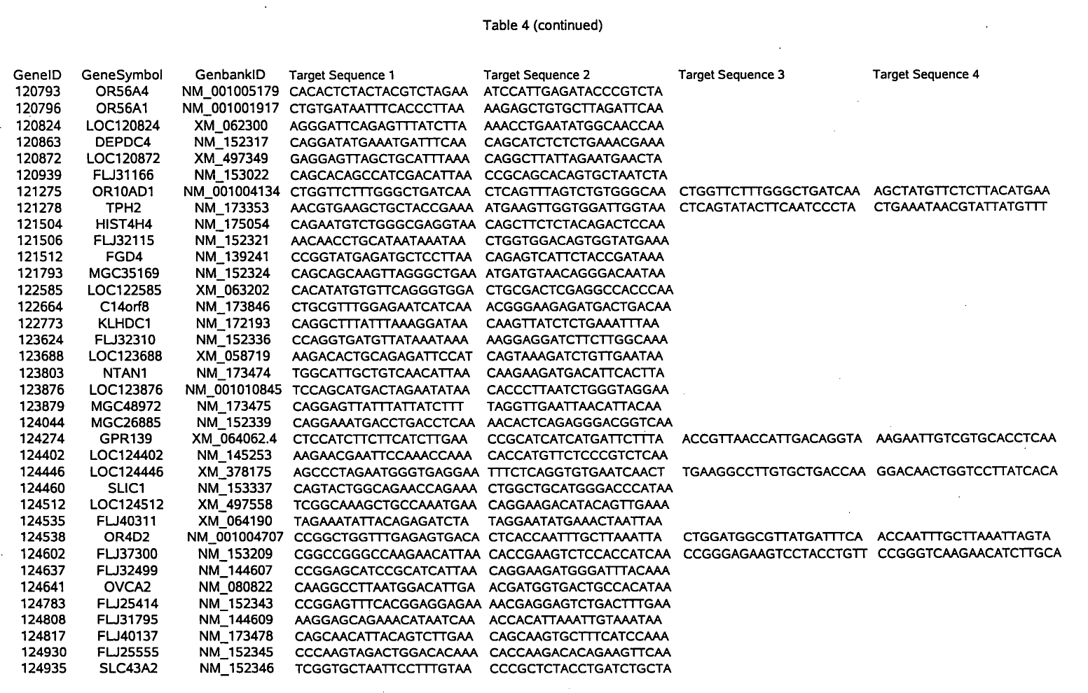

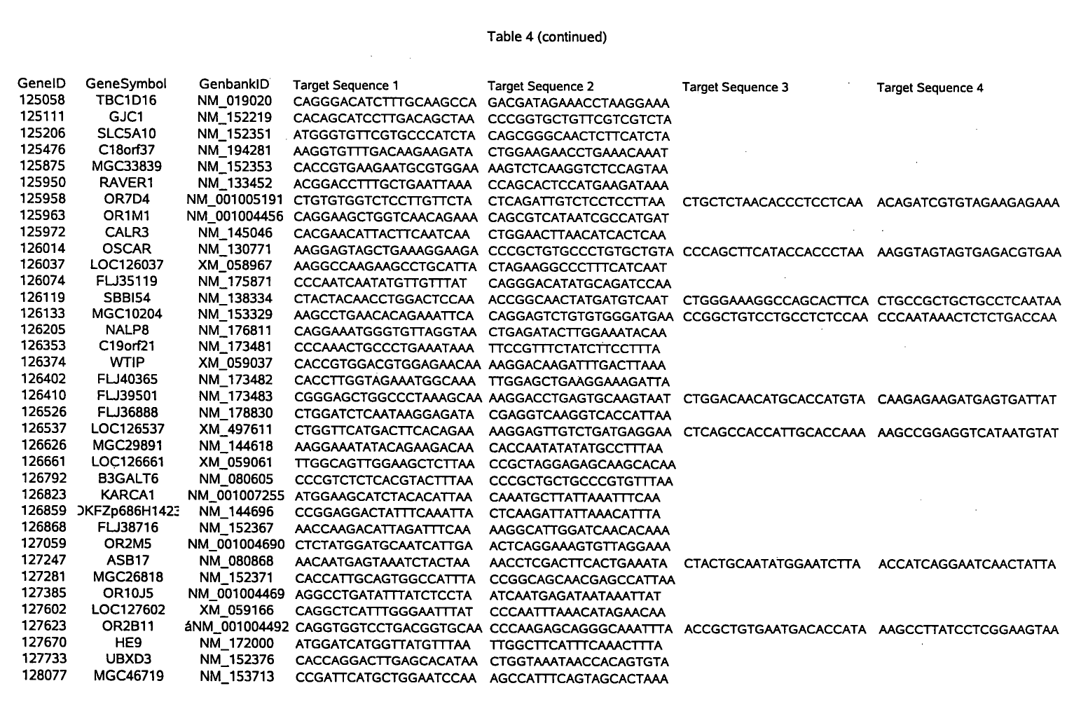

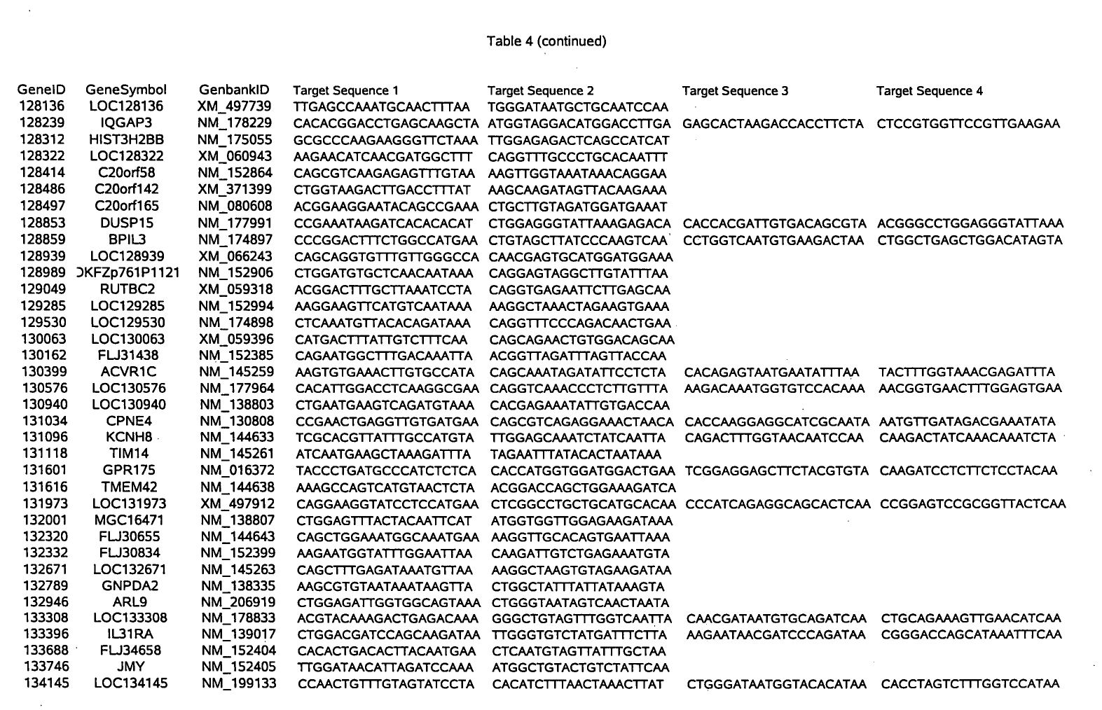

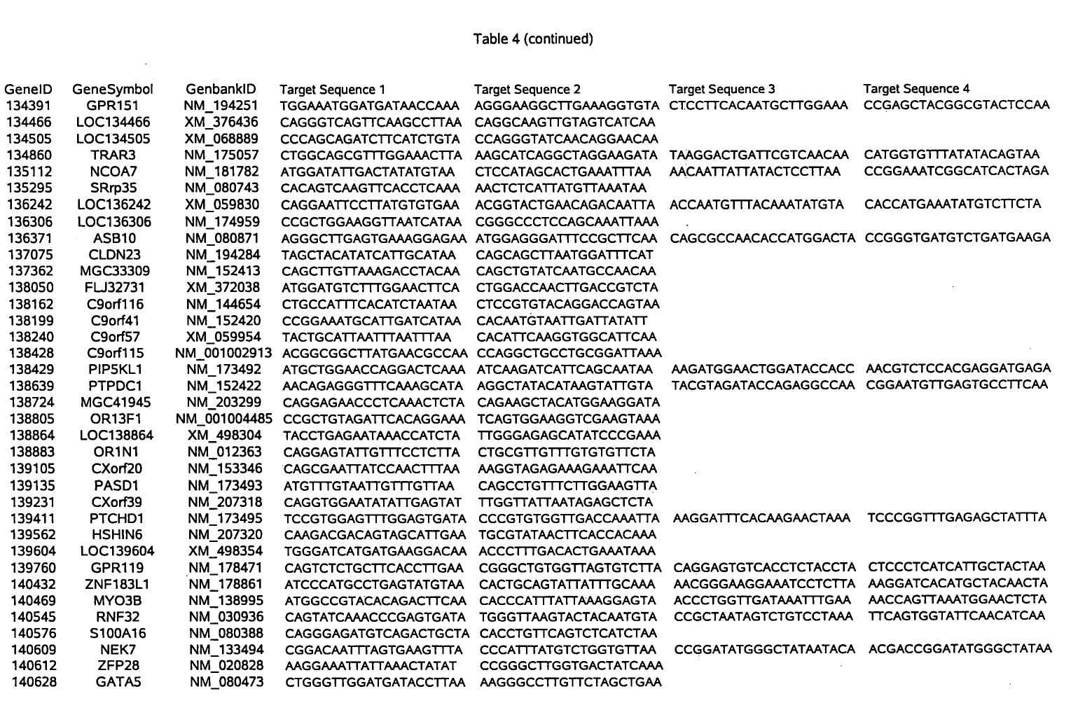

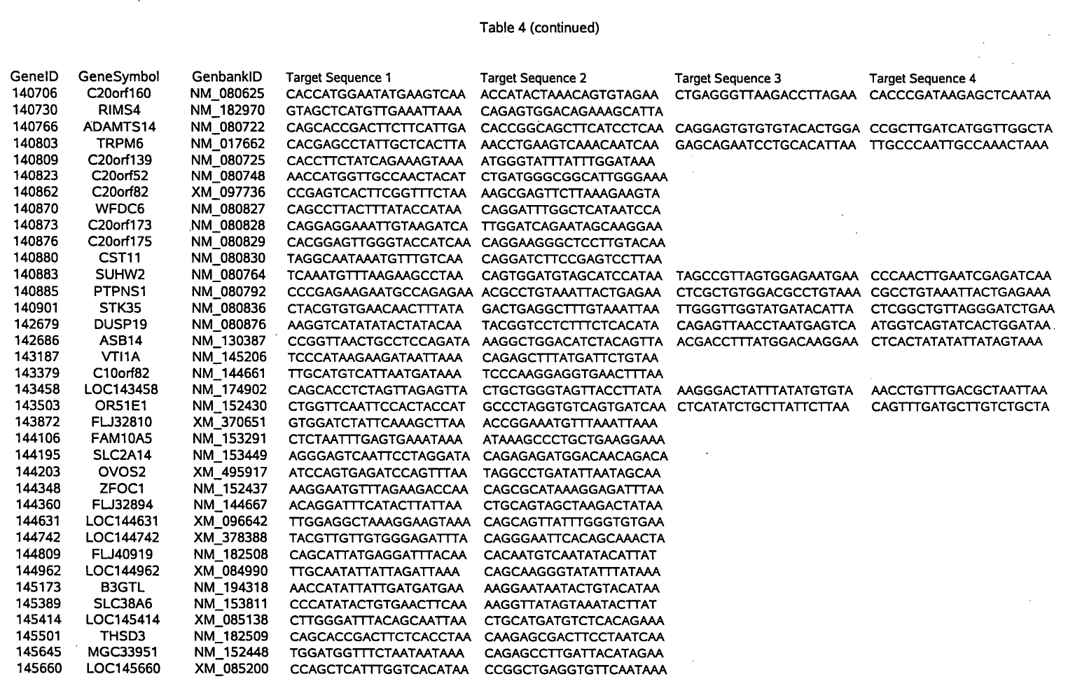

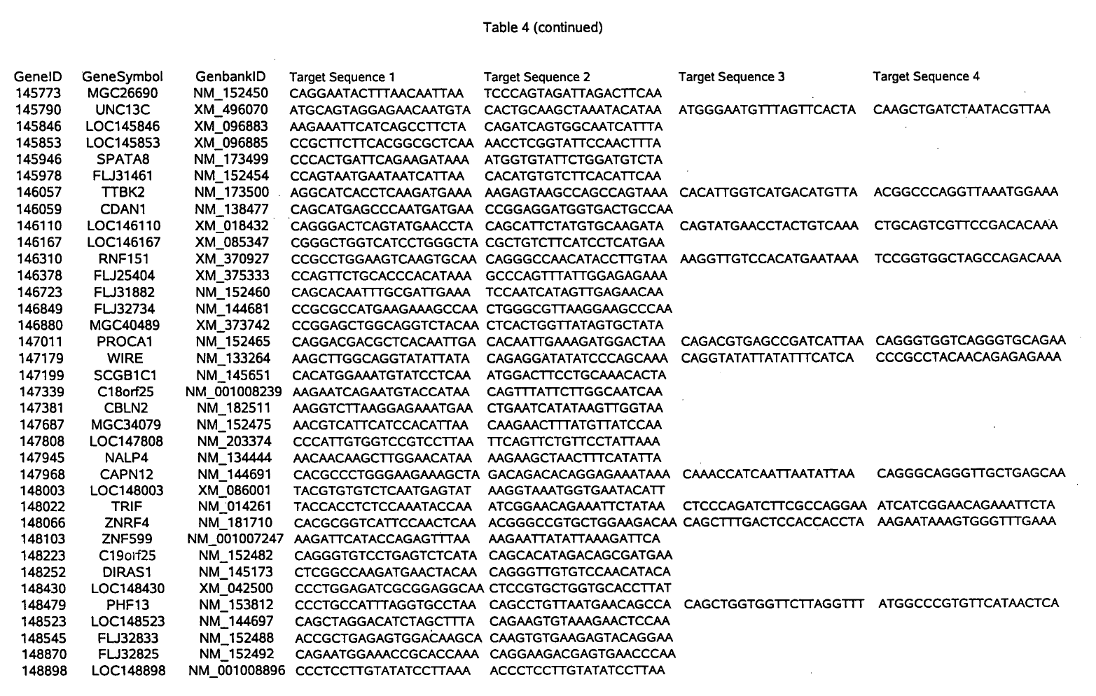

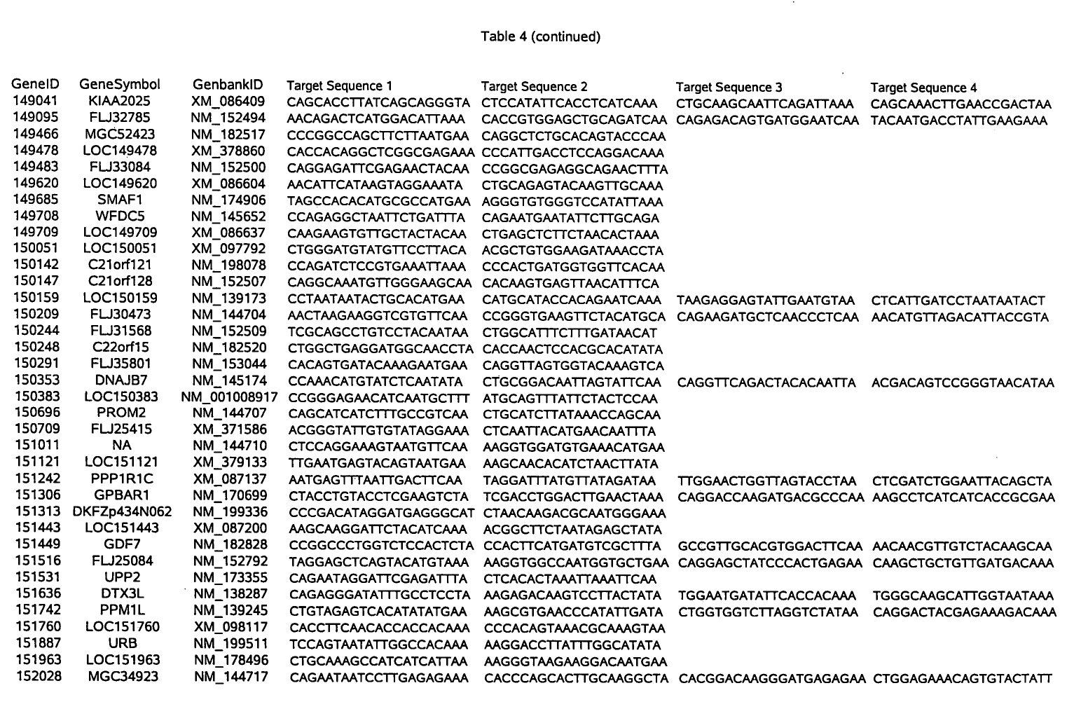

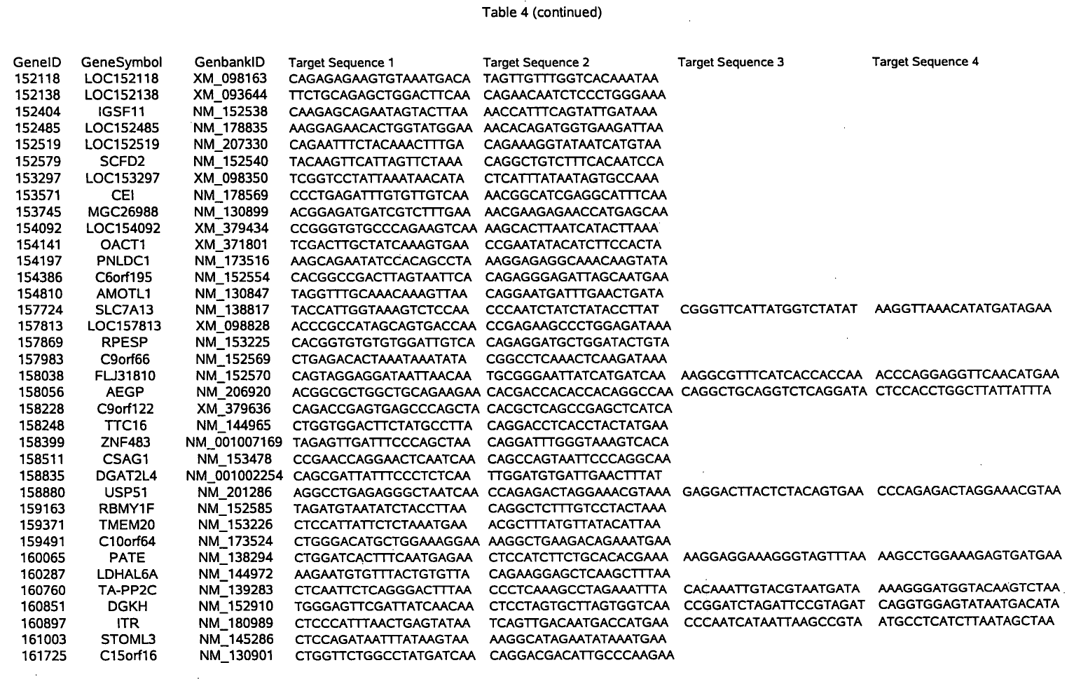

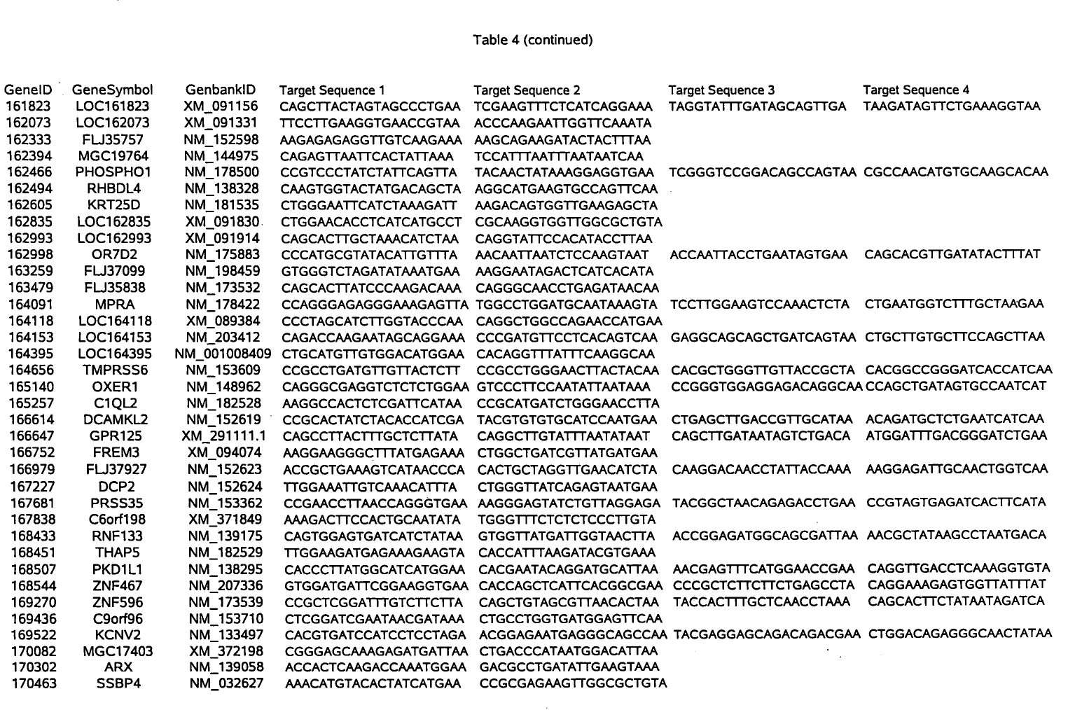

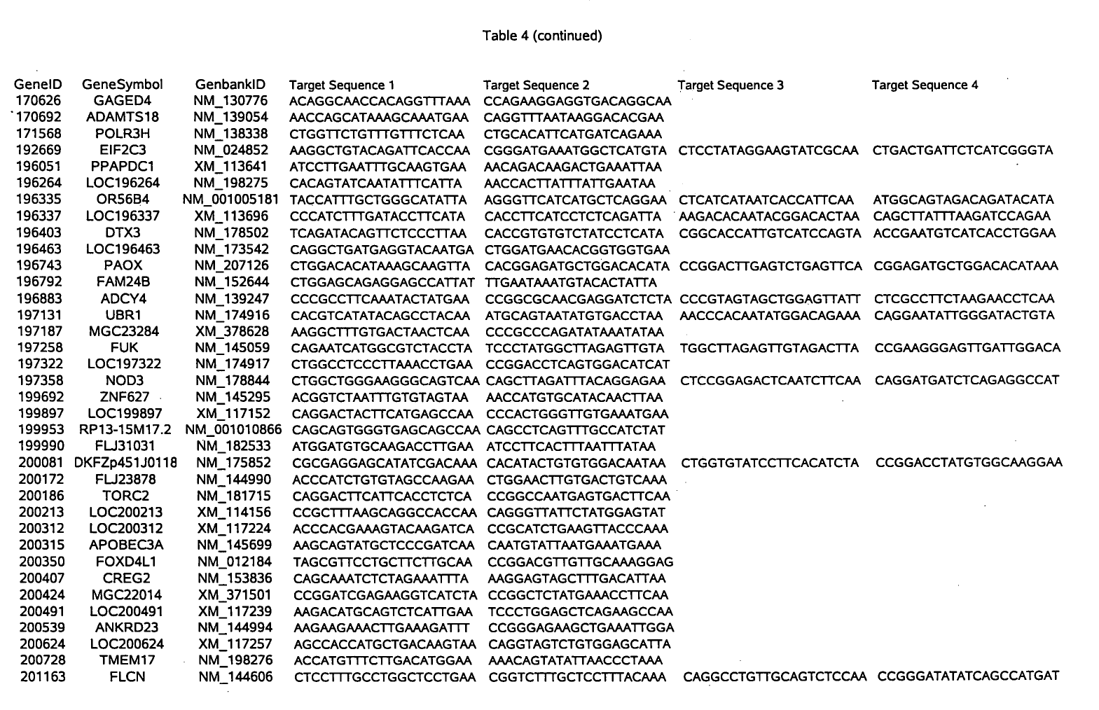

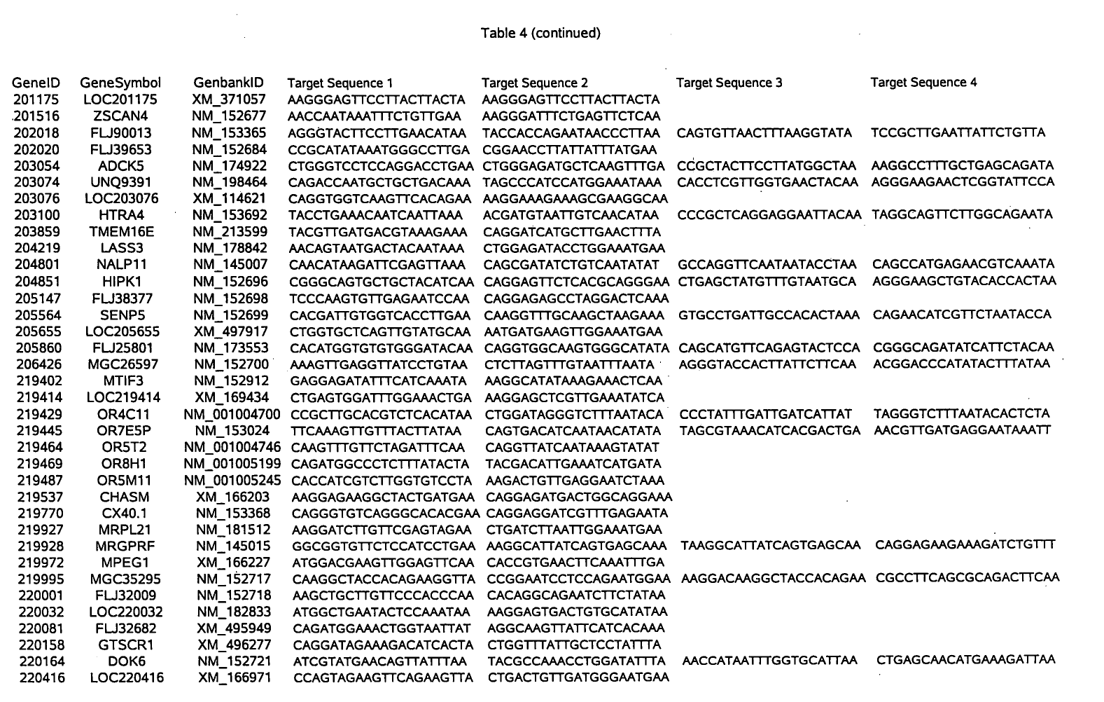

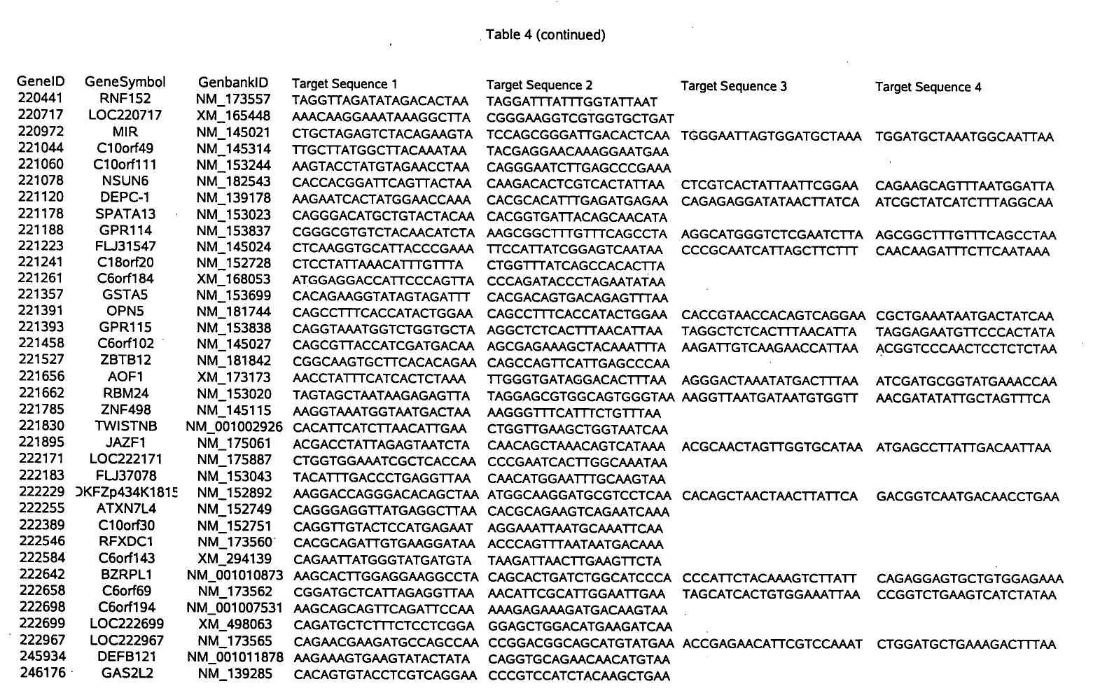

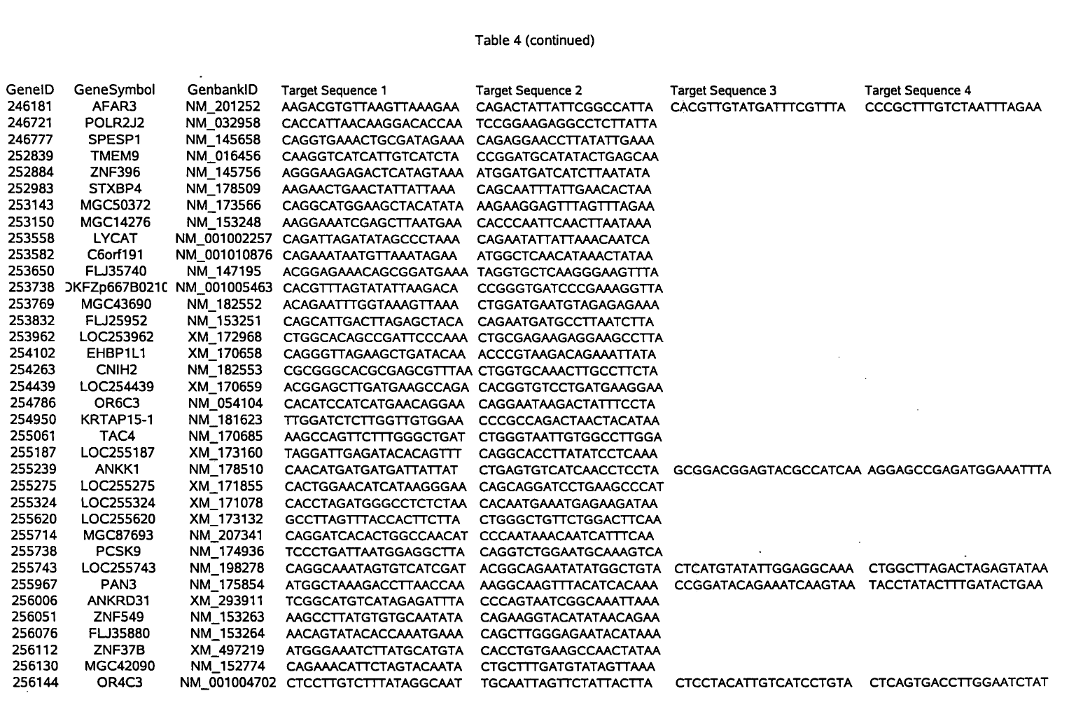

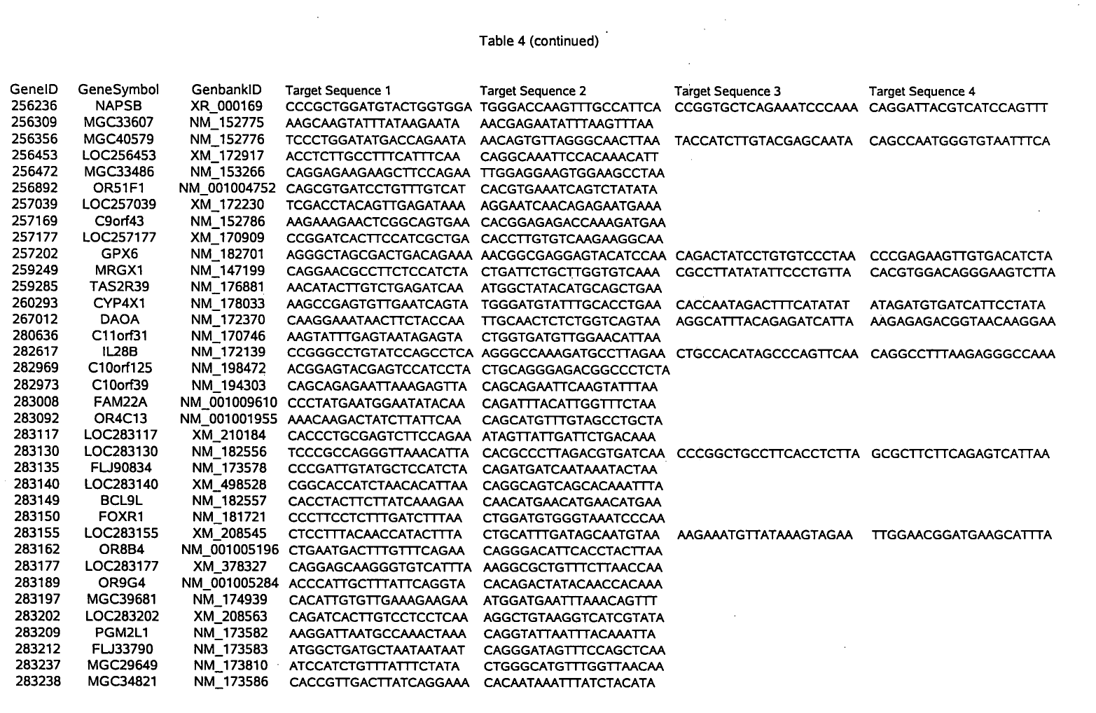

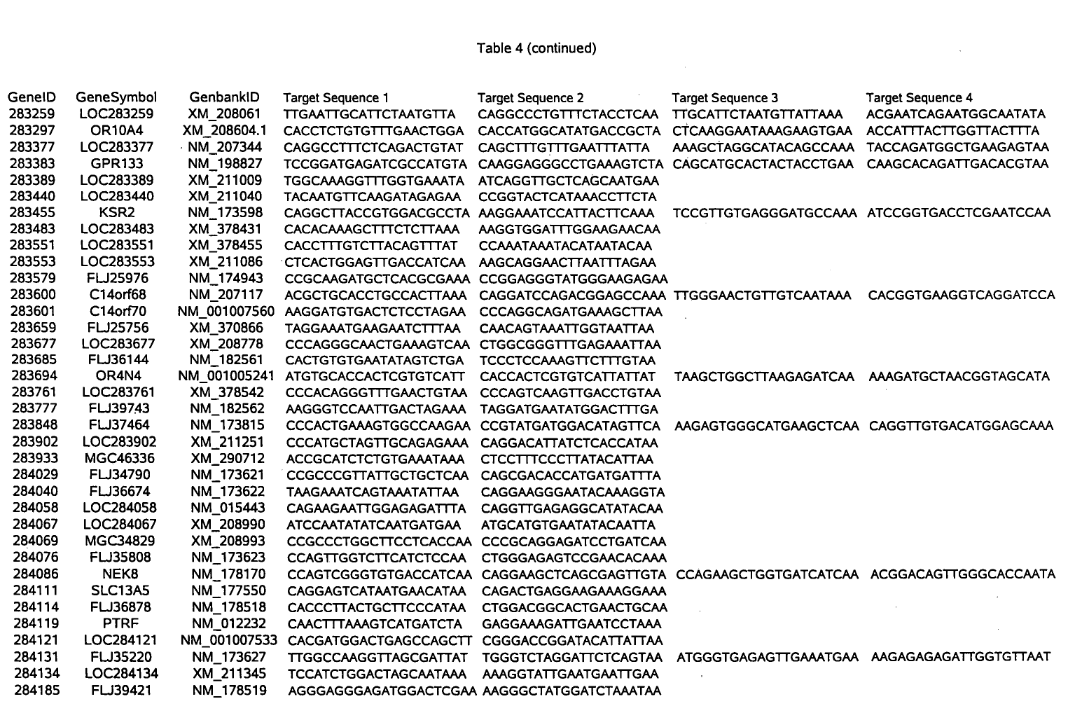

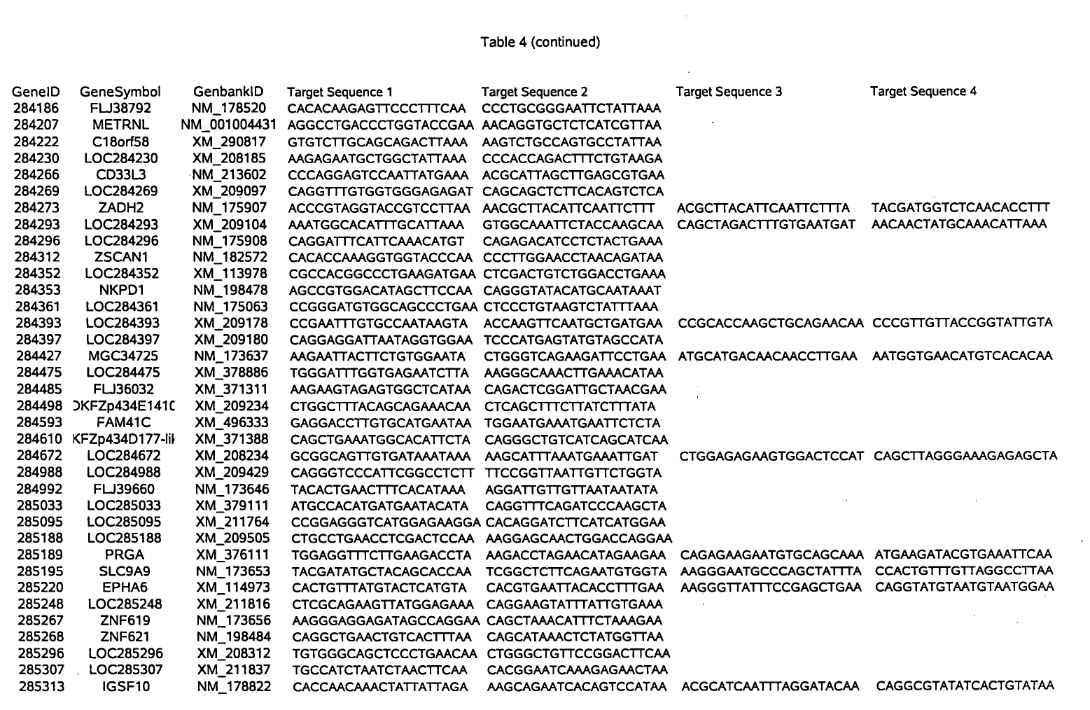

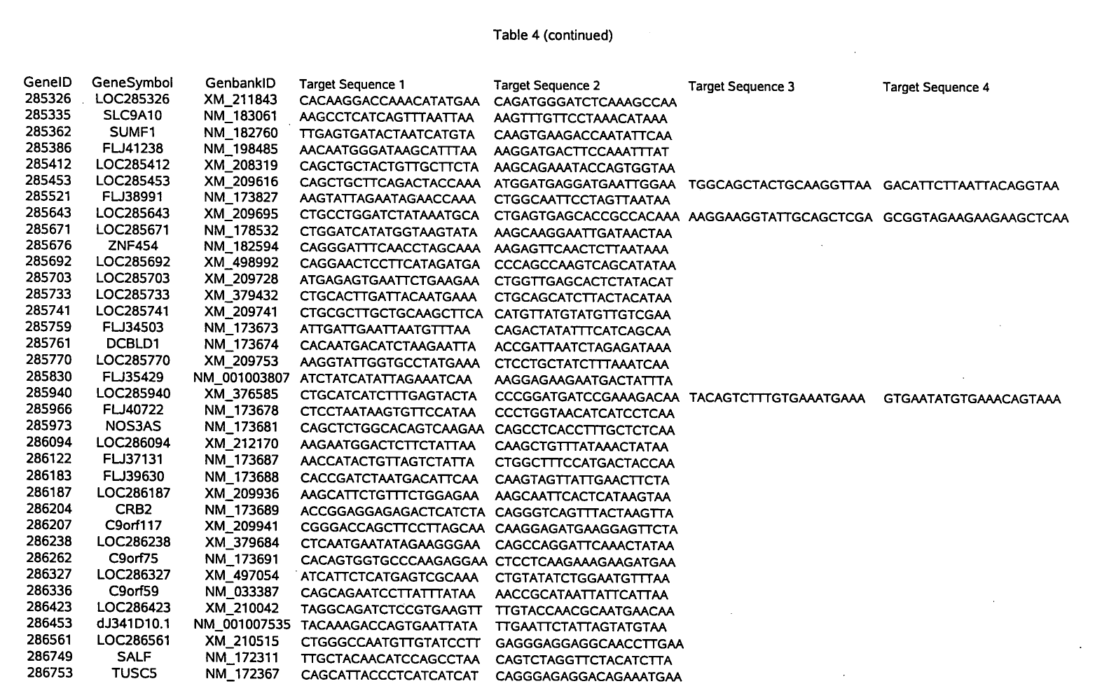

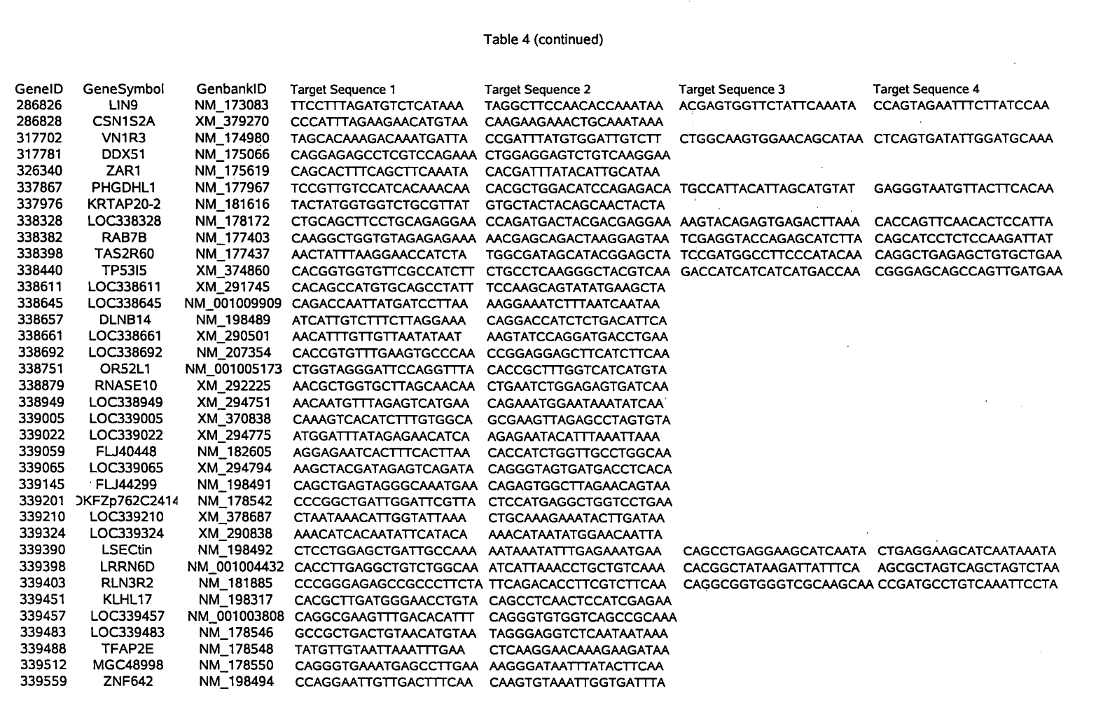

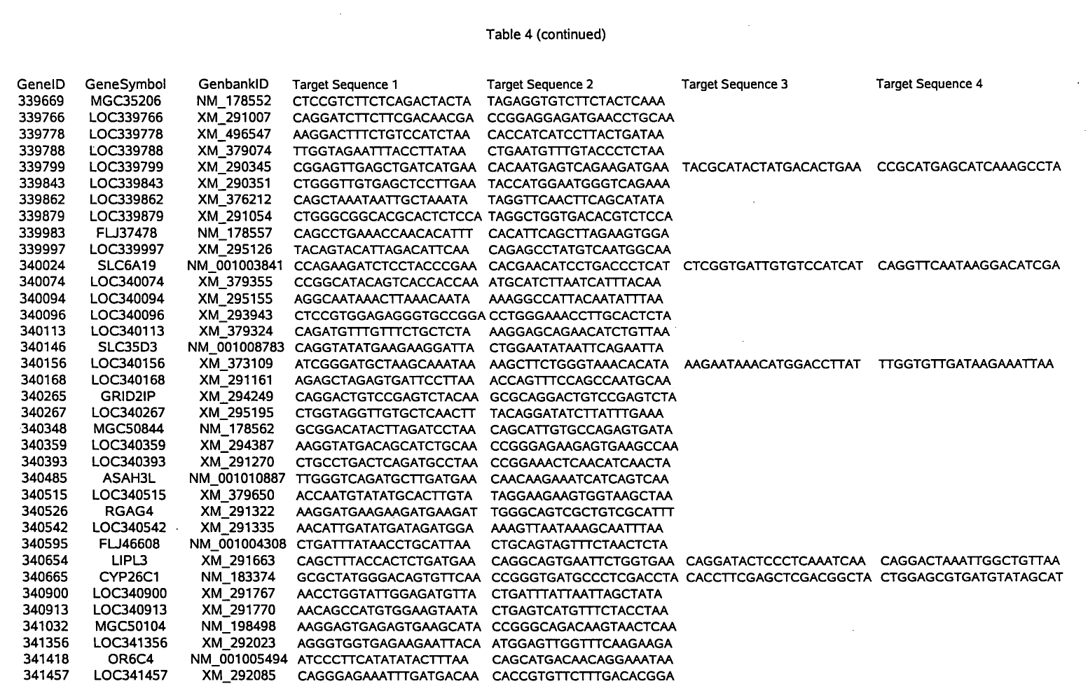

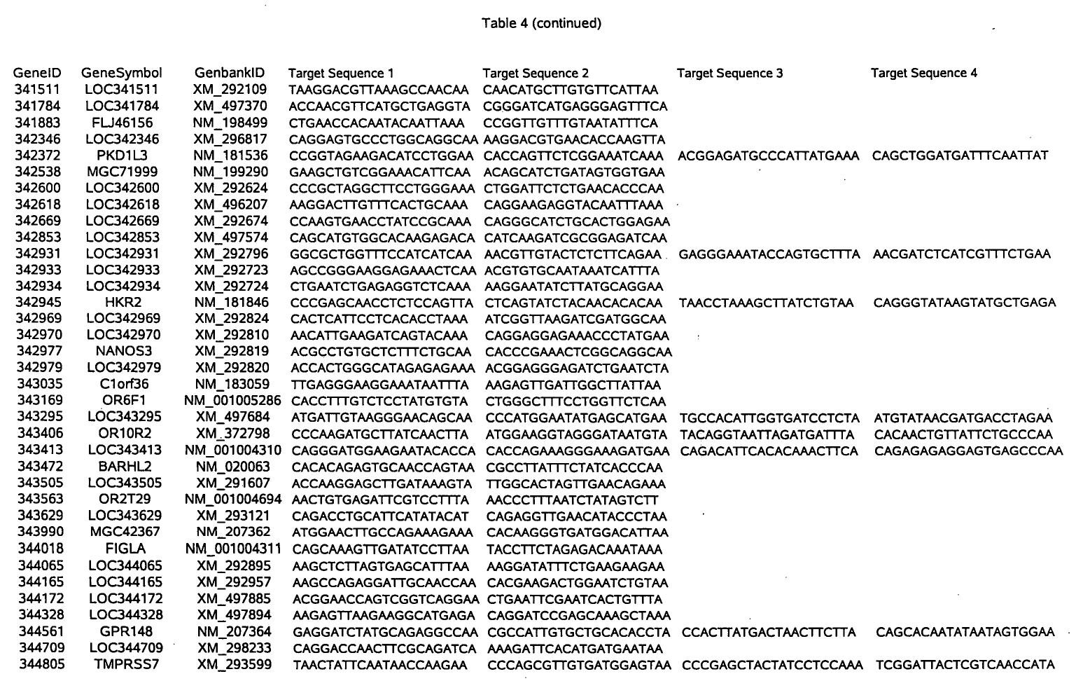

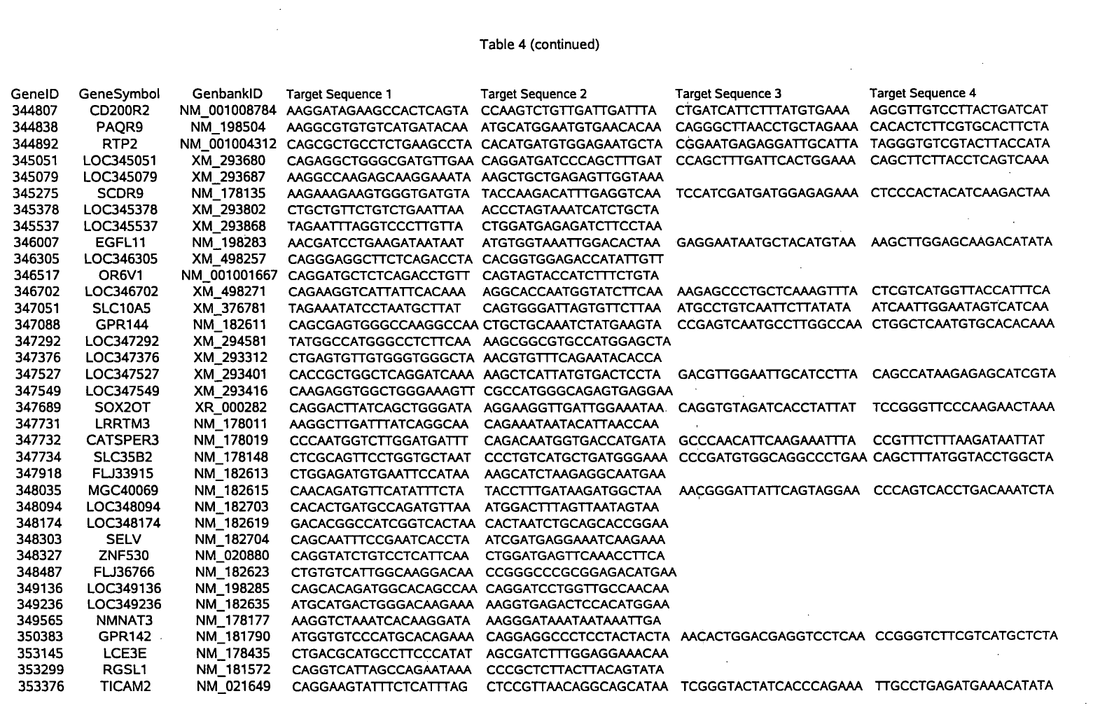

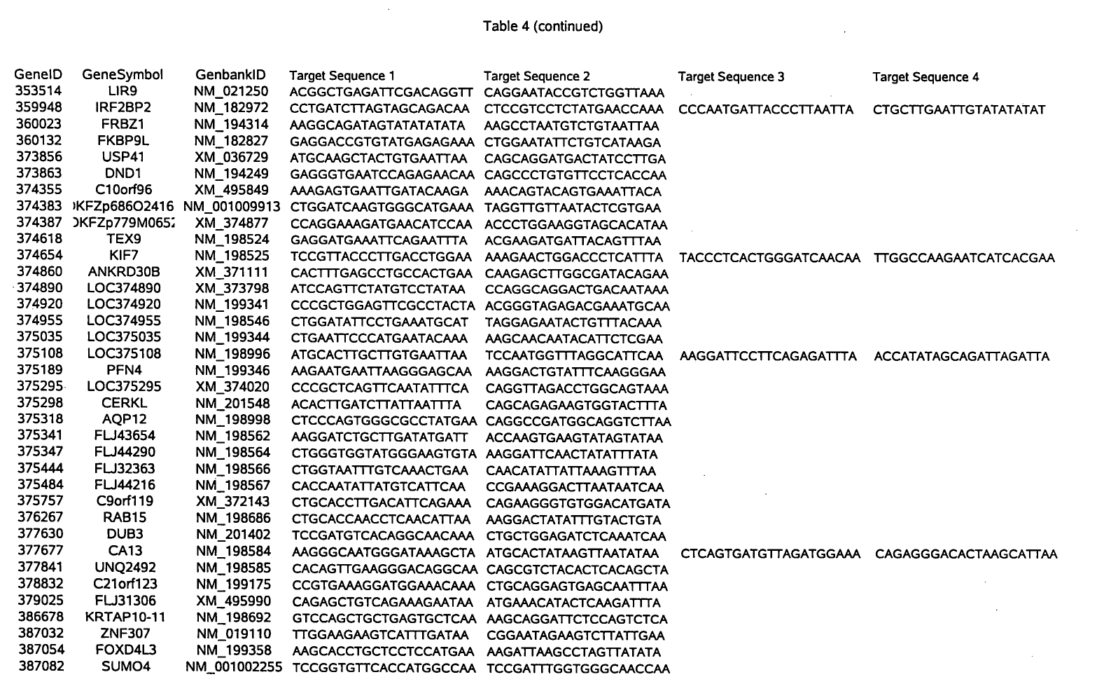

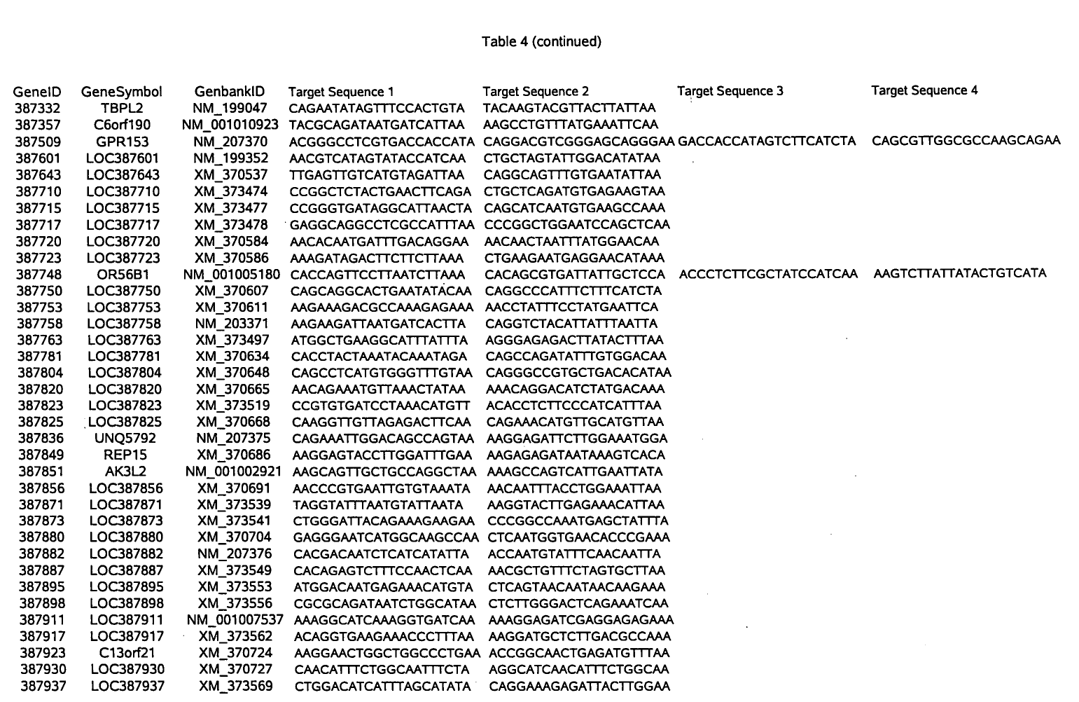

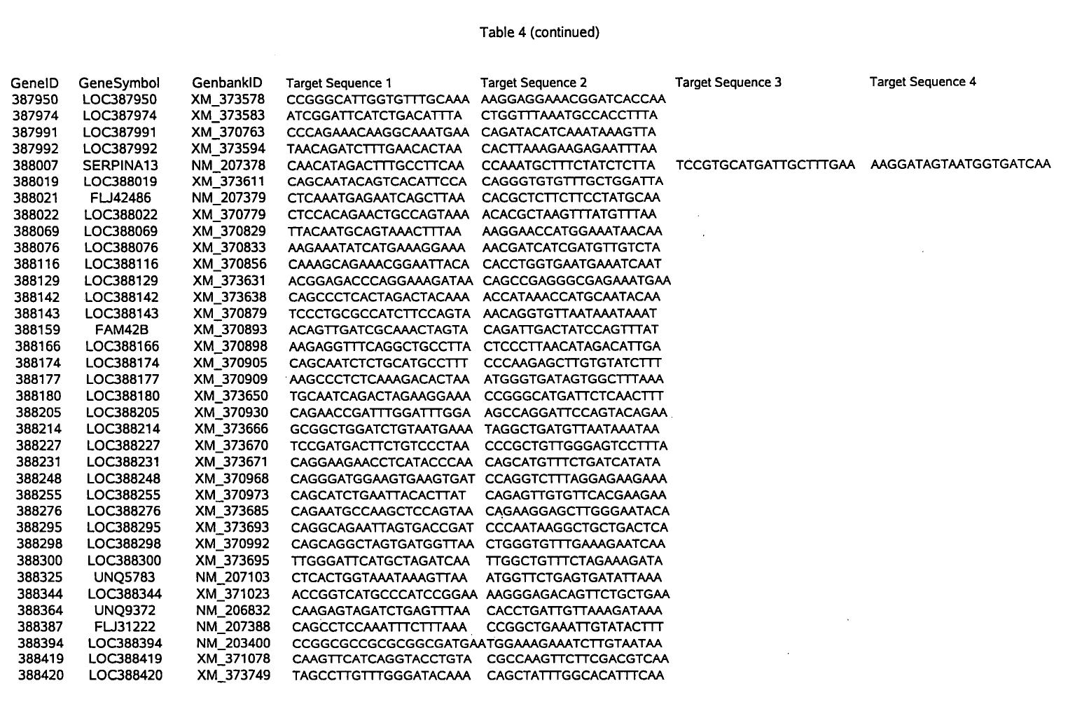

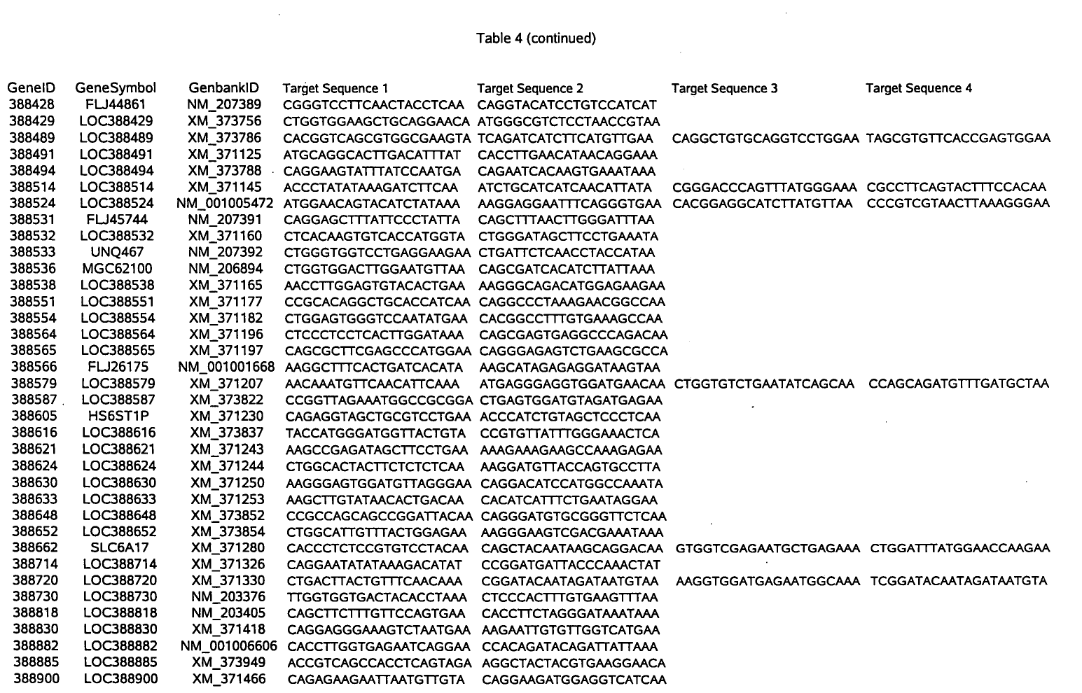

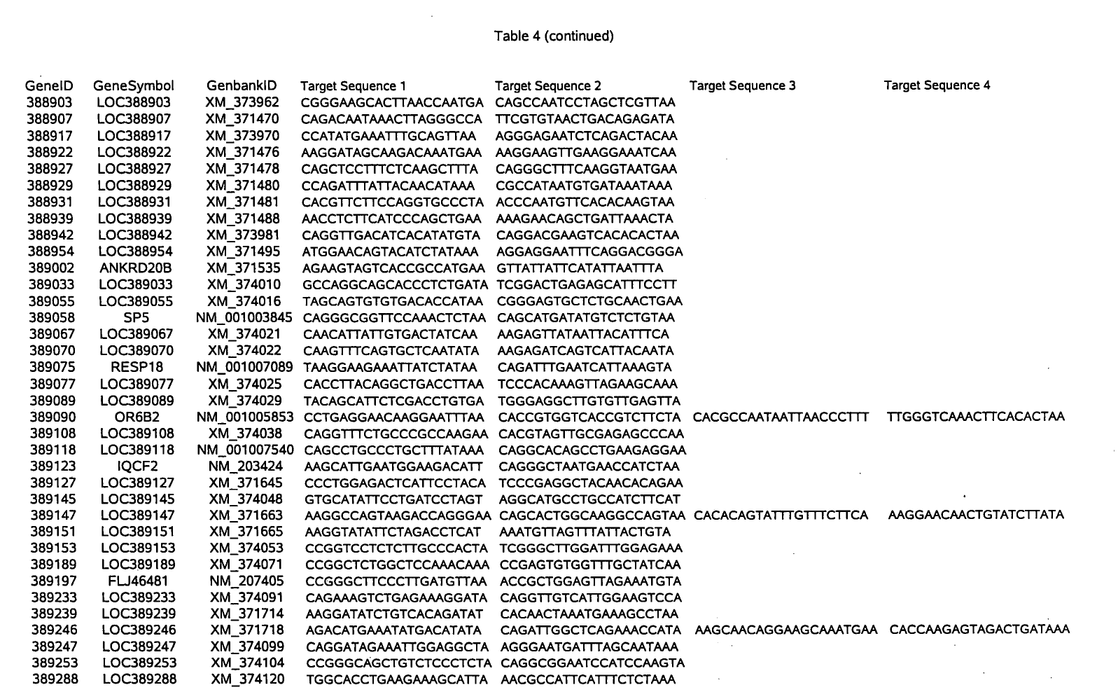

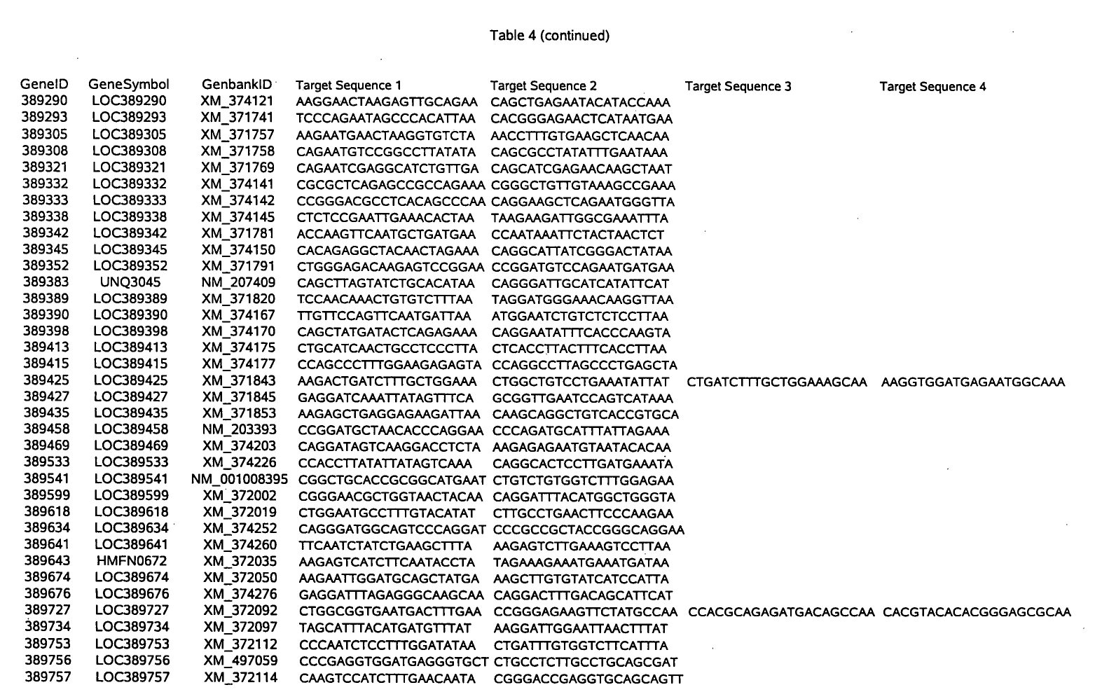

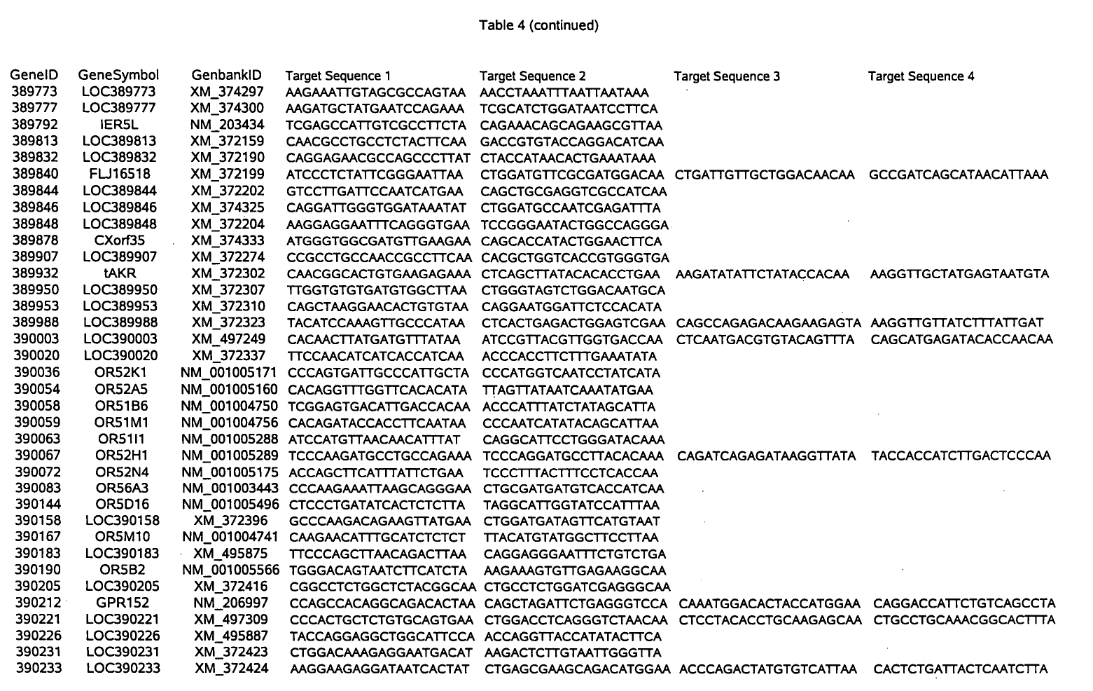

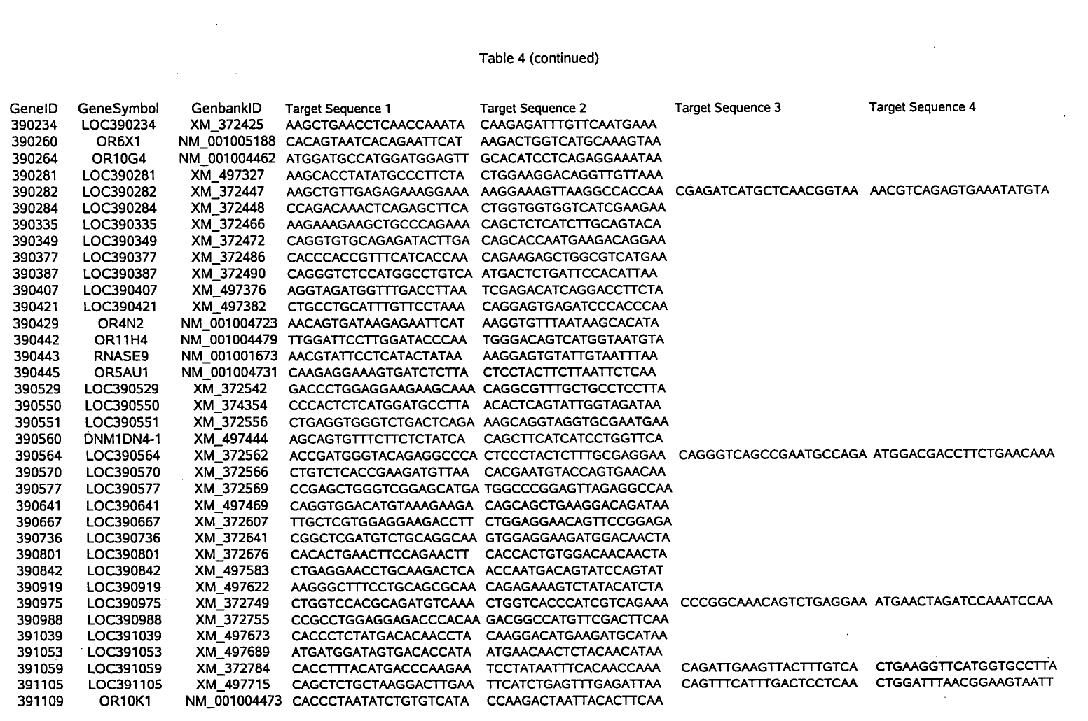

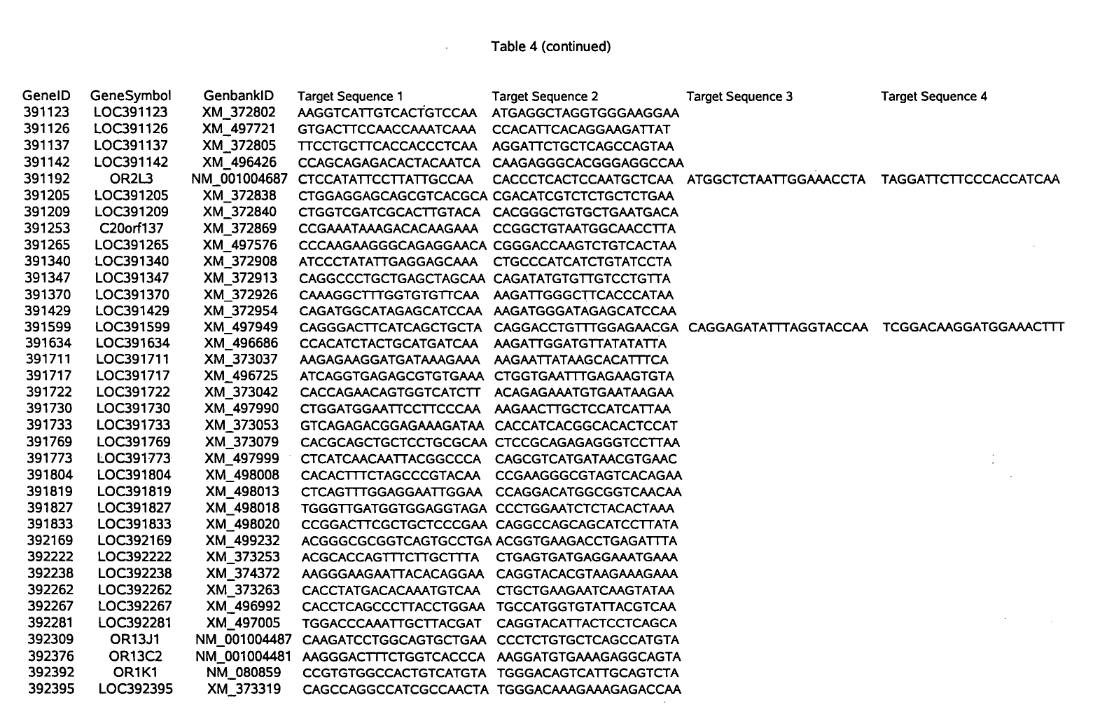

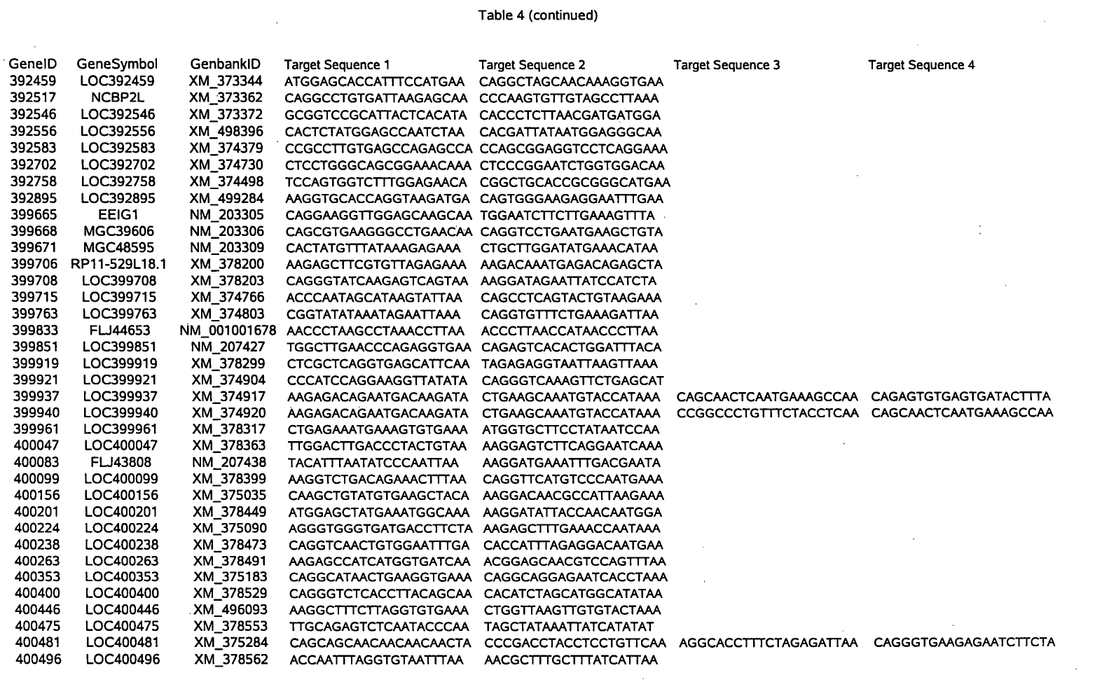

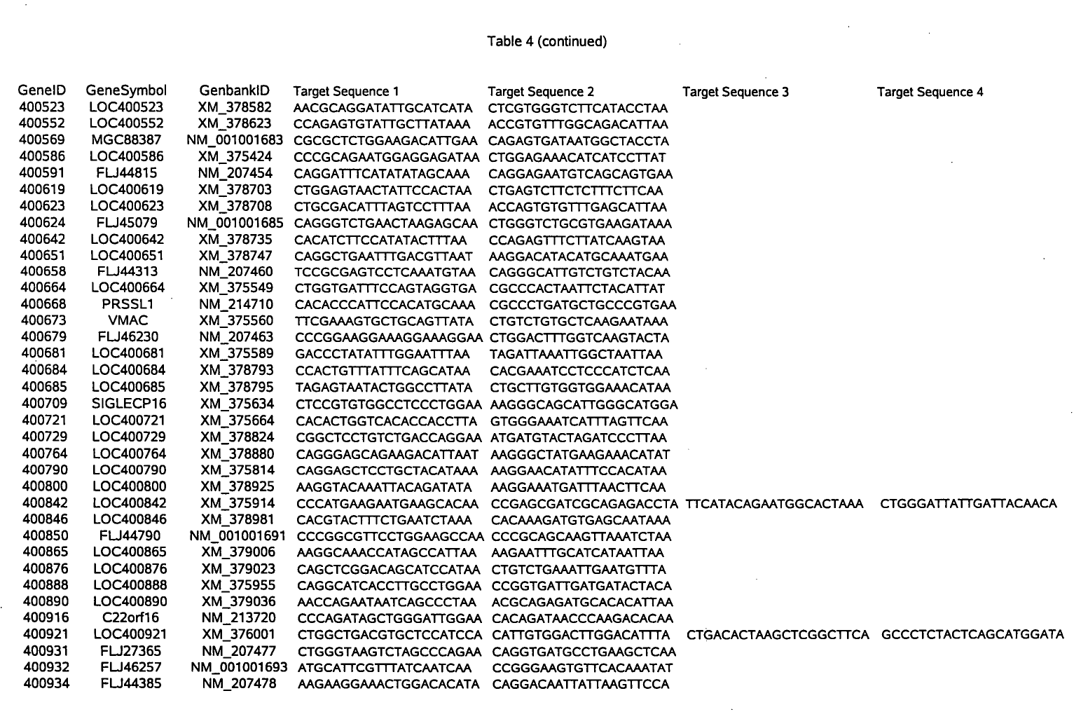

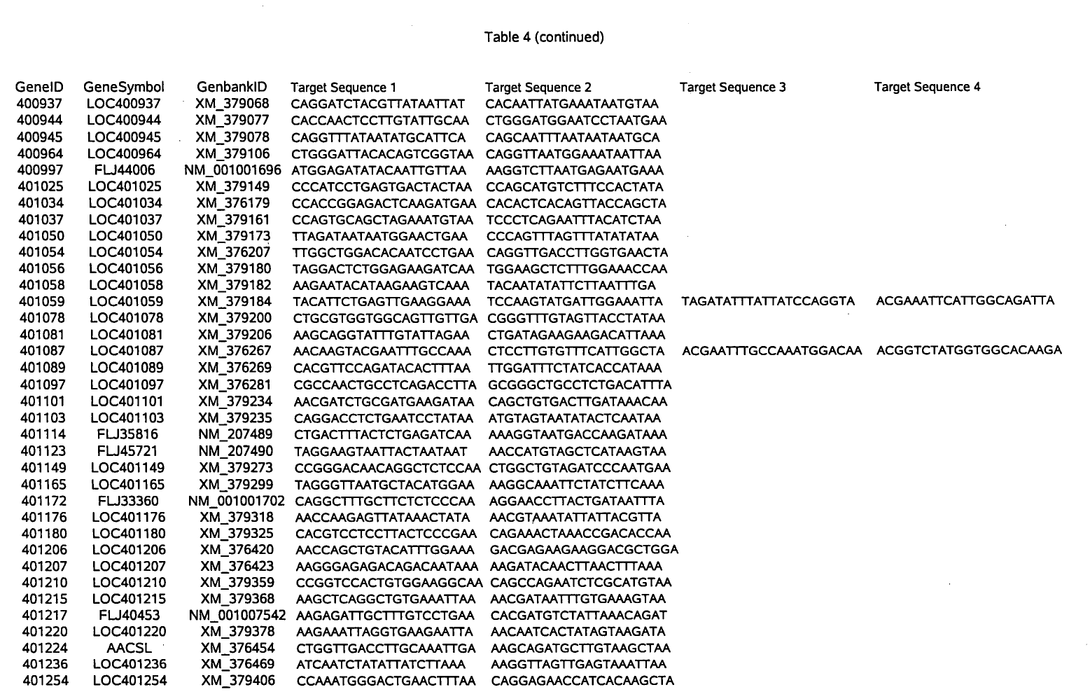

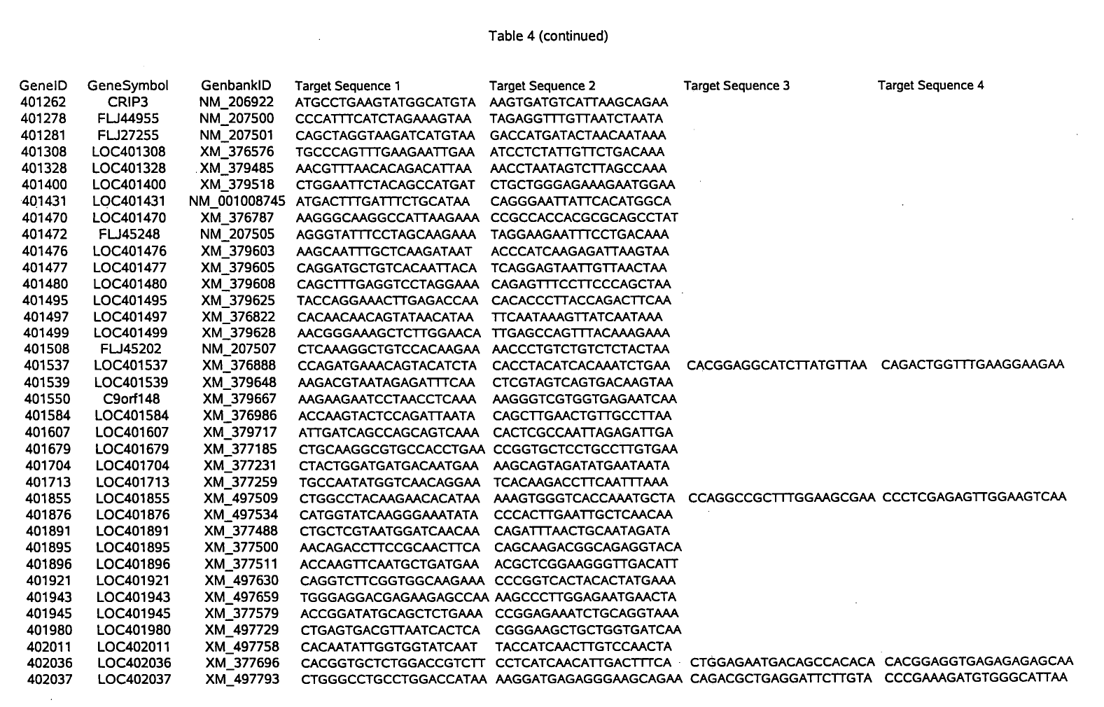

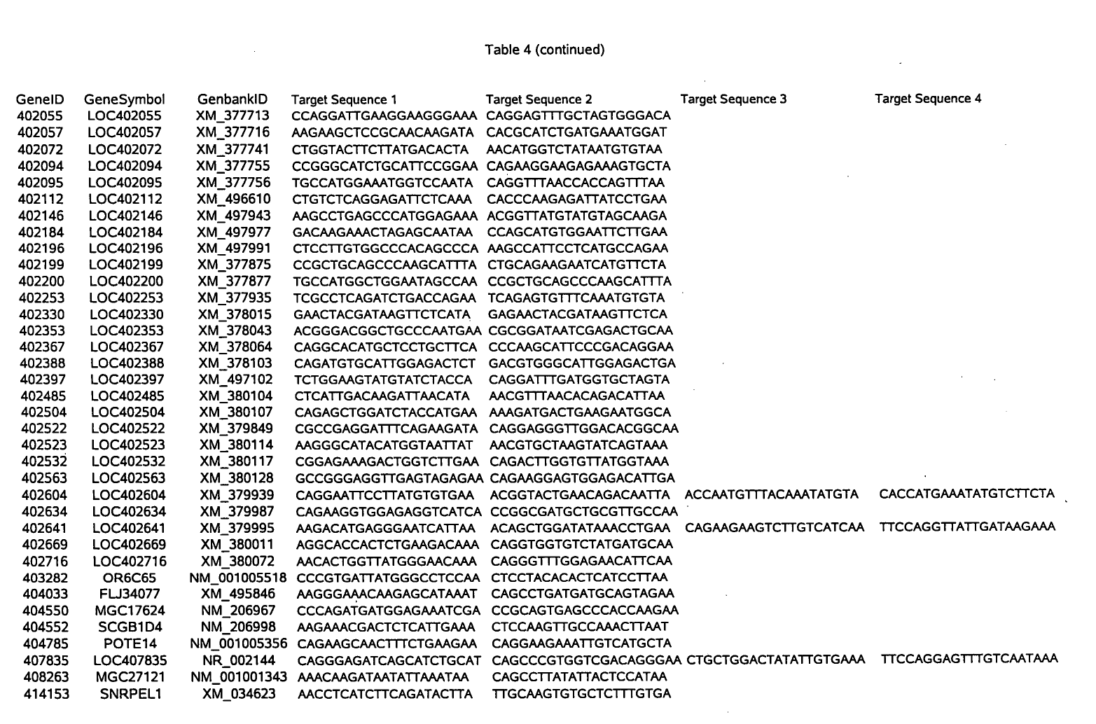

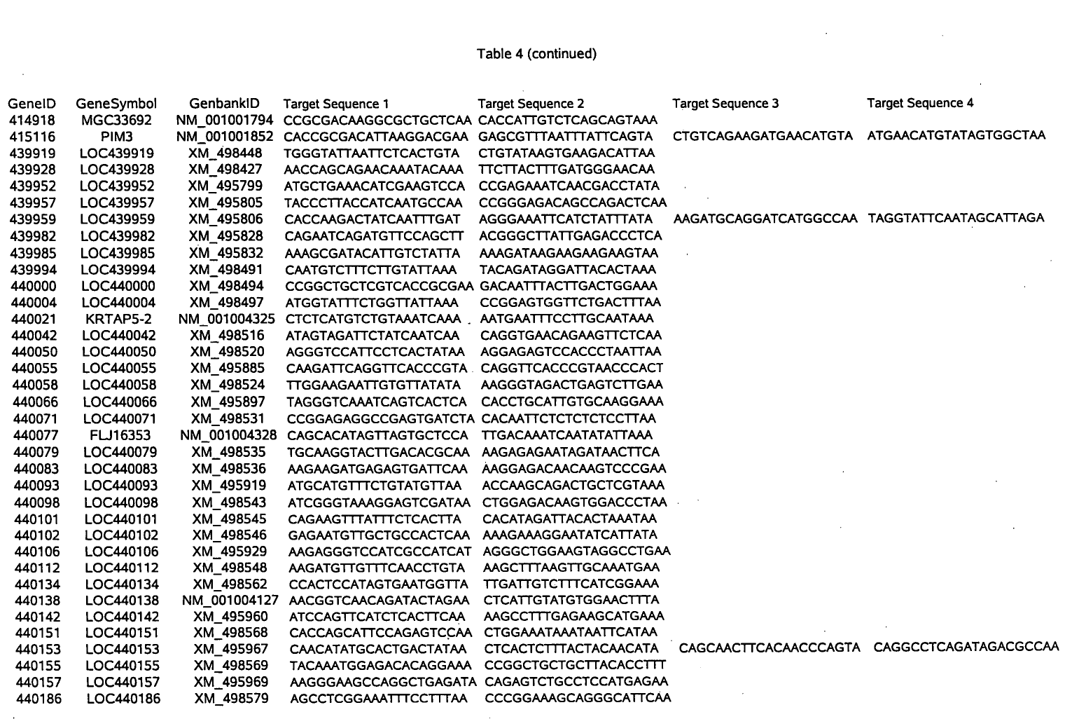

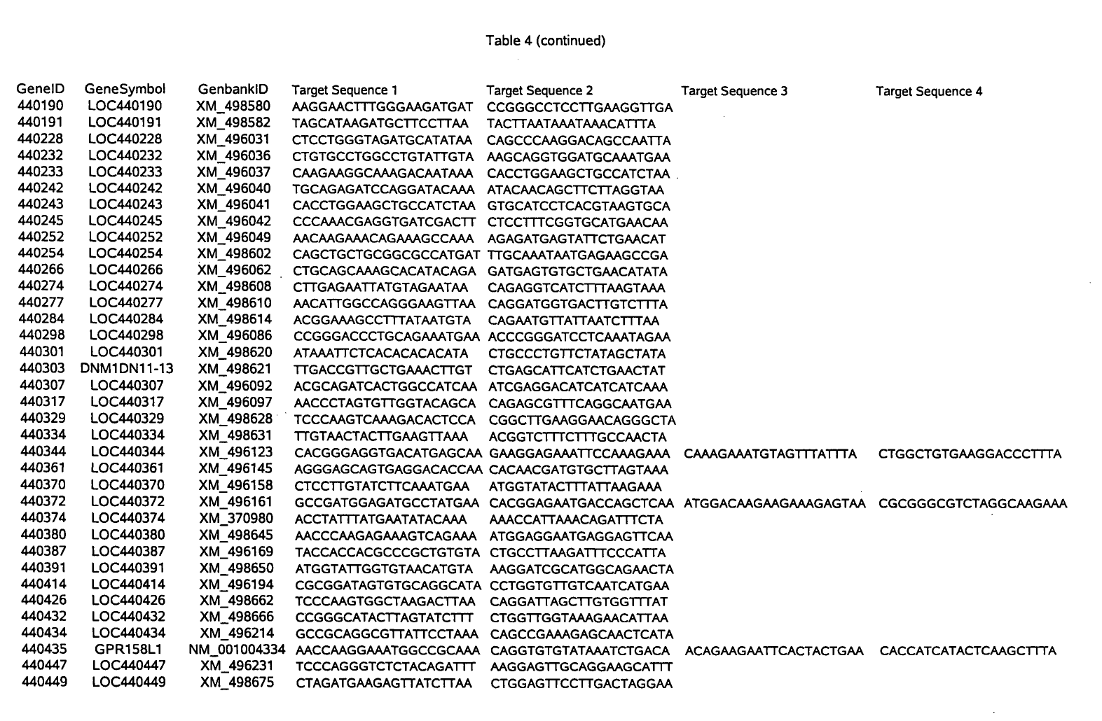

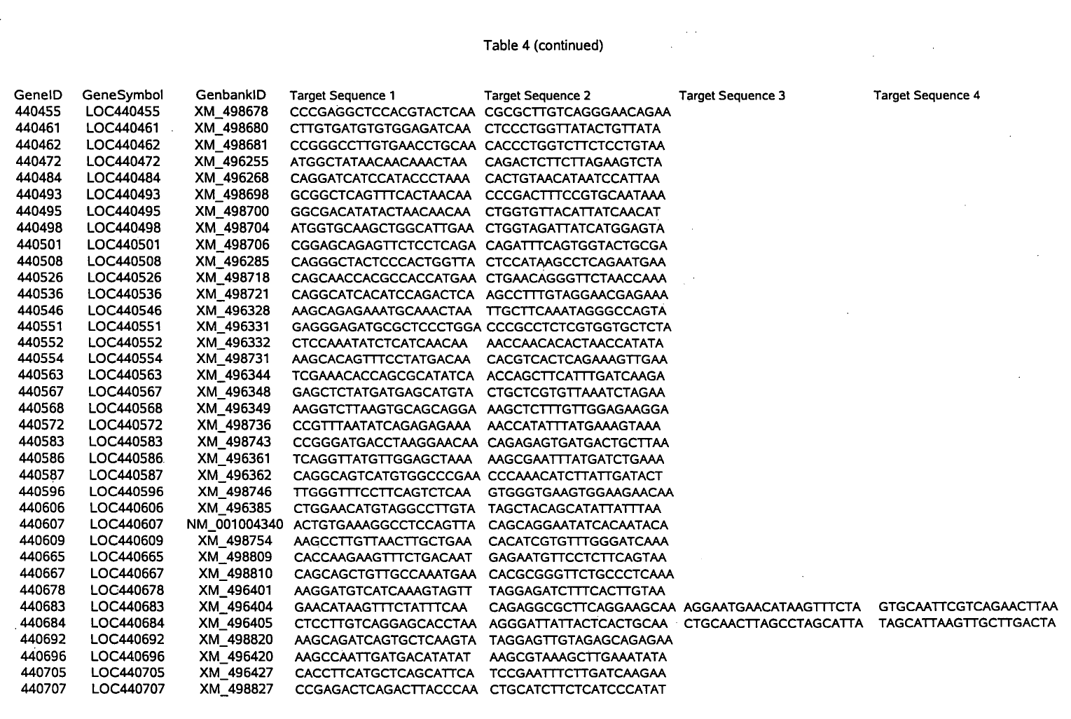

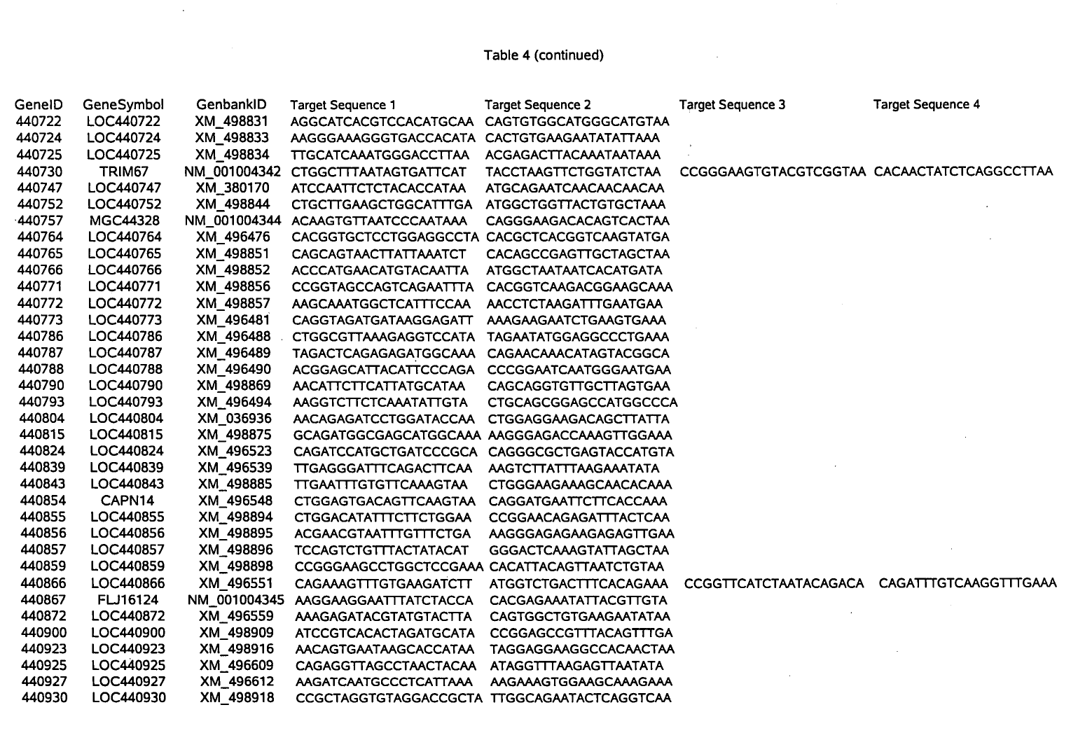

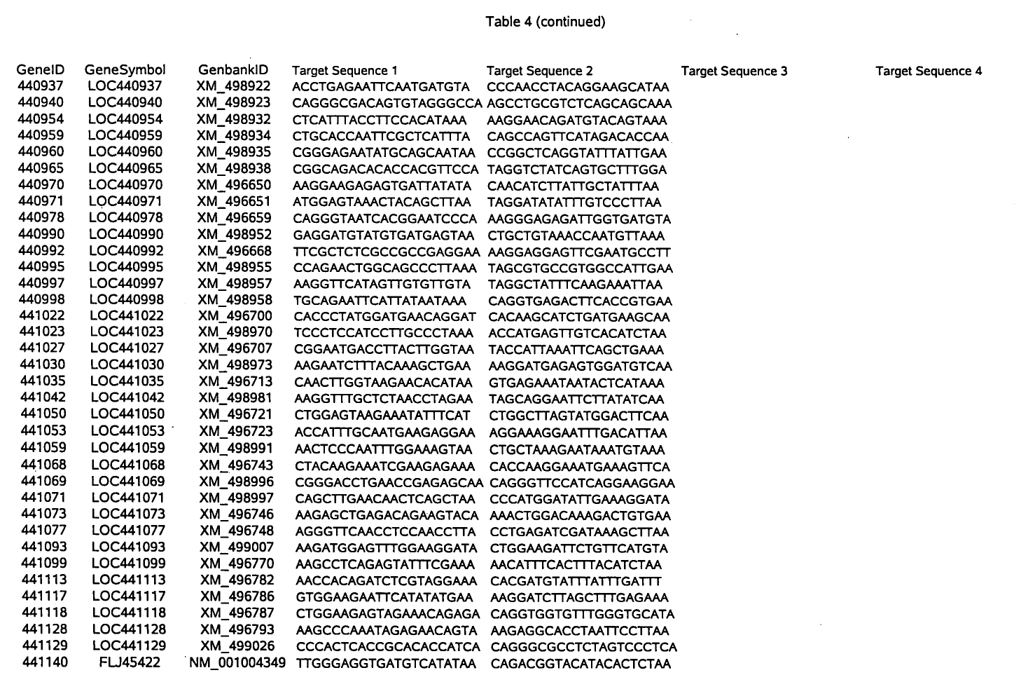

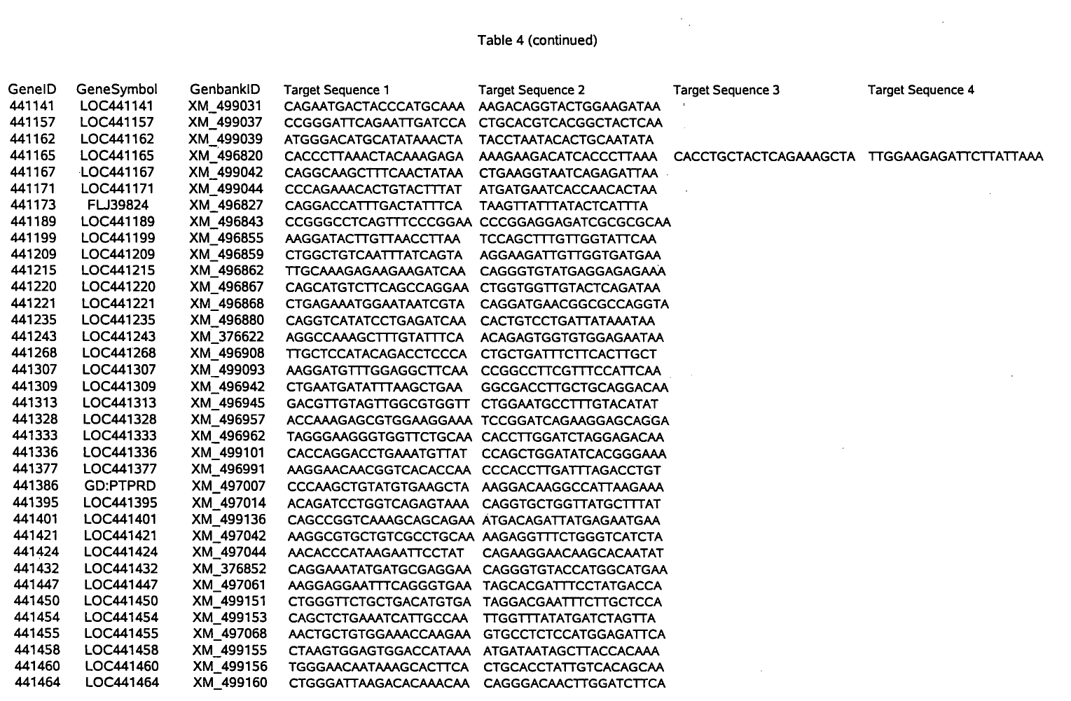

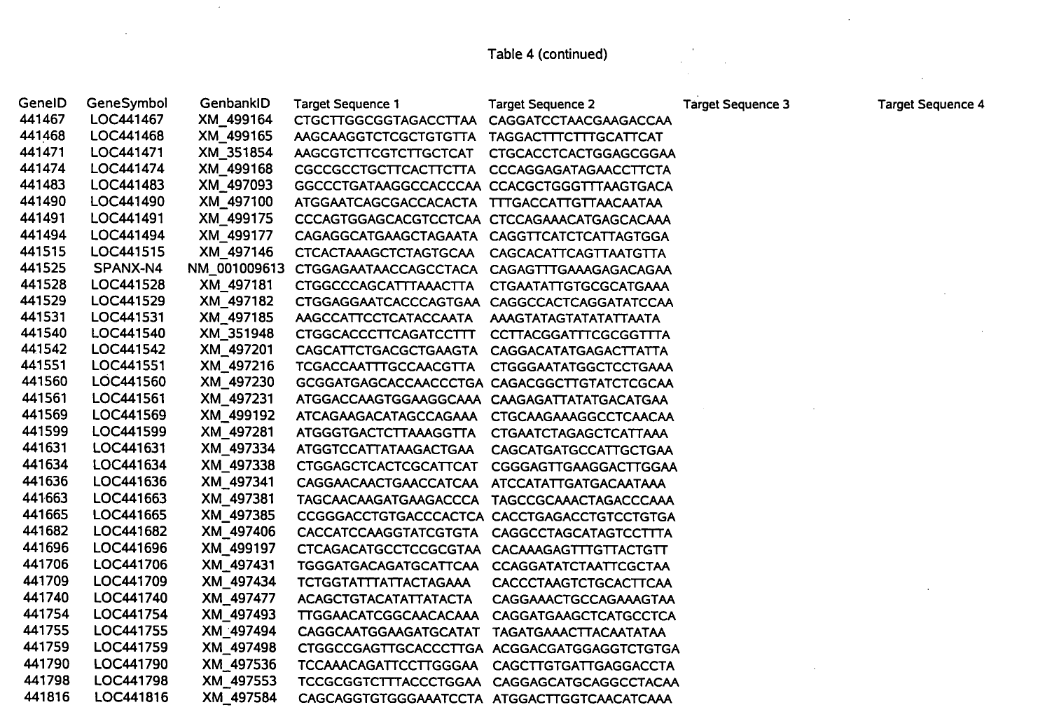

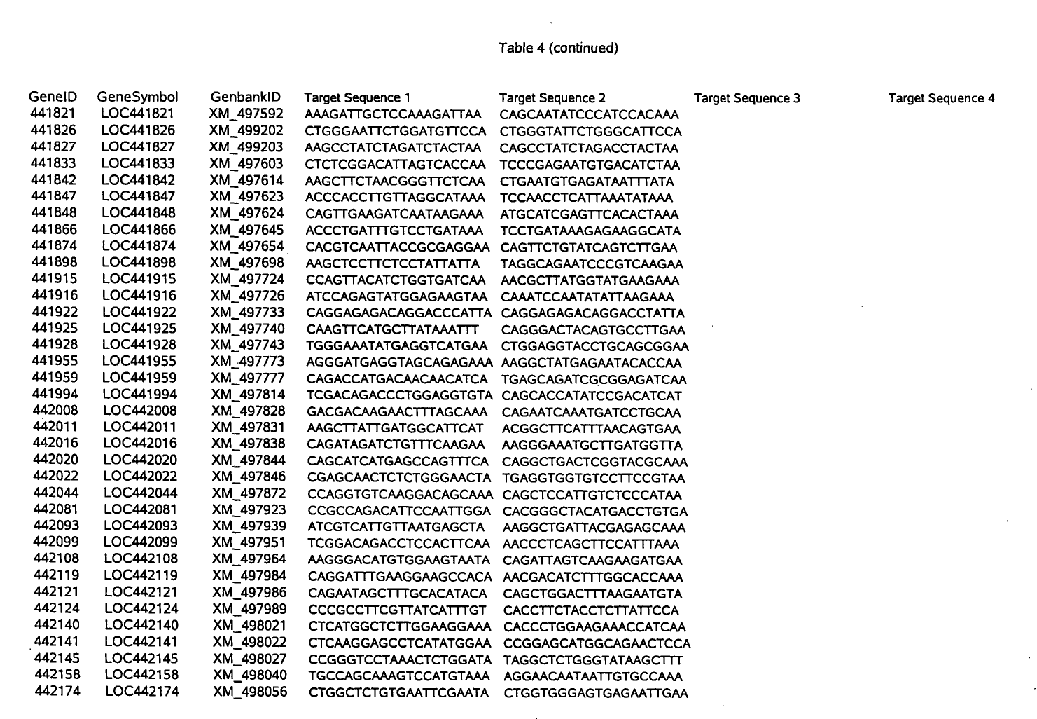

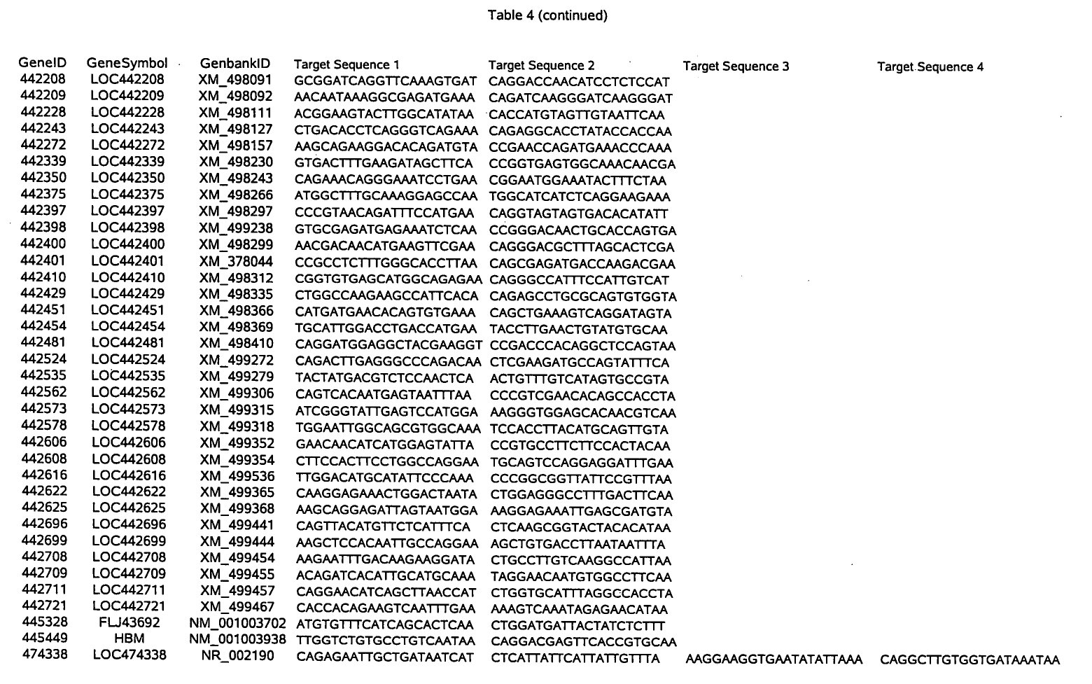

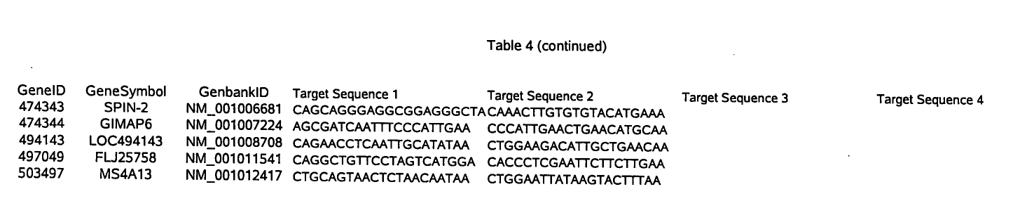

The cell based assay comprised the following steps (see also FIG. 5 which describes the experimental setup of the genome wide siRNA screen:
Day 1: Seeding of A549 cells (lung epithelial cells)+reverse transfection of siRNAs
Day 3: Infection with influenza A/WSN/33+transfection of 293T cells zq with indicator plasmid
Day 4: Infection of 293T cells with the supernatant of A549 cells+fixation of A549 cells with formaldehyde
Day 5: Luciferase Assay to quantify virus replication in 293T cells
Day x: Determination of infection rate by the automated microscope.

1. A pharmaceutical composition comprising at least one modulator of a kinase or/and at least one modulator of a kinase binding polypeptide optionally together with pharmaceutically acceptable carriers, adjuvants, diluents or/and additives, for the prevention, alleviation or/and treatment of an influenza virus infection.

2. The pharmaceutical composition as claimed in claim 1, wherein the influenza virus infection is an influenza A virus infection, more preferably with strain Puerto Rico/8/34 or an Avian Influenza virus isolate such as H5N1.

3. The pharmaceutical composition as claimed in claim 1, wherein the at least one modulator is selected from the group consisting of nucleic acids, nucleic acid analogues such as ribozymes, peptides, polypeptides, and antibodies.

4. The pharmaceutical composition as claimed in claim 1, wherein the at least one kinase or/and kinase binding polypeptide is encoded by a nucleic acid or/and gene selected from Table 1A and Table 1B.

5. The pharmaceutical composition as claimed in claim 1, wherein the at least one modulator is an activator.

6. The pharmaceutical composition as claimed in claim 5, wherein the at least one activator comprises
(a) a nucleotide sequence selected from Table 1A or a fragment thereof, or/and
(b) a fragment which is at least 70%, preferably at least 80%, more preferably at least 90% identical to the sequence of (a),
or/and the at least one activator is capable of activating expression or/and gene product activity of a gene comprising sequence (a) or/and (b).

7. The pharmaceutical composition as claimed in claim 1, wherein the at least one modulator is an inhibitor.

8. The pharmaceutical composition as claimed in claim 7, wherein the at least one inhibitor comprises
(a) a nucleotide sequence selected from Table 1B or/and a fragment thereof, or
(b) a sequence which is at least 70%, preferably at least 80%, more preferably at least 90% identical to the sequence of (a),
or/and the at least one inhibitor is capable of inhibiting expression or/and gene product activity of a gene comprising sequence (a) or/and (b).

9. The pharmaceutical composition as claimed in claim 3, wherein the nucleic acid comprises a nucleotide sequence selected from the sequences of Table 2 and fragments thereof.

10. Pharmaceutical composition comprising at least one inhibitor of influenza virus replication optionally together with pharmaceutically acceptable carriers, adjuvants, diluents or/and additives, for the prevention, alleviation or/and treatment of an influenza virus infection.

11. The pharmaceutical composition as claimed in claim 10, wherein the at least one inhibitor comprises
(a) a nucleotide sequence selected from Table 1B and 4 or/and a fragment thereof, or
(b) a sequence which is at least 70%, preferably at least 80%, more preferably at least 90% identical to the sequence of (a),
or/and the at least one inhibitor is capable of inhibiting expression or/and gene product activity of a gene comprising sequence (a) or/and (b).

12. The pharmaceutical composition as claimed in claim 10, wherein the nucleic acid comprises a nucleotide sequence selected from the sequences of Table 4 and fragments thereof.

13. The pharmaceutical composition as claimed in claim 3, wherein the nucleic acid is RNA or DNA.

14. The pharmaceutical composition as claimed in claim 7, wherein the nucleic acid is
(i) an RNA molecule capable of RNA interference,
(ii) a precursor of the RNA molecule (i), or/and
(iii) a DNA molecule encoding the RNA molecule (i) or/and the precursor (ii).

15. The pharmaceutical composition as claimed in claim 7, wherein the RNA molecule is a double-stranded RNA molecule, preferably a double-stranded siRNA molecule with or without a single-stranded overhang alone at one end or at both ends.

16. The pharmaceutical composition as claimed in claim 13, wherein the RNA molecule comprises at least one nucleotide analogue or/and deoxyribonucleotide.

17. The pharmaceutical composition as claimed in claim 3, wherein the nucleic acid is an antisense nucleic acid or a DNA encoding the antisense nucleic acid.

18. The pharmaceutical composition as claimed in claim 3, wherein the nucleic acid has a length of at least 15, preferably at least 17, more preferably at least 19, most preferably at least 21 nucleotides.

19. The pharmaceutical composition as claimed in claim 3, wherein the nucleic acid has a length of at the maximum 29, preferably at the maximum 27, more preferably at the maximum 25, especially more preferably at the maximum 23, most preferably at the maximum 21 nucleotides.

20. The pharmaceutical composition as claimed in claim 3, wherein the antibody is directed against a kinase or/and kinase binding polypeptide.

21. The pharmaceutical composition as claimed in claim 20, wherein the antibody is directed against a kinase or/and kinase binding polypeptide comprising
(a) an amino acid sequence encoded by a nucleic acid or/and gene selected from Table 1A and Table 1B, or/and
(b) an amino acid sequence which is at least 70%, preferably at least 80%, more preferably at least 90% identical to the sequence of (a).

22. The pharmaceutical composition as claimed in claim 10 comprising an antibody, wherein the antibody is preferably directed against a polypeptide comprising
(a) an amino acid sequence encoded by a nucleic acid or/and gene selected from Table 4, or/and
(b) an amino acid sequence which is at least 70%, preferably at least 80%, more preferably at least 90% identical to the sequence of (a).

23. The pharmaceutical composition of claim 1 further comprising an agent suitable of transportation of the at least one modulator of a kinase or/and a kinase binding polypeptide into a cell, in particular into a lung epithelial cell.

24. The pharmaceutical composition of claim 23, wherein the further agent is chitosan, which preferably is formulated in nanoparticles.

25. A screening method for identification of a compound suitable for modulation of a kinase or/and a kinase binding polypeptide, comprising the steps
(a) providing a kinase or/and a kinase binding polypeptide,
(b) contacting a compound with the kinase or/and a kinase binding polypeptide of (a),
(c) determining the activity of the kinase or/and the kinase binding polypeptide,
(d) selecting a compound which modulates the activity of the kinase or/and the kinase binding polypeptide.

26. The method of claim 25 which comprises a cellular screening assay or/and a molecular screening assay.

27. The method of claim 25, wherein the kinase or/and the kinase binding polypeptide is provided by a cell or/and a non-human organism capable of expressing the kinase or/and the kinase binding polypeptide.

28. The method of claim 25, wherein the kinase or/and the kinase binding polypeptide is provided in an isolated form.

29. A screening method for identification of a kinase modulator suitable for prevention, alleviation or/and treatment of an influenza virus infection, comprising the steps
(I) providing a cell or/and a non-human organism capable of being infected with an influenza virus and capable of expressing a kinase or/and a kinase binding polypeptide,
(II) contacting the cell or/and the organism of (I) with an influenza virus and with a compound known to be capable of modulating the expression or/and activity of the kinase or/and kinase binding polypeptide,
(III) determining the amount of influenza virus produced by the cell or/and the organism, and
(IV) selecting a compound which reduces the amount of the influenza virus produced by the cell or/and the organism.

30. A screening method for identification of compound suitable for the prevention, alleviation or/and treatment of an influenza virus infection, comprising the steps
(A) providing a cell or/and a non-human organism capable of being infected with an influenza virus and capable of expressing a gene, wherein the gene or/and gene product thereof is capable of modulating an influenza virus replication,
(B) contacting the cell or/and the organism of (A) with an influenza virus and with a compound known to be capable of modulating the expression or/and activity of the gene of (A) or/and the gene product thereof,
(C) determining the amount of influenza virus produced by the cell or/and the organism, and
(D) selecting a compound which reduces the amount of the influenza virus produced by the cell or/and the organism.

31. The method of claim 30, wherein the gene of (A) is selected from table 1A, 1B and 4.

32. A screening method for identification of a compound suitable for prevention, alleviation or/and treatment of an influenza virus infection, comprising the steps
(i) providing a cell or/and a non-human organism capable of expressing a kinase or/and a kinase binding polypeptide,
(ii) contacting a compound with the cell or/and the organism of (i),
(iii) determining the amount or/and the activity of kinase or/and a kinase binding polypeptide, and
(iv) selecting a compound which modulates the amount or/and the activity of the kinase or/and the kinase binding polypeptide.

33. The screening method as claimed in claim 32, wherein the kinase activity in steps (iii) or/and (iv) is determined by measuring kinase expression.

34. A screening method for identification of a compound suitable for prevention, alleviation or/and treatment of an influenza virus infection, comprising the steps
i. providing a cell or/and a non-human organism capable of expressing a gene, wherein the gene or/and gene product thereof is capable of modulating an influenza virus replication,
ii. contacting a compound with the cell or/and the organism of i.,
iii. determining the amount or/and the activity of gene product of the gene of (i), and
iv. selecting a compound which modulates the amount or/and the activity of the gene product of i.

35. The method of claim 34, wherein the gene of i. is preferably selected from Table 1A, 1B and 4.

36. A screening method for identification of genes suitable as targets for the prevention, alleviation or/and prevention of an influenza virus infection, comprising the steps
(1) providing a cell or/and a transgenic non-human animal capable of expressing, particularly over- or underexpressing a gene,
(2) contacting the cell or/and the organism of (1) with an influenza virus,
(3) modulating the expression or/and activity of the gene and determining the amount of influenza virus produced by the cell or/and the organism, and
(4) selecting a gene the expression or/and activity of which is positively or negatively correlated with reduction of the amount of the influenza virus produced by the cell or/and the organism.

37. The method of claim 36, wherein modulating the expression of a gene is downregulation or upregulation.

38. The method of claim 36, wherein modulation of the activity of a gene is decreasing or increasing of the activity.

39. The screening method of claim 25, wherein the influenza is selected from influenza A viruses, more preferably from strain Puerto Rico/8/34 and Avian Influenza virus isolates such as H5N1.

40. The screening method of claim 25, wherein the cell is a mammalian cell.

41. A method for the prevention, alleviation or/and prevention of an influenza virus infection comprising administering a kinase modulator or/and a kinase binding protein modulator.

42. A method for the prevention, alleviation or/and prevention of an influenza virus infection comprising administering an inhibitor of influenza virus replication capable of inhibiting the expression of a gene selected from Table 1b and 4, or/and of inhibiting a gene product thereof.

43. A method for screening for compounds or/and targets suitable for the prevention, alleviation or/and treatment of an influenza virus infection comprising using a nucleic acid comprising a gene sequence or/and a nucleotide sequence selected from Table 1a, 1b, 2, or/and 4 and fragments thereof in said screening method.

44. A method of claim 43, wherein a combination of at least two nucleic acids is used.

45. A method of claim 43, wherein the nucleic acid or the combination is selected from Table 1b and 4.

46. A method of claim 45, wherein the combination of nucleic acids inhibits expression or/and activity of one gene.
